(19) United States
(12) Patent Application Publication
(10) Pub. No.: US 2024/0124406 A1
(43) Pub. Date: Apr. 18, 2024

(54) INHIBITORS OF TRANSCRIPTIONAL ENHANCED ASSOCIATE DOMAIN (TEAD) AND USES THEREOF

(71) Applicant: Dana-Farber Cancer Institute, Inc., Boston, MA (US)

(72) Inventors: Nathanael S. Gray, Boston, MA (US); Tinghu Zhang, Brookline, MA (US); Nicholas Paul Kwiatkowski, Brookline, MA (US); Mengyang Fan, Allston, MA (US); Jianwei Che, Sharon, MA (US); Wenchao Lu, Brookline, MA (US)

(21) Appl. No.: 18/000,049

(22) PCT Filed: Jun. 2, 2021
(86) PCT No.: PCT/US2021/035343

§ 371 Date: Nov. 28, 2022

Related U.S. Application Data
(60) Provisional application No. 63/183,094, filed on May 3, 2021.
(60) Provisional application No. 63/034,248, filed on Jun. 3, 2020.

(51) Int. Cl. C07D 257/04; A61P 35/00; C07D 213/64; C07D 233/64; C07D 237/08; C07D 239/26; C07D 239/34; C07D 271/10; C07D 277/28; C07D 401/04; C07D 403/04; C07D 405/12; C07D 513/04

(52) U.S. Cl. CPC C07D 257/04; A61P 35/00; C07D 213/64; C07D 233/64; C07D 237/08; C07D 239/26; C07D 239/34; C07D 271/10; C07D 277/28; C07D 401/04; C07D 403/04; C07D 405/12; C07D 513/04

(57) ABSTRACT

Provided herein are compounds of Formula (I″), Formula (I′), Formula (I), Formula (II′), and Formula (II), and pharmaceutically acceptable salts, solvates, hydrates, polymorphs, co-crystals, tautomers, stereoisomers, isotopically labeled derivatives, and prodrugs thereof. Also provided are methods, uses, and kits involving the disclosed compounds and pharmaceutical compositions thereof for treating and/or preventing diseases (e.g., proliferative diseases (e.g., cancers), inflammatory diseases (e.g., fibrosis), autoimmune diseases (e.g., sclerosis)) in a subject. Provided are methods of inhibiting the activity of a transcription factor (e.g., TEAD, such as TEAD1, TEAD2, TEAD3, TEAD4) and/or inhibiting the transcription of a gene (e.g., a gene controlled or regulated by a transcription factor (e.g., TEAD)) in a subject.

### RELATED APPLICATIONS

The present application claims priority under 35 U.S.C. § 119(e) to U.S. provisional application, U.S. Ser. No. 63/034,248, filed Jun. 3, 2020, and U.S. provisional application, U.S. Ser. No. 63/183,094, filed May 3, 2021, each of which is incorporated herein by reference.

### BACKGROUND OF THE INVENTION

The Hippo signaling pathway plays key roles in organ size control and tumor suppression. YAP and transcriptional enhanced associate domain (TEAD) are major effectors of the Hippo signaling pathway. Signal transduction involves a core kinase cascade, leading to YAP (Yes 1-associated protein)/TAZ (transcriptional co-activator with PDZ-binding motif) phosphorylation. Physiological or pathological inactivation leads to dephosphorylation and nuclear accumulation. Nuclear YAP/TAZ binds to TEADs to mediate target gene expression. The TEAD-YAP complex regulates organ development and amplification of oncogenic factors in many cancers (e.g., sarcoma, lung cancer, thyroid cancer, skin cancer, ovarian cancer, colorectal cancer, prostate cancer, pancreatic cancer, esophageal cancer, liver cancer, breast cancer). Several genes in the Hippo signaling pathway have been identified as tumor suppressors, and mutations in these genes have been associated with different human cancers. Additionally, elevated YAP levels have been associated with certain human cancers.

The attachment of the fatty acid palmitate to cysteine residues regulates protein trafficking, membrane localization, and signaling activities. TEAD transcription factors have been found to possess intrinsic palmitoylating enzyme-like activity and undergo autopalmitoylation. TEAD transcription factors serve as canonical partners for the Hippo pathway effector YAP, which has been associated with resistance to targeted therapy in several contexts, including resistance to epidermal growth factor receptor (EGFR) tyrosine kinase inhibitors (TKI's) in EGFR-mutant NSCLC (Chaib, I. et al. “Co-activation of STAT3 and YES-Associated Protein 1 (YAP1) Pathway in EGFR-Mutant NSCLC” J. Natl. Cancer Inst. 2017, 109, 1-12; Hsu et al. “YAP promotes erlotinib resistance in human non-small cell lung cancer cells” Oncotarget 5. 2016). EGFR tyrosine kinase inhibitors (TKI's) are the standard of care for patients with advanced EGFR mutant non-small cell lung cancer (NSCLC) (Mok, T. S., et al. “Gefitinib or Carboplatin-Paclitaxel in Pulmonary Adenocarcinoma.” N. Engl. J. Med. 2009 361, 947-957; Rosell, R., et al. “Erlotinib versus standard chemotherapy as first-line treatment for European patients with advanced EGFR mutation-positive non-small-cell lung cancer (EURTAC): a multicentre, open-label, randomised phase 3 trial.” Lancet Oncol. 2012 13, 239-246; Soria, J.-C., et al. “Osimertinib in Untreated EGFR-Mutated Advanced Non-Small-Cell Lung Cancer” N. Engl. J. Med. 2018, 378, 113-125). However, within months to a few years, acquired resistance mechanisms inevitably develop, limiting the clinical efficacy of EGFR inhibitor treatment (Cortot, A. B., and Jänne, P. A. “Molecular mechanisms of resistance in epidermal growth factor receptor-mutant lung adenocarcinomas.” Eur. Respir. Rev. 2014 23, 356-366). In most cases, drug resistance to EGFR-targeted therapy arises after a dramatic initial clinical response followed by an extended time in a non-proliferative minimal residual disease (MRD), or dormant, state, with subsequent gradual emergence and growth of a drug resistant tumor. Prior preclinical studies suggest that following EGFR TKI treatment, EGFR mutant tumor cells can enter a drug tolerant state, reminiscent of dormancy in patients, allowing cells to evade apoptosis and survive under drug treatment (Hata, A. N., et al. “Tumor cells can follow distinct evolutionary paths to become resistant to epidermal growth factor receptor inhibition.” Nat. Med. 2016 22, 262-269; Sharma, S. et al. “A Chromatin-Mediated Reversible Drug-Tolerant State in Cancer Cell Subpopulations.” Cell 2010, 141, 69-80). Over time, the drug tolerant cells can acquire drug resistance through either mutational or non-mutational mechanisms (Hata et al., 2016). While it has been proposed that the establishment of this state is largely stochastic and dictated mostly by epigenetic mechanisms (Guler, G. D., et al. “Repression of Stress-Induced LINE-1 Expression Protects Cancer Cell Subpopulations from Lethal Drug Exposure.” Cancer Cell 2017, 32, 221-237.e13; Sharma et al., 2010), the mechanistic bases of how cancer cells evade the initial apoptosis in response to drug treatment—the absolute requirement to enter the drug tolerant state—or maintain tolerance in the presence of drug treatment are poorly understood. Previous work demonstrated that despite sustained EGFR inhibition following EGFR TKI treatment of EGFR-mutant cells, reactivation of ERK 1/2 occurs within just a few days (Ercan, D., et al. “Reactivation of ERK signaling causes resistance to EGFR kinase inhibitors.” Cancer Discov. 2012, 2, 934-947; Tricker, E. M., et al. “Combined EGFR/MEK Inhibition Prevents the Emergence of Resistance in EGFR-Mutant Lung Cancer.” Cancer Discov. 2015, 5, 960-971). Concomitant inhibition of MEK effectively prevents reactivation of ERK 1/2, results in a greater initial apoptotic response, and leads to a more durable tumor control in vitro and in vivo than single agent EGFR inhibition (Ercan et al., 2012; Tricker et al., 2015). The EGFR (osimertinib) and MEK (selumetinib) inhibitor combination has been studied in patients resistant to prior EGFR TKI's and is also under evaluation as an initial therapy for advanced EGFR-mutant NSCLC in a phase II clinical trial (NCT03392246; Ramalingam, S., et al. “Osimertinib plus selumetinib for patients with EGFR-mutant (EGFRm) NSCLC following disease progression on an EGFR-TKI: results from the Phase Ib TATTON study. In AACR” Annual Meeting 2019, (Atlanta (GA): AACR). However, even with this combination, acquired resistance still develops either through bypass of EGFR/MEK inhibition, or by unknown mechanisms that do not involve reactivation of EGFR downstream signaling (Tricker et al., 2015). Due to the key regulatory functions of transcription factors TEAD, YAP, and the TEAD-YAP complex in development, cell growth and proliferation, tissue homeostasis, and regeneration, it is important to develop modulators of the activity of these transcription factors (e.g., TEAD, YAP), including selective modulators (e.g., selective inhibitors), for use as research tools as well as therapeutic agents in the treatment of various diseases associated with these transcription factors. It is also important to develop therapeutic agents for treating proliferative diseases associated with these transcription factors (e.g., TEAD, YAP) that may be resistant to certain anti-proliferative agents (e.g., cancers resistant to inhibitors of EGFR and/or MEK) via combination therapies using modulators of the transcription factors TEAD, YAP, EGFR, and/or MEK.

### SUMMARY OF THE INVENTION

This disclosure is based in part on the discovery that eradicating tumor dormancy that develops following oncogene-targeted therapy, including after epidermal growth factor receptor (EGFR) tyrosine kinase inhibitor (TKI) treatment of a cancer, for example, lung cancer (e.g., EGFR-mutant non-small cell lung cancer (NSCLC)), is an attractive therapeutic strategy. However, the mechanisms governing the establishment of tumor dormancy are poorly understood. It was recently observed that the blockade of ERK1/2 reactivation following EGFR TKI treatment by combined EGFR/MEK inhibition uncovers cells that survive by entering a senescence-like dormant state, characterized by extensive epigenetic remodeling and high YAP/TEAD activity. YAP/TEAD trigger an epithelial-to-mesenchymal transition (EMT) program and engage the EMT transcription factor SLUG to directly repress pro-apoptotic BMF, limiting drug-induced apoptosis. Pharmacological co-inhibition of YAP or TEAD, or genetic deletion of YAP1, all deplete dormant cells by enhancing EGFR/MEK inhibitor-induced apoptosis. Thus, YAP activation can promote the survival of EGFR-mutant NSCLC cells in the chronic absence of EGFR signaling. Eradicating this surviving cell population, for example, by inhibiting TEAD and/or YAP, enhances the efficacy of targeted therapies which could ultimately lead to prolonged treatment responses in cancer patients.

Described herein are compounds of Formula (I), Formula (I′), Formula (I″), Formula (II′), and Formula (II), and pharmaceutically acceptable salts, co-crystals, tautomers, stereoisomers, solvates, hydrates, polymorphs, isotopically enriched derivatives, and prodrugs thereof, and mixtures thereof. In certain embodiments, the compounds of Formula (I), Formula (I′), Formula (I″), and Formula (II) inhibit the activity of the transcription factor TEAD.

The compounds of Formula (I), Formula (I′), Formula (I″), Formula (II′), and Formula (II), and pharmaceutically acceptable salts, co-crystals, tautomers, stereoisomers, solvates, hydrates, polymorphs, isotopically enriched derivatives, prodrugs, and compositions thereof, may inhibit the activity of a transcription factor (e.g., TEAD), for example, in vitro or in vivo, such as in a biological sample or in a subject. In certain embodiments, the transcription factor is a transcriptional enhanced associate domain (TEAD). In certain embodiments, the compound of Formula (I), Formula (I′), Formula (I″), Formula (II′), or Formula (II) is selective for a specific TEAD (e.g., TEAD1, TEAD2, TEAD3, TEAD4) compared to other TEADs. Described herein are methods of using the disclosed compounds, and pharmaceutically acceptable salts, co-crystals, tautomers, stereoisomers, solvates, hydrates, polymorphs, isotopically enriched derivatives, prodrugs, and compositions thereof, to study the inhibition of a transcription factor (e.g., TEAD, such as TEAD1, TEAD2, TEAD3, TEAD4). The disclosed compounds described herein may also be used as therapeutics for the prevention and/or treatment of diseases associated with the overexpression, increased activity, and/or aberrant (e.g., increased, unregulated, or unwanted) activity of a transcription factor (e.g., TEAD, such as TEAD1, TEAD2, TEAD3, TEAD4). The compounds described herein may be useful in treating and/or preventing a disease or condition, e.g., in treating and/or preventing a disease (e.g., proliferative disease (e.g., cancer, benign neoplasm), inflammatory disease (e.g., fibrosis), autoimmune disease (e.g., sclerosis)), in a subject in need thereof. For treating and/or preventing a disease described herein (e.g., proliferative disease (e.g., cancer, benign neoplasm, for example, a cancer resistant to a modulator of another transcription factor (e.g., YAP, EGFR, MEK)), inflammatory disease (e.g., fibrosis), autoimmune disease (e.g., sclerosis)), the compounds described herein may be optionally administered in combination with an additional pharmaceutical agent, for example, modulators of other transcription factors (e.g., YAP, EGFR, MEK), for inhibiting a transcription factor (e.g., TEAD, such as TEAD1, TEAD2, TEAD3, TEAD4) in a subject and/or biological sample, and for inhibiting the transcription of a gene (e.g., a gene controlled or regulated by a transcription factor (e.g., TEAD, such as TEAD1, TEAD2, TEAD3, TEAD4)) in a subject and/or biological sample. Also provided are uses, pharmaceutical compositions, and kits including a compound described herein.

In one aspect, the present disclosure provides compounds of Formula (I″):

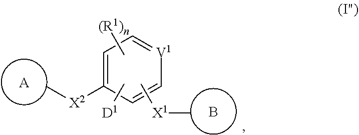

and pharmaceutically acceptable salts, co-crystals, tautomers, stereoisomers, solvates, hydrates, polymorphs, isotopically enriched derivatives, and prodrugs thereof, wherein R¹, V¹, X¹, Ring A, Ring B, and D¹ are as defined herein. D¹ is a warhead which in some embodiments binds a TEAD (e.g., TEAD1, TEAD2, TEAD3, TEAD4). In certain embodiments, the compound non-covalently binds to a TEAD, e.g., TEAD1, TEAD2, TEAD3, TEAD4. In certain embodiments, the compound covalently binds to a TEAD, e.g., TEAD1, TEAD2, TEAD3, TEAD4.

In one aspect, the present disclosure provides compounds of Formula (I):

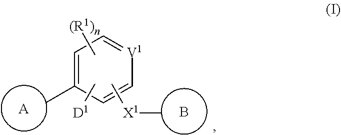

and pharmaceutically acceptable salts, co-crystals, tautomers, stereoisomers, solvates, hydrates, polymorphs, isotopically enriched derivatives, and prodrugs thereof, wherein R¹, V¹, X¹, Ring A, Ring B, and D¹ are as defined herein. D¹ is a warhead which in some embodiments binds a TEAD (e.g., TEAD1, TEAD2, TEAD3, TEAD4). In certain embodiments, the compound non-covalently binds to a TEAD, e.g., TEAD1, TEAD2, TEAD3, TEAD4. In certain embodiments, the compound covalently binds to a TEAD, e.g., TEAD1, TEAD2, TEAD3, TEAD4.

In one aspect, the present disclosure provides compounds of Formula (I′):

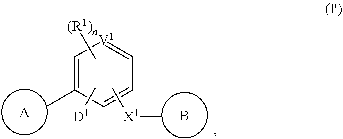

and pharmaceutically acceptable salts, co-crystals, tautomers, stereoisomers, solvates, hydrates, polymorphs, isotopically enriched derivatives, and prodrugs thereof, wherein R¹, V¹, X¹, Ring A, Ring B, and D¹ are as defined herein. D¹ is a warhead which in some embodiments binds a TEAD (e.g., TEAD1, TEAD2, TEAD3, TEAD4). In certain embodiments, the compound non-covalently binds to a TEAD, e.g., TEAD1, TEAD2, TEAD3, TEAD4. In certain embodiments, the compound covalently binds to a TEAD, e.g., TEAD1, TEAD2, TEAD3, TEAD4.

Exemplary compounds of Formula (I″), include, but are not limited to:

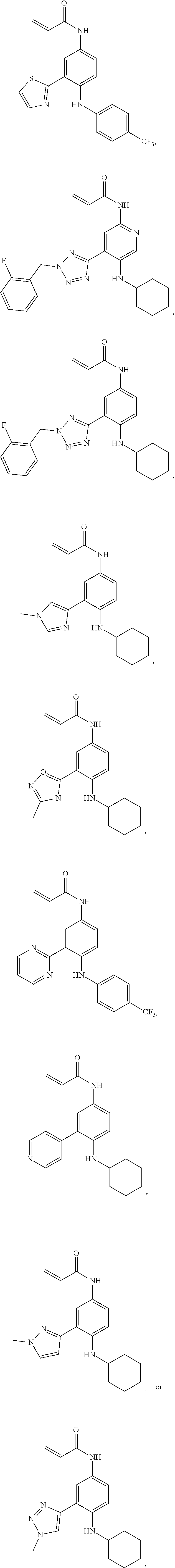

and pharmaceutically acceptable salts, co-crystals, tautomers, stereoisomers, solvates, hydrates, polymorphs, isotopically enriched derivatives, and prodrugs thereof.

In one aspect, the present disclosure provides compounds of Formula (II′):

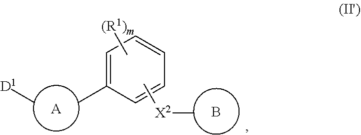

and pharmaceutically acceptable salts, co-crystals, tautomers, stereoisomers, solvates, hydrates, polymorphs, isotopically enriched derivatives, and prodrugs thereof, wherein R², X², Ring A, Ring B, and D¹ are as defined herein. D¹ is a warhead which in some embodiments binds a TEAD (e.g., TEAD1, TEAD2, TEAD3, TEAD4). In certain embodiments, the warhead non-covalently binds to a TEAD, e.g., TEAD1, TEAD2, TEAD3, TEAD4. In certain embodiments, the warhead covalently binds to a TEAD, e.g., TEAD1, TEAD2, TEAD3, TEAD4.

Exemplary compounds of Formula (II′) include, but are not limited to:

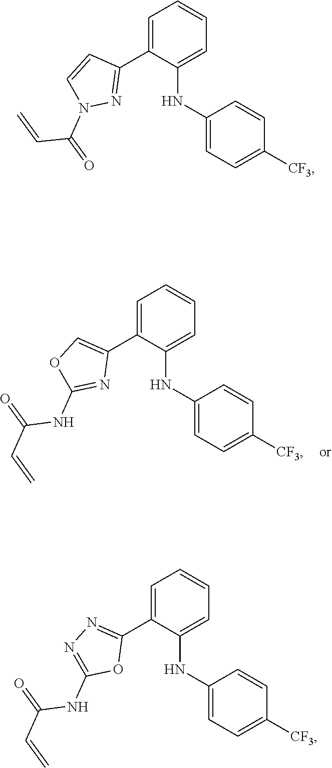 [SMILES: C=CC(=O)n1ccc(-c2ccccc2Nc2ccc(C(F)(F)F)cc2)n1 ; C=CC(=O)Nc1nc(-c2ccccc2Nc2ccc(C(F)(F)F)cc2)co1 ; C=CC(=O)Nc1nnc(-c2ccccc2Nc2ccc(C(F)(F)F)cc2)o1]

and pharmaceutically acceptable salts, co-crystals, tautomers, stereoisomers, solvates, hydrates, polymorphs, isotopically enriched derivatives, and prodrugs thereof.

In another aspect, the present disclosure provides pharmaceutical compositions including a compound described herein, and optionally a pharmaceutically acceptable excipient. In certain embodiments, the pharmaceutical compositions described herein include a therapeutically or prophylactically effective amount of a compound described herein. The pharmaceutical composition may be useful for treating and/or preventing a disease (e.g., a proliferative disease, inflammatory disease, autoimmune disease) in a subject in need thereof, inhibiting the activity of a transcription factor (e.g., TEAD, such as TEAD1, TEAD2, TEAD3, TEAD4), or inhibiting the transcription of a gene (e.g., a gene controlled or regulated by a transcription factor (e.g., TEAD, such as TEAD1, TEAD2, TEAD3, TEAD4) in a subject, and/or biological sample (e.g., tissue, cell). In certain embodiments, the proliferative disease is cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma). In certain embodiments, the cancer is a sarcoma (e.g., Kaposi's sarcoma). In certain embodiments, the cancer is a carcinoma. In certain embodiments, the cancer is lung cancer (e.g., non-small cell lung cancer, mesothelioma). In certain embodiments, the cancer has a mutation in a gene of the Hippo signaling pathway. In certain embodiments, the cancer has a mutation in EGFR. In certain embodiments, the cancer has a mutation in MEK. In certain embodiments, the cancer is an EGFR-mutant non-small cell lung cancer. In certain embodiments, the cancer is resistant to certain anti-proliferative agents (e.g., cancers resistant to inhibitors of EGFR and/or MEK). In certain embodiments, the cancer is resistant to tyrosine kinase inhibitors (TKI's). In certain embodiments, the cancer is resistant to inhibitors of EGFR (e.g., osimertinib) and/or inhibitors of MEK (e.g., trametinib). In certain embodiments, the disease is an inflammatory disease (e.g., fibrosis). In certain embodiments, the disease is an autoimmune disease (e.g., sclerosis).

In another aspect, described herein are methods for treating and/or preventing a disease (e.g., a proliferative disease, inflammatory disease, autoimmune disease) using a compound described herein, which may be optionally administered in combination with an additional pharmaceutical agent, for example, modulators of other transcription factors (e.g., YAP, EGFR, MEK). Exemplary proliferative diseases which may be treated include diseases associated with the overexpression or the increased activity of a TEAD, e.g., a proliferative disease, such as cancer, or a cancer resistant to a modulator (e.g., inhibitor) of another transcription factor (e.g., YAP, EGFR, MEK). In certain embodiments, the cancer is selected from the group consisting of sarcoma, lung cancer, thyroid cancer, breast cancer, liver cancer, pancreatic cancer, gastric cancer, ovarian cancer, colon cancer, colorectal cancer, skin cancer, esophageal cancer; and carcinoma.

Another aspect relates to methods of inhibiting the activity of a transcription factor (e.g., TEAD, such as TEAD1, TEAD2, TEAD3, TEAD4) using a compound described herein in a biological sample (e.g., cell, tissue), which may be optionally administered or used in combination with an additional pharmaceutical agent, for example, modulators of other transcription factors (e.g., YAP, EGFR, MEK). In another aspect, described herein are methods of inhibiting the activity of a transcription factor (e.g., TEAD, such as TEAD1, TEAD2, TEAD3, TEAD4) using a compound described herein in a subject. In certain embodiments, the method involves the inhibition of TEAD (e.g., TEAD2). Another aspect relates to methods of inhibiting the transcription of a gene (e.g., a gene controlled or regulated by a transcription factor (e.g., TEAD, such as TEAD1, TEAD2, TEAD3, TEAD4))) using a compound described herein, which may be optionally administered in combination with an additional pharmaceutical agent, for example, modulators of other transcription factors (e.g., YAP, EGFR, MEK).

Described herein are methods for administering to a subject in need thereof an effective amount of a compound, or pharmaceutical composition thereof, as described herein, which may be optionally administered in combination with an additional pharmaceutical agent, for example, modulators of other transcription factors (e.g., YAP, EGFR, MEK). Also described are methods for contacting a biological sample (e.g., tissue, cell) with an effective amount of a compound, or pharmaceutical composition thereof, as described herein, which may be optionally administered in combination with an additional pharmaceutical agent, for example, modulators of other transcription factors (e.g., YAP, EGFR, MEK). In certain embodiments, a method described herein further includes administering to the subject an additional pharmaceutical agent. In certain embodiments, a method described herein further includes contacting the biological sample (e.g., tissue, cell) with an additional pharmaceutical agent (e.g., an anti-proliferative agent). In certain embodiments, the additional pharmaceutical agent is a modulator of another transcription factor (e.g., YAP, EGFR, MEK). In certain embodiments, the additional pharmaceutical agent is a transcription inhibitor (e.g., an inhibitor of EGFR and/or MEK). In certain embodiments, the additional pharmaceutical agent is a kinase inhibitor. In certain embodiments, the additional pharmaceutical agent is an agent for treating lung cancer (e.g., non-small cell lung cancer (NSCLC)).

In yet another aspect, the present disclosure provides compounds of Formula (I), Formula (I′), Formula (I″), Formula (II′), and Formula (II), and pharmaceutically acceptable salts, solvates, hydrates, polymorphs, co-crystals, tautomers, stereoisomers, isotopically labeled derivatives, prodrugs, and compositions thereof, which may be optionally administered in combination with an additional pharmaceutical agent for use in the treatment of a disease (e.g., a proliferative disease, inflammatory disease, autoimmune disease) in a subject. In certain embodiments, the additional pharmaceutical agent is a modulator of another transcription factor (e.g., YAP, EGFR, MEK). In yet another aspect, the present disclosure provides compounds of Formula (I), Formula (I′), Formula (I″), Formula (II′), and Formula (II), and pharmaceutically acceptable salts, solvates, hydrates, polymorphs, co-crystals, tautomers, stereoisomers, isotopically labeled derivatives, prodrugs, and compositions thereof, which may be optionally administered in combination with an additional pharmaceutical agent, for example, modulators of other transcription factors (e.g., YAP, EGFR, MEK), for use in inhibiting the activity of a transcription factor (e.g., TEAD, such as TEAD1, TEAD2, TEAD3, TEAD4)), or inhibiting the transcription of a gene (e.g., a gene controlled or regulated by a transcription factor (e.g., TEAD, such as TEAD1, TEAD2, TEAD3, TEAD4))) in a subject and/or biological sample (e.g., tissue, cell).

Another aspect of the present disclosure relates to kits comprising a container with a compound, or pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of one or more embodiments of the invention are set forth herein. Other features, objects, and advantages of the invention will be apparent from the Detailed Description, Examples, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“Hydrocarbon chain” refers to a substituted or unsubstituted divalent alkyl, alkenyl, or alkynyl group. A hydrocarbon chain includes at least one chain, each node (“carbon unit”) of which including at least one carbon atom, between the two radicals of the hydrocarbon chain. For example, hydrocarbon chain —C^{A}H(C^{B}H₂C^{C}H₃)— includes only one carbon unit C^{A}. The term “Cₓ hydrocarbon chain,” wherein x is a positive integer, refers to a hydrocarbon chain that includes x number of carbon unit(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

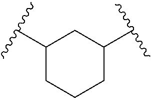 [SMILES: *C1CCCC(*)C1]

is a C₃ hydrocarbon chain. When a range of values is used, e.g., a C₁₋₆ hydrocarbon chain, the meaning of the range is as described herein. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of an unsubstituted and unsaturated hydrocarbon chain. In certain embodiments, the hydrocarbon chain is unsubstituted (e.g., —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance

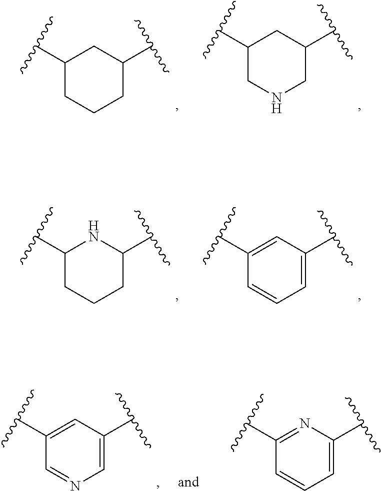 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments

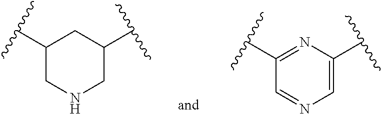 [SMILES: *C1CNCC(*)C1 ; *c1cncc(*)n1]

are not within the scope of the hydrocarbon chains described herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl group is an unsubstituted C₃₋₁₄ carbocyclyl. In certain embodiments, the carbocyclyl group is a substituted C₃₋₁₄ carbocyclyl. In certain embodiments, the carbocyclyl includes 0, 1, or 2 C═C double bonds in the carbocyclic ring system, as valency permits.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. In certain embodiments, the carbocyclyl group is an unsubstituted C₃₋₁₄ carbocyclyl. In certain embodiments, the carbocyclyl group is a substituted C₃₋₁₄ carbocyclyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from the group consisting of nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from the group consisting of nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from the group consisting of nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from the group consisting of nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from the group consisting of nitrogen, oxygen, and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from the group consisting of nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from the group consisting of nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from the group consisting of nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined herein. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

The term “optionally substituted” refers to substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkoxy” or “alkoxyl” refers to a radical of the formula: —O-alkyl.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′, 8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′, 4″-tris(4,5-dichlorophthalimidophenyl)methyl, 4,4′, 4″-tris(levulinoyloxyphenyl)methyl, 4,4′, 4″-tris(benzoyloxyphenyl)methyl, 3-(imidazol-1-yl)bis(4′, 4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in a heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{cc})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, amines, ammonia, alcohols, ether moieties, sulfur-containing moieties, thioether moieties, zinc halides, magnesium moieties, diazonium salts, and copper moieties.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), Formula (I′), Formula (I″), Formula (II′) or Formula (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I″), Formula (I′), Formula (I), Formula (II′), and Formula (II), which have cleavable groups and become by solvolysis or under physiological conditions the compounds of Formula (I″), Formula (I′), Formula (I), Formula (II′), and Formula (II), which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. A non-human animal may be a transgenic animal.

The terms “administer,” “administering,” or “administration” refer to implanting, absorbing, ingesting, injecting, inhaling, or otherwise introducing an inventive compound or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who does not have and did not have a disease but is at risk of developing the disease or is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

The term “inhibition,” “inhibiting,” “inhibit,” or “inhibitor” refer to the ability of a compound to reduce, slow, halt, or prevent activity of a particular biological process (e.g., a transcription factor) in a cell relative to vehicle.

An “effective amount” of a compound of Formula (I), Formula (I′), Formula (I″), Formula (II′), or Formula (II) refers to an amount sufficient to elicit the desired biological response, i.e., treating the condition, for example, inhibiting TEAD. As will be appreciated by those of ordinary skill in this art, the effective amount of a compound of Formula (I), Formula (I′), Formula (I″), Formula (II′), or Formula (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I), Formula (I′), Formula (I″), Formula (II′), or Formula (II) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a transcription factor (e.g., TEAD, such as TEAD1, TEAD2, TEAD3, TEAD4) and/or inhibiting the transcription factor (e.g., TEAD, such as TEAD1, TEAD2, TEAD3, TEAD4). In certain embodiments, a prophylactically effective amount is an amount sufficient for binding a transcription factor (e.g., TEAD, such as TEAD1, TEAD2, TEAD3, TEAD4) and/or inhibiting the transcription factor (e.g., TEAD, such as TEAD1, TEAD2, TEAD3, TEAD4). In certain embodiments, a prophylactically effective amount is an amount sufficient for preventing a disease and/or condition (e.g., proliferative disease, inflammatory disease, autoimmune disease). In certain embodiments, a prophylactically effective amount is an amount sufficient for binding a transcription factor (e.g., TEAD, such as TEAD1, TEAD2, TEAD3, TEAD4), and treating and/or preventing a disease and/or condition (e.g., proliferative disease, inflammatory disease, autoimmune disease). In certain embodiments, a prophylactically effective amount is an amount sufficient for binding a transcription factor (e.g., TEAD, such as TEAD1, TEAD2, TEAD3, TEAD4) and/or inhibiting the transcription factor (e.g., TEAD, such as TEAD1, TEAD2, TEAD3, TEAD4).

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes, such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, lymphoma, non-Hodgkin's lymphoma, Waldenström's macroglobulinemia, MYD88-mutated Waldenström's macroglobulinemia, activated B-cell diffuse large B-cell lymphoma, leukemia, sarcoma, lung cancer, thyroid cancer, breast cancer, liver cancer, pancreatic cancer, gastric cancer, ovarian cancer, colon cancer, colorectal cancer, skin cancer, esophageal cancer, and carcinoma. Exemplary proliferative diseases include cancers (i.e., “malignant neoplasms,”, sarcoma, lung cancer, thyroid cancer, breast cancer, liver cancer, pancreatic cancer, gastric cancer, ovarian cancer, colon cancer, colorectal cancer, skin cancer, esophageal cancer; carcinoma), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary original tumor to another organ or tissue and is typically identifiable by the presence of a “secondary tumor” or “secondary cell mass” of the tissue type of the primary original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), MYD88-mutated Waldenström's macroglobulinemia, activated B-cell (ABC) diffuse large B-cell lymphoma, mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatic (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation. In certain embodiments, the inflammatory disorder is fibrosis, and the fibrosis is idiopathic pulmonary fibrosis, liver cirrhosis, cystic fibrosis, systemic sclerosis, progressive kidney disease, or cardiovascular fibrosis.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy. In certain embodiments, the autoimmune disorder is sclerosis. In certain embodiments, the sclerosis is systemic sclerosis (scleroderma) or multiple sclerosis.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

A “transcription factor” is a type of protein that is involved in the process of transcribing DNA into RNA, and/or modulating the transcription of one or more genes. Transcription factors can work independently or with other proteins in a complex to either stimulate or repress transcription. Transcription factors contain at least one DNA-binding domain that give them the ability to bind to specific sequences of DNA. Other proteins such as coactivators, chromatin remodelers, histone acetyltransferases, histone deacetylases, kinases, and methylases are also essential to gene regulation, but lack DNA-binding domains, and therefore are not transcription factors. These exemplary human transcription factors include, but are not limited to, YAP, EGFR, MEK, TEAD (e.g., TEAD1, TEAD2, TEAD3, TEAD4), AC008770.3, AC023509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMP1, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK3, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLI1, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES3, HES4, HES5, HES6, HES7, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT3, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERF1, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, and ZZZ3.

The term “TEAD” refers to transcriptional enhanced associate domain (TEAD) transcription factors. TEADs are primary transcription factors for the Yes-associated protein (YAP)/PDZ-binding domain (TAZ) transcription coactivators of the Hippo signaling pathway. Examples of TEADs include, but are not limited to, TEAD1, TEAD2, TEAD3, and TEAD4. For TEAD2, exemplary NCBI sequences from GenBank are: NM_001256660.2 (Homo sapiens) and NM_001256659.2 (Homo sapiens). Exemplary genes controlled or regulated by TEADs include, but are not limited to, TGF, CYR61, WNT5A/B, DKK1, TGFB2, BMP4, AREG, EGFR, PD-L1, MYC, LATS2, amino acid transporters SLC38A1/SLC7A5, and glucose transporter GLUT3. TEADs bind to DNA sequences including, but not limited to, MCAT DNA sequences, and the 5′-GGAATG-3′ consensus sequence.

### BRIEF DESCRIPTION OF THE DRAWINGS

FIG. 1 shows an anti-palmitoylation assay using TEAD4 protein.

FIG. 2 shows the IC₅₀ values of some exemplary compounds of Formula (I″) and an anti-proliferation assay using NCI-H226 cells.

### DETAILED DESCRIPTION OF CERTAIN EMBODIMENTS OF THE INVENTION

Certain aspects of the present disclosure relate to the compounds described herein, which inhibit the activity of a transcription factor (e.g., TEAD, such as TEAD1, TEAD2, TEAD3, TEAD4). The compounds described herein may be useful in treating and/or preventing diseases (e.g., proliferative diseases (e.g., cancers), inflammatory diseases (e.g., fibrosis), autoimmune diseases (e.g., sclerosis), or diseases associated with the activity of a transcription factor (e.g., TEAD, such as TEAD1, TEAD2, TEAD3, TEAD4)) in a subject, inhibiting the activity of a transcription factor (e.g., TEAD, such as TEAD1, TEAD2, TEAD3, TEAD4), or inhibiting the transcription of a gene (e.g., a gene controlled or regulated by a transcription factor (e.g., TEAD, such as TEAD1, TEAD2, TEAD3, TEAD4)) in a subject or biological sample. In certain embodiments, a compound described herein is a compound of Formula (I), Formula (I′), Formula (I″), Formula (II′), or Formula (II), or a pharmaceutically acceptable salt, solvate, hydrate, polymorph, co-crystal, tautomer, stereoisomer, isotopically labeled derivative (e.g., deuterated form), prodrug, composition, or mixture thereof. In certain embodiments, a compound described herein is a compound of Formula (I), Formula (I′), Formula (I″), Formula (II′), or Formula (II), or a pharmaceutically acceptable salt thereof. In certain embodiments, a compound described herein is a compound of Formula (I), or a pharmaceutically acceptable salt thereof. In certain embodiments, a compound described herein is a compound of Formula (I′), or a pharmaceutically acceptable salt thereof. In certain embodiments, a compound described herein is a compound of Formula (I″), or a pharmaceutically acceptable salt thereof. In certain embodiments, a compound described herein is a compound of Formula (II′), or a pharmaceutically acceptable salt thereof. In certain embodiments, a compound described herein is a compound of Formula (II), or a pharmaceutically acceptable salt thereof.

### Compounds

In certain embodiments, a compound described herein is of Formula (I′):

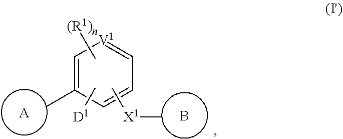

or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom, or a bicyclic heteroaryl;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —C(R^{c})₂O—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
V¹ is ═N— or ═C(R¹)—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
n is 0, 1, 2, or 3; and
D¹ is a warhead of any one of Formulae (i-1) to (i-43):

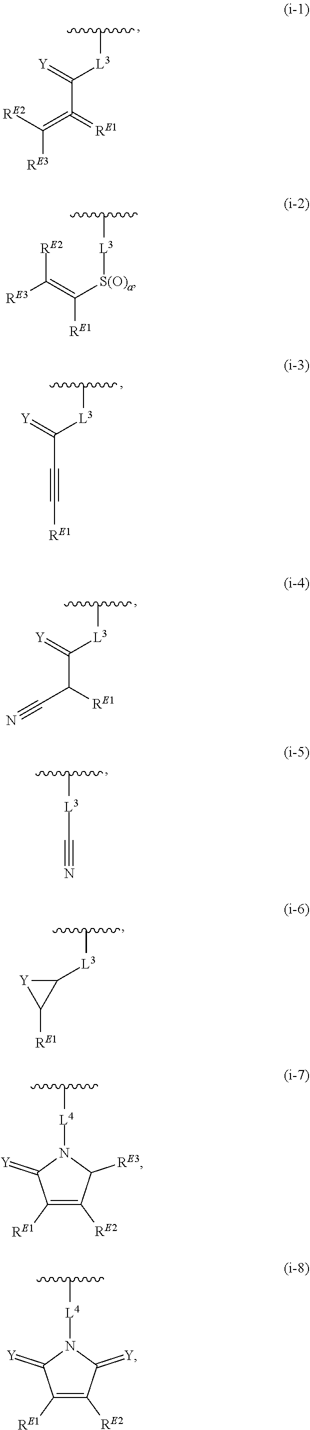

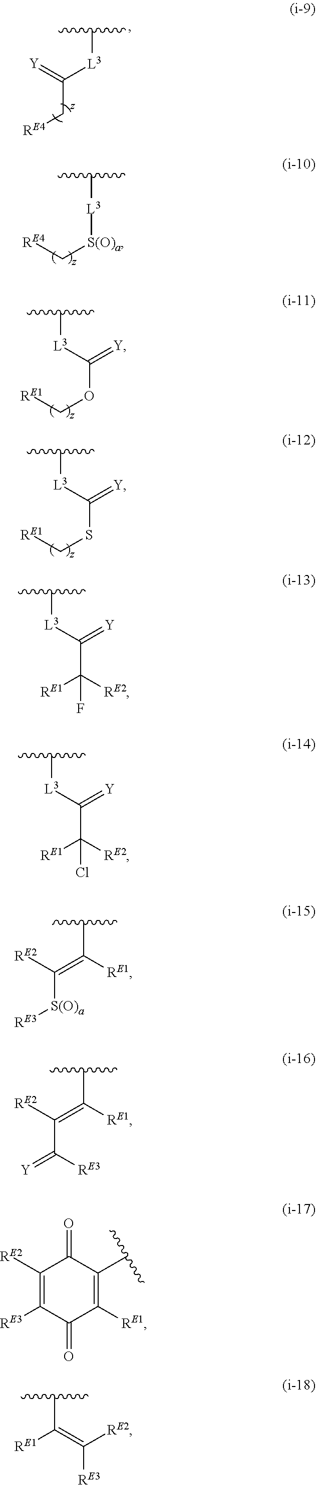

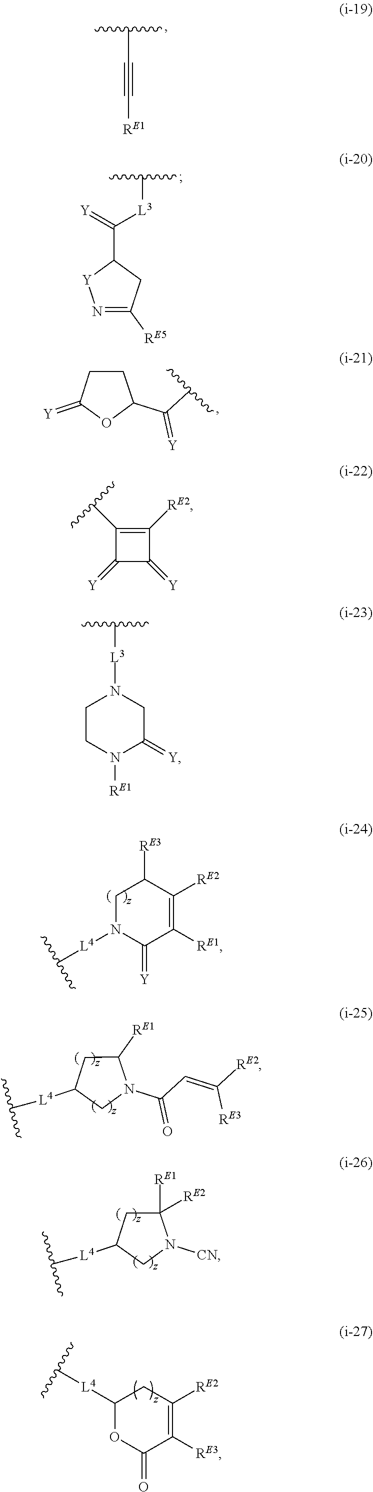

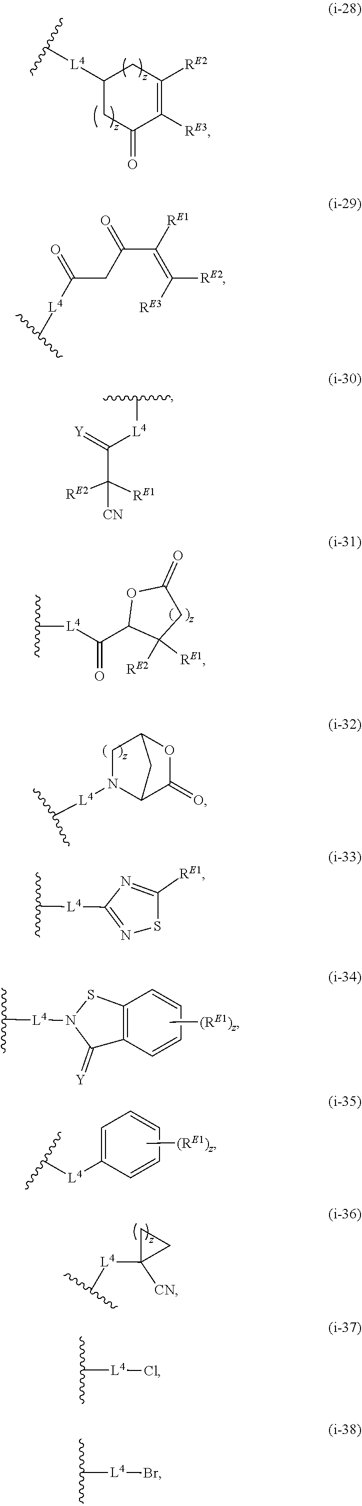

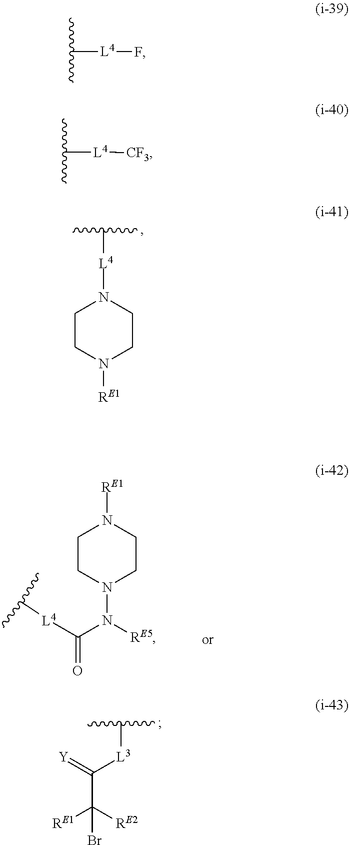

wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═))O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

In certain embodiments, the compound of Formula (I′) is of the Formula (I′-a):

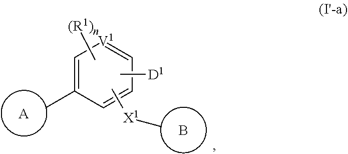

or a pharmaceutically acceptable salt, solvate, hydrate, polymorph, co-crystal, tautomer, stereoisomer, isotopically labeled compound, or prodrug thereof.

In certain embodiments, the compound of Formula (I′) is of the Formula (I′-b):

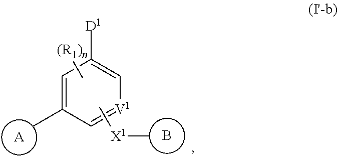

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-c):

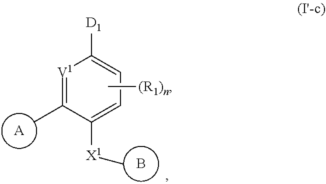

or a pharmaceutically acceptable salt, solvate, hydrate, polymorph, co-crystal, tautomer, stereoisomer, isotopically labeled compound, or prodrug thereof.

In certain embodiments, the compound of Formula (I′) is of the Formula (I′-d):

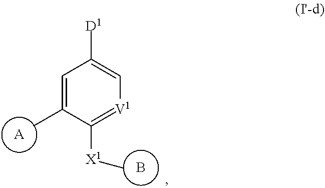

or a pharmaceutically acceptable salt, solvate, hydrate, polymorph, co-crystal, tautomer, stereoisomer, isotopically labeled compound, or prodrug thereof.

In certain embodiments, a compound described herein is of Formula (I″):

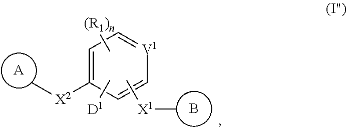

or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered optionally substituted heteroaryl, or optionally substituted heterocyclyl comprising at least one nitrogen atom, or a bicyclic heteroaryl;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —C(R^{c})₂O—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
X² is a bond, —C(R^{c})₂—; —CO—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
V¹ is ═N— or ═C(R^{d})—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl;
n is 0, 1, 2, or 3; and
D¹ is a warhead of any one of Formulae (i-1) to (i-43):

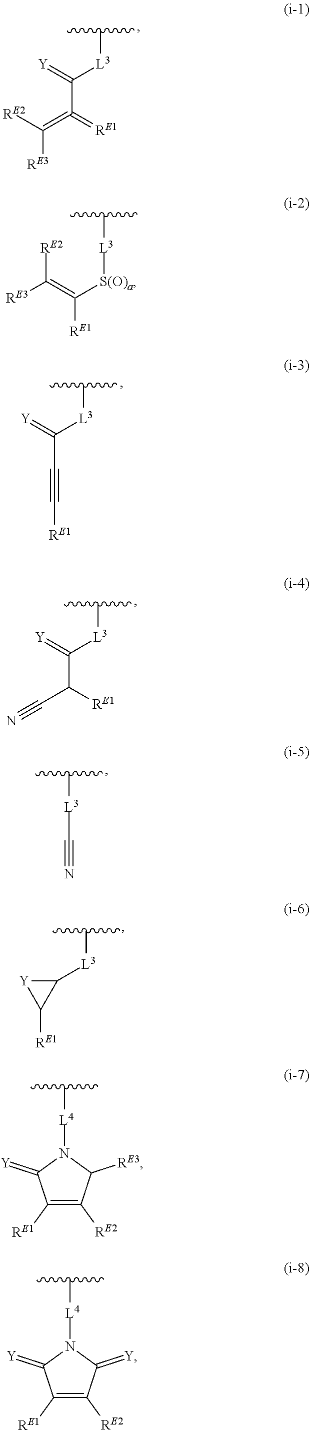

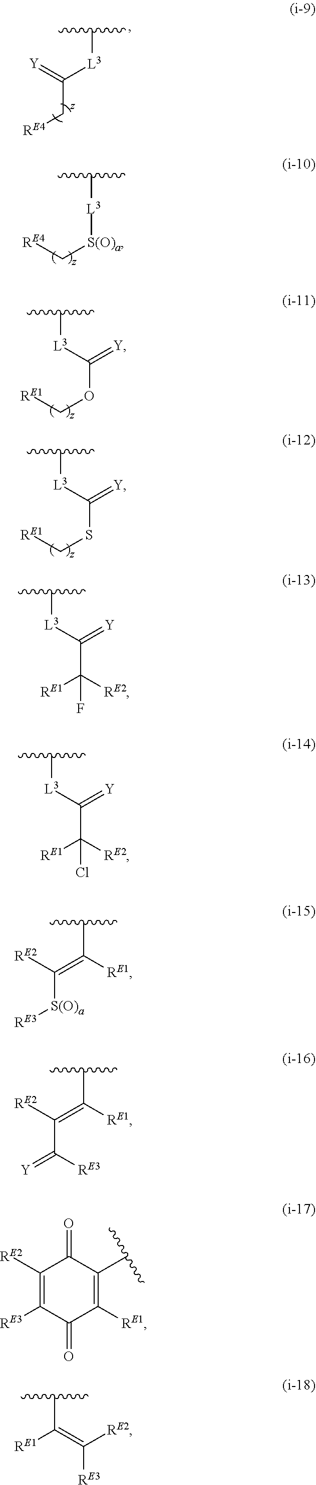

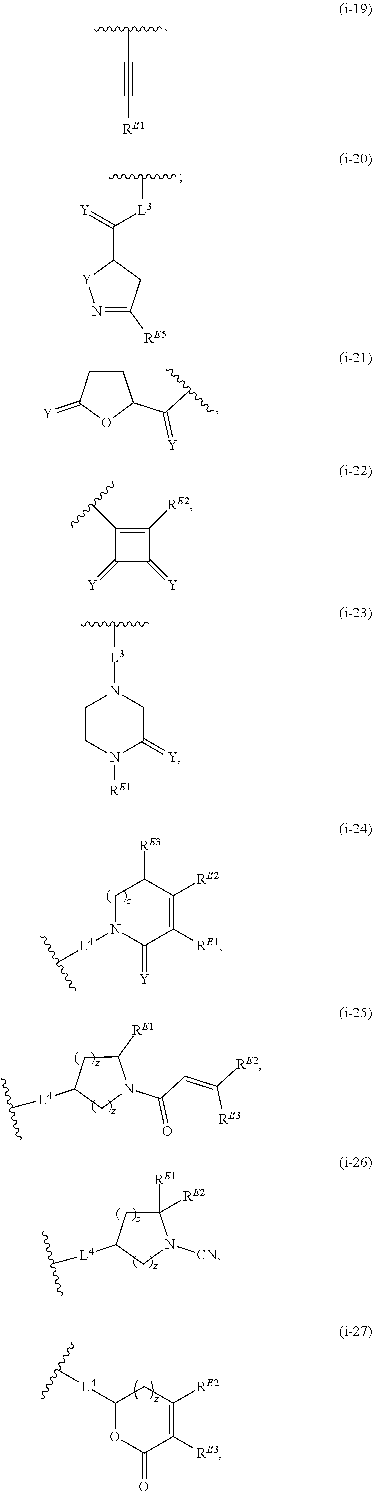

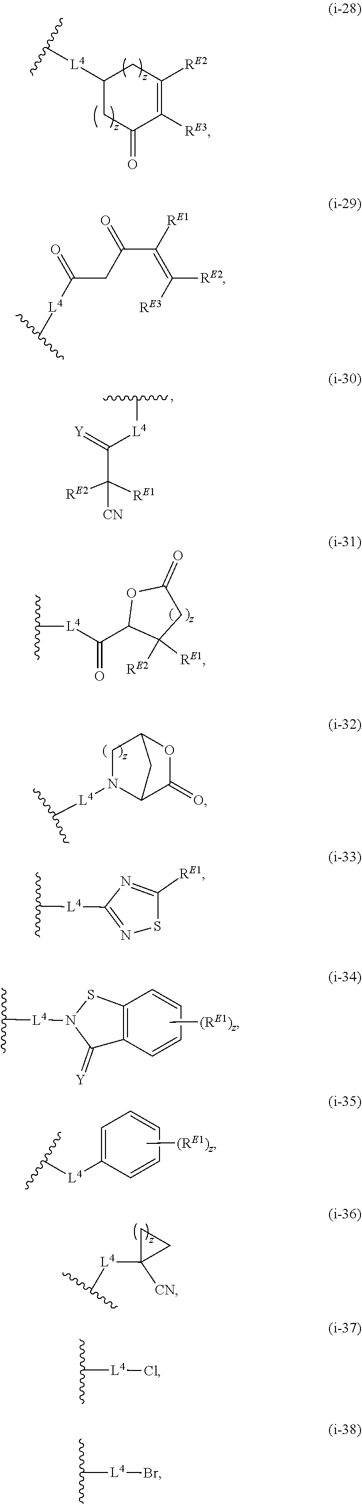

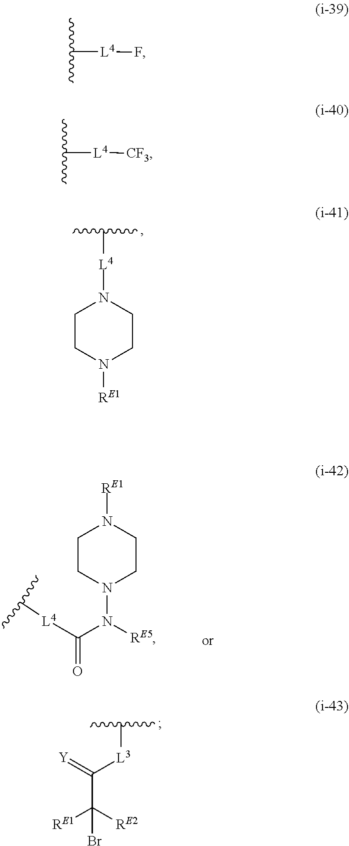

wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═))O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
   each instance of z is independently 0, 1, 2, 3, 4, 5, or 6.

In certain embodiments, X² is a bond. In certain embodiments, X² is —CH₂O—. In certain embodiments, X² is —OCH₂—. In certain embodiments, X² is —O—, —N(R^{c})—, —S—, —C(═O), —C(═O)O—, —C(═O)N(R^{c})—, —OC(═O)—, or —N(R^{c})C(═O)—. In certain embodiments, X² is —O—. In certain embodiments, X² is —S—. In certain embodiments, X² is —C(═O)—. In certain embodiments, X² is —C(═O)O—. In certain embodiments, X² is —C(═O)N(R^{c})—. In certain embodiments, X² is —OC(═O)—. In certain embodiments, X² is —N(R^{c})C(═O)—. In certain embodiments, X² is —N(R^{c})—. In certain embodiments, X² is —N(H)—. In certain embodiments, X is —N(Me)-.

In certain embodiments, a compound of Formula (I″) is described herein as a compound of Formula (I′-1):

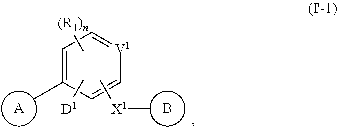

or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom, or a bicyclic heteroaryl;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —C(R^{c})₂O—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
V¹ is ═N— or ═C(R^{d})—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl;
n is 0, 1, 2, or 3; and
D¹ is a warhead of any one of Formulae (i-1) to (i-43):

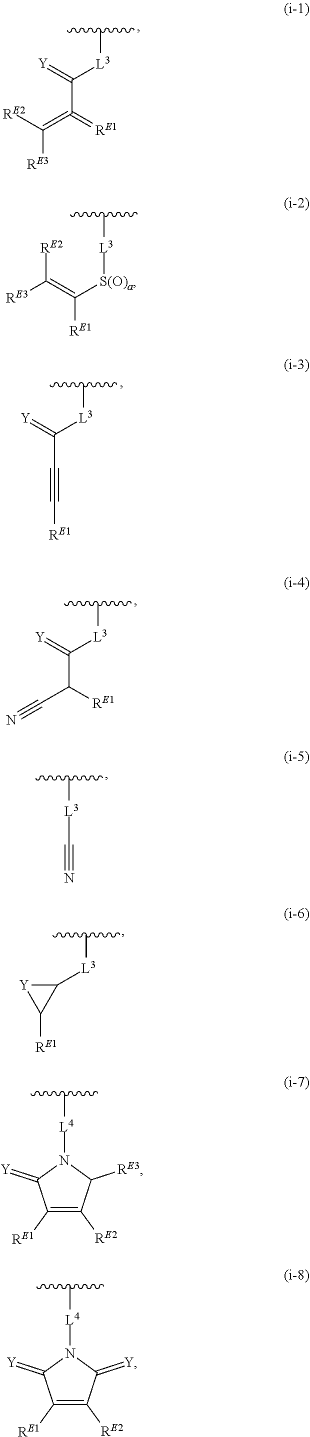

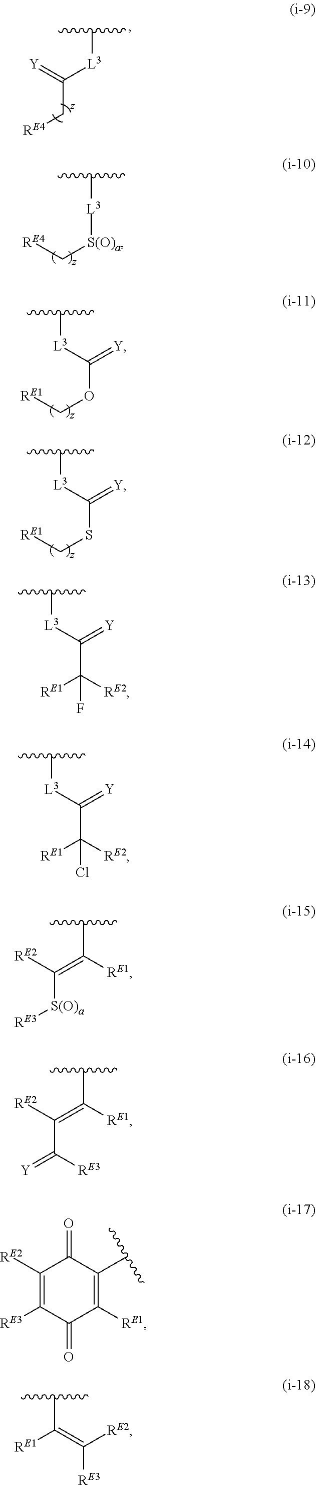

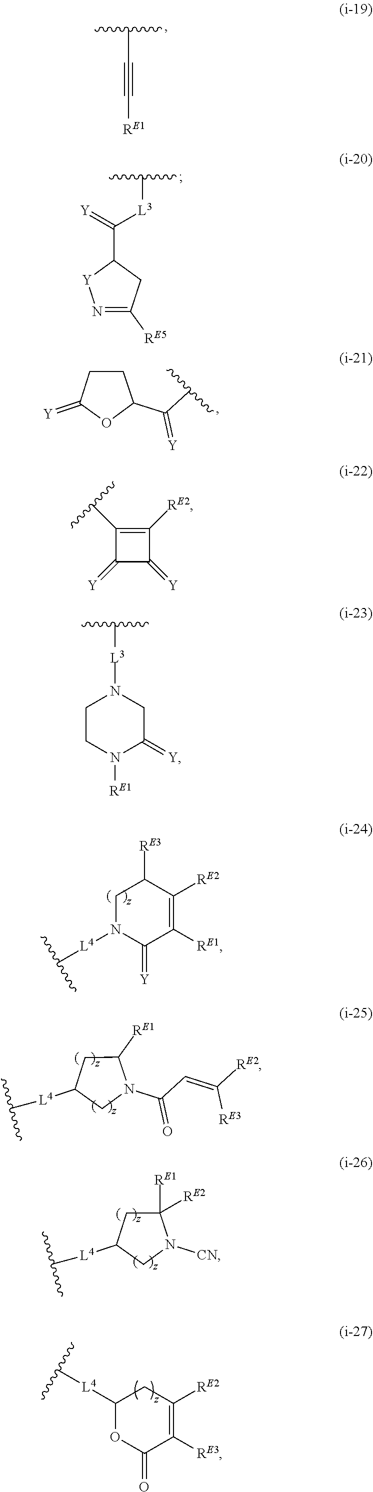

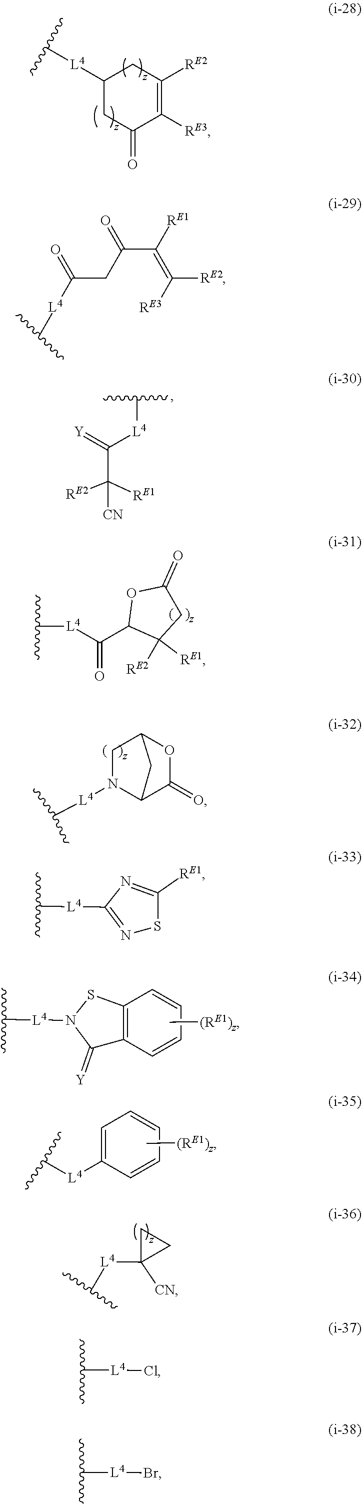

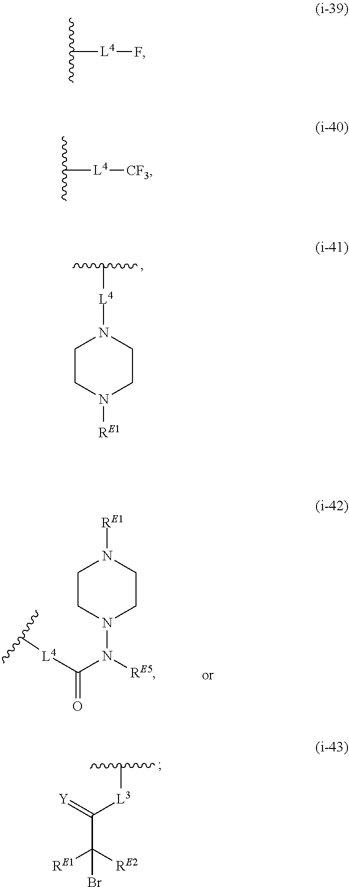

wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═))O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

In certain embodiments, a compound of Formula (I″) is described herein as a compound of Formula (I):

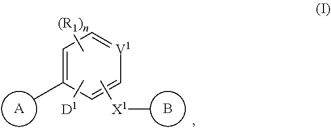

or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —O—, —N(R^{c})—, —S—, —C(═O)—, —C(═O)O—, —C(═O)N(R^{c})—, —OC(═O)—, or —N(R^{c})C(═O)—;
V¹ is ═N— or ═C(R^{d})—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl;
n is 0, 1, 2, or 3; and
D¹ is a warhead of any one of Formulae (i-1) to (i-43):

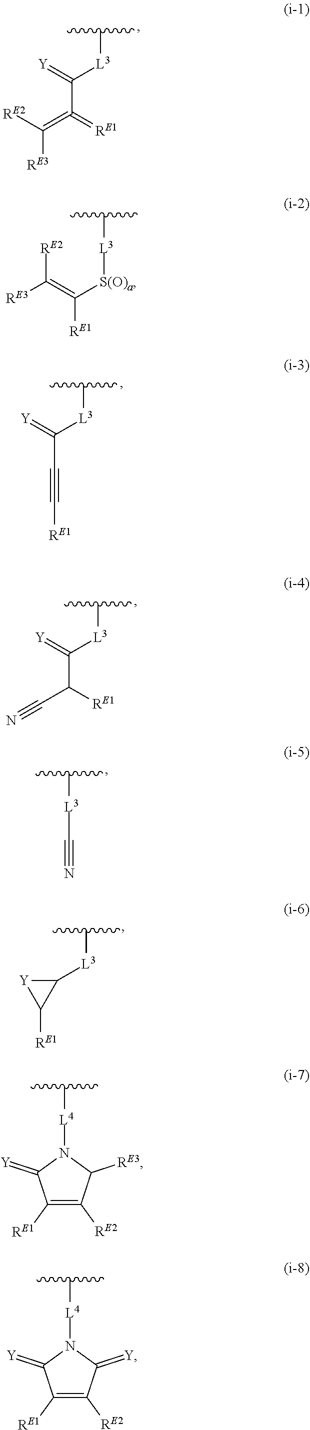

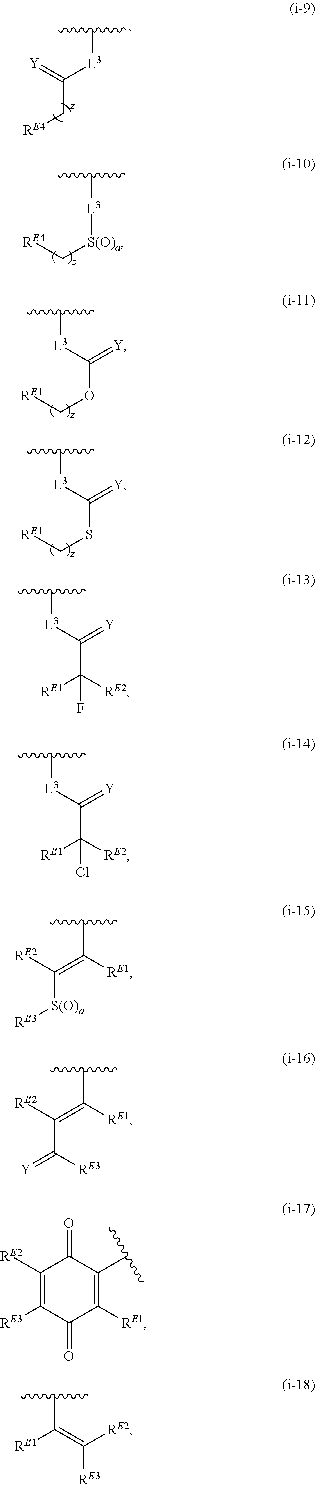

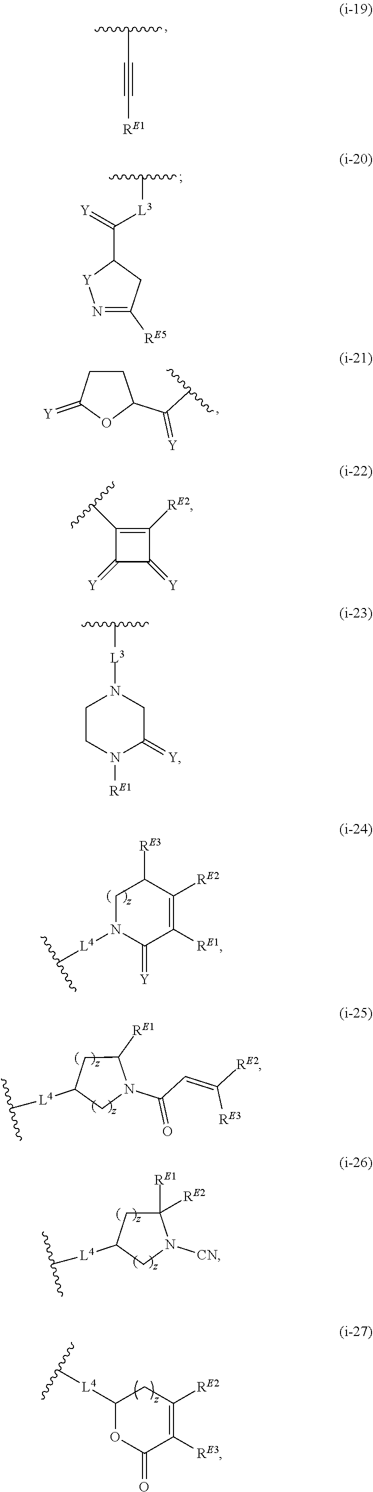

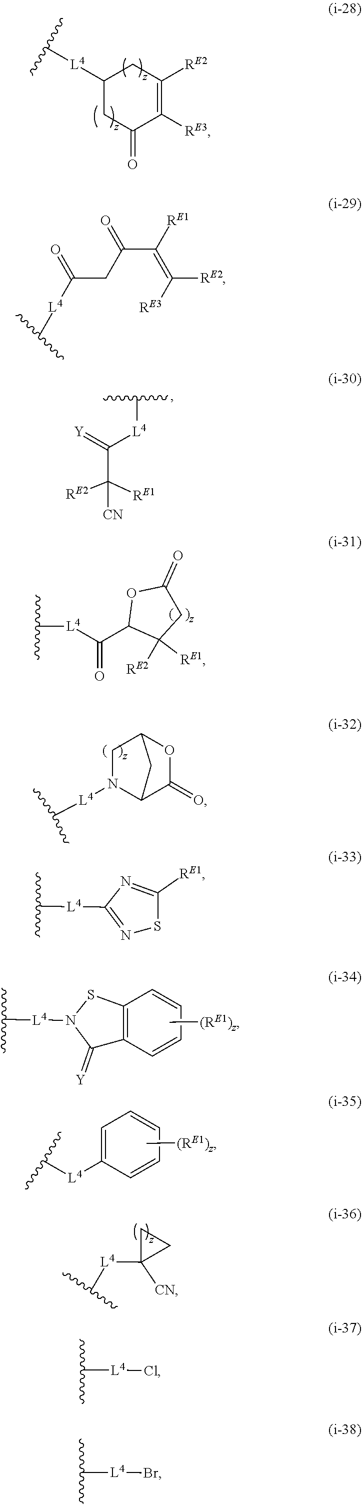

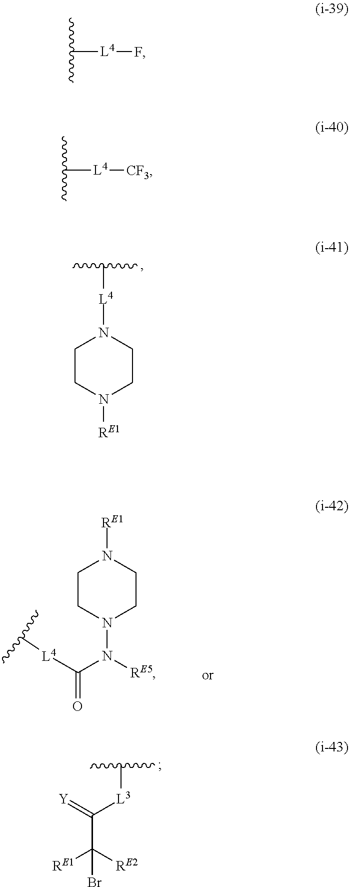

wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═))O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

In certain embodiments, the compound of Formula (I) is of the Formula (I-a):

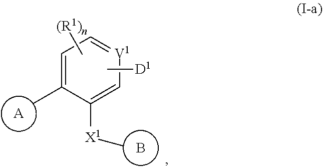

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-b):

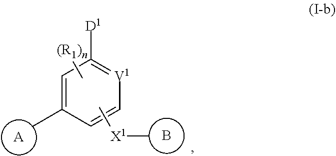

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-c):

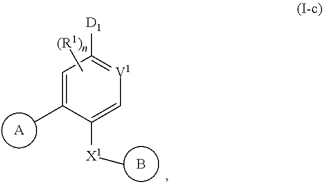

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-d):

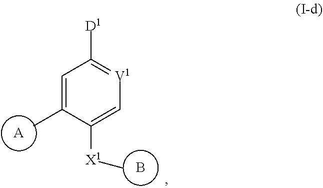

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-e):

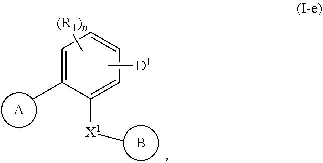

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-f):

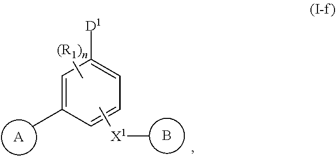

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-g):

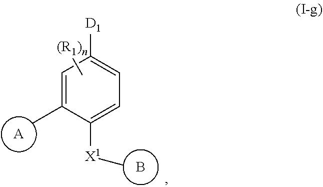

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-h):

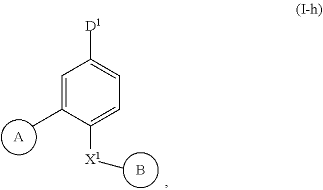

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-i):

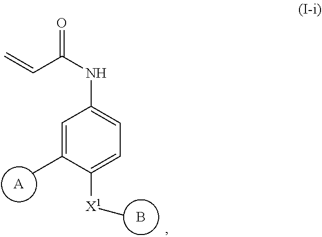

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-j):

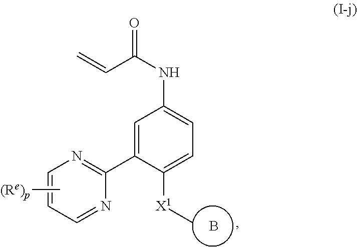

or a pharmaceutically acceptable salt, solvate, hydrate, polymorph, co-crystal, tautomer, stereoisomer, isotopically labeled compound, or prodrug thereof.

As generally defined herein, Formula (I″), Formula (I′), or Formula (I) contains Ring A. In certain embodiments, Ring A is an optionally substituted 5- or 6-membered heteroaryl comprising at least one nitrogen atom. In certain embodiments, Ring A is optionally substituted pyrrole. In certain embodiments, Ring A is optionally substituted furan. In certain embodiments, Ring A is optionally substituted thiophene. In certain embodiments, Ring A is optionally substituted imidazole. In certain embodiments, Ring A is optionally substituted pyrazole. In certain embodiments, Ring A is optionally substituted oxazole. In certain embodiments, Ring A is optionally substituted isoxazole. In certain embodiments, Ring A is optionally substituted thiazole. In certain embodiments, Ring A is optionally substituted isothiazole. In certain embodiments, Ring A is optionally substituted triazole. In certain embodiments, Ring A is optionally substituted oxadiazole. In certain embodiments, Ring A is optionally substituted thiadiazole. In certain embodiments, Ring A is optionally substituted tetrazolyl. In certain embodiments, Ring A is optionally substituted pyridine. In certain embodiments, Ring A is optionally substituted pyridazine. In certain embodiments, Ring A is optionally substituted pyrimidine. In certain embodiments, Ring A is or optionally substituted pyrazine.

In certain embodiments, Ring A is of the formula:

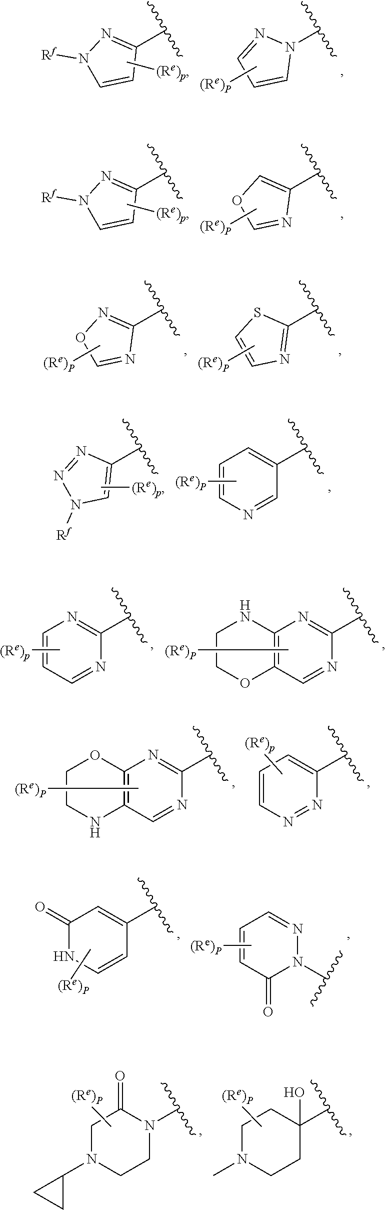

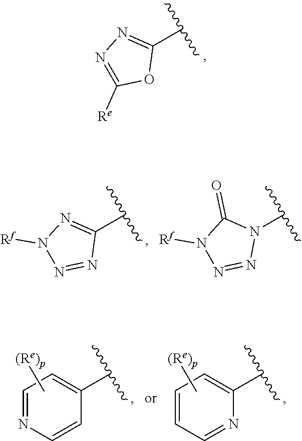

wherein each instance of R^{e} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}, —C(═O)R^{g}, —C(═O)OR^{g}, —C(═O)SR^{g}, —C(═O)N(R^{g})₂, —OC(═O)R^{g}, —OC(═O)OR^{g}, —OC(═O)SR^{g}, —OC(═O)N(R^{g})₂, —N(R^{g})C(═O)R^{g}, —N(R^{g})C(═O)OR^{g}, —N(R^{g})C(═O)SR^{g}, —N(R^{g})C(═O)N(R^{g})₂, —SC(═O)R^{g}, —SC(═O)OR^{g}, —SC(═O)SR^{g}, —SC(═O)N(R^{g})₂, —C(═NR^{g})R^{g}, —C(═NR^{g})OR^{g}, —C(═NR^{g})SR^{g}, —C(═NR^{g})N(R^{g})₂, —OC(═NR^{g})R^{g}, —OC(═NR^{g})OR^{g}, —OC(═NR^{g})SR^{g}, —OC(═NR^{g})N(R^{g})₂, —N(R^{g})C(═NR^{g})R^{g}, —N(R^{g})C(═NR^{g})OR^{g}, —N(R^{g})C(═NR^{g})SR^{g}, —N(R^{g}) C(═NR^{g})N(R^{g})₂, —SC(═NR^{g})R^{g}, —SC(═NR^{g})OR^{g}, —SC(═NR^{g})SR^{g}, —SC(═NR^{g})N(R^{g})₂, —C(═S)R^{g}, —C(═S)OR^{g}, —C(═S)SR^{g}, —C(═S)N(R^{g})₂, —S(═O)R^{g}, —SO₂R^{g}, —NR^{g}SO₂R^{g}, —SO₂N(R^{g})₂, —CN, —SCN, or —NO₂;
each occurrence of R^{f} or R^{g} is independently D¹, hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
p is 0, 1, 2, 3, or 4.

In certain embodiments, Ring A is of the formula:

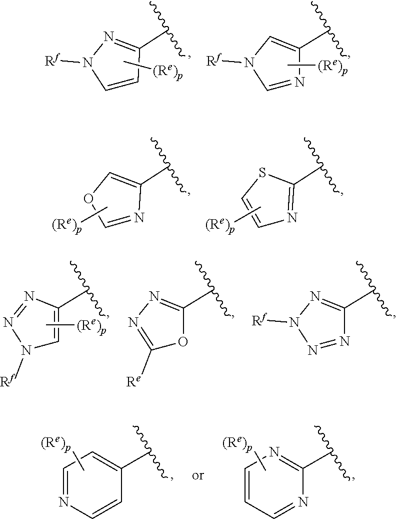

wherein each instance of R^{e} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}, —C(═O)R^{g}, —C(═O)OR^{g}, —C(═O)SR^{g}, —C(═O)N(R^{g})₂, —OC(═O)R^{g}, —OC(═O)OR^{g}, —OC(═O)SR^{g}, —OC(═O)N(R^{g})₂, —N(R^{g})C(═O)R^{g}, —N(R^{g})C(═O)OR^{g}, —N(R^{g})C(═O)SR^{g}, —N(R^{g})C(═O)N(R^{g})₂, —SC(═O)R^{g}, —SC(═O)OR^{g}, —SC(═O)SR^{g}, —SC(═O)N(R^{g})₂, —C(═NR^{g})R^{g}, —C(═NR^{g})OR^{g}, —C(═NR^{g})SR^{g}, —C(═NR^{g})N(R^{g})₂, —OC(═NR^{g})R^{g}, —OC(═NR^{g})OR^{g}, —OC(═NR^{g})SR^{g}, —OC(═NR^{g})N(R^{g})₂, —N(R^{g})C(═NR^{g})R^{g}, —N(R^{g})C(═NR^{g})OR^{g}, —N(R^{g})C(═NR^{g})SR^{g}, —N(R^{g}) C(═NR^{g})N(R^{g})₂, —SC(═NR^{g})R^{g}, —SC(═NR^{g})OR^{g}, —SC(═NR^{g})SR^{g}, —SC(═NR^{g})N(R^{g})₂, —C(═S)R^{g}, —C(═S)OR^{g}, —C(═S)SR^{g}, —C(═S)N(R^{g})₂, —S(═O)R^{g}, —SO₂R^{g}, —NR^{g}SO₂R^{g}, —SO₂N(R^{g})₂, —CN, —SCN, or —NO₂;
   each occurrence of R^{f} or R^{g} is independently D¹, hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
   p is 0, 1, 2, 3, or 4.

In certain embodiments, Ring A is of the formula:

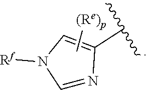

In certain embodiments, Ring A is of the formula:

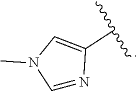 [SMILES: *c1cn(C)cn1]

In certain embodiments, Ring A is of the formula:

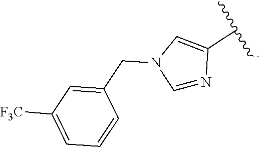 [SMILES: *c1cn(Cc2cccc(C(F)(F)F)c2)cn1]

In certain embodiments, Ring A is of the formula:

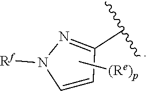

In certain embodiments, Ring A is of the formula:

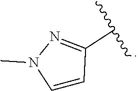 [SMILES: *c1ccn(C)n1]

In certain embodiments, Ring A is of the formula:

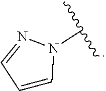 [SMILES: *n1cccn1]

In certain embodiments, Ring A is of the formula:

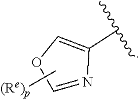

In certain embodiments, Ring A is of the formula:

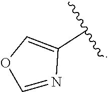 [SMILES: *c1cocn1]

In certain embodiments, Ring A is of the formula:

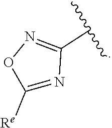

In certain embodiments, Ring A is of the formula:

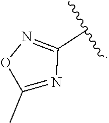 [SMILES: *c1noc(C)n1]

In certain embodiments, Ring A is of the formula:

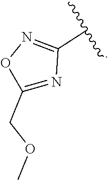 [SMILES: *c1noc(COC)n1]

In certain embodiments, Ring A is of the formula:

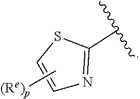

In certain embodiments, Ring A is of the formula:

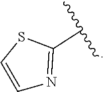 [SMILES: *c1nccs1]

In certain embodiments, Ring A is of the formula:

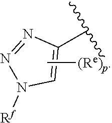

In certain embodiments, Ring A is of the formula:

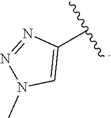 [SMILES: *c1cn(C)nn1]

In certain embodiments, Ring A is of the formula:

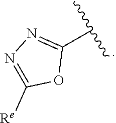

In certain embodiments, Ring A is of the formula:

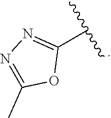 [SMILES: *c1nnc(C)o1]

In certain embodiments, Ring A is of the formula:

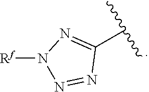

In certain embodiments, Ring A is of the formula:

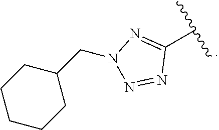 [SMILES: *c1nnn(CC2CCCCC2)n1]

In certain embodiments, Ring A is of the formula:

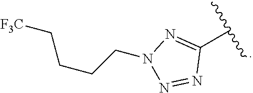 [SMILES: *c1nnn(CCCCC(F)(F)F)n1]

In certain embodiments, Ring A is of the formula:

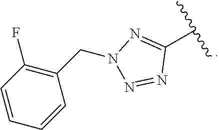 [SMILES: *c1nnn(Cc2ccccc2F)n1]

In certain embodiments, Ring A is of the formula:

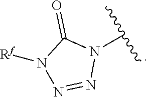

In certain embodiments, Ring A is of the formula:

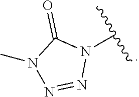 [SMILES: *n1nnn(C)c1=O]

In certain embodiments, Ring A is of the formula:

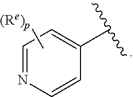

In certain embodiments, Ring A is of the formula:

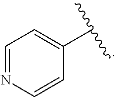 [SMILES: *c1ccncc1]

In certain embodiments, Ring A is of the formula:

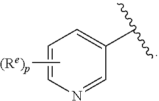

In certain embodiments, Ring A is of the formula:

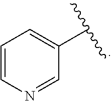 [SMILES: *c1cccnc1]

In certain embodiments, Ring A is of the formula:

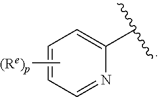

In certain embodiments, Ring A is of the formula:

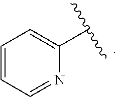 [SMILES: *c1ccccn1]

In certain embodiments, Ring A is of the formula:

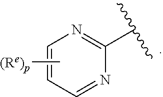

In certain embodiments, Ring A is of the formula:

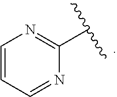 [SMILES: *c1ncccn1]

In certain embodiments, Ring A is of the formula:

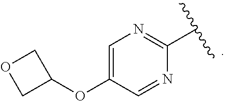 [SMILES: *c1ncc(OC2COC2)cn1]

In certain embodiments, Ring A is of the formula:

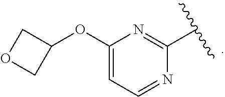 [SMILES: *c1nccc(OC2COC2)n1]

In certain embodiments, Ring A is of the formula:

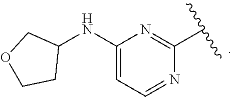 [SMILES: *c1nccc(NC2CCOC2)n1]

In certain embodiments, Ring A is of the formula:

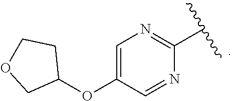 [SMILES: *c1ncc(OC2CCOC2)cn1]

In certain embodiments, Ring A is of the formula:

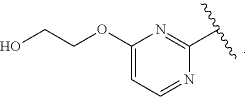 [SMILES: *c1nccc(OCCO)n1]

In certain embodiments, Ring A is of the formula:

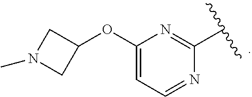 [SMILES: *c1nccc(OC2CN(C)C2)n1]

In certain embodiments, Ring A is of the formula:

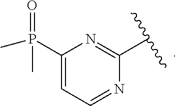 [SMILES: *c1nccc(P(C)(C)=O)n1]

In certain embodiments, Ring A is of the formula:

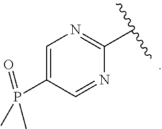 [SMILES: *c1ncc(P(C)(C)=O)cn1]

In certain embodiments, Ring A is of the formula:

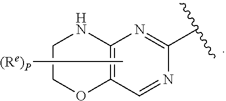

In certain embodiments, Ring A is of the formula:

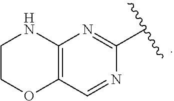 [SMILES: *c1ncc2c(n1)NCCO2]

In certain embodiments, Ring A is of the formula:

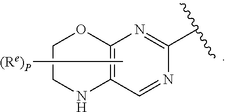

In certain embodiments, Ring A is of the formula:

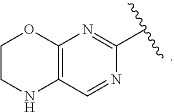 [SMILES: *c1ncc2c(n1)OCCN2]

In certain embodiments, Ring A is of the formula:

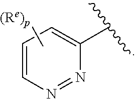

In certain embodiments, Ring A is of the formula:

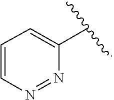 [SMILES: *c1cccnn1]

In certain embodiments, Ring A is of the formula:

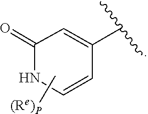

In certain embodiments, Ring A is of the formula:

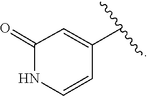 [SMILES: *c1cc[nH]c(=O)c1]

In certain embodiments, Ring A is of the formula:

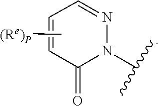

In certain embodiments, Ring A is of the formula:

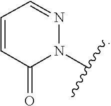 [SMILES: *n1ncccc1=O]

In certain embodiments, Ring A is of the formula:

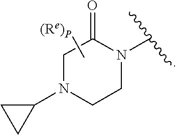

In certain embodiments, Ring A is of the formula:

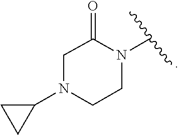 [SMILES: *N1CCN(C2CC2)CC1=O]

In certain embodiments, Ring A is of the formula:

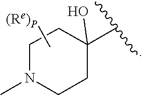

In certain embodiments, Ring A is of the formula:

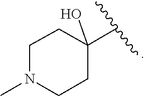 [SMILES: *C1(O)CCN(C)CC1]

In certain embodiments, Ring A is of the formula:

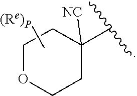

In certain embodiments, Ring A is of the formula:

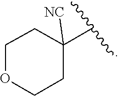 [SMILES: *C1(C#N)CCOCC1]

As generally defined herein, the compound of Formula (I″), Formula (I′), or Formula (I) contains the substituent R¹. In certain embodiments, R¹ is hydrogen. In certain embodiments, R¹ is halogen. In certain embodiments, R¹ is optionally substituted alkyl. In certain embodiments, R¹ is optionally substituted alkenyl. In certain embodiments, R¹ is optionally substituted alkynyl. In certain embodiments, R¹ is optionally substituted carbocyclyl. In certain embodiments, R¹ is optionally substituted heterocyclyl. In certain embodiments, R¹ is optionally substituted aryl. In certain embodiments, R¹ is optionally substituted heteroaryl. In certain embodiments, R¹ is —OR^{a}. In certain embodiments, R¹ is —N(R^{b})₂. In certain embodiments, R¹ is —SR^{a}. In certain embodiments, R¹ is —C(═O)R^{a}. In certain embodiments, R¹ is —C(═O)OR^{a}. In certain embodiments, R¹ is —C(═O)SR^{a}. In certain embodiments, R¹ is —C(═O)N(R^{b})₂. In certain embodiments, R¹ is —OC(═O)R^{a}. In certain embodiments, R¹ is —OC(═O)OR^{a}. In certain embodiments, R¹ is —OC(═O)SR^{a}. In certain embodiments, R¹ is —OC(═O)N(R^{b})₂. In certain embodiments, R¹ is —N(R^{b})C(═O)R^{a}. In certain embodiments, R¹ is —N(R^{b})C(═O)OR^{a}. In certain embodiments, R¹ is —N(R^{b})C(═O)SR^{a}. In certain embodiments, R¹ is —N(R^{b})C(═O)N(R^{b})₂. In certain embodiments, R¹ is —SC(═O)R^{a}. In certain embodiments, R¹ is —SC(═O)OR^{a}. In certain embodiments, R¹ is —SC(═O)SR^{a}. In certain embodiments, R¹ is —SC(═O)N(R^{b})₂. In certain embodiments, R¹ is —C(═NR^{b})R^{b}. In certain embodiments, R¹ is —C(═NR^{b})OR^{a}. In certain embodiments, R¹ is —C(═NR^{b})SR^{a}. In certain embodiments, R¹ is —C(═NR^{b})N(R^{b})₂. In certain embodiments, R¹ is —OC(═NR^{b})R^{b}. In certain embodiments, R¹ is —OC(═NR^{b})OR^{a}. In certain embodiments, R¹ is —OC(═NR^{b})SR^{a}. In certain embodiments, R¹ is —OC(═NR^{b})N(R^{b})₂. In certain embodiments, R¹ is —N(R^{b})C(═NR^{b})R^{b}. In certain embodiments, R¹ is —N(R^{b})C(═NR^{b})OR^{a}. In certain embodiments, R¹ is —N(R^{b})C(═NR^{b})SR^{a}. In certain embodiments, R¹ is —N(R^{b})C(═NR^{b})N(R^{b})₂. In certain embodiments, R¹ is —SC(═NR^{b})R^{b}. In certain embodiments, R¹ is —SC(═NR^{b})OR^{a}. In certain embodiments, R¹ is —SC(═NR^{b})SR^{a}. In certain embodiments, R¹ is —SC(═NR^{b})N(R^{b})₂. In certain embodiments, R¹ is —C(═S)R^{a}. In certain embodiments, R¹ is —C(═S)OR^{a}. In certain embodiments, R¹ is —C(═S)SR^{a}. In certain embodiments, R¹ is —C(═S)N(R^{b})₂. In certain embodiments, R¹ is —S(═O)R^{a}. In certain embodiments, R¹ is —SO₂R^{a}. In certain embodiments, R¹ is —NR^{b}SO₂R^{a}. In certain embodiments, R¹ is —SO₂N(R^{b})₂. In certain embodiments, R¹ is —CN or —SCN. In certain embodiments, R¹ is —NO₂.

In certain embodiments, the substituent R¹ contains the substituent R^{a}. In certain embodiments, each occurrence of R^{a}, when present, is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom. In certain embodiments, R^{a} is hydrogen. In certain embodiments, R^{a} is optionally substituted alkyl. In certain embodiments, R^{a} is optionally substituted alkenyl. In certain embodiments, R^{a} is optionally substituted alkynyl. In certain embodiments, R^{a} is optionally substituted carbocyclyl. In certain embodiments, R^{a} is optionally substituted heterocyclyl. In certain embodiments, R^{a} is optionally substituted aryl. In certain embodiments, R^{a} is optionally substituted heteroaryl. In certain embodiments, R^{a} is an oxygen protecting group when attached to an oxygen atom. In certain embodiments, R^{a} is a sulfur protecting group when attached to a sulfur atom.

In certain embodiments, the substituent R¹ contains the substituent R^{b}. In certain embodiments, each occurrence of R^{b}, when present, is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring. In certain embodiments, each occurrence of R^{b}, when present, is independently hydrogen. In certain embodiments, each occurrence of R^{b}, when present, is independently optionally substituted alkyl. In certain embodiments, each occurrence of R^{b}, when present, is independently optionally substituted alkenyl. In certain embodiments, each occurrence of R^{b}, when present, is independently optionally substituted alkynyl. In certain embodiments, each occurrence of R^{b}, when present, is independently optionally substituted carbocyclyl. In certain embodiments, each occurrence of R^{b}, when present, is independently optionally substituted heterocyclyl. In certain embodiments, each occurrence of R^{b}, when present, is independently optionally substituted aryl. In certain embodiments, each occurrence of R^{b}, when present, is independently optionally substituted heteroaryl. In certain embodiments, each occurrence of R^{b}, when present, is independently a nitrogen protecting group. In certain embodiments, two instances of R^{b}, when present, can be joined together with the heteroatom to which they are attached to form an optionally substituted heterocyclic ring.

As generally defined herein, Formula (I″), Formula (I′), or Formula (I) contains the substituent X¹. In certain embodiments, X¹ is —CH₂O—. In certain embodiments, X¹ is —OCH₂—. In certain embodiments, X¹ is —O—, —N(R^{c})—, —S—, —C(═O)—, —C(═O)O—, —C(═O)N(R^{c})—, —OC(═O)—, or —N(R^{c})C(═O)—. In certain embodiments, X¹ is —O—. In certain embodiments, X¹ is —S—. In certain embodiments, X¹ is —C(═O)—. In certain embodiments, X¹ is —C(═O)O—. In certain embodiments, X¹ is —C(═O)N(R^{c})—. In certain embodiments, X¹ is —OC(═O)—. In certain embodiments, X¹ is —N(R^{c})C(═O)—. In certain embodiments, X¹ is —N(R^{c})—. In certain embodiments, X¹ is —N(H)—. In certain embodiments, X¹ is —N(Me)-.

In certain embodiments, the substituent X¹ contains the substituent R^{c}. In certain embodiments, each occurrence of R^{c}, when present, is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, each occurrence of R^{c}, when present, is independently hydrogen. In certain embodiments, each occurrence of R^{c}, when present, is independently optionally substituted alkyl. In certain embodiments, each occurrence of R^{c}, when present, is independently optionally substituted alkenyl. In certain embodiments, each occurrence of R^{c}, when present, is independently optionally substituted alkynyl. In certain embodiments, each occurrence of R^{c}, when present, is independently optionally substituted carbocyclyl. In certain embodiments, each occurrence of R^{c}, when present, is independently optionally substituted heterocyclyl. In certain embodiments, each occurrence of R^{c}, when present, is independently optionally substituted aryl. In certain embodiments, each occurrence of R^{c}, when present, is independently optionally substituted heteroaryl. In certain embodiments, each occurrence of R^{c}, when present, is independently a nitrogen protecting group.

As generally defined herein, the compound of Formula (I″), Formula (I′), or Formula (I) contains the substituent V¹. In certain embodiments, V¹ is ═N— or ═C(R^{c})—. In certain embodiments, V¹ is ═N—. In certain embodiments, V¹ is ═C(R^{c})—. In certain embodiments, V¹ is ═C(H)—.

In certain embodiments, n is 0. In certain embodiments, n is 1. In certain embodiments, n is 2. In certain embodiments, n is 3.

In certain embodiments, a compound described herein is of Formula (II′):

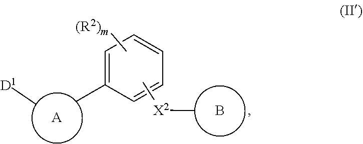

or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom, or a bicyclic heteroaryl;
Ring B is an optionally substituted bicyclic or monocyclic aryl, or an optionally substituted carbocyclic ring;
R² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X² is a bond, —C(R^{c})₂—; —CO—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
m is 0, 1, 2, 3, or 4; and
D¹ is a warhead of any one of Formulae (i-1) to (i-43):

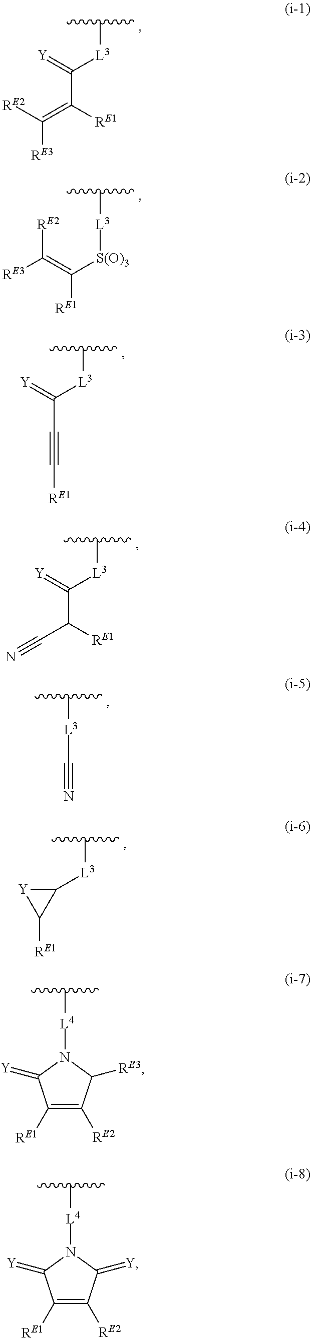

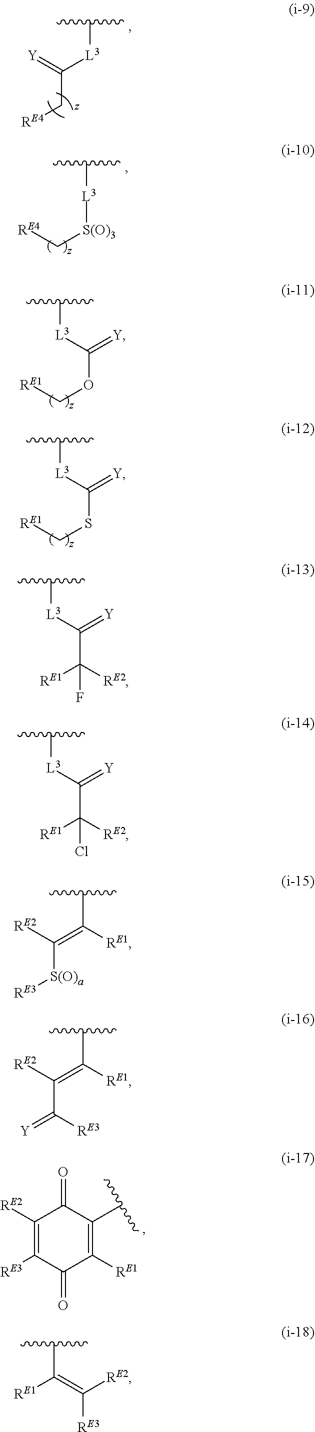

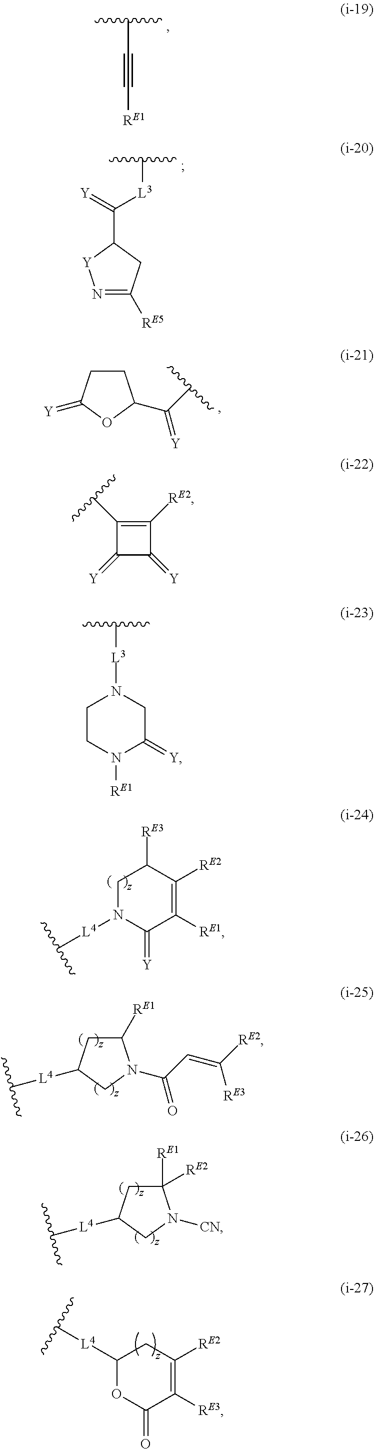

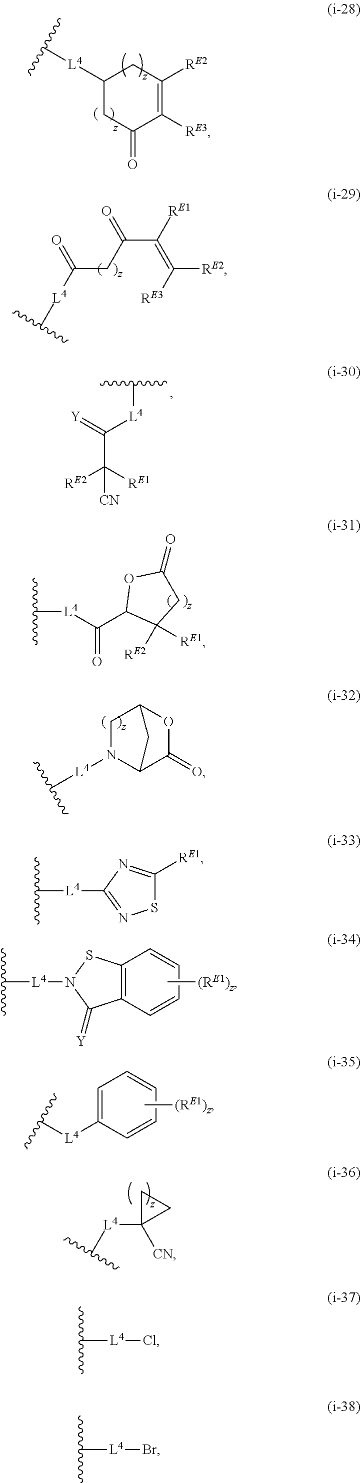

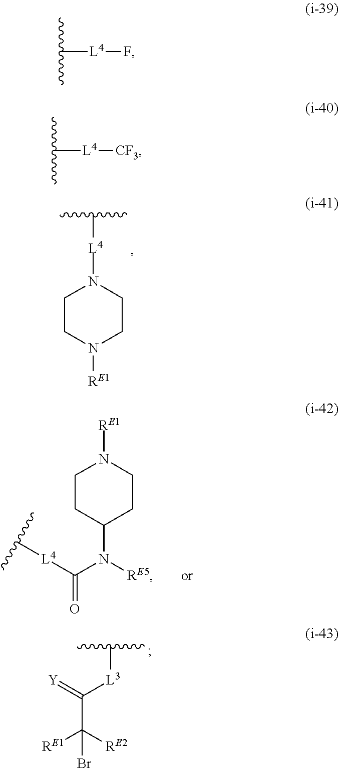

wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═))O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

In certain embodiments, the compound of Formula (II′) is of the Formula (II′-a):

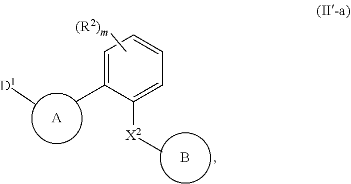

or a pharmaceutically acceptable salt, solvate, hydrate, polymorph, co-crystal, tautomer, stereoisomer, isotopically labeled compound, or prodrug thereof.

In certain embodiments, the compound of Formula (II′) is of the Formula (II′-b):

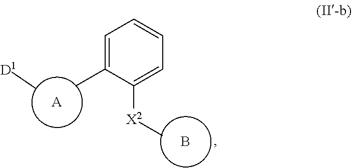

or a pharmaceutically acceptable salt, solvate, hydrate, polymorph, co-crystal, tautomer, stereoisomer, isotopically labeled compound, or prodrug thereof.

In certain embodiments, a compound of Formula (II′) as described herein is of Formula (II):

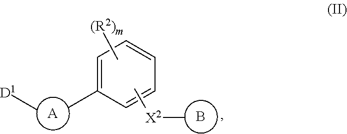

or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom;
Ring B is an optionally substituted bicyclic or monocyclic aryl, or an optionally substituted carbocyclic ring;
R² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X² is —O—, —N(R^{c})—, —S—, —C(═O)—, —C(═O)O—, —C(═O)N(R^{c})—, —OC(═O)—, or —N(R^{c})C(═O)—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{b}, when present, can be joined together with the heteroatom to which they are attached to form an optionally substituted heterocyclic ring;
m is 0, 1, 2, 3, or 4; and
D¹ is a warhead of any one of Formulae (i-1) to (i-43):

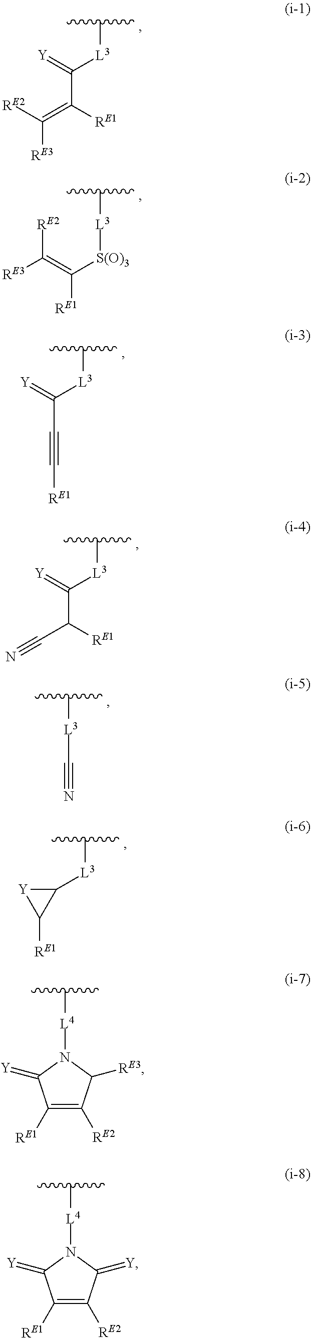

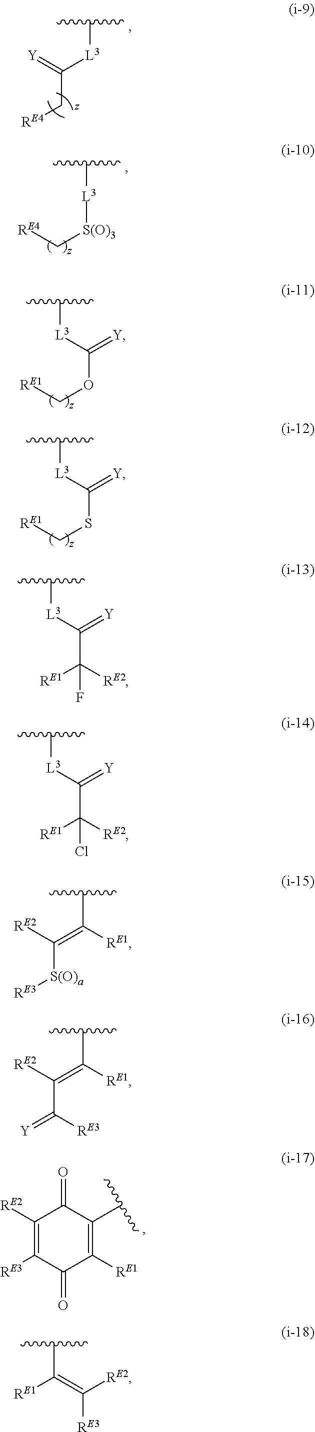

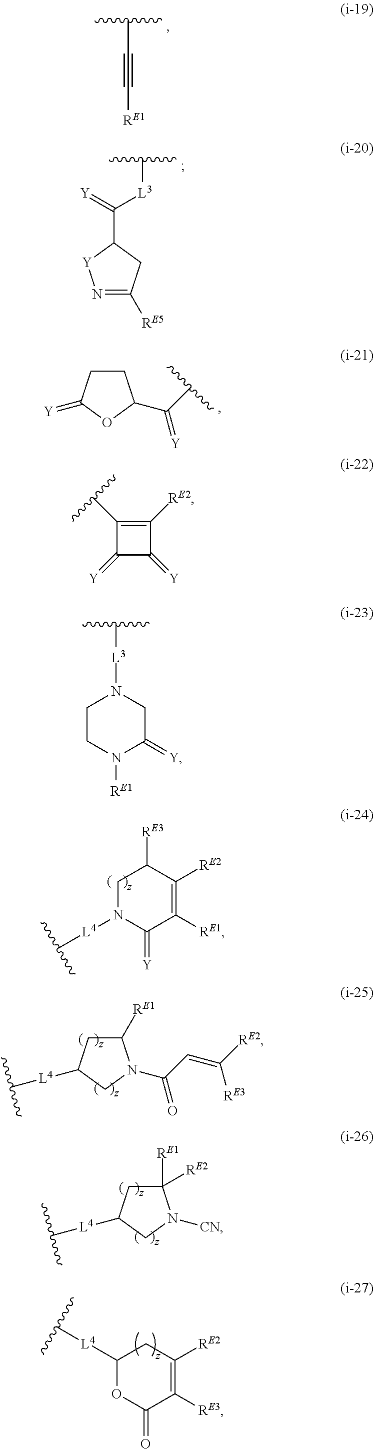

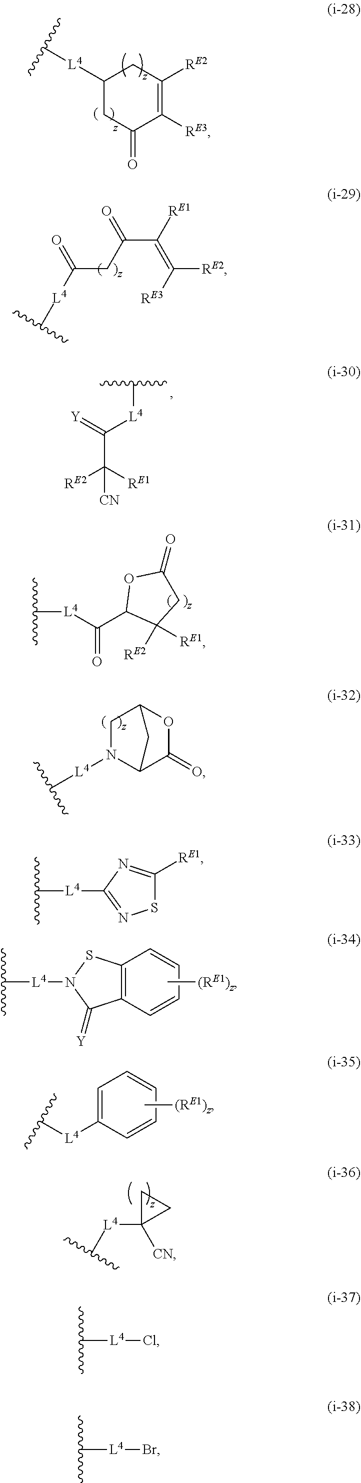

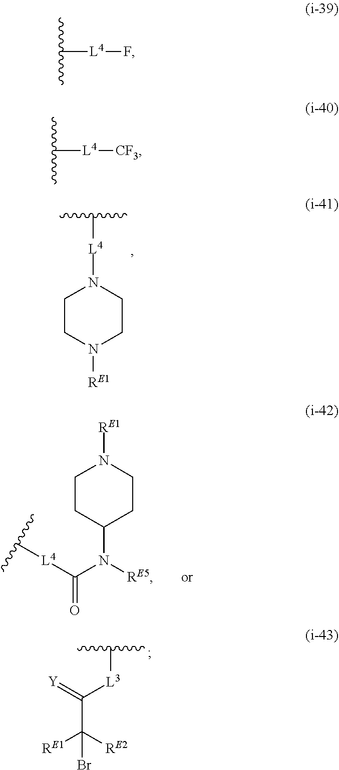

wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═))O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}—, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

In certain embodiments, the compound of Formula (II) is of the Formula (II-a):

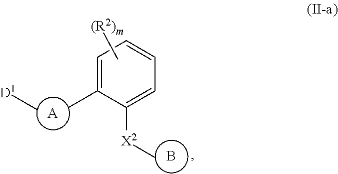

or a pharmaceutically acceptable salt, solvate, hydrate, polymorph, co-crystal, tautomer, stereoisomer, isotopically labeled compound, or prodrug thereof.

In certain embodiments, the compound of Formula (II) is of the Formula (II-b):

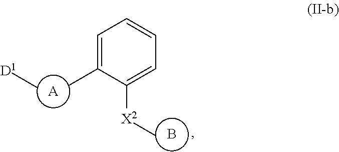

or a pharmaceutically acceptable salt, solvate, hydrate, polymorph, co-crystal, tautomer, stereoisomer, isotopically labeled compound, or prodrug thereof.

Formula (II′) contains Ring A. In certain embodiments, Ring A is an optionally substituted 5- or 6-membered heteroaryl comprising at least one nitrogen atom. In certain embodiments, Ring A is an optionally substituted pyrrole, optionally substituted furan, optionally substituted thiophene, optionally substituted imidazole, optionally substituted pyrazole, optionally substituted oxazole, optionally substituted isoxazole, optionally substituted thiazole, optionally substituted isothiazole, optionally substituted triazole, optionally substituted oxadiazole, optionally substituted thiadiazole, optionally substituted tetrazolyl, optionally substituted pyridine, optionally substituted pyridazine, optionally substituted pyrimidine, or optionally substituted pyrazine. In certain embodiments, Ring A is of the formula:

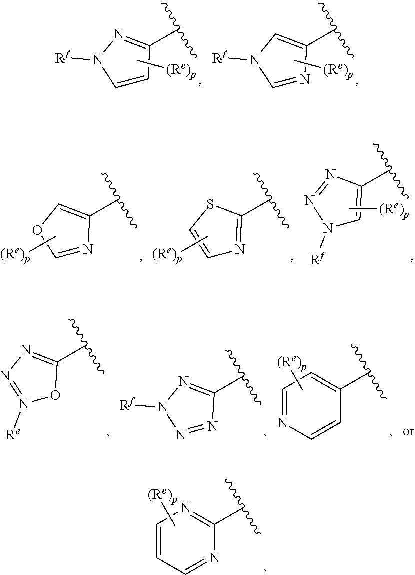

wherein each instance of R^{e} is independently D¹, hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}, —C(═O)R^{g}, —C(═O)OR^{g}, —C(═O)SR^{g}, —C(═O)N(R^{g})₂, —OC(═O)R^{g}, —OC(═O)OR^{g}, —OC(═O)SR^{g}, —OC(═O)N(R^{g})₂, —N(R^{g})C(═O)R^{g}, —N(R^{g})C(═O)OR^{g}, —N(R^{g})C(═O)SR^{g}, —N(R^{g})C(═O)N(R^{g})₂, —SC(═O)R^{g}, —SC(═O)OR^{g}, —SC(═O)SR^{g}, —SC(═O)N(R^{g})₂, —C(═NR^{g})R^{g}, —C(═NR^{g})OR^{g}, —C(═NR^{g})SR^{g}, —C(═NR^{g})N(R^{g})₂, —OC(═NR^{g})R^{g}, —OC(═NR^{g})OR^{g}, —OC(═NR^{g})SR^{g}, —OC(═NR^{g})N(R^{g})₂, —N(R^{g})C(═NR^{g})R^{g}, —N(R^{g})C(═NR^{g})OR^{g}, —N(R^{g})C(═NR^{g})SR^{g}, —N(R^{g}) C(═NR^{g})N(R^{g})₂, —SC(═NR^{g})R^{g}, —SC(═NR^{g})OR^{g}, —SC(═NR^{g})SR^{g}, —SC(═NR^{g})N(R^{g})₂, —C(═S)R^{g}, —C(═S)OR^{g}, —C(═S)SR^{g}, —C(═S)N(R^{g})₂, —S(═O)R^{g}, —SO₂R^{g}, —NR^{g}SO₂R^{g}, —SO₂N(R^{g})₂, —CN, —SCN, or —NO₂;
each occurrence of R^{f} or R^{g} is independently D¹, hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
p is 0, 1, 2, 3, or 4.

In certain embodiments, Ring A is of the formula:

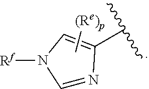

In certain embodiments, Ring A is of the formula:

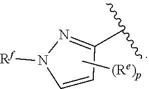

In certain embodiments, Ring A is of the formula:

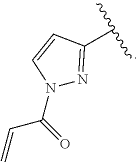 [SMILES: *c1ccn(C(=O)C=C)n1]

In certain embodiments, Ring A is of the formula:

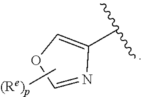

In certain embodiments, Ring A is of the formula:

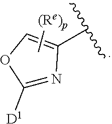

In certain embodiments, Ring A is of the formula:

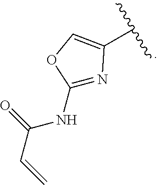 [SMILES: *c1coc(NC(=O)C=C)n1]

In certain embodiments, Ring A is of the formula:

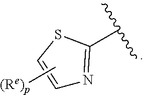

In certain embodiments, Ring A is of the formula:

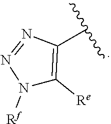

In certain embodiments, Ring A is of the formula:

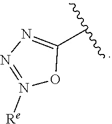

In certain embodiments, Ring A is of the formula:

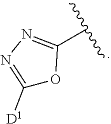

In certain embodiments, Ring A is of the formula:

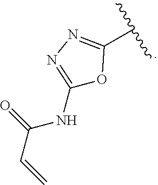 [SMILES: *c1nnc(NC(=O)C=C)o1]

In certain embodiments, Ring A is of the formula:

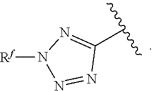

In certain embodiments, Ring A is of the formula:

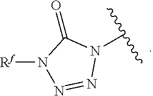

In certain embodiments, Ring A is of the formula:

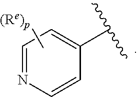

In certain embodiments, Ring A is of the formula:

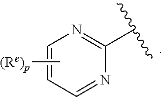

Formula (II′) contains the substituent R². In certain embodiments, at least one instance of R² is hydrogen. In certain embodiments, at least one instance of R² is halogen. In certain embodiments, at least one instance of R² is optionally substituted alkyl. In certain embodiments, at least one instance of R² is optionally substituted alkenyl. In certain embodiments, at least one instance of R² is optionally substituted alkynyl. In certain embodiments, at least one instance of R² is optionally substituted carbocyclyl. In certain embodiments, at least one instance of R² is optionally substituted heterocyclyl. In certain embodiments, at least one instance of R² is optionally substituted aryl. In certain embodiments, at least one instance of R² is optionally substituted heteroaryl. In certain embodiments, at least one instance of R² is —OR^{a}. In certain embodiments, at least one instance of R² is —N(R^{b})₂. In certain embodiments, at least one instance of R² is —SR^{a}. In certain embodiments, at least one instance of R² is —C(═O)R^{a}. In certain embodiments, at least one instance of R² is —C(═O)OR^{a}. In certain embodiments, at least one instance of R² is —C(═O)SR^{a}. In certain embodiments, at least one instance of R² is —C(═O)N(R^{b})₂. In certain embodiments, at least one instance of R² is —OC(═O)R^{a}. In certain embodiments, at least one instance of R² is —OC(═O)OR^{a}. In certain embodiments, at least one instance of R² is —OC(═O)SR^{a}. In certain embodiments, at least one instance of R² is —OC(═O)N(R^{b})₂. In certain embodiments, at least one instance of R² is —N(R^{b})C(═O)R^{a}. In certain embodiments, at least one instance of R² is —N(R^{b})C(═O)OR^{a}. In certain embodiments, at least one instance of R² is —N(R^{b})C(═O)SR^{a}. In certain embodiments, at least one instance of R² is —N(R^{b})C(═O)N(R^{b})₂. In certain embodiments, at least one instance of R² is —SC(═O)R^{a}. In certain embodiments, at least one instance of R² is —SC(═O)OR^{a}. In certain embodiments, at least one instance of R² is —SC(═O)SR^{a}. In certain embodiments, at least one instance of R² is —SC(═O)N(R^{b})₂. In certain embodiments, at least one instance of R² is —C(═NR^{b})R^{b}. In certain embodiments, at least one instance of R² is —C(═NR^{b})OR^{a}. In certain embodiments, at least one instance of R² is —C(═NR^{b})SR^{a}. In certain embodiments, at least one instance of R² is —C(═NR^{b})N(R^{b})₂. In certain embodiments, at least one instance of R² is —OC(═NR^{b})R^{b}. In certain embodiments, at least one instance of R² is —OC(═NR^{b})OR^{a}. In certain embodiments, at least one instance of R² is —OC(═NR^{b})SR^{a}. In certain embodiments, at least one instance of R² is —OC(═NR^{b})N(R^{b})₂. In certain embodiments, at least one instance of R² is —N(R^{b})C(═NR^{b})R^{b}. In certain embodiments, at least one instance of R² is —N(R^{b})C(═NR^{b})OR^{a}. In certain embodiments, at least one instance of R² is —N(R^{b})C(═NR^{b})SR^{a}. In certain embodiments, at least one instance of R² is —N(R^{b})C(═NR^{b})N(R^{b})₂. In certain embodiments, at least one instance of R² is —SC(═NR^{b})R^{b}. In certain embodiments, at least one instance of R² is —SC(═NR^{b})OR^{a}. In certain embodiments, at least one instance of R² is —SC(═NR^{b})SR^{a}. In certain embodiments, at least one instance of R² is —SC(═NR^{b})N(R^{b})₂. In certain embodiments, at least one instance of R² is —C(═S)R^{a}. In certain embodiments, at least one instance of R² is —C(═S)OR^{a}. In certain embodiments, at least one instance of R² is —C(═S)SR^{a}. In certain embodiments, at least one instance of R² is —C(═S)N(R^{b})₂. In certain embodiments, at least one instance of R² is —S(═O)R^{a}. In certain embodiments, at least one instance of R² is —SO₂R^{a}. In certain embodiments, at least one instance of at least one instance of R² is —NR^{b}SO₂R^{a}. In certain embodiments, at least one instance of R² is —SO₂N(R^{b})₂. In certain embodiments, at least one instance of R² is —CN. In certain embodiments, at least one instance of R² is —SCN. In certain embodiments, at least one instance of R² is —NO₂.

In certain embodiments, the substituent R² contains the substituent R^{a}. In certain embodiments, each occurrence of R^{a}, when present, is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom. In certain embodiments, R^{a} is hydrogen. In certain embodiments, R^{a} is optionally substituted alkyl. In certain embodiments, R^{a} is optionally substituted alkenyl. In certain embodiments, R^{a} is optionally substituted alkynyl. In certain embodiments, R^{a} is optionally substituted carbocyclyl. In certain embodiments, R^{a} is optionally substituted heterocyclyl. In certain embodiments, R^{a} is optionally substituted aryl. In certain embodiments, R^{a} is optionally substituted heteroaryl. In certain embodiments, R^{a} is an oxygen protecting group when attached to an oxygen atom. In certain embodiments, R^{a} is a sulfur protecting group when attached to a sulfur atom.

As generally defined herein, Formula (II′) contains the substituent X². In certain embodiments, X² is —O—. In certain embodiments, X² is —N(R^{c})—. In certain embodiments, X² is —S—. In certain embodiments, X² is —C(═O)—. In certain embodiments, X² is —C(═O)O—. In certain embodiments, X² is —C(═O)N(R^{c})—. In certain embodiments, X² is —OC(═O)—. In certain embodiments, X² is —N(R^{c})C(═O)—. In certain embodiments, X² is —N(H)—.

In certain embodiments, each occurrence of R^{b} or R^{c}, when present, is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring. In certain embodiments, each occurrence of R^{b} or R^{c}, when present, is independently hydrogen. In certain embodiments, each occurrence of R^{b} or R^{c}, when present, is independently optionally substituted alkyl. In certain embodiments, each occurrence of R^{b} or R^{c}, when present, is independently optionally substituted alkenyl. In certain embodiments, each occurrence of R^{b} or R^{c}, when present, is independently optionally substituted alkynyl. In certain embodiments, each occurrence of R^{b} or R^{c}, when present, is independently optionally substituted carbocyclyl. In certain embodiments, each occurrence of R^{b} or R^{c}, when present, is independently optionally substituted heterocyclyl. In certain embodiments, each occurrence of R^{b} or R^{c}, when present, is independently optionally substituted aryl. In certain embodiments, each occurrence of R^{b} or R^{c}, when present, is independently optionally substituted heteroaryl. In certain embodiments, each occurrence of R^{b} or R^{c}, when present, is independently a nitrogen protecting group. In certain embodiments, two instances of R^{b}, when present, can be joined together with the heteroatom to which they are attached to form an optionally substituted heterocyclic ring.

In certain embodiments, m is 0. In certain embodiments, m is 1. In certain embodiments, m is 2. In certain embodiments, m is 3. In certain embodiments, m is 4.

As generally defined herein, Formula (I″), Formula (I′), Formula (I), Formula (II′), and Formula (II) contain Ring B. In certain embodiments, Ring B is an optionally substituted bicyclic or monocyclic aryl, or an optionally substituted carbocyclic ring. In certain embodiments, Ring B is an optionally substituted bicyclic or monocyclic aryl. In certain embodiments, Ring B is an optionally substituted carbocyclic ring. In certain embodiments, Ring B is an optionally substituted phenyl.

In certain embodiments, Ring B is of the formula:

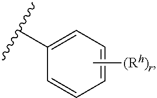

wherein each instance of R^{h} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —ORⁱ, —N(Rⁱ)₂, —SRⁱ, —C(═O)Rⁱ, —C(═O)ORⁱ, —C(═O)SRⁱ, —C(═O)N(Rⁱ)₂, —OC(═O)Rⁱ, —OC(═O)ORⁱ, —OC(═O)SRⁱ, —OC(═O)N(Rⁱ)₂, —N(Rⁱ)C(═O)Rⁱ, —N(Rⁱ)C(═O)ORⁱ, —N(Rⁱ)C(═O)SRⁱ, —N(Rⁱ)C(═O)N(Rⁱ)₂, —SC(═O)Rⁱ, —SC(═O)ORⁱ, —SC(═O)SRⁱ, —SC(═O)N(Rⁱ)₂, —C(═NRⁱ)Rⁱ, —C(═NRⁱ)ORⁱ, —C(═NRⁱ)SRⁱ, —C(═NRⁱ)N(Rⁱ)₂, —OC(═NRⁱ)Rⁱ, —OC(═NRⁱ)ORⁱ, —OC(═NRⁱ)SRⁱ, —OC(═NRⁱ)N(Rⁱ)₂, —N(Rⁱ)C(═NRⁱ)Rⁱ, —N(Rⁱ)C(═NRⁱ)ORⁱ, —N(Rⁱ)C(═NRⁱ)SRⁱ, —N(Rⁱ) C(═NRⁱ)N(Rⁱ)₂, —SC(═NRⁱ)Rⁱ, —SC(═NRⁱ)ORⁱ, —SC(═NRⁱ)SRⁱ, —SC(═NRⁱ)N(Rⁱ)₂, —C(═S)Rⁱ, —C(═S)ORⁱ, —C(═S)SRⁱ, —C(═S)N(Rⁱ)₂, —S(═O)Rⁱ, —SO₂Rⁱ, —NRⁱSO₂Rⁱ, —SO₂N(Rⁱ)₂, —CN, —SCN, or —NO₂;
each occurrence of Rⁱ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
r is 0, 1, 2, 3, 4, 5, 6, 7, or 8.

In certain embodiments, at least on instance of R^{h} is independently hydrogen. In certain embodiments, at least on instance of R^{h} is independently halogen. In certain embodiments, at least on instance of R^{h} is independently optionally substituted alkyl. In certain embodiments, at least on instance of R^{h} is independently optionally substituted alkenyl. In certain embodiments, at least on instance of R^{h} is independently optionally substituted alkynyl. In certain embodiments, at least on instance of R^{h} is independently optionally substituted carbocyclyl. In certain embodiments, at least on instance of R^{h} is independently optionally substituted heterocyclyl. In certain embodiments, at least on instance of R^{h} is independently optionally substituted aryl. In certain embodiments, at least on instance of R^{h} is independently optionally substituted heteroaryl. In certain embodiments, at least on instance of R^{h} is independently —OR. In certain embodiments, at least on instance of R^{h} is independently —N(Rⁱ)₂. In certain embodiments, at least on instance of R^{h} is independently —SRⁱ. In certain embodiments, at least on instance of R^{h} is independently —C(═O)Rⁱ. In certain embodiments, at least on instance of R^{h} is independently —C(═O)ORⁱ. In certain embodiments, at least on instance of R^{h} is independently —C(═O)SRⁱ. In certain embodiments, at least on instance of R^{h} is independently —C(═O)N(Rⁱ)₂. In certain embodiments, at least on instance of R^{h} is independently —OC(═O)Rⁱ. In certain embodiments, at least on instance of R^{h} is independently —OC(═O)ORⁱ. In certain embodiments, at least on instance of R^{h} is independently —OC(═O)SRⁱ. In certain embodiments, at least on instance of R^{h} is independently —OC(═O)N(Rⁱ)₂. In certain embodiments, at least on instance of R^{h} is independently —N(Rⁱ)C(═O)Rⁱ. In certain embodiments, at least on instance of R^{h} is independently —N(Rⁱ)C(═O)ORⁱ. In certain embodiments, at least on instance of R^{h} is independently —N(Rⁱ)C(═O)SRⁱ. In certain embodiments, at least on instance of R^{h} is independently —N(Rⁱ)C(═O)N(Rⁱ)₂. In certain embodiments, at least on instance of R^{h} is independently —SC(═O)Rⁱ. In certain embodiments, at least on instance of R^{h} is independently —SC(═O)ORⁱ. In certain embodiments, at least on instance of R^{h} is independently —SC(═O)SRⁱ. In certain embodiments, at least on instance of R^{h} is independently —SC(═O)N(Rⁱ)₂. In certain embodiments, at least on instance of R^{h} is independently —C(═NRⁱ)Rⁱ. In certain embodiments, at least on instance of R^{h} is independently —C(═NRⁱ)ORⁱ. In certain embodiments, at least on instance of R^{h} is independently —C(═NRⁱ)SRⁱ. In certain embodiments, at least on instance of R^{h} is independently —C(═NRⁱ)N(Rⁱ)₂. In certain embodiments, at least on instance of R^{h} is independently —OC(═NRⁱ)Rⁱ. In certain embodiments, at least on instance of R^{h} is independently —OC(═NRⁱ)ORⁱ. In certain embodiments, at least on instance of R^{h} is independently —OC(═NRⁱ)SRⁱ. In certain embodiments, at least on instance of R^{h} is independently —OC(═NRⁱ)N(Rⁱ)₂. In certain embodiments, at least on instance of R^{h} is independently —N(Rⁱ)C(═NRⁱ)Rⁱ. In certain embodiments, at least on instance of R^{h} is independently —N(Rⁱ)C(═NRⁱ)ORⁱ. In certain embodiments, at least on instance of R^{h} is independently —N(Rⁱ)C(═NRⁱ)SRⁱ. In certain embodiments, at least on instance of R^{h} is independently —N(Rⁱ)C(═NRⁱ)N(Rⁱ)₂. In certain embodiments, at least on instance of R^{h} is independently —SC(═NRⁱ)Rⁱ. In certain embodiments, at least on instance of R^{h} is independently —SC(═NRⁱ)ORⁱ. In certain embodiments, at least on instance of R^{h} is independently —SC(═NRⁱ)SRⁱ. In certain embodiments, at least on instance of R^{h} is independently —SC(═NRⁱ)N(Rⁱ)₂. In certain embodiments, at least on instance of R^{h} is independently —C(═S)Rⁱ. In certain embodiments, at least on instance of R^{h} is independently —C(═S)ORⁱ. In certain embodiments, at least on instance of R^{h} is independently —C(═S)SRⁱ. In certain embodiments, at least on instance of R^{h} is independently —C(═S)N(Rⁱ)₂. In certain embodiments, at least on instance of R^{h} is independently —S(═O)Rⁱ. In certain embodiments, at least on instance of R^{h} is independently —SO₂Rⁱ. In certain embodiments, at least on instance of R^{h} is independently —NRⁱSO₂Rⁱ. In certain embodiments, at least on instance of R^{h} is independently —SO₂N(Rⁱ)₂. In certain embodiments, at least on instance of R^{h} is independently —CN. In certain embodiments, at least on instance of R^{h} is independently —SCN. In certain embodiments, at least on instance of R^{h} is independently —NO₂.

In certain embodiments, R^{h} contains the substituent Rⁱ. In certain embodiments, each occurrence of Rⁱ, when present, is independently hydrogen. In certain embodiments, each occurrence of Rⁱ, when present, is independently optionally substituted alkyl. In certain embodiments, each occurrence of Rⁱ, when present, is independently optionally substituted alkenyl. In certain embodiments, each occurrence of Rⁱ, when present, is independently optionally substituted alkynyl. In certain embodiments, each occurrence of Rⁱ, when present, is independently optionally substituted carbocyclyl. In certain embodiments, each occurrence of Rⁱ, when present, is independently optionally substituted heterocyclyl. In certain embodiments, each occurrence of Rⁱ, when present, is independently optionally substituted aryl. In certain embodiments, each occurrence of Rⁱ, when present, is independently optionally substituted heteroaryl. In certain embodiments, each occurrence of Rⁱ, when present, is independently an oxygen protecting group when attached to an oxygen atom. In certain embodiments, each occurrence of Rⁱ, when present, is independently a sulfur protecting group when attached to a sulfur atom.

In certain embodiments, r is 0. In certain embodiments, r is 1. In certain embodiments, r is 2. In certain embodiments, r is 3. In certain embodiments, r is 4. In certain embodiments, r is 5. In certain embodiments, r is 6. In certain embodiments, r is 7. In certain embodiments, r is 8.

In certain embodiments, Ring B is of the formula:

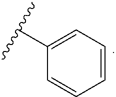 [SMILES: *c1ccccc1]

In certain embodiments, Ring B is of the formula:

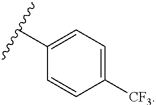 [SMILES: *c1ccc(C(F)(F)F)cc1]

In certain embodiments, Ring B is an optionally substituted carbocyclic ring. In certain embodiments, Ring B is an optionally substituted cyclohexyl ring. In certain embodiments, Ring B is of the formula:

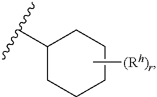

wherein each instance of R^{h} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —ORⁱ, —N(Rⁱ)₂, —SRⁱ, —C(═O)Rⁱ, —C(═O)ORⁱ, —C(═O)SRⁱ, —C(═O)N(Rⁱ)₂, —OC(═O)Rⁱ, —OC(═O)ORⁱ, —OC(═O)SRⁱ, —OC(═O)N(Rⁱ)₂, —N(Rⁱ)C(═O)Rⁱ, —N(Rⁱ)C(═O)ORⁱ, —N(Rⁱ)C(═O)SRⁱ, —N(Rⁱ)C(═O)N(Rⁱ)₂, —SC(═O)Rⁱ, —SC(═O)ORⁱ, —SC(═O)SRⁱ, —SC(═O)N(Rⁱ)₂, —C(═NRⁱ)Rⁱ, —C(═NRⁱ)ORⁱ, —C(═NRⁱ)SRⁱ, —C(═NRⁱ)N(Rⁱ)₂, —OC(═NRⁱ)Rⁱ, —OC(═NRⁱ)ORⁱ, —OC(═NRⁱ)SRⁱ, —OC(═NRⁱ)N(Rⁱ)₂, —N(Rⁱ)C(═NRⁱ)Rⁱ, —N(Rⁱ)C(═NRⁱ)ORⁱ, —N(Rⁱ)C(═NRⁱ)SRⁱ, —N(Rⁱ) C(═NRⁱ)N(Rⁱ)₂, —SC(═NRⁱ)Rⁱ, —SC(═NRⁱ)ORⁱ, —SC(═NRⁱ)SRⁱ, —SC(═NRⁱ)N(Rⁱ)₂, —C(═S)Rⁱ, —C(═S)ORⁱ, —C(═S)SRⁱ, —C(═S)N(Rⁱ)₂, —S(═O)Rⁱ, —SO₂Rⁱ, —NRⁱSO₂Rⁱ, —SO₂N(Rⁱ)₂, —CN, —SCN, or —NO₂;
each occurrence of Rⁱ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
r is 0, 1, 2, 3, 4, 5, 6, 7, or 8.

In certain embodiments, Ring B is of the formula:

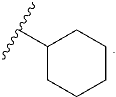 [SMILES: *C1CCCCC1]

As generally defined herein, Formula (I″), Formula (I′), Formula (I), and Formula (II) include the substituent D¹, wherein D¹ is a warhead of any one of Formulae (i-1) to (i-43):

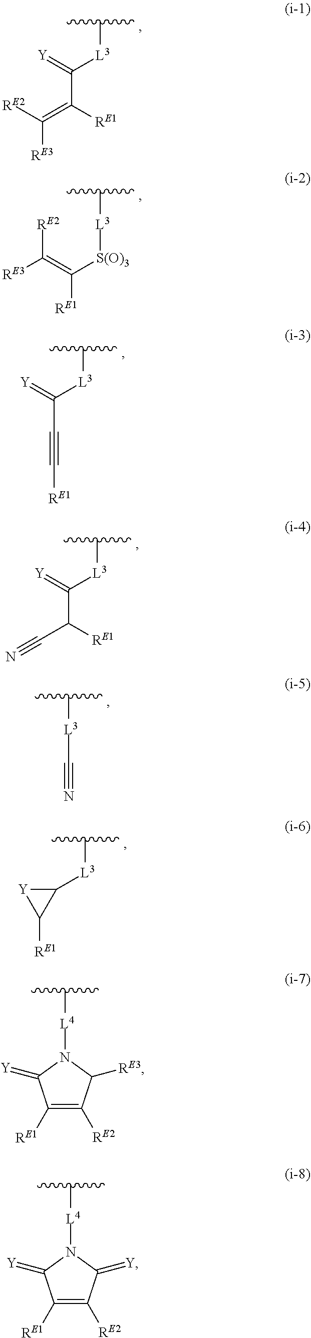

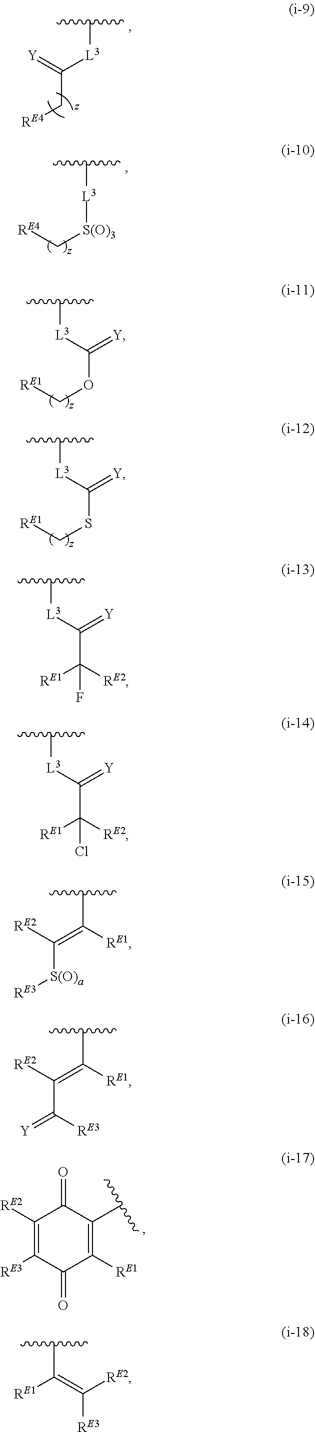

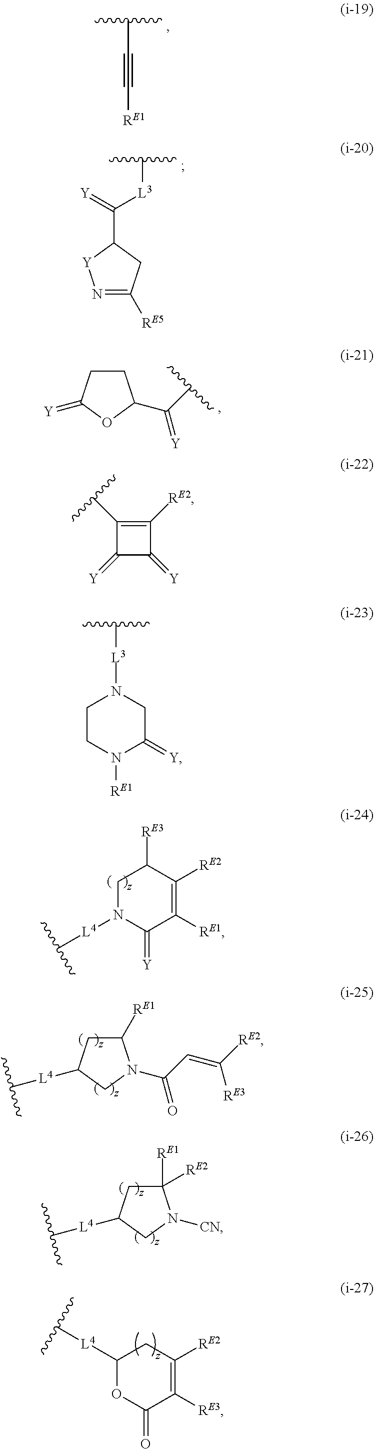

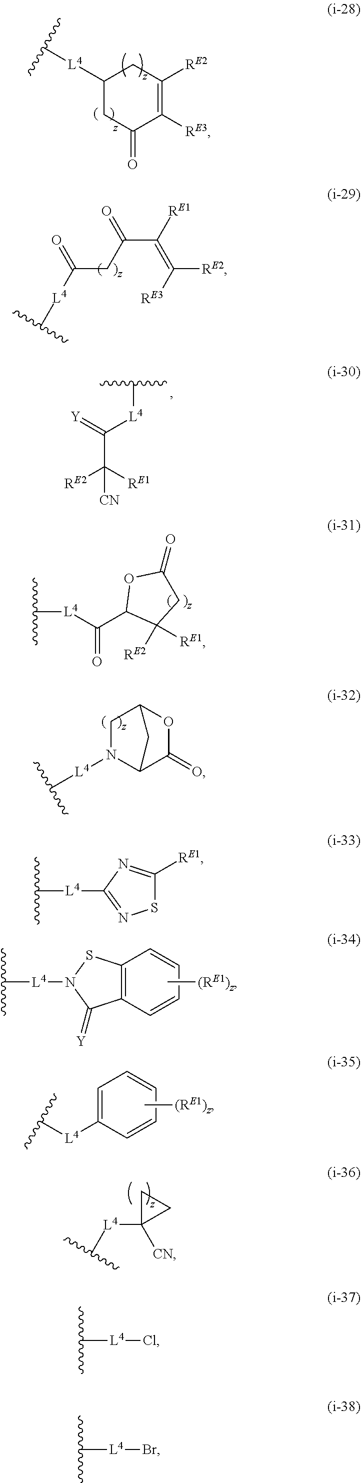

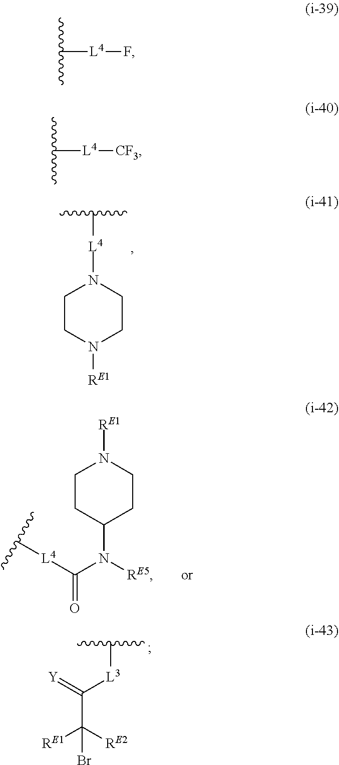

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

In certain embodiments, D¹ is a warhead of formula

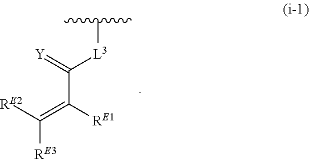

In certain embodiments, D¹ is a warhead of formula:

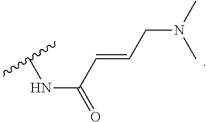 [SMILES: *NC(=O)/C=C/CN(C)C]

In certain embodiments, D¹ is a warhead of formula:

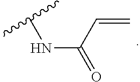 [SMILES: *NC(=O)C=C]

In certain embodiments, D¹ is a warhead of formula:

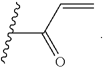 [SMILES: *C(=O)C=C]

In certain embodiments, D¹ is of formula:

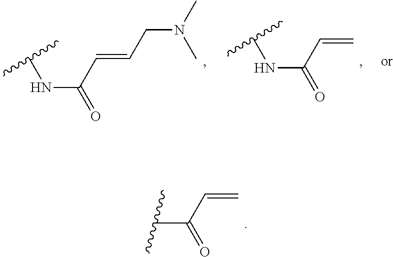

In certain embodiments, D¹ is of formula:

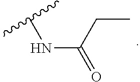 [SMILES: *NC(=O)CC]

In certain embodiments, L3 is a bond. In certain embodiments, L3 is —NH—. In certain embodiments, R^{E1} and R^{E2} are hydrogen. In certain embodiments, R^{E1}, R^{E2}, and R^{E3} are all hydrogen. In certain embodiments, R^{E3} is —CH₂NMe₂.

In certain embodiments, the warhead is of formula:

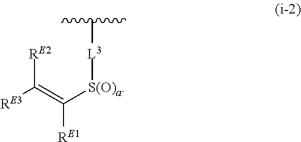

In certain embodiments, the warhead is of formula:

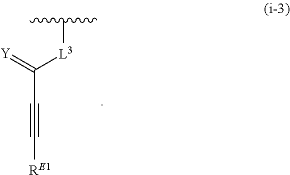

In certain embodiments, the warhead is of formula:

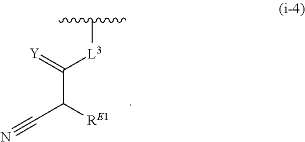

In certain embodiments, the warhead is of formula:

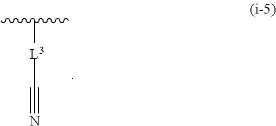

In certain embodiments, the warhead is of formula:

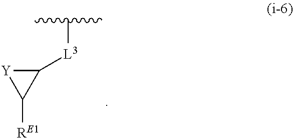

In certain embodiments, the warhead is of formula:

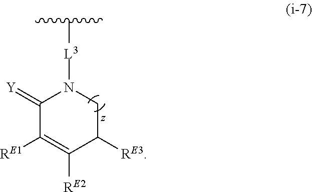

In certain embodiments, the warhead is of formula:

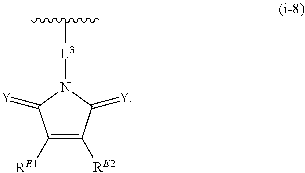

In certain embodiments, the warhead is of formula:

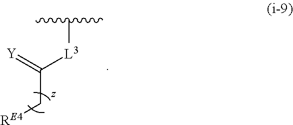

In certain embodiments, the warhead is of formula:

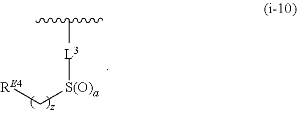

In certain embodiments, the warhead is of formula:

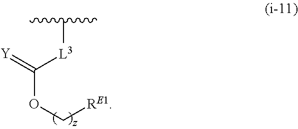

In certain embodiments, the warhead is of formula:

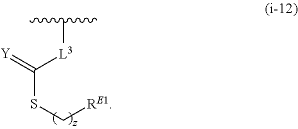

In certain embodiments, the warhead is of formula:

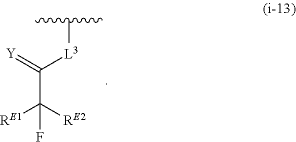

In certain embodiments, the warhead is of formula:

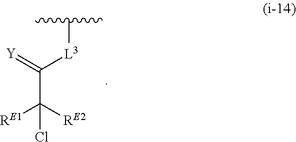

In certain embodiments, the warhead is of formula:

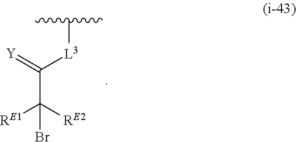

In certain embodiments, the warhead is of formula

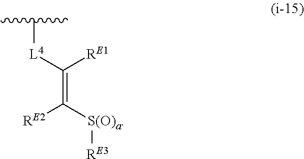

In certain embodiments, the warhead is of formula:

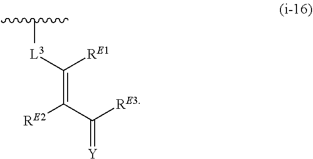

In certain embodiments, the warhead is of formula:

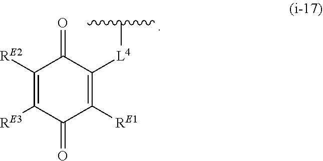

In certain embodiments, the warhead is of formula:

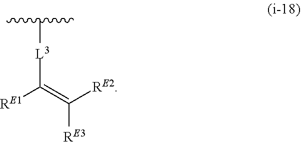

In certain embodiments, the warhead is of formula

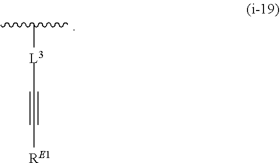

In certain embodiments, the warhead is of formula:

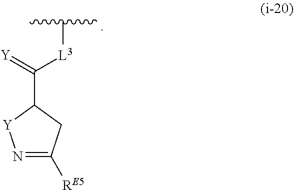

In certain embodiments, the warhead is

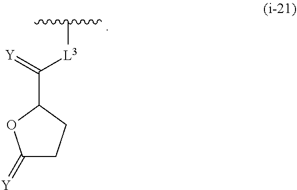

In certain embodiments, the warhead is of formula:

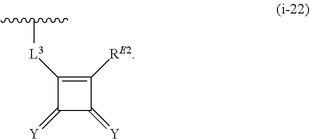

In certain embodiments, the warhead is of formula:

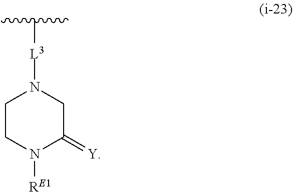

In certain embodiments, the warhead is of formula:

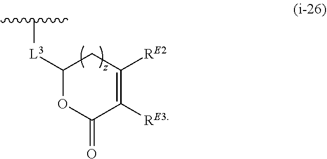

In certain embodiments, the warhead is of formula:

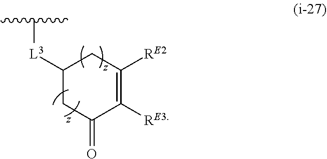

In certain embodiments, the warhead is of formula:

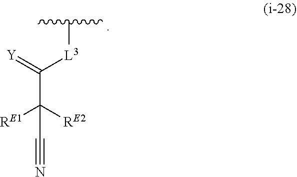

In certain embodiments, the warhead is of formula

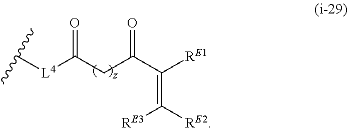

In certain embodiments, the warhead is of formula:

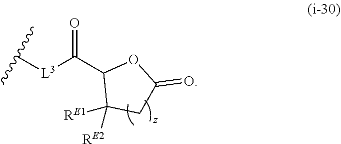

In certain embodiments, the warhead is of formula:

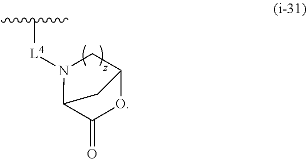

In certain embodiments, the warhead is of formula:

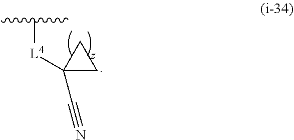

In certain embodiments, the warhead is of formula:

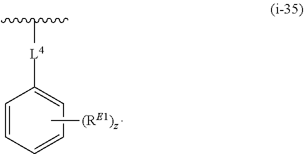

In certain embodiments, the warhead is of formula:

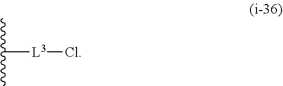

In certain embodiments, the warhead is of formula:

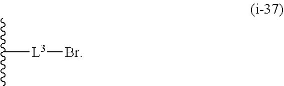

In certain embodiments, D¹ is a warhead is of formula:

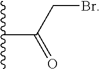 [SMILES: *C(=O)CBr]

In certain embodiments, the warhead is of formula:

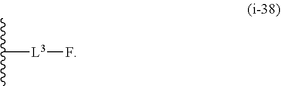

In certain embodiments, the warhead is of formula:

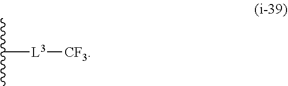

In certain embodiments, the warhead is of formula:

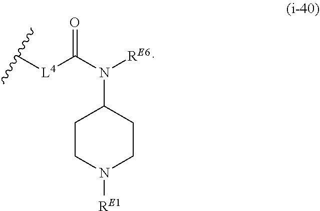

In certain embodiments, the warhead is of formula:

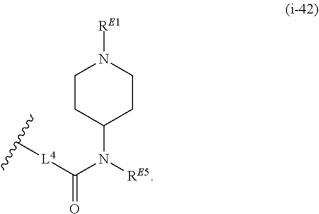

In certain embodiments, the warhead is of formula:

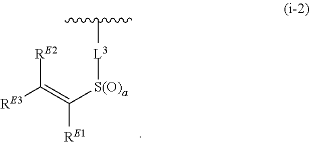

In certain embodiments, the warhead is of formula

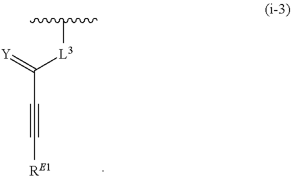

In certain embodiments, the warhead is of formula:

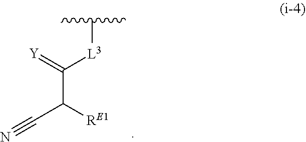

In certain embodiments, the warhead is of formula:

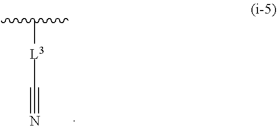

In certain embodiments, the warhead is of formula:

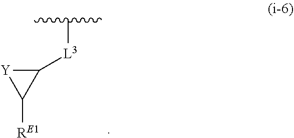

In certain embodiments, the warhead is of formula:

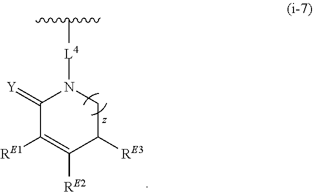

In certain embodiments, the warhead is of formula:

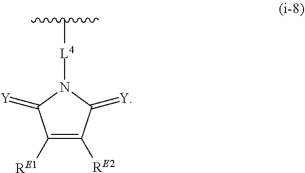

In certain embodiments, the warhead is of formula:

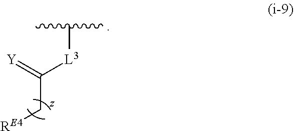

In certain embodiments, the warhead is of formula:

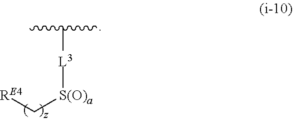

In certain embodiments, the warhead is

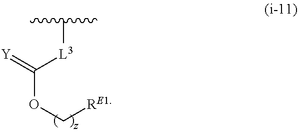

(i-11). In certain embodiments, the warhead is of formula:

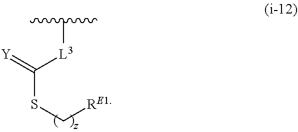

In certain embodiments, the warhead is of formula:

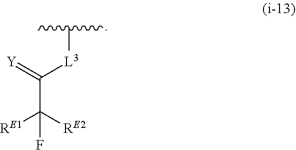

In certain embodiments, the warhead is of formula:

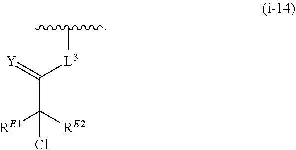

In certain embodiments, D¹ is a warhead is of formula:

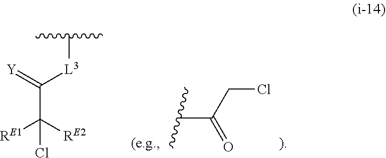

In certain embodiments, D¹ is a warhead is of formula:

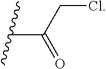 [SMILES: *C(=O)CCl]

In certain embodiments, the warhead is of formula:

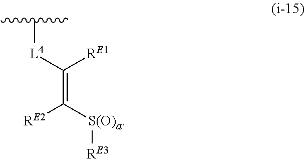

In certain embodiments, the warhead is of formula:

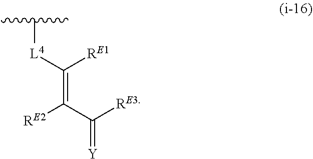

In certain embodiments, the warhead is of formula:

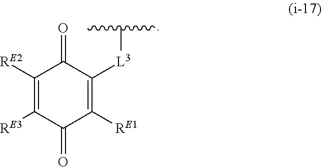

In certain embodiments, the warhead is of formula:

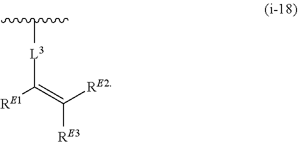

In certain embodiments, the warhead is of formula:

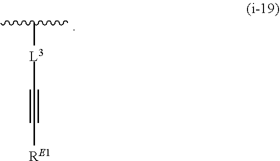

In certain embodiments, the warhead is of formula:

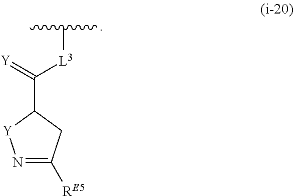

In certain embodiments, the warhead is of formula:

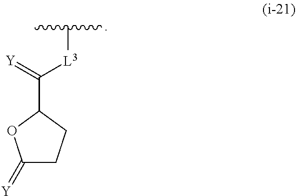

In certain embodiments, the warhead is of formula:

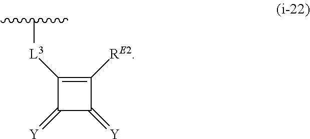

In certain embodiments, the warhead is of formula:

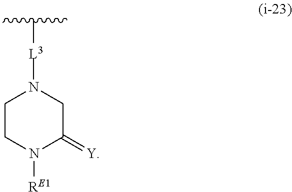

In certain embodiments, the warhead is of formula:

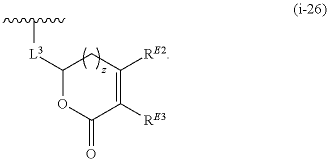

In certain embodiments, the warhead is of formula:

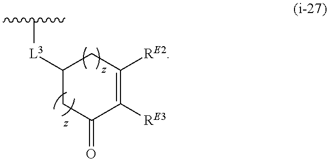

In certain embodiments, the warhead is of formula:

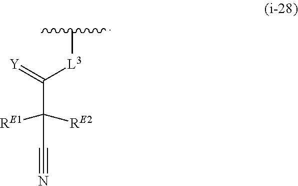

In certain embodiments, the warhead is of formula:

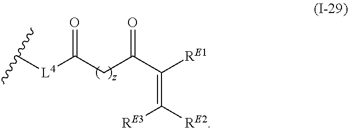

In certain embodiments, the warhead is of formula:

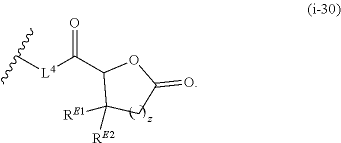

In certain embodiments, the warhead is of formula:

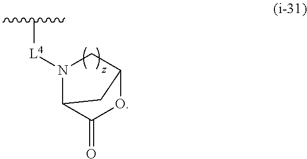

In certain embodiments, the warhead is of formula:

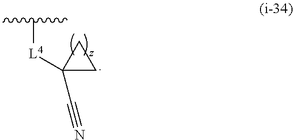

In certain embodiments, the warhead is of formula:

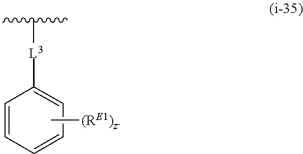

In certain embodiments, the warhead is of formula:

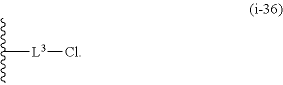

In certain embodiments, D¹ is a warhead is of formula:

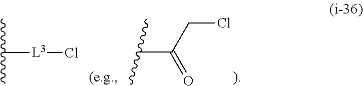

In certain embodiments, D¹ is a warhead is of formula:

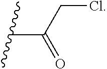 [SMILES: *C(=O)CCl]

In certain embodiments, the warhead is of formula:

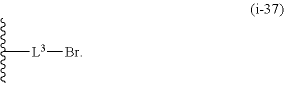

In certain embodiments, D¹ is a warhead is of formula:

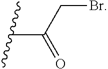 [SMILES: *C(=O)CBr]

In certain embodiments, the warhead is of formula

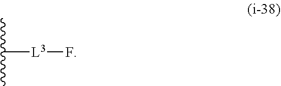

In certain embodiments, the warhead is of formula:

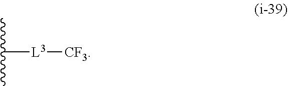

In certain embodiments, the warhead is of formula:

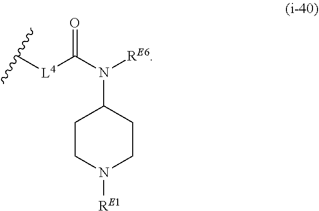

In certain embodiments, the warhead is of formula:

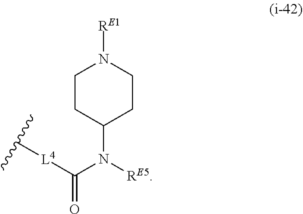

In certain embodiments, the warhead is of formula:

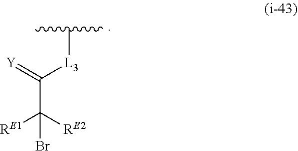

In certain embodiments, L³ is a bond (e.g., a single bond, a double bond, or a triple bond). In certain embodiments, L³ is a single bond. In certain embodiments, L³ is a double bond. In certain embodiments, L³ is a triple bond. In certain embodiments, L³ is an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}, —C≡C—, —S(═O)—, —S(═O)O—, —OS(═O)—, —S(═O)NR^{L3a}, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring. In certain embodiments, L⁴ is a bond (e.g., a single bond, a double bond, or a triple bond). In certain embodiments, L⁴ is an optionally substituted branched C₁₋₆ hydrocarbon chain (e.g., i-Pr). In certain embodiments, L⁴ is an optionally substituted unbranched C₁₋₆ hydrocarbon chain (e.g., n-Pr, or n-Bu). In certain embodiments, at least one instance of R^{E1} is H. In certain embodiments, at least one instance of R^{E1} is halogen (e.g., F, Cl, Br, or I). In certain embodiments, at least one instance of R^{E1} is optionally substituted alkyl (e.g., Me, or Et). In certain embodiments, at least one instance of R^{E1} is optionally substituted alkenyl (e.g., optionally substituted vinyl). In certain embodiments, at least one instance of R^{E1} is optionally substituted alkynyl. In certain embodiments, at least one instance of R^{E1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{E1} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, at least one instance of R^{E1} is substituted or unsubstituted aryl (e.g., substituted or unsubstituted, 6- to 10-membered aryl). In certain embodiments, at least one instance of R^{E1} is substituted or unsubstituted phenyl. In certain embodiments, at least one instance of R^{E1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, at least one instance of R^{E1} is —CN. In certain embodiments, at least one instance of R^{E1} is —CH₂OR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R^{E1} is —CH₂N(R^{EF})₂ or —N(R^{EF})₂, wherein each instance of R^{EF} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, optionally wherein two R^{EF} groups are joined to form an optionally substituted heterocyclic ring. In certain embodiments, at least one instance of R^{E1} is —CH₂SR^{EE} or —SR^{EE} (e.g., —CH₂SMe or —SMe). In certain embodiments, at least one instance of R^{E1} is —OR^{EE} (e.g., —OMe). In certain embodiments, at least one instance of R^{E1} is —Si(R^{EG})₃, wherein each instance of R^{EG} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl (e.g., —Si(Me)₃).

In certain embodiments, at least one instance of R^{E2} is H. In certain embodiments, at least one instance of R^{E2} is halogen (e.g., F, Cl, Br, or I). In certain embodiments, at least one instance of R^{E2} is optionally substituted alkyl (e.g., Me, or Et). In certain embodiments, at least one instance of R^{E2} is optionally substituted alkenyl (e.g., optionally substituted vinyl). In certain embodiments, at least one instance of R^{E2} is optionally substituted alkynyl. In certain embodiments, at least one instance of R^{E2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{E2} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, at least one instance of R^{E2} is substituted or unsubstituted aryl (e.g., substituted or unsubstituted, 6- to 10-membered aryl). In certain embodiments, at least one instance of R^{E2} is substituted or unsubstituted phenyl. In certain embodiments, at least one instance of R^{E2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, at least one instance of R^{E2} is —CN. In certain embodiments, at least one instance of R^{E2} is —CH₂OR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R^{E2} is —CH₂N(R^{EF})₂ or N(R^{EF})₂, wherein each instance of R^{EF} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, optionally wherein two R^{EF} groups are joined to form an optionally substituted heterocyclic ring. In certain embodiments, at least one instance of R^{E2} is —CH₂SR^{EE} or —SR^{EE} (e.g., —CH₂SMe or —SMe). In certain embodiments, at least one instance of R^{E2} is —OR^{EE} (e.g., —OMe). In certain embodiments, at least one instance of R^{E2} is —Si(R^{EG})₃, wherein each instance of R^{EG} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl (e.g., —Si(Me)₃). In certain embodiments, at least one instance of R^{E3} is H. In certain embodiments, at least one instance of R^{E3} is halogen (e.g., F, Cl, Br, or I). In certain embodiments, at least one instance of R^{E3} is optionally substituted alkyl (e.g., Me, or Et). In certain embodiments, at least one instance of R^{E3} is optionally substituted alkenyl (e.g., optionally substituted vinyl). In certain embodiments, at least one instance of R^{E3} is optionally substituted alkynyl. In certain embodiments, at least one instance of R^{E3} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{E3} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, at least one instance of R^{E3} is substituted or unsubstituted aryl (e.g., substituted or unsubstituted, 6- to 10-membered aryl). In certain embodiments, at least one instance of R^{E3} is substituted or unsubstituted phenyl. In certain embodiments, at least one instance of R^{E3} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, at least one instance of R^{E3} is —CN. In certain embodiments, at least one instance of R^{E3} is —CH₂OR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R^{E3} is —CH₂N(R^{EF})₂ or —N(R^{EF})₂, wherein each instance of R^{EF} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, optionally wherein two R^{EF} groups are joined to form an optionally substituted heterocyclic ring. In certain embodiments, at least one instance of R^{E3} is —CH₂SR^{EE} or —SR^{EE} (e.g., —CH₂SMe or —SMe). In certain embodiments, at least one instance of R^{E3} is —OR^{EE} (e.g., —OMe). In certain embodiments, at least one instance of R^{E3} is —Si(R^{EG})₃, wherein each instance of R^{EG} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl (e.g., —Si(Me)₃). In certain embodiments, R^{E1} and R^{E3} are joined to form an optionally substituted carbocyclic ring (e.g., substituted or unsubstituted, 3- to 7-membered, monocyclic carbocyclyl comprising zero, one, or two double bonds in the carbocyclic ring system). In certain embodiments, R^{E1} and R^{E3} are joined to form an optionally substituted heterocyclic ring (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{E2} and R^{E3} are joined to form an optionally substituted carbocyclic ring (e.g., substituted or unsubstituted, 3- to 7-membered, monocyclic carbocyclyl comprising zero, one, or two double bonds in the carbocyclic ring system). In certain embodiments, R^{E2} and R^{E3} are joined to form an optionally substituted heterocyclic ring (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{E1} and R^{E2} are joined to form an optionally substituted carbocyclic ring (e.g., substituted or unsubstituted, 3- to 7-membered, monocyclic carbocyclyl comprising zero, one, or two double bonds in the carbocyclic ring system). In certain embodiments, R^{E1} and R^{E2} are joined to form an optionally substituted heterocyclic ring (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{E4} is a leaving group (e.g., halogen, or a sulfonic acid ester, e.g., —O(tosylate) or —O(mesylate)). In certain embodiments, R^{E5} is halogen (e.g., F, Cl, Br, or I). In certain embodiments, R^{E6} is H. In certain embodiments, R^{E6} is substituted or unsubstituted C₁₋₆ alkyl (e.g., Me, is —CF₃, Bn, Et, perfluoroethyl, Pr, perfluoropropyl, Bu, or perfluorobutyl). In certain embodiments, R^{E6} is a nitrogen protecting group (e.g., Bn, Boc, Cbz, Fmoc, trifluoroacetyl, triphenylmethyl, acetyl, or Ts). In certain embodiments, at least one instance of Y is O. In certain embodiments, at least one instance of Y is S. In certain embodiments, at least one instance of Y is NR^{E7}, wherein R^{E7} is hydrogen, substituted or unsubstituted C₁₋₆ alkyl, or a nitrogen protecting group (e.g., NMe). In certain embodiments, a is 1. In certain embodiments, a is 2. In certain embodiments, at least one instance of z is 0. In certain embodiments, at least one instance of z is 1. In certain embodiments, at least one instance of z is 2. In certain embodiments, at least one instance of z is 3. In certain embodiments, at least one instance of z is 4. In certain embodiments, at least one instance of z is 5. In certain embodiments, at least one instance of z is 6.

In certain embodiments, D¹ is a warhead of formula:

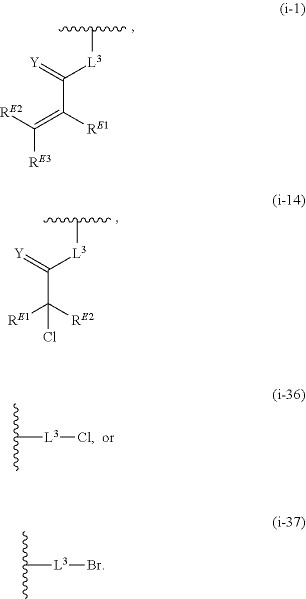

In certain embodiments, D¹ is a warhead of formula:

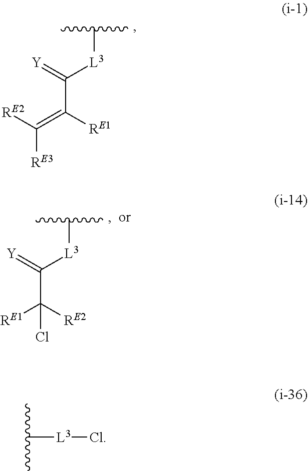

In certain embodiments, D¹ is a warhead of formula:

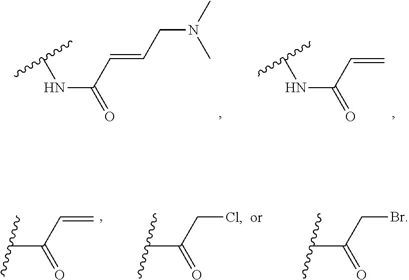

In certain embodiments, D¹ is a warhead of formula:

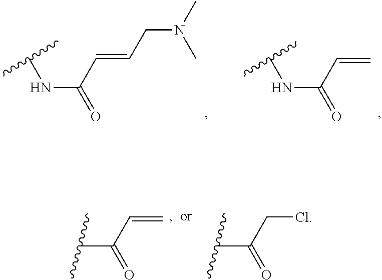

In certain embodiments, D¹ is a warhead of formula:

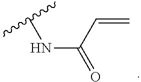 [SMILES: *NC(=O)C=C]

In certain embodiments, the compound of Formula (I) is of the Formula (I-i):

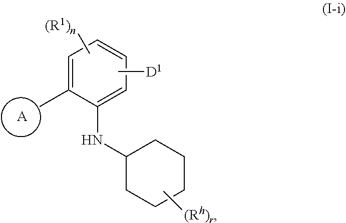

or a pharmaceutically acceptable salt, solvate, hydrate, polymorph, co-crystal, tautomer, stereoisomer, isotopically labeled compound, or prodrug thereof.

In certain embodiments, the compound of Formula (I-i) is of the formula:

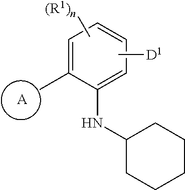

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-j):

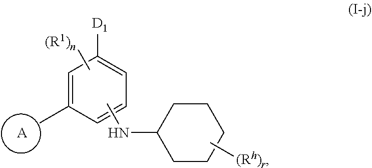

or a pharmaceutically acceptable salt, solvate, hydrate, polymorph, co-crystal, tautomer, stereoisomer, isotopically labeled compound, or prodrug thereof.

In certain embodiments, the compound of Formula (I-j) is of the formula:

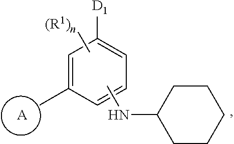

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-k):

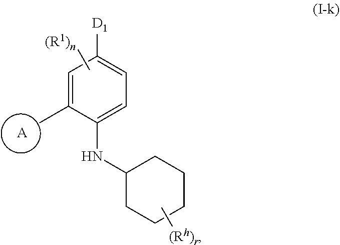

or a pharmaceutically acceptable salt, solvate, hydrate, polymorph, co-crystal, tautomer, stereoisomer, isotopically labeled compound, or prodrug thereof.

In certain embodiments, the compound of Formula (I-k) is of the formula:

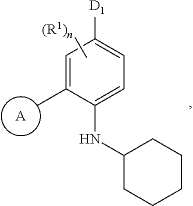

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-l):

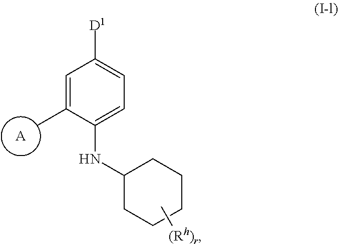

or a pharmaceutically acceptable salt, solvate, hydrate, polymorph, co-crystal, tautomer, stereoisomer, isotopically labeled compound, or prodrug thereof.

In certain embodiments, the compound of Formula (I-l) is of the formula:

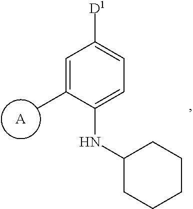

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-m):

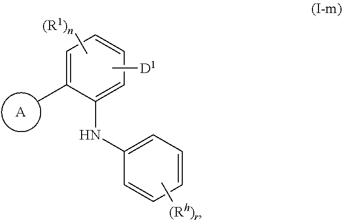

or a pharmaceutically acceptable salt, solvate, hydrate, polymorph, co-crystal, tautomer, stereoisomer, isotopically labeled compound, or prodrug thereof.

In certain embodiments, the compound of Formula (I-m) is of the formula:

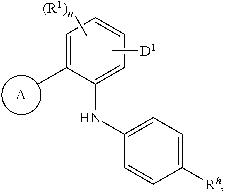

or a pharmaceutically acceptable salt, solvate, hydrate, polymorph, co-crystal, tautomer, stereoisomer, isotopically labeled compound, or prodrug thereof.

In certain embodiments, the compound of Formula (I-m) is of the formula:

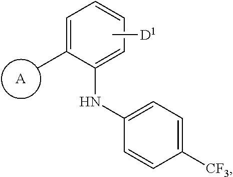

or a pharmaceutically acceptable salt, solvate, hydrate, polymorph, co-crystal, tautomer, stereoisomer, isotopically labeled compound, or prodrug thereof.

In certain embodiments, the compound of Formula (I-m) is of the formula:

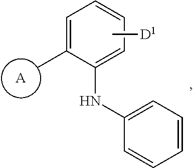

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-n):

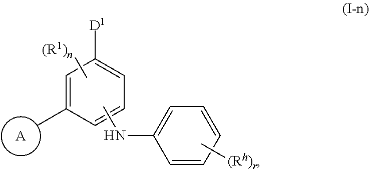

or a pharmaceutically acceptable salt, solvate, hydrate, polymorph, co-crystal, tautomer, stereoisomer, isotopically labeled compound, or prodrug thereof.

In certain embodiments, the compound of Formula (I-n) is of the formula:

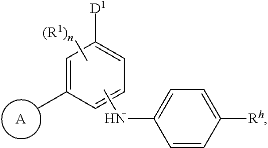

or a pharmaceutically acceptable salt, solvate, hydrate, polymorph, co-crystal, tautomer, stereoisomer, isotopically labeled compound, or prodrug thereof.

In certain embodiments, the compound of Formula (I-n) is of the formula:

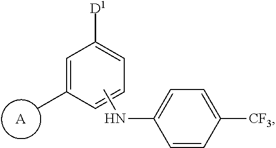

or a pharmaceutically acceptable salt, solvate, hydrate, polymorph, co-crystal, tautomer, stereoisomer, isotopically labeled compound, or prodrug thereof.

In certain embodiments, the compound of Formula (I-n) is of the formula:

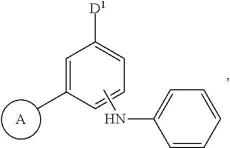

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-o):

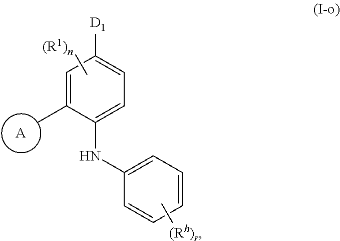

or a pharmaceutically acceptable salt, solvate, hydrate, polymorph, co-crystal, tautomer, stereoisomer, isotopically labeled compound, or prodrug thereof.

In certain embodiments, the compound of Formula (I-o) is of the formula:

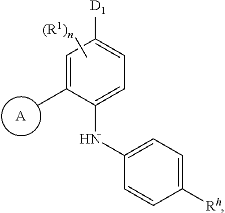

or a pharmaceutically acceptable salt, solvate, hydrate, polymorph, co-crystal, tautomer, stereoisomer, isotopically labeled compound, or prodrug thereof.

In certain embodiments, the compound of Formula (I-o) is of the formula:

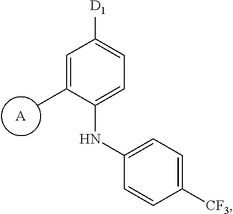

or a pharmaceutically acceptable salt, solvate, hydrate, polymorph, co-crystal, tautomer, stereoisomer, isotopically labeled compound, or prodrug thereof.

In certain embodiments, the compound of Formula-(I-o) is of the formula:

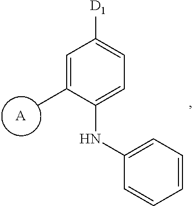

or a pharmaceutically acceptable salt, solvate, hydrate, polymorph, co-crystal, tautomer, stereoisomer, isotopically labeled compound, or prodrug thereof.

In certain embodiments, the compound of Formula (I) is of the Formula (I-p):

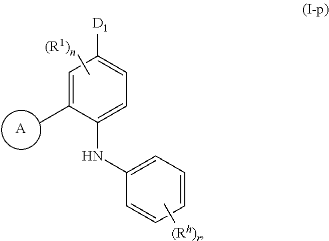

or a pharmaceutically acceptable salt, solvate, hydrate, polymorph, co-crystal, tautomer, stereoisomer, isotopically labeled compound, or prodrug thereof.

In certain embodiments, the compound of Formula (I-p) is of the formula:

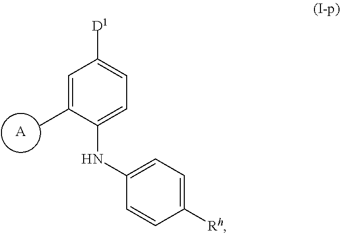

or a pharmaceutically acceptable salt, solvate, hydrate, polymorph, co-crystal, tautomer, stereoisomer, isotopically labeled compound, or prodrug thereof.

In certain embodiments, the compound of Formula (I-p) is of the formula:

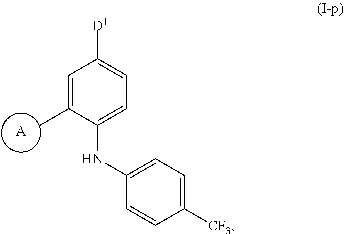

or a pharmaceutically acceptable salt, solvate, hydrate, polymorph, co-crystal, tautomer, stereoisomer, isotopically labeled compound, or prodrug thereof.

In certain embodiments, the compound of Formula (I-p) is of the formula:

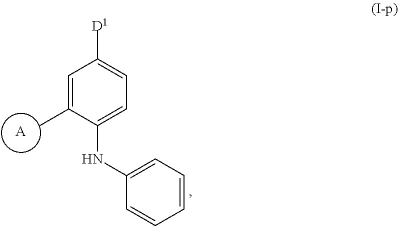

or a pharmaceutically acceptable salt, solvate, hydrate, polymorph, co-crystal, tautomer, stereoisomer, isotopically labeled compound, or prodrug thereof.

In certain embodiments, the compound of Formula (II) is of the Formula (II-c):

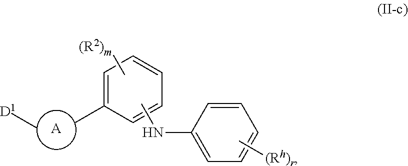

or a pharmaceutically acceptable salt, solvate, hydrate, polymorph, co-crystal, tautomer, stereoisomer, isotopically labeled compound, or prodrug thereof.

In certain embodiments, the compound of Formula (II-c) is of the formula:

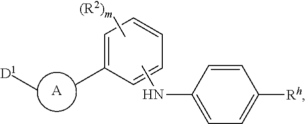

or a pharmaceutically acceptable salt, solvate, hydrate, polymorph, co-crystal, tautomer, stereoisomer, isotopically labeled compound, or prodrug thereof.

In certain embodiments, the compound of Formula (II-c) is of the formula:

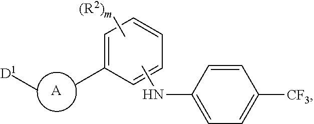

or a pharmaceutically acceptable salt, solvate, hydrate, polymorph, co-crystal, tautomer, stereoisomer, isotopically labeled compound, or prodrug thereof.

In certain embodiments, the compound of Formula (II-c) is of the formula:

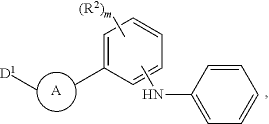

or a pharmaceutically acceptable salt, solvate, hydrate, polymorph, co-crystal, tautomer, stereoisomer, isotopically labeled compound, or prodrug thereof.

In certain embodiments, the compound of Formula (II) is of the Formula (II-d):

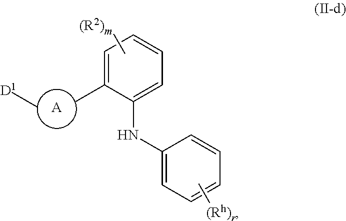

or a pharmaceutically acceptable salt, solvate, hydrate, polymorph, co-crystal, tautomer, stereoisomer, isotopically labeled compound, or prodrug thereof.

In certain embodiments, the compound of Formula (II-d) is of the formula:

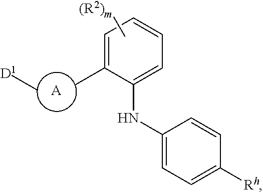

or a pharmaceutically acceptable salt, solvate, hydrate, polymorph, co-crystal, tautomer, stereoisomer, isotopically labeled compound, or prodrug thereof.

In certain embodiments, the compound of Formula (II-d) is of the formula:

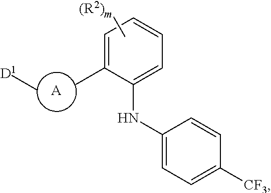

or a pharmaceutically acceptable salt, solvate, hydrate, polymorph, co-crystal, tautomer, stereoisomer, isotopically labeled compound, or prodrug thereof.

In certain embodiments, the compound of Formula (II-d) is of the formula:

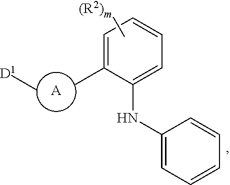

or a pharmaceutically acceptable salt, solvate, hydrate, polymorph, co-crystal, tautomer, stereoisomer, isotopically labeled compound, or prodrug thereof.

In certain embodiments, the compound of Formula (II) is of the Formula (II-e):

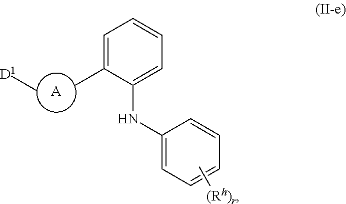

or a pharmaceutically acceptable salt, solvate, hydrate, polymorph, co-crystal, tautomer, stereoisomer, isotopically labeled compound, or prodrug thereof.

In certain embodiments, the compound of Formula (II-e) is of the formula:

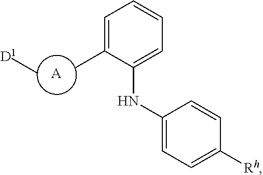

or a pharmaceutically acceptable salt, solvate, hydrate, polymorph, co-crystal, tautomer, stereoisomer, isotopically labeled compound, or prodrug thereof.

In certain embodiments, the compound of Formula (II-e) is of the formula:

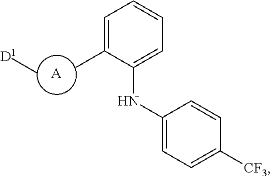

or a pharmaceutically acceptable salt, solvate, hydrate, polymorph, co-crystal, tautomer, stereoisomer, isotopically labeled compound, or prodrug thereof.

In certain embodiments, the compound of Formula (II-e) is of the formula:

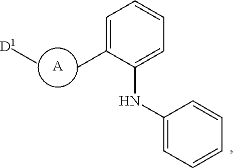

or a pharmaceutically acceptable salt, solvate, hydrate, polymorph, co-crystal, tautomer, stereoisomer, isotopically labeled compound, or prodrug thereof.

In certain embodiments, the compound of Formula (I′) is of the formula:

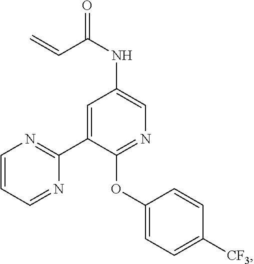 [SMILES: C=CC(=O)Nc1cnc(Oc2ccc(C(F)(F)F)cc2)c(-c2ncccn2)c1]

or a pharmaceutically acceptable salt, solvate, hydrate, polymorph, co-crystal, tautomer, stereoisomer, isotopically labeled compound, or prodrug thereof.

In certain embodiments, the compound of Formula (I″), Formula (I′), Formula (I), Formula (II′), or Formula (II), is a compound provided in any one of the Examples below.

In certain embodiments, a compound described herein is a compound of Formula (I″), Formula (I′), Formula (I), Formula (II′), or Formula (II), or a pharmaceutically acceptable salt, co-crystal, tautomer, stereoisomer, solvate, hydrate, polymorph, isotopically enriched derivative, or prodrug thereof. In certain embodiments, a compound described herein is a compound of Formula (I″), Formula (I′), Formula (I), Formula (II′), or Formula (II), or a pharmaceutically acceptable salt thereof. In certain embodiments, a compound described herein is a compound of Formula (I), or a pharmaceutically acceptable salt thereof. In certain embodiments, a compound described herein is a compound of Formula (I″), or a pharmaceutically acceptable salt thereof. In certain embodiments, a compound described herein is a compound of Formula (I′), or a pharmaceutically acceptable salt thereof. In certain embodiments, a compound described herein is a compound of Formula (II′), or a pharmaceutically acceptable salt thereof. In certain embodiments, a compound described herein is a compound of Formula (II), or a pharmaceutically acceptable salt thereof.

Certain compounds described herein bind, covalently modify, and/or inhibit a transcription factor. In certain embodiments, the compounds described herein irreversibly inhibit a transcription factor. In certain embodiments, the compounds described herein reversibly inhibit a transcription factor. In certain embodiments, the transcription factor is TEAD. In certain embodiments, the transcription factor is TEAD1. In certain embodiments, the transcription factor is TEAD2. In certain embodiments, the transcription factor is TEAD3. In certain embodiments, the transcription factor is TEAD4. In certain embodiments, the compounds described herein covalently bind to the transcription factor (e.g., TEAD, such as TEAD1, TEAD2, TEAD3, TEAD4). In certain embodiments, the compounds described herein reversibly bind to the transcription factor (e.g., TEAD, such as TEAD1, TEAD2, TEAD3, TEAD4). In certain embodiments, the compounds described herein non-reversibly bind to the transcription factor (e.g., TEAD, such as TEAD1, TEAD2, TEAD3, TEAD4). In certain embodiments, the compounds described herein modulate the activity of a transcription factor (e.g., TEAD, such as TEAD1, TEAD2, TEAD3, TEAD4). In certain embodiments, the compounds described herein inhibit a transcription factor (e.g., TEAD, such as TEAD1, TEAD2, TEAD3, TEAD4). In certain embodiments, the compounds described herein inhibit the activity of a transcription factor (e.g., TEAD, such as TEAD1, TEAD2, TEAD3, TEAD4). In certain embodiments, the compounds described herein reversibly inhibit the activity of a transcription factor (e.g., TEAD, such as TEAD1, TEAD2, TEAD3, TEAD4).

The binding affinity of a compound described herein to a transcription factor (e.g., TEAD, such as TEAD1, TEAD2, TEAD3, TEAD4) may be measured by the dissociation constant (K_{d}) value of an adduct of the compound and the TEAD using methods known in the art (e.g., isothermal titration calorimetry (ITC)). In certain embodiments, the K_{d} value of the adduct is not more than about 100 μM, not more than about 10 μM, not more than about 1 μM, not more than about 100 nM, not more than about 10 nM, or not more than about 1 nM.

In certain embodiments, the activity of a transcription factor (e.g., TEAD, such as TEAD1, TEAD2, TEAD3, TEAD4) is inhibited by a compound described herein. The inhibition of the activity of a transcription factor (e.g., TEAD, such as TEAD1, TEAD2, TEAD3, TEAD4) by a compound described herein may be measured by determining the half maximal inhibitory concentration (IC₅₀) of the compound when the compound, or a pharmaceutical composition thereof, is contacted with the transcription factor (e.g., TEAD, such as TEAD1, TEAD2, TEAD3, TEAD4). The IC₅₀ values may be obtained using methods known in the art (e.g., by a competition binding assay). In certain embodiments, the IC₅₀ value of a compound described herein is not more than about 1 mM, not more than about 100 μM, not more than about 10 μM, not more than about 1 μM, not more than about 100 nM, not more than about 10 nM, or not more than about 1 nM.

The compounds described herein may selectively modulate the activity of a transcription factor (e.g., TEAD, such as TEAD1, TEAD2, TEAD3, TEAD4). In certain embodiments, the compounds selectively inhibit a transcription factor (e.g., TEAD, such as TEAD1, TEAD2, TEAD3, TEAD4). In certain embodiments, the compounds selectively inhibit the activity of a transcription factor (e.g., TEAD, such as TEAD1, TEAD2, TEAD3, TEAD4). In certain embodiments, the compounds inhibit the activity of two or more transcription factors (e.g., TEAD, such as TEAD1, TEAD2, TEAD3, TEAD4) to the same extent.

The selectivity of a compound described herein in inhibiting the activity of a first transcription factor (e.g., TEAD, such as TEAD1, TEAD2, TEAD3, TEAD4) over a second transcription factor different from the first transcription factor (e.g., a different TEAD) may be measured by the quotient of the IC₅₀ value of the compound in inhibiting the activity of the second transcription factor different from the first transcription factor (e.g., a different TEAD) over the IC₅₀ value of the compound in inhibiting the activity of the first transcription factor (e.g., TEAD). The selectivity of a compound described herein in modulating the activity of a first transcription factor (e.g., TEAD) over a second transcription factor different from the first transcription factor (e.g., a different TEAD) may also be measured by the quotient of the K_{d} value of an adduct of the compound and the second transcription factor different from the first transcription factor (e.g., a different TEAD) over the K_{d} value of an adduct of the compound and the first transcription factor different from the first transcription factor (e.g., a different TEAD). In certain embodiments, the selectivity is at least about 1-fold, at least about 3-fold, at least about 10-fold, at least about 30-fold, at least about 100-fold, at least about 300-fold, at least about 1,000-fold, at least about 3,000-fold, at least about 10,000-fold, at least about 30,000-fold, or at least about 100,000-fold. In certain embodiments, the selectivity is at least about 2-fold, about 5-fold, about 10-fold, or more.

It is expected that the compounds described herein may be useful in treating and/or preventing diseases associated with aberrant activity (e.g., increased activity, undesired activity, abnormal activity) of a transcription factor (e.g., TEAD, such as TEAD1, TEAD2, TEAD3, TEAD4). It is known in the art that transcription factors are implicated in a wide range of diseases and conditions, such as proliferative diseases, inflammatory diseases, and autoimmune diseases. Therefore, the compounds described herein are expected to be useful in treating and/or preventing diseases (e.g., proliferative diseases, inflammatory diseases, autoimmune diseases).

In certain embodiments, a compound of Formula (I″) is of the formula:

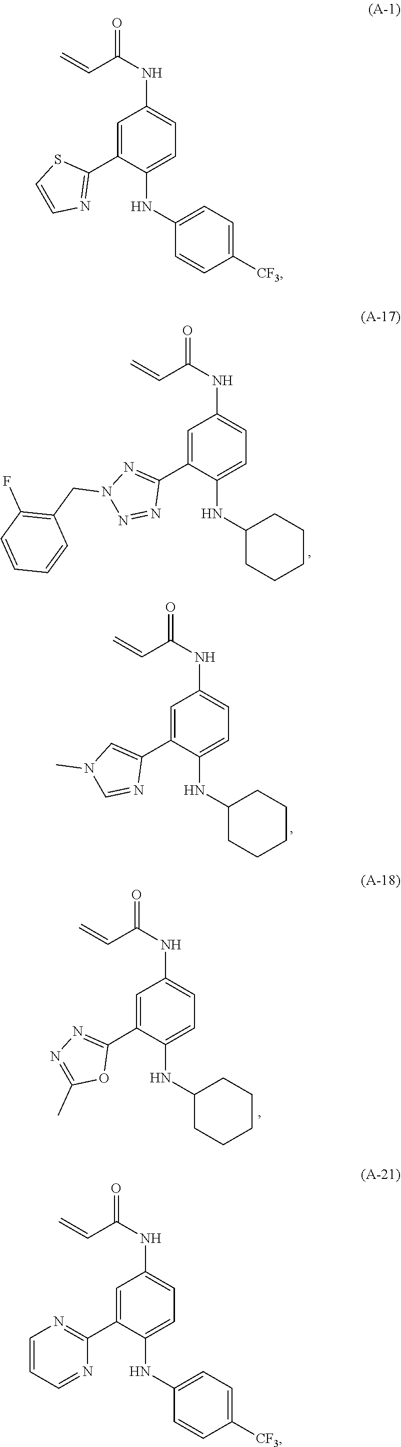

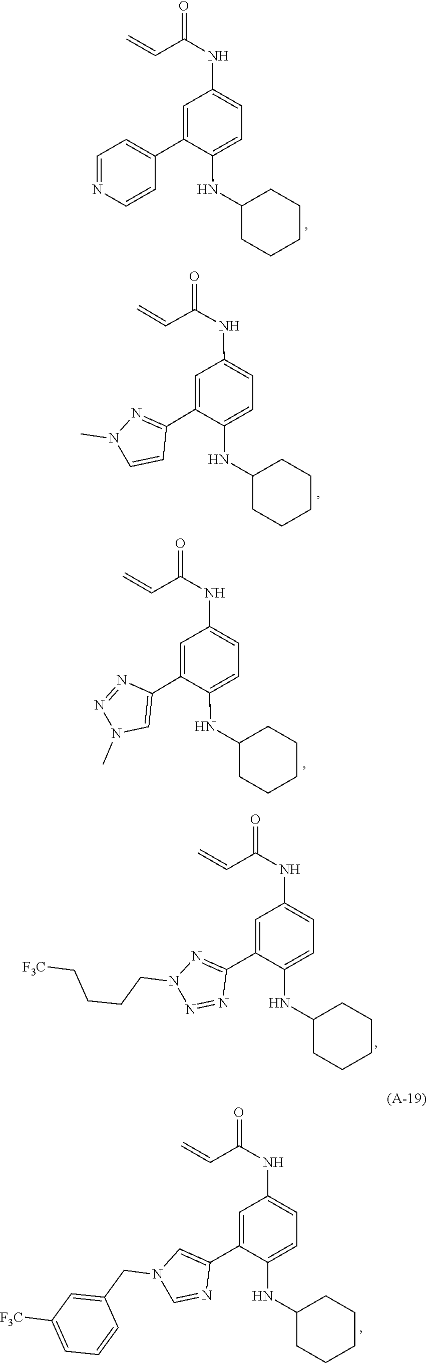

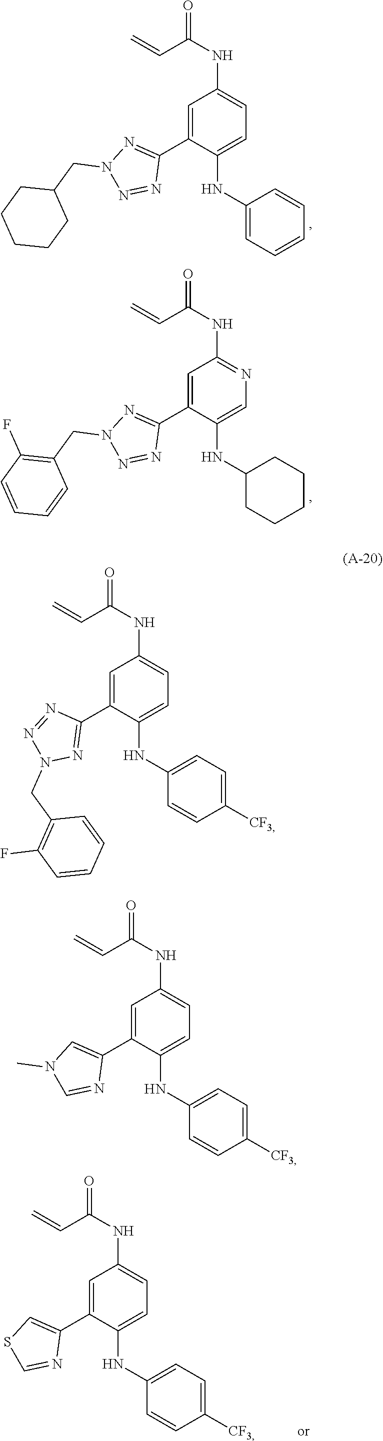

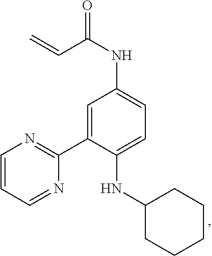 [SMILES: C=CC(=O)Nc1ccc(NC2CCCCC2)c(-c2ncccn2)c1]

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (I″) is of the formula:

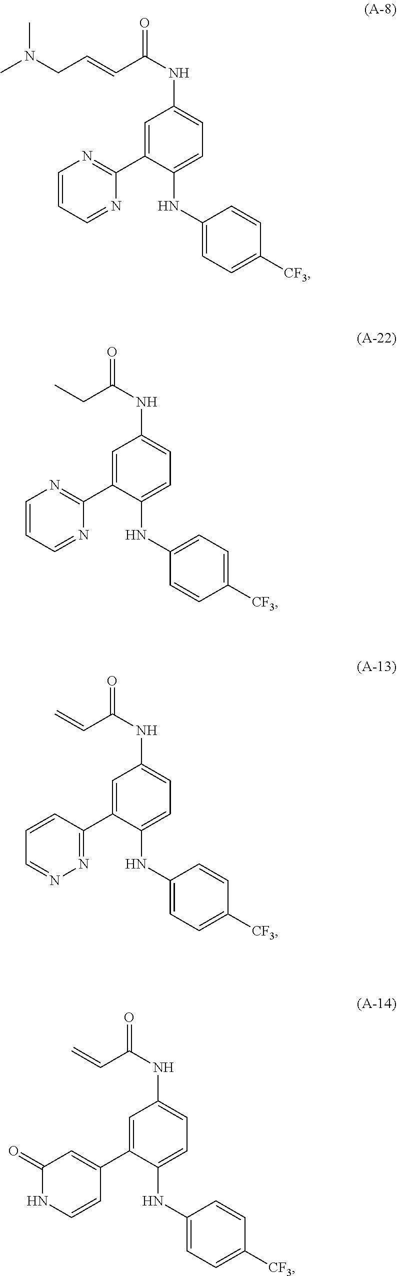 [SMILES: CN(C)C/C=C/C(=O)Nc1ccc(Nc2ccc(C(F)(F)F)cc2)c(-c2ncccn2)c1 ; CCC(=O)Nc1ccc(Nc2ccc(C(F)(F)F)cc2)c(-c2ncccn2)c1 ; C=CC(=O)Nc1ccc(Nc2ccc(C(F)(F)F)cc2)c(-c2cccnn2)c1 ; C=CC(=O)Nc1ccc(Nc2ccc(C(F)(F)F)cc2)c(-c2cc[nH]c(=O)c2)c1]

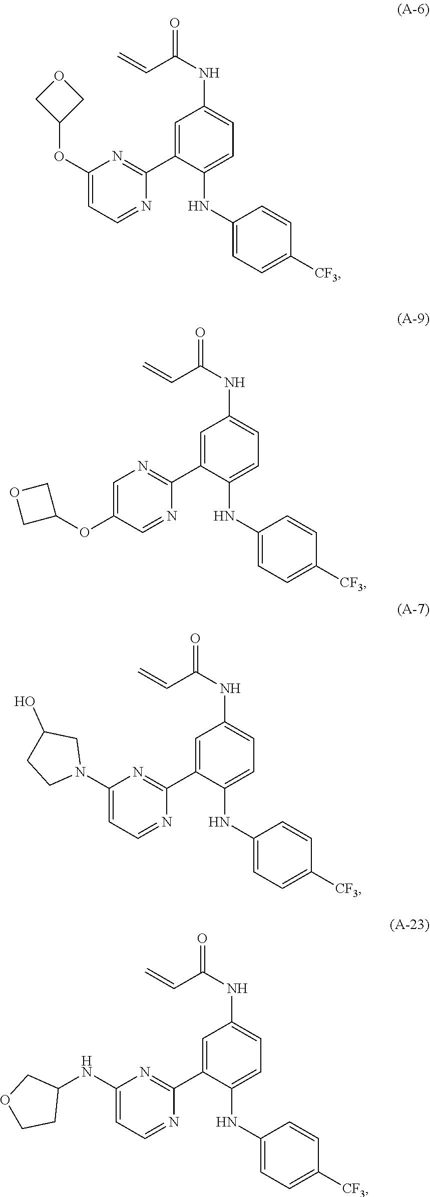 [SMILES: C=CC(=O)Nc1ccc(Nc2ccc(C(F)(F)F)cc2)c(-c2nccc(OC3COC3)n2)c1 ; C=CC(=O)Nc1ccc(Nc2ccc(C(F)(F)F)cc2)c(-c2ncc(OC3COC3)cn2)c1 ; C=CC(=O)Nc1ccc(Nc2ccc(C(F)(F)F)cc2)c(-c2nccc(N3CCC(O)C3)n2)c1 ; C=CC(=O)Nc1ccc(Nc2ccc(C(F)(F)F)cc2)c(-c2nccc(NC3CCOC3)n2)c1]

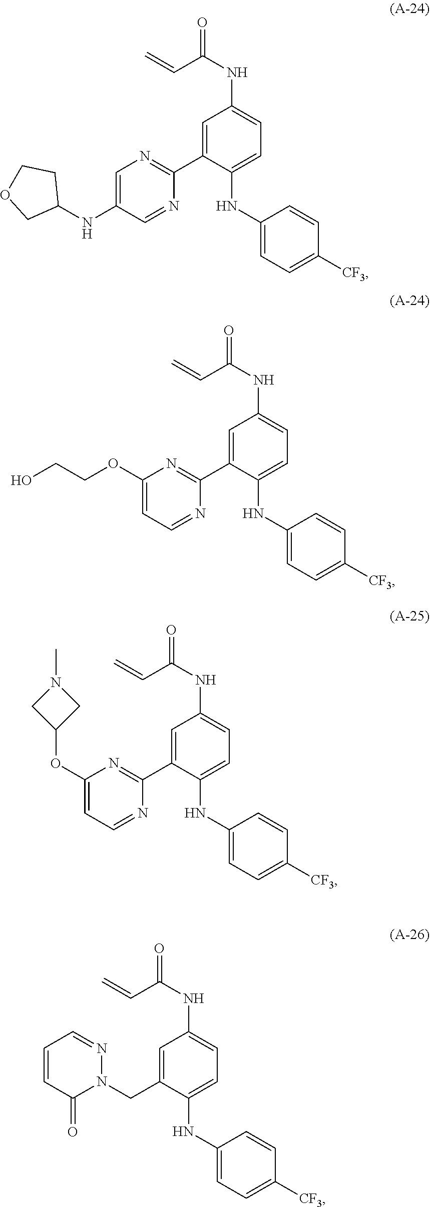 [SMILES: C=CC(=O)Nc1ccc(Nc2ccc(C(F)(F)F)cc2)c(-c2ncc(NC3CCOC3)cn2)c1 ; C=CC(=O)Nc1ccc(Nc2ccc(C(F)(F)F)cc2)c(-c2nccc(OCCO)n2)c1 ; C=CC(=O)Nc1ccc(Nc2ccc(C(F)(F)F)cc2)c(-c2nccc(OC3CN(C)C3)n2)c1 ; C=CC(=O)Nc1ccc(Nc2ccc(C(F)(F)F)cc2)c(Cn2ncccc2=O)c1]

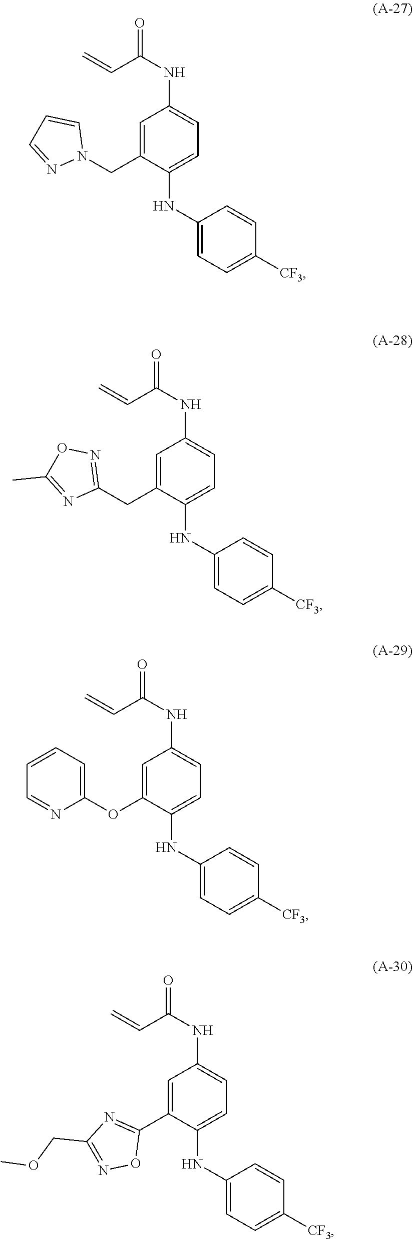 [SMILES: C=CC(=O)Nc1ccc(Nc2ccc(C(F)(F)F)cc2)c(Cn2cccn2)c1 ; C=CC(=O)Nc1ccc(Nc2ccc(C(F)(F)F)cc2)c(Cc2noc(C)n2)c1 ; C=CC(=O)Nc1ccc(Nc2ccc(C(F)(F)F)cc2)c(Oc2ccccn2)c1 ; C=CC(=O)Nc1ccc(Nc2ccc(C(F)(F)F)cc2)c(-c2nc(COC)no2)c1]

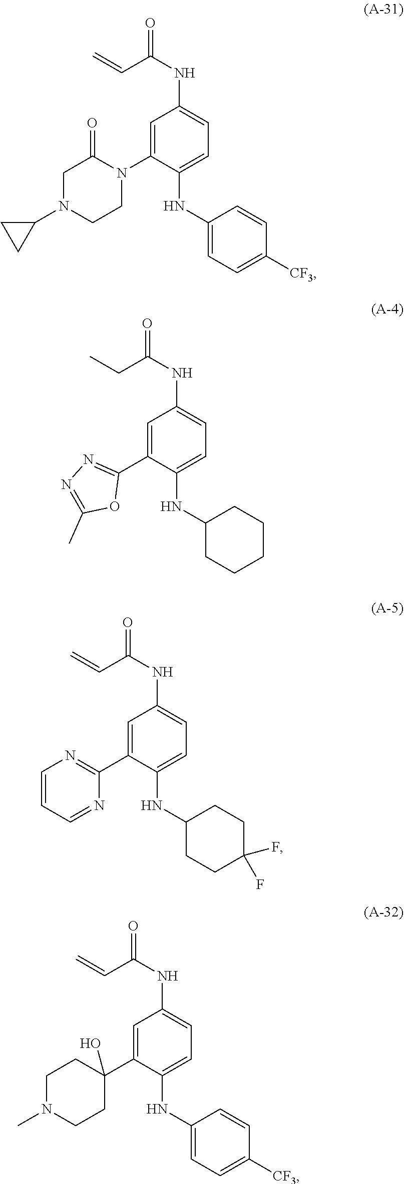 [SMILES: C=CC(=O)Nc1ccc(Nc2ccc(C(F)(F)F)cc2)c(N2CCN(C3CC3)CC2=O)c1 ; CCC(=O)Nc1ccc(NC2CCCCC2)c(-c2nnc(C)o2)c1 ; C=CC(=O)Nc1ccc(NC2CCC(F)(F)CC2)c(-c2ncccn2)c1 ; C=CC(=O)Nc1ccc(Nc2ccc(C(F)(F)F)cc2)c(C2(O)CCN(C)CC2)c1]

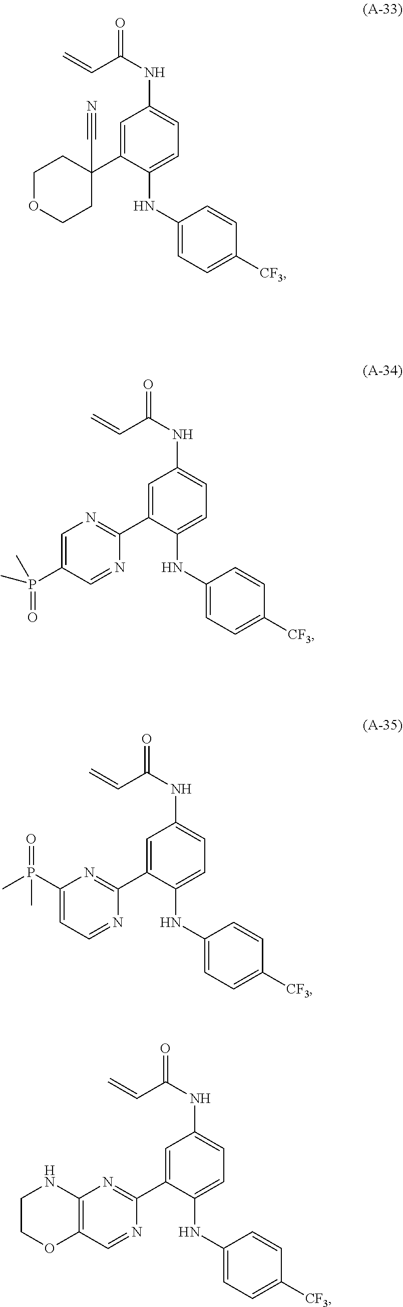 [SMILES: C=CC(=O)Nc1ccc(Nc2ccc(C(F)(F)F)cc2)c(C2(C#N)CCOCC2)c1 ; C=CC(=O)Nc1ccc(Nc2ccc(C(F)(F)F)cc2)c(-c2ncc(P(C)(C)=O)cn2)c1 ; C=CC(=O)Nc1ccc(Nc2ccc(C(F)(F)F)cc2)c(-c2nccc(P(C)(C)=O)n2)c1 ; C=CC(=O)Nc1ccc(Nc2ccc(C(F)(F)F)cc2)c(-c2ncc3c(n2)NCCO3)c1]

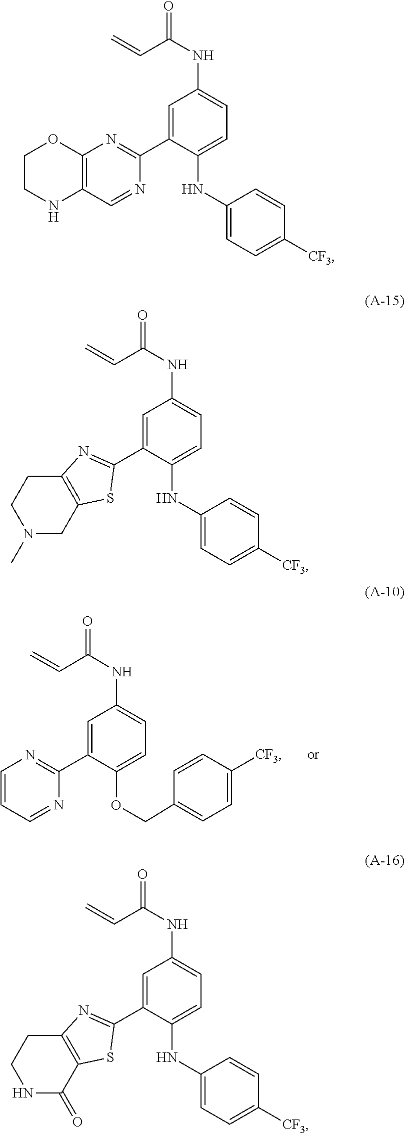 [SMILES: C=CC(=O)Nc1ccc(Nc2ccc(C(F)(F)F)cc2)c(-c2ncc3c(n2)OCCN3)c1 ; C=CC(=O)Nc1ccc(Nc2ccc(C(F)(F)F)cc2)c(-c2nc3c(s2)CN(C)CC3)c1 ; C=CC(=O)Nc1ccc(OCc2ccc(C(F)(F)F)cc2)c(-c2ncccn2)c1 ; C=CC(=O)Nc1ccc(Nc2ccc(C(F)(F)F)cc2)c(-c2nc3c(s2)C(=O)NCC3)c1]

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (I″) is of the formula:

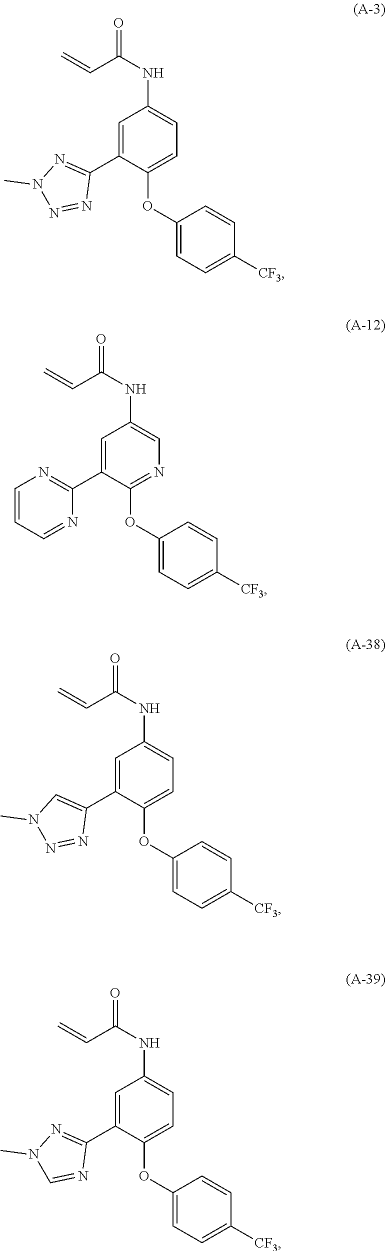 [SMILES: C=CC(=O)Nc1ccc(Oc2ccc(C(F)(F)F)cc2)c(-c2nnn(C)n2)c1 ; C=CC(=O)Nc1cnc(Oc2ccc(C(F)(F)F)cc2)c(-c2ncccn2)c1 ; C=CC(=O)Nc1ccc(Oc2ccc(C(F)(F)F)cc2)c(-c2cn(C)nn2)c1 ; C=CC(=O)Nc1ccc(Oc2ccc(C(F)(F)F)cc2)c(-c2ncn(C)n2)c1]

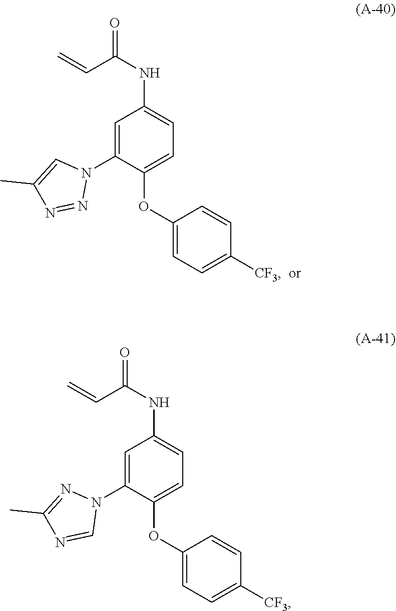

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (II′) is of the formula:

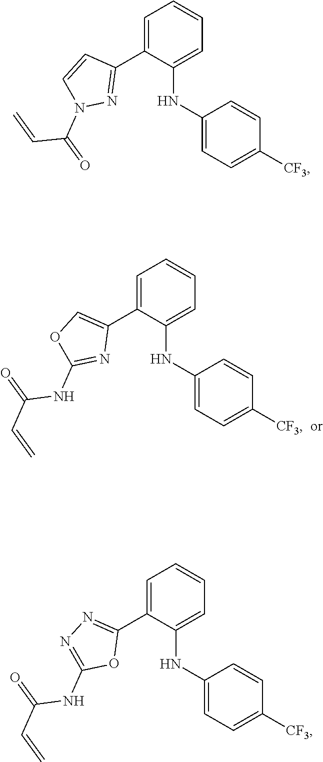

or a pharmaceutically acceptable salt, solvate, hydrate, polymorph, co-crystal, tautomer, stereoisomer, isotopically labeled derivative, prodrug, composition, or mixture thereof.

### Pharmaceutical Compositions, Kits, and Administration

The present disclosure also provides pharmaceutical compositions comprising a compound described herein and optionally a pharmaceutically acceptable excipient. In certain embodiments, a compound described herein is a compound of Formula (I″), Formula (I′), Formula (I), Formula (II′), or Formula (II), or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for inhibiting the aberrant activity of a transcription factor (e.g., TEAD, such as TEAD1, TEAD2, TEAD3, TEAD4). In certain embodiments, a therapeutically effective amount is an amount effective for treating a disease (e.g., a disease associated with aberrant activity of a transcription factor (e.g., TEAD (e.g., proliferative diseases, inflammatory diseases, autoimmune diseases)). In certain embodiments, a therapeutically effective amount is an amount effective for inhibiting the aberrant activity of a transcription factor (e.g., TEAD, such as TEAD1, TEAD2, TEAD3, TEAD4) and treating a disease (e.g., a disease associated with aberrant activity of a transcription factor (e.g., TEAD, such as TEAD1, TEAD2, TEAD3, TEAD4) (e.g., proliferative disease, inflammatory disease, autoimmune disease))). In certain embodiments, a therapeutically effective amount is an amount effective for inhibiting the transcription of a gene (e.g., a gene controlled or regulated by a transcription factor (e.g., TEAD, such as TEAD1, TEAD2, TEAD3, TEAD4)) in a subject and/or biological sample. In certain embodiments, a prophylactically effective amount is an amount effective for inhibiting the aberrant activity of a transcription factor (e.g., TEAD, such as TEAD1, TEAD2, TEAD3, TEAD4). In certain embodiments, a prophylactically effective amount is an amount effective for preventing or keeping a subject in need thereof in remission of a disease (e.g., a disease associated with aberrant activity of a transcription factor (e.g., TEAD) (e.g., proliferative disease, inflammatory disease, autoimmune disease)). In certain embodiments, a prophylactically effective amount is an amount effective for inhibiting the aberrant activity of a transcription factor (e.g., TEAD, such as TEAD1, TEAD2, TEAD3, TEAD4), and preventing or keeping a subject in need thereof in remission of a disease (e.g., a disease associated with aberrant activity of a transcription factor (e.g., TEAD, such as TEAD1, TEAD2, TEAD3, TEAD4) (e.g., proliferative disease, inflammatory disease, autoimmune disease)). In certain embodiments, a prophylactically effective amount is an amount effective for inhibiting the transcription of a gene (e.g., a gene controlled or regulated by a transcription factor (e.g., TEAD, such as TEAD1, TEAD2, TEAD3, TEAD4)) in a subject and/or biological sample.

In certain embodiments, the effective amount is an amount effective for inhibiting the activity of a transcription factor (e.g., TEAD, such as TEAD1, TEAD2, TEAD3, TEAD4) by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a transcription factor (e.g., TEAD, such as TEAD1, TEAD2, TEAD3, TEAD4) by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for increasing the activity of a transcription factor (e.g., TEAD, such as TEAD1, TEAD2, TEAD3, TEAD4) by at least 10%, at least 20%, at least 30%, at least 40%, at least 50%, at least 60%, at least 70%, at least 80%, at least 90%, at least 95%, or at least 98%. In certain embodiments, the effective amount is an amount effective for increasing the activity of a transcription factor (e.g., TEAD, such as TEAD1, TEAD2, TEAD3, TEAD4) by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or pharmaceutical composition thereof described herein is in vitro. In certain embodiments, the cell being contacted with a compound or pharmaceutical composition thereof described herein is in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragées, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample (e.g., tissue, cell), any two doses of the multiple doses include different or substantially the same amounts of a compound described herein. In certain embodiments, when multiple doses are administered to a subject or applied to a biological sample (e.g., tissue, cell), the frequency of administering the multiple doses to the subject or applying the multiple doses to the biological sample (e.g., tissue, cell) is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample (e.g., tissue, cell) is one dose per day. In certain embodiments, the frequency of administering the multiple doses to the subject or applying the multiple doses to the biological sample (e.g., tissue, cell) is two doses per day. In certain embodiments, the frequency of administering the multiple doses to the subject or applying the multiple doses to the biological sample (e.g., tissue, cell) is three doses per day. In certain embodiments, when multiple doses are administered to a subject or applied to a biological sample (e.g., tissue, cell), the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or pharmaceutical composition thereof, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity of a transcription factor (e.g., TEAD, such as TEAD1, TEAD2, TEAD3, TEAD4)) in a subject and/or biological sample (e.g., tissue, cell)), improve bioavailability, improve safety, reduce drug resistance, reduce and/or modify metabolism, inhibit excretion, and/or modify distribution in a subject and/or biological sample (e.g., tissue, cell). It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compounds described herein and the additional pharmaceutical agent, but not both.

The compound or pharmaceutical composition thereof can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, inflammatory disease, autoimmune disease). Each additional pharmaceutical agent may be administered at a dose and/or on a time schedule determined for that pharmaceutical agent. The additional pharmaceutical agents may also be administered together with each other and/or with the compound or pharmaceutical composition thereof described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine 1131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is ibrutinib. In certain embodiments, the additional pharmaceutical agent is a transcription factor inhibitor (e.g., inhibitor of EGFR and/or MEK). In certain embodiments, the additional pharmaceutical agent is an inhibitor of a gene and/or protein in the Hippo signaling pathway. In certain embodiments, the additional pharmaceutical agent is an inhibitor of EGFR (e.g., osimertinib, gefitinib) and/or an inhibitor of MEK (e.g., trametinib, selumetinib). In certain embodiments, the additional pharmaceutical agent is an inhibitor of EGFR (e.g., osimertinib, gefitinib). In certain embodiments, the additional pharmaceutical agent comprises an inhibitor of MEK (e.g., trametinib, selumetinib). In certain embodiments, the additional pharmaceutical agent comprises an inhibitor of tankyrase inhibitor and/or an indirect inhibitor of YAP (e.g., compound XAV939). In certain embodiments, the additional pharmaceutical agent is an anti-proliferative agent (e.g., anti-cancer agent, such as an inhibitor of EGFR, an inhibitor of MEK, or an inhibitor of EGFR and an inhibitor of MEK). In certain embodiments, the additional pharmaceutical agent is a transcription factor inhibitor (e.g., inhibitor of EGFR and/or MEK). In certain embodiments, the additional pharmaceutical agent is an agent for treating lung cancer (e.g., non-small cell lung cancer (NSCLC)). In certain embodiments, the additional pharmaceutical agent is an agent for treating lung cancer (e.g., non-small cell lung cancer (NSCLC)), such as NSCLC with a mutation in a gene and/or protein in the Hippo signaling pathway (e.g., mutation in EGFR). In certain embodiments, the additional pharmaceutical agent is a kinase inhibitor. In certain embodiments, the additional pharmaceutical agent is a tyrosine kinase inhibitor (TKI). In certain embodiments, the additional pharmaceutical agent is an agent for treating a cancer that has a mutation in a gene of the Hippo signaling pathway. In certain embodiments, the additional pharmaceutical agent is an agent for treating a cancer that has a mutation in EGFR. In certain embodiments, the additional pharmaceutical agent is an agent for treating a cancer that has a mutation in MEK. In certain embodiments, the additional pharmaceutical agent is an agent for treating a cancer that is an EGFR-mutant non-small cell lung cancer. In certain embodiments, the additional pharmaceutical agent is an agent for treating a cancer that is resistant to certain anti-proliferative agents (e.g., cancers resistant to inhibitors of EGFR, such as osimertinib, and/or inhibitors of MEK, such as trametinib). In certain embodiments, the additional pharmaceutical agent is an agent for treating a cancer that is resistant to inhibitors of EGFR and/or MEK. In certain embodiments, the additional pharmaceutical agent is an agent for treating a cancer that is resistant to osimertinib and trametinib. In certain embodiments, the additional pharmaceutical agent is an agent for treating a cancer that is resistant to tyrosine kinase inhibitors (TKI's).

In certain embodiments, the additional pharmaceutical agent is a binder or inhibitor of TEAD (e.g., TEAD1, TEAD2, TEAD3, TEAD4)). In certain embodiments, the additional pharmaceutical agent is a binder or inhibitor of TEAD. In certain embodiments, the additional pharmaceutical agent is a binder or inhibitor of TEAD1. In certain embodiments, the additional pharmaceutical agent is a binder or inhibitor of TEAD2. In certain embodiments, the additional pharmaceutical agent is a binder or inhibitor of TEAD3. In certain embodiments, the additional pharmaceutical agent is a binder or inhibitor of TEAD4. In certain embodiments, the additional pharmaceutical agent is a binder or inhibitor of Bruton's tyrosine kinase (BTK). In certain embodiments, the additional pharmaceutical agent is selected from the group consisting of epigenetic and transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, inflammatory disease, autoimmune disease) in a subject in need thereof. In certain embodiments, the kits are useful for preventing a disease (e.g., proliferative disease, inflammatory disease, autoimmune disease) in a subject in need thereof. In certain embodiments, the kits are useful for inhibiting the activity (e.g., aberrant or unwanted activity, such as increased activity) of a transcription factor (e.g., TEAD, such as TEAD1, TEAD2, TEAD3, TEAD4) in a subject and/or biological sample (e.g., tissue, cell). In certain embodiments, the kits are useful for inhibiting the transcription of a gene (e.g., a gene controlled or regulated by a transcription factor (e.g., TEAD, such as TEAD1, TEAD2, TEAD3, TEAD4)) in a subject and/or biological sample.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, inflammatory disease, autoimmune disease) in a subject in need thereof. In certain embodiments, the kits and instructions provide for preventing a disease (e.g., proliferative disease, inflammatory disease, autoimmune disease) in a subject in need thereof. In certain embodiments, the kits and instructions provide for modulating (e.g., inhibiting) the activity (e.g., aberrant activity, such as increased activity) of a transcription factor (e.g., TEAD, such as TEAD1, TEAD2, TEAD3, TEAD4) in a subject and/or biological sample (e.g., tissue, cell). In certain embodiments, the kits and instructions provide for inhibiting the transcription of a gene (e.g., a gene controlled or regulated by a transcription factor (e.g., TEAD, such as TEAD1, TEAD2, TEAD3, TEAD4)) in a subject and/or biological sample. A kit described herein may include one or more additional pharmaceutical agents described herein as a separate composition.

### Methods of Treatment and Uses

The present disclosure provides methods of modulating (e.g., inhibiting or increasing) the activity (e.g., aberrant activity, such as increased or decreased activity) of a transcription factor (e.g., TEAD, such as TEAD1, TEAD2, TEAD3, TEAD4) using compounds described herein, which may be optionally administered in combination with an additional pharmaceutical agent, for example, modulators of other transcription factors (e.g., YAP, EGFR, MEK). The present disclosure provides methods of modulating (e.g., inhibiting or increasing) the activity (e.g., aberrant activity, such as increased or decreased activity) of a transcription factor (e.g., TEAD, such as TEAD1, TEAD2, TEAD3, TEAD4) in a subject and/or biological sample (e.g., tissue, cell), using compounds described herein, which may be optionally administered in combination with an additional pharmaceutical agent, for example, modulators of other transcription factors (e.g., YAP, EGFR, MEK). The present disclosure also provides methods for the treatment of a wide range of diseases, such as diseases associated with the aberrant activity (e.g., increased activity) of a transcription factor (e.g., TEAD, such as TEAD1, TEAD2, TEAD3, TEAD4), using compounds described herein, which may be optionally administered in combination with an additional pharmaceutical agent, for example, modulators of other transcription factors (e.g., YAP, EGFR, MEK), for example, for treating proliferative diseases, inflammatory diseases, and/or autoimmune diseases in a subject in need thereof. The present disclosure provides methods for the treatment and/or prevention of a proliferative disease (e.g., cancers (e.g., carcinoma, sarcoma); lung cancer, breast cancer, liver cancer, pancreatic cancer, gastric cancer, ovarian cancer, colon cancer, colorectal cancer, skin cancer, esophageal cancer)), inflammatory disease (e.g., fibrosis), or autoimmune disease (e.g., sclerosis), using compounds described herein, which may be optionally administered in combination with an additional pharmaceutical agent, for example, modulators of other transcription factors (e.g., YAP, EGFR, MEK). The present disclosure provides methods for inhibiting the transcription of a gene (e.g., a gene controlled or regulated by a transcription factor (e.g., TEAD, such as TEAD1, TEAD2, TEAD3, TEAD4)) in a subject and/or biological sample (e.g., tissue, cell), using compounds described herein, which may be optionally administered in combination with an additional pharmaceutical agent, for example, modulators of other transcription factors (e.g., YAP, EGFR, MEK).

The present disclosure also provides a compound of Formula (I″), Formula (I′), Formula (I), Formula (II′), or Formula (II), or a pharmaceutically acceptable salt, solvate, hydrate, polymorph, co-crystal, tautomer, stereoisomer, isotopically labeled derivative, prodrug, or pharmaceutical composition thereof, which may be optionally administered in combination with an additional pharmaceutical agent, for example, modulators of other transcription factors (e.g., YAP, EGFR, MEK), for use in the treatment of diseases, such as proliferative diseases, inflammatory diseases, and autoimmune diseases, in a subject in need thereof.

The present disclosure also provides uses of a compound of Formula (I″), Formula (I′), Formula (I), Formula (II′), or Formula (II), or a pharmaceutically acceptable salt, solvate, hydrate, polymorph, co-crystal, tautomer, stereoisomer, isotopically labeled derivative, prodrug, or pharmaceutical composition thereof, which may be optionally administered in combination with an additional pharmaceutical agent, for example, modulators of other transcription factors (e.g., YAP, EGFR, MEK), in the manufacture of a medicament for the treatment of various diseases, such as proliferative diseases, inflammatory diseases, and autoimmune diseases, in a subject in need thereof.

In another aspect, the present disclosure provides methods of modulating the activity of a transcription factor (e.g., TEAD, such as TEAD1, TEAD2, TEAD3, TEAD4) in a subject and/or biological sample (e.g., cell, tissue) using compounds described herein, which may be optionally administered in combination with an additional pharmaceutical agent, for example, modulators of other transcription factors (e.g., YAP, EGFR, MEK). In certain embodiments, provided are methods of inhibiting the activity of a transcription factor (e.g., TEAD) in a subject. In certain embodiments, provided are methods of inhibiting the activity of a transcription factor (e.g., TEAD) in a cell. In certain embodiments, provided are methods of increasing the activity of a transcription factor (e.g., TEAD, such as TEAD1, TEAD2, TEAD3, TEAD4) in a subject. The compounds described herein may exhibit transcription factor inhibitory activity; the ability to inhibit TEAD; the ability to inhibit TEAD1, without inhibiting another transcription factor (e.g., a different TEAD); the ability to inhibit TEAD2, without inhibiting another transcription factor (e.g., a different TEAD); the ability to inhibit TEAD3, without inhibiting another transcription factor (e.g., a different TEAD); the ability to inhibit TEAD4, without inhibiting another transcription factor (e.g., a different TEAD); a therapeutic effect and/or preventative effect in the treatment of cancers; a therapeutic effect and/or preventative effect in the treatment of proliferative diseases, inflammatory diseases, and/or autoimmune diseases; and/or a therapeutic profile (e.g., optimum safety and curative effect) that is superior to existing chemotherapeutic agents, or agents for treating inflammatory diseases and/or autoimmune diseases.

In certain embodiments, provided are methods of decreasing the activity of a transcription factor (e.g., TEAD, such as TEAD1, TEAD2, TEAD3, TEAD4) in a subject or biological sample (e.g., cell, tissue) by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a transcription factor (e.g., TEAD, such as TEAD1, TEAD2, TEAD3, TEAD4) in a subject or biological sample (e.g., cell, tissue) is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a transcription factor (e.g., TEAD, such as TEAD1, TEAD2, TEAD3, TEAD4) in a subject or biological sample (e.g., cell, tissue) is selectively inhibited by the compound. In some embodiments, the activity of a transcription factor (e.g., TEAD, such as TEAD1, TEAD2, TEAD3, TEAD4) in a subject or biological sample (e.g., cell, tissue) is selectively decreased by the compound.

Without wishing to be bound by any particular theory, the compounds described herein are able to bind (e.g., covalently modify) the transcription factor being inhibited. In certain embodiments, a compound described herein is able to bind (e.g., covalently modify) the transcription factor. In certain embodiments, the compound described herein is able to covalently bind a cysteine residue of the transcription factor. In certain embodiments, the compound described herein is able to covalently bind a cysteine residue of TEAD. In certain embodiments, the compound described herein is able to covalently bind a cysteine residue of TEAD1. In certain embodiments, the compound is capable of covalently binding cysteine 359 of TEAD1. In certain embodiments, the compound described herein is able to covalently bind a cysteine residue of TEAD2. In certain embodiments, the compound is capable of covalently binding cysteine 380 of TEAD2. In certain embodiments, the compound is capable of covalently binding TEAD1. In certain embodiments, the compound is capable of covalently binding TEAD2. In certain embodiments, the compound is capable of covalently binding TEAD3. In certain embodiments, the compound described herein is able to covalently bind a cysteine residue of TEAD4. In certain embodiments, the compound is capable of covalently binding TEAD4. In certain embodiments, the compound is capable of binding the YAP/TAZ domain of a TEAD family transcription factor. In certain embodiments, the compound is capable of covalently modifying TEAD1 (e.g., C359 of TEAD1). In certain embodiments, the compound is capable of covalently modifying TEAD2 (e.g., C380 of TEAD2). In certain embodiments, the compound is capable of covalently modifying C359 (cysteine 359) of TEAD1. In certain embodiments, the compound is capable of covalently modifying C380 (cysteine 380) of TEAD2. In certain embodiments, the compound is capable of covalently modifying TEAD3. In certain embodiments, the compound is capable of covalently modifying TEAD4. In certain embodiments, the compound is capable of covalently modifying TEAD1. In certain embodiments, the compound is capable of covalently modifying TEAD2. In certain embodiments, the compound is capable of covalently modifying TEAD3. In certain embodiments, the compound is capable of covalently modifying TEAD4. In certain embodiments, the compound is capable of non-covalently inhibiting TEAD1. In certain embodiments, the compound is capable of non-covalently inhibiting TEAD2. In certain embodiments, the compound is capable of non-covalently inhibiting TEAD3. In certain embodiments, the compound is capable of non-covalently inhibiting TEAD4.

In another aspect, the present disclosure provides methods of inhibiting a transcription factor (e.g., TEAD, such as TEAD1, TEAD2, TEAD3, TEAD4) in a subject, the methods comprising administering to the subject an effective amount (e.g., therapeutically effective amount) of a compound, or pharmaceutical composition thereof, as described herein. In another aspect, the present disclosure provides methods of inhibiting the activity of a transcription factor (e.g., TEAD, such as TEAD1, TEAD2, TEAD3, TEAD4) in a subject, the methods comprising administering to the subject an effective amount (e.g., therapeutically effective amount) of a compound, or pharmaceutical composition thereof, as described herein. In another aspect, the present disclosure provides methods of inhibiting the activity of a transcription factor (e.g., TEAD, such as TEAD1, TEAD2, TEAD3, TEAD4) in a biological sample, the methods comprising contacting the biological sample with an effective amount of a compound, or pharmaceutical composition thereof, as described herein. In another aspect, the present disclosure provides methods of inhibiting the activity of a transcription factor (e.g., TEAD, such as TEAD1, TEAD2, TEAD3, TEAD4) in a biological sample (e.g., tissue, cell), the methods comprising contacting the biological sample (e.g., tissue, cell) with an effective amount of a compound, or pharmaceutical composition thereof, as described herein.

In another aspect, the present disclosure provides methods of inhibiting the activity of a transcription factor (e.g., TEAD, such as TEAD1, TEAD2, TEAD3, TEAD4) in a cell, the methods comprising contacting the cell with an effective amount of a compound, or pharmaceutical composition thereof, as described herein.

In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a subject, the methods comprising administering to the subject an effective amount (e.g., therapeutically effective amount) of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a subject, the methods comprising administering to the subject an effective amount (e.g., therapeutically effective amount) of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a biological sample, the methods comprising contacting the biological sample with an effective amount of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a biological sample (e.g., tissue, cell), the methods comprising contacting the biological sample (e.g., tissue, cell) with an effective amount of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a cell, the methods comprising contacting the cell with an effective amount of a compound, or pharmaceutical composition thereof, as described herein.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal, such as a transgenic mouse or transgenic pig.

In certain embodiments, the biological sample being contacted with the compound or pharmaceutical composition thereof is breast tissue, bone marrow, lymph node, lymph tissue, spleen, or blood. In certain embodiments, the biological sample being contacted with the compound or pharmaceutical composition thereof is a tumor or cancerous tissue. In certain embodiments, the biological sample being contacted with the compound or pharmaceutical composition thereof is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from the biological sample.

In certain embodiments, the cell or tissue being contacted with the compound or pharmaceutical composition thereof is present in vitro. In certain embodiments, the cell or tissue being contacted with the compound or pharmaceutical composition thereof is present in vivo. In certain embodiments, the cell or tissue being contacted with the compound or pharmaceutical composition thereof is present ex vivo. In certain embodiments, the cell or tissue being contacted with the compound or pharmaceutical composition thereof is a malignant cell (e.g., malignant blood cell). In certain embodiments, the cell being contacted with the compound or pharmaceutical composition thereof is a malignant hematopoietic stem cell (e.g., malignant myeloid cell or malignant lymphoid cell). In certain embodiments, the cell being contacted with the compound or pharmaceutical composition thereof is a malignant lymphocyte (e.g., malignant T-cell or malignant B-cell). In certain embodiments, the cell being contacted with the compound or pharmaceutical composition thereof is a malignant white blood cell. In certain embodiments, the cell being contacted with the compound or pharmaceutical composition thereof is a malignant neutrophil, malignant macrophage, or malignant plasma cell. In certain embodiments, the cell being contacted with the compound or pharmaceutical composition thereof is a carcinoma cell. In certain embodiments, the cell being contacted with the compound or pharmaceutical composition thereof is a breast carcinoma cell. In certain embodiments, the cell being contacted with the compound or pharmaceutical composition thereof is a sarcoma cell. In certain embodiments, the cell being contacted with the compound or pharmaceutical composition thereof is a sarcoma cell from breast tissue. In certain embodiments, the biological sample is from tissue or cells with cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma). In certain embodiments, the biological sample is from tissue or cells with an inflammatory disease or autoimmune disease. In certain embodiments, the biological sample is from tissue or cells with cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma), an inflammatory disease, or an autoimmune disease.

The disease (e.g., proliferative disease, inflammatory disease, autoimmune disease) to be treated or prevented using the compounds described herein may be associated with increased activity of a transcription factor, such as TEAD (e.g., TEAD1, TEAD2, TEAD3, TEAD4). The disease (e.g., proliferative disease, inflammatory disease, autoimmune disease) to be treated or prevented using the compounds described herein may be associated with the overexpression of a transcription factor, such as TEAD (e.g., TEAD1, TEAD2, TEAD3, TEAD4).

In certain embodiments, the disease (e.g., proliferative disease, inflammatory disease, autoimmune disease) to be treated or prevented using the compounds described herein may be associated with the overexpression of a transcription factor (e.g., TEAD, such as TEAD1, TEAD2, TEAD3, TEAD4). A disease (e.g., proliferative disease, inflammatory disease, autoimmune disease) may be associated with aberrant activity of a transcription factor (e.g., TEAD, such as TEAD1, TEAD2, TEAD3, TEAD4). Aberrant activity of a transcription factor (e.g., TEAD, such as TEAD1, TEAD2, TEAD3, TEAD4) may be elevated and/or inappropriate or undesired activity of the transcription factor (e.g., TEAD). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a transcription factor (e.g., TEAD, such as TEAD1, TEAD2, TEAD3, TEAD4) and be useful in treating and/or preventing diseases (e.g., proliferative diseases, inflammatory diseases, autoimmune diseases). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a transcription factor (e.g., TEAD) and be useful in treating and/or preventing diseases (e.g., proliferative disease, inflammatory disease, autoimmune disease). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a transcription factor (e.g., TEAD) and be useful in treating and/or preventing a disease (e.g., proliferative disease, inflammatory disease, autoimmune disease).

All types of biological samples described herein or known in the art are contemplated as being within the scope of the invention. In certain embodiments, the disease (e.g., proliferative disease, inflammatory disease, autoimmune disease) to be treated or prevented using the compounds described herein is cancer. All types of cancers disclosed herein or known in the art are contemplated as being within the scope of the invention. In certain embodiments, the proliferative disease is a hematological malignancy. In certain embodiments, the proliferative disease is a blood cancer. In certain embodiments, the proliferative disease is a hematological malignancy. In certain embodiments, the proliferative disease is leukemia. In certain embodiments, the proliferative disease is chronic lymphocytic leukemia (CLL). In certain embodiments, the proliferative disease is acute lymphoblastic leukemia (ALL). In certain embodiments, the proliferative disease is T-cell acute lymphoblastic leukemia (T-ALL). In certain embodiments, the proliferative disease is chronic myelogenous leukemia (CML). In certain embodiments, the proliferative disease is acute myeloid leukemia (AML). In certain embodiments, the proliferative disease is acute monocytic leukemia (AMoL). In certain embodiments, the proliferative disease is Waldenström's macroglobulinemia. In certain embodiments, the proliferative disease is Waldenström's macroglobulinemia associated with the MYD88 L265P somatic mutation. In certain embodiments, the proliferative disease is myelodysplastic syndrome (MDS). In certain embodiments, the proliferative disease is a carcinoma. In certain embodiments, the proliferative disease is lymphoma. In certain embodiments, the proliferative disease is T-cell lymphoma. In some embodiments, the proliferative disease is Burkitt's lymphoma. In certain embodiments, the proliferative disease is a Hodgkin's lymphoma. In certain embodiments, the proliferative disease is a non-Hodgkin's lymphoma. In certain embodiments, the proliferative disease is multiple myeloma. In certain embodiments, the proliferative disease is melanoma. In certain embodiments, the proliferative disease is colorectal cancer. In certain embodiments, the proliferative disease is colon cancer. In certain embodiments, the proliferative disease is breast cancer. In certain embodiments, the proliferative disease is recurring breast cancer. In certain embodiments, the proliferative disease is mutant breast cancer. In certain embodiments, the proliferative disease is HER2+ breast cancer. In certain embodiments, the proliferative disease is HER2− breast cancer. In certain embodiments, the proliferative disease is triple-negative breast cancer (TNBC). In certain embodiments, the proliferative disease is a bone cancer. In certain embodiments, the proliferative disease is osteosarcoma. In certain embodiments, the proliferative disease is Ewing's sarcoma. In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is non-small cell lung cancer (NSCLC). In certain embodiments, the lung cancer is mesothelioma. In certain embodiments, the cancer is a thyroid cancer. In certain embodiments, the cancer is a sarcoma. In certain embodiments, the sarcoma is Kaposi's sarcoma. In certain embodiments, the cancer is fallopian tube cancer. In certain embodiments, the cancer is a carcinoma. In certain embodiments, the carcinoma is fallopian tube carcinoma. In some embodiments, the proliferative disease is liver cancer. In some embodiments, the proliferative disease is prostate cancer. In some embodiments, the proliferative disease is pancreatic cancer. In some embodiments, the proliferative disease is gastric cancer. In some embodiments, the proliferative disease is ovarian cancer. In some embodiments, the proliferative disease is ovarian cancer. In some embodiments, the cancer is skin cancer. In some embodiments, the cancer is esophageal cancer. In certain embodiments, the cancer has a mutation in a gene of the Hippo signaling pathway. In certain embodiments, the cancer has a mutation in EGFR. In certain embodiments, the cancer has a mutation in MEK. In certain embodiments, the cancer is an EGFR-mutant non-small cell lung cancer. In certain embodiments, the cancer is resistant to certain anti-proliferative agents (e.g., cancers resistant to inhibitors of EGFR and/or MEK). In certain embodiments, the cancer is resistant to inhibitors of EGFR and/or MEK. In certain embodiments, the cancer is resistant to tyrosine kinase inhibitors (TKI's). In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention. In certain embodiments, the cancer is a sarcoma, lung cancer, thyroid cancer, breast cancer, liver cancer, pancreatic cancer, gastric cancer, ovarian cancer, colon cancer, colorectal cancer, skin cancer, esophageal cancer; a carcinoma; has a mutation in a gene of the Hippo signaling pathway (e.g., has a mutation in EGFR, such as an EGFR-mutant non-small cell lung cancer, or has a mutation in MEK), is a cancer resistant to certain anti-proliferative agents (e.g., cancers resistant to inhibitors of EGFR and/or MEK), or is a cancer is resistant to tyrosine kinase inhibitors (TKI's). In certain embodiments, the cancer to be treated with a compound described herein along with an additional pharmaceutical agent, for example, a modulator of another transcription factors (e.g., YAP, EGFR, MEK), is a cancer that has a mutation in a gene of the Hippo signaling pathway (e.g., has a mutation in EGFR, such as an EGFR-mutant non-small cell lung cancer, or has a mutation in MEK). In certain embodiments, the cancer to be treated with a compound described herein along with an additional pharmaceutical agent, for example, a modulator of another transcription factors (e.g., YAP, EGFR, MEK), is a cancer resistant to certain anti-proliferative agents (e.g., cancers resistant to inhibitors of EGFR and/or MEK). In certain embodiments, the cancer to be treated with a compound described herein along with an additional pharmaceutical agent, for example, a modulator of another transcription factors (e.g., YAP, EGFR, MEK), is a cancer is resistant to tyrosine kinase inhibitors (TKI's).

In certain embodiments, the inflammatory disease to be treated or prevented using the compounds described herein is fibrosis (e.g., idiopathic pulmonary fibrosis, liver cirrhosis, cystic fibrosis, systemic sclerosis, progressive kidney disease, or cardiovascular fibrosis). In certain embodiments, the autoimmune disease to be treated or prevented using the compounds described herein is sclerosis (e.g., systemic sclerosis (scleroderma) or multiple sclerosis). In certain embodiments, the autoimmune disease is amyotrophic lateral sclerosis.

Another aspect of the disclosure relates to methods of inhibiting the activity of a transcription factor (e.g., TEAD, such as TEAD1, TEAD2, TEAD3, TEAD4) in a biological sample (e.g., tissue, cell), or subject. In certain embodiments, the transcription factor is a TEAD. In certain embodiments, the TEAD is TEAD1. In certain embodiments, the TEAD is TEAD2. In certain embodiments, the TEAD is TEAD3. In certain embodiments, the TEAD is TEAD4. In certain embodiments, the activity of the transcription factor (e.g., TEAD, such as TEAD1, TEAD2, TEAD3, TEAD4) is aberrant activity of the transcription factor (e.g., TEAD, such as TEAD1, TEAD2, TEAD3, TEAD4). In certain embodiments, the activity of the transcription factor is increased activity of the transcription factor (e.g., TEAD). In certain embodiments, the inhibition of the activity of the transcription factor (e.g., TEAD, such as TEAD1, TEAD2, TEAD3, TEAD4) is irreversible. In other embodiments, the inhibition of the activity of the transcription factor (e.g., TEAD, such as TEAD1, TEAD2, TEAD3, TEAD4) is reversible. In certain embodiments, the methods of inhibiting the activity of the transcription factor (e.g., TEAD, such as TEAD1, TEAD2, TEAD3, TEAD4) include attaching a compound described herein to the transcription factor (e.g., TEAD, such as TEAD1, TEAD2, TEAD3, TEAD4). In certain embodiments, the methods comprise covalently modifying a transcription factor (e.g., TEAD, such as TEAD1, TEAD2, TEAD3, TEAD4) by attaching a compound described herein to the transcription factor (e.g., TEAD, such as TEAD1, TEAD2, TEAD3, TEAD4). In certain embodiments, the methods comprise covalently inhibiting a transcription factor (e.g., TEAD, such as TEAD1, TEAD2, TEAD3, TEAD4). In certain embodiments, the methods comprise reversibly inhibiting a transcription factor (e.g., TEAD, such as TEAD1, TEAD2, TEAD3, TEAD4). The present invention provides methods of inhibiting cell growth in a biological sample (e.g., tissue, cell), or subject. Another aspect of the disclosure relates to methods of inhibiting the transcription of a gene (e.g., a gene controlled or regulated by a transcription factor (e.g., TEAD, such as TEAD1, TEAD2, TEAD3, TEAD4)) in a biological sample (e.g., tissue, cell), or subject.

In certain embodiments, the methods described herein include administering to a subject or contacting a biological sample with an effective amount of a compound described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the methods described herein include administering to a subject or contacting a biological sample with an effective amount of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein. The additional pharmaceutical agent may be an anti-proliferative agent. In certain embodiments, the additional pharmaceutical agent is an anti-cancer agent. The additional pharmaceutical agent may also be a transcription factor inhibitor. In certain embodiments, the additional pharmaceutical agent is a transcription factor inhibitor (e.g., inhibitor of EGFR and/or MEK). In certain embodiments, the additional pharmaceutical agent comprises an inhibitor of EGFR and an inhibitor of MEK. In certain embodiments, the additional pharmaceutical agent is a binder or inhibitor of TEAD (e.g., TEAD1, TEAD2, TEAD3, TEAD4)). In certain embodiments, the additional pharmaceutical agent is a binder or inhibitor of TEAD. In certain embodiments, the additional pharmaceutical agent is a binder or inhibitor of TEAD1. In certain embodiments, the additional pharmaceutical agent is a binder or inhibitor of TEAD2. In certain embodiments, the additional pharmaceutical agent is a binder or inhibitor of TEAD3. In certain embodiments, the additional pharmaceutical agent is a binder or inhibitor of TEAD4. In certain embodiments, the additional pharmaceutical agent is a selective binder of TEAD. In certain embodiments, the additional pharmaceutical agent is a selective binder of TEAD1. In certain embodiments, the additional pharmaceutical agent is a selective binder of TEAD2. In certain embodiments, the additional pharmaceutical agent is a selective binder of TEAD3. In certain embodiments, the additional pharmaceutical agent is a selective binder of TEAD4. In certain embodiments, the additional pharmaceutical agent is a selective inhibitor of TEAD. In certain embodiments, the additional pharmaceutical agent is a selective inhibitor of TEAD1. In certain embodiments, the additional pharmaceutical agent is a selective inhibitor of TEAD2. In certain embodiments, the additional pharmaceutical agent is a selective inhibitor of TEAD3. In certain embodiments, the additional pharmaceutical agent is a selective inhibitor of TEAD4. In certain embodiments, the additional pharmaceutical agent is a non-selective binder of TEAD1. In certain embodiments, the additional pharmaceutical agent is a non-selective binder of TEAD2. In certain embodiments, the additional pharmaceutical agent is a non-selective binder of TEAD3. In certain embodiments, the additional pharmaceutical agent is a non-selective binder of TEAD4. In certain embodiments, the additional pharmaceutical agent is a non-selective inhibitor of TEAD. In certain embodiments, the additional pharmaceutical agent is a non-selective inhibitor of TEAD1. In certain embodiments, the additional pharmaceutical agent is a non-selective inhibitor of TEAD2. In certain embodiments, the additional pharmaceutical agent is a non-selective inhibitor of TEAD3. In certain embodiments, the additional pharmaceutical agent is a non-selective inhibitor of TEAD4. In certain embodiments, the additional pharmaceutical agent is a selective inhibitor of EGFR. In certain embodiments, the additional pharmaceutical agent is a selective inhibitor of MEK. In certain embodiments, the additional pharmaceutical agent is a non-selective inhibitor of EGFR and/or MEK. In certain embodiments, the additional pharmaceutical agent includes an anti-cancer agent (e.g., chemotherapeutics), anti-inflammatory agent, steroids, immunosuppressant, radiation therapy, or other agents. In certain embodiments, the additional pharmaceutical agent is an anti-proliferative agent. In certain embodiments, the additional pharmaceutical agent is an inhibitor of a kinase. In certain embodiments, the additional pharmaceutical agent is a non-selective inhibitor of a kinase. In certain embodiments, the additional pharmaceutical agent is an immunotherapy agent (e.g., PD1 inhibitor, PDL1 inhibitor). In certain embodiments, the additional pharmaceutical agent is an immune checkpoint inhibitor.

In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. Exemplary chemotherapeutic agents include alkylating agents such as nitrogen mustards, ethylenimines, methylmelamines, alkyl sulfonates, nitrosoureas, and triazenes; antimetabolites such as folic acid analogs, pyrimidine analogs, in particular fluorouracil and cytosine arabinoside, and purine analogs; natural products such as vinca alkaloids epi-podophyllotoxins, antibiotics, enzymes, and biological response modifiers; and miscellaneous products such as platinum coordination complexes, anthracenedione, substituted urea such as hydroxyurea, methyl hydrazine derivatives, and adrenocorticoid suppressant, including ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Exemplary chemotherapeutic agents also include anthracycline antibiotics, actinomycin D, plicamycin, puromycin, gramicidin D, paclitaxel, colchicine, cytochalasin B, emetine, maytansine, amsacrine, cisplatin, carboplatin, mitomycin, altretamine, cyclophosphamide, lomustine, and carmustine. In certain embodiments, a pharmaceutical composition described herein further comprises a combination of the additional pharmaceutical agents described herein.

The disclosed compounds or pharmaceutical compositions thereof used with an additional pharmaceutical agent may synergistically augment inhibition (e.g., increase the degree of inhibition) of a transcription factor (e.g., TEAD, such as TEAD1, TEAD2, TEAD3, TEAD4) induced by the additional pharmaceutical agent(s) in the biological sample or subject. For example, use of the disclosed compounds or pharmaceutical compositions thereof with an additional pharmaceutical agent may increase the degree of inhibition of a transcription factor (e.g., TEAD, such as TEAD1, TEAD2, TEAD3, TEAD4) compared to the degree of inhibition of a transcription factor (e.g., TEAD, such as TEAD1, TEAD2, TEAD3, TEAD4) induced by the additional pharmaceutical agent alone. Thus, the combination of the disclosed compounds or compositions and the additional pharmaceutical agent(s) may be useful in treating proliferative diseases resistant to a treatment using the additional pharmaceutical agent(s) without the disclosed compounds or compositions.

In some embodiments, the activity of a transcription factor (e.g., TEAD, such as TEAD1, TEAD2, TEAD3, TEAD4) is non-selectively inhibited by the compounds or pharmaceutical compositions described herein. In some embodiments, the activity of the transcription factor (e.g., TEAD, such as TEAD1, TEAD2, TEAD3, TEAD4) being inhibited is selectively inhibited by the compounds or pharmaceutical compositions described herein, compared to the activity of a different protein (e.g., a different transcription factor (e.g., TEAD, such as TEAD1, TEAD2, TEAD3, TEAD4)). In certain embodiments, the activity of a transcription factor (e.g., TEAD, such as TEAD1, TEAD2, TEAD3, TEAD4)) is selectively inhibited by a compound or pharmaceutical composition described herein, compared to the activity of a different protein. In certain embodiments, the activity of TEAD1 is selectively inhibited by a compound or pharmaceutical composition described herein, compared to the activity of another TEAD (e.g., TEAD2, TEAD3, TEAD4). In certain embodiments, the activity of TEAD2 is selectively inhibited by a compound or pharmaceutical composition described herein, compared to the activity of another TEAD (e.g., TEAD1, TEAD3, TEAD4). In certain embodiments, the activity of TEAD3 is selectively inhibited by a compound or pharmaceutical composition described herein, compared to the activity of another TEAD (e.g., TEAD1, TEAD2, TEAD4). In certain embodiments, the activity of TEAD4 is selectively inhibited by a compound or pharmaceutical composition described herein, compared to the activity of another TEAD (e.g., TEAD1, TEAD2, TEAD3).

The selectivity of a compound or pharmaceutical composition described herein in inhibiting the activity of a transcription factor (e.g., TEAD, such as TEAD1, TEAD2, TEAD3, TEAD4) over a different protein (e.g., a different transcription factor (e.g., TEAD)) may be measured by the quotient of the IC₅₀ value of the compound or pharmaceutical composition in inhibiting the activity of the different protein over the IC₅₀ value of the compound or pharmaceutical composition in inhibiting the activity of the transcription factor (e.g., TEAD). The selectivity of a compound or pharmaceutical composition described herein for a protein transcription factor (e.g., TEAD) over a different protein may also be measured by the quotient of the K_{d} value of an adduct of the compound or pharmaceutical composition and the different protein over the K_{d} value of an adduct of the compound or pharmaceutical composition and the transcription factor (e.g., TEAD). In certain embodiments, the selectivity is at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

In certain embodiments, a kit described herein includes a first container comprising a compound or pharmaceutical composition described herein. In certain embodiments, a kit described herein is useful in treating and/or preventing a disease, such as a proliferative disease (e.g., cancers (e.g., carcinoma, sarcoma); lung cancer, breast cancer, liver cancer, pancreatic cancer, gastric cancer, ovarian cancer, colon cancer, colorectal cancer, skin cancer, esophageal cancer)), inflammatory disease (e.g., fibrosis), or autoimmune disease (e.g., sclerosis), in a subject in need thereof, inhibiting the activity of a transcription factor (e.g., TEAD, such as TEAD1, TEAD2, TEAD3, TEAD4), and/or inhibiting the transcription of a gene (e.g., a gene controlled or regulated by a transcription factor (e.g., TEAD, such as TEAD1, TEAD2, TEAD3, TEAD4)) in a subject, and/or biological sample (e.g., tissue, cell).

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a proliferative disease in a subject in need thereof, preventing a disease, such as a proliferative disease, inflammatory disease, autoimmune disease in a subject in need thereof, inhibiting the activity of a transcription factor (e.g., TEAD, such as TEAD1, TEAD2, TEAD3, TEAD4)) in a subject and/or biological sample (e.g., tissue, cell), and/or inhibiting the transcription of a gene (e.g., a gene controlled or regulated by a transcription factor (e.g., TEAD, such as TEAD1, TEAD2, TEAD3, TEAD4)). A kit described herein may include one or more additional pharmaceutical agents described herein as a separate composition.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Example 1. Biological Assays

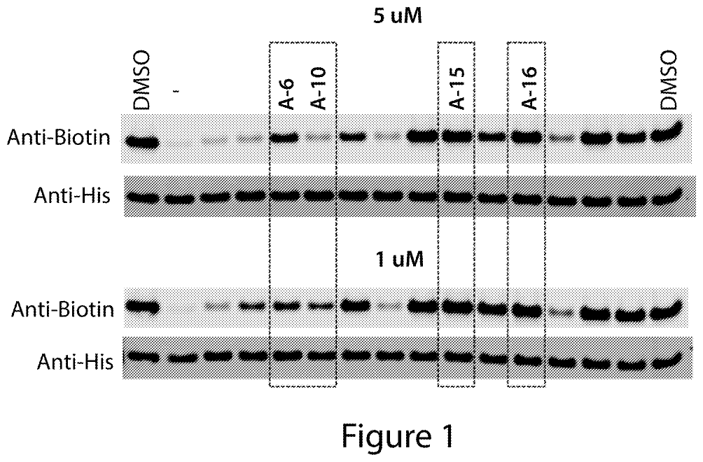

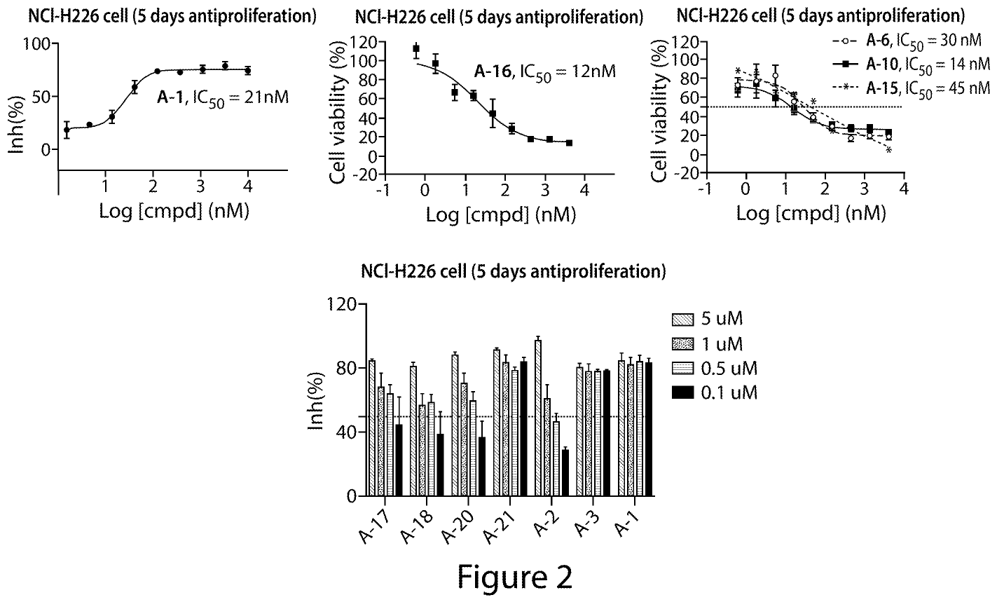

The compounds were evaluated in vitro. An anti-palmitoylation assay was conducted, and the results are shown in FIG. 1. In addition, an anti-proliferation assay was performed using NCI-H226 cells (FIG. 2). The IC50 values of certain compounds are shown below in Table 1.

TABLE 1
IC₅₀ values of certain compounds of the disclosure.

| | IC50 (nM) |
|---|---|
| Compound | (NCI-H226 cells) |
| A-7 | 17 |
| A-11 | 40 |
| A-13 | 31 |
| A-14 | 1100 |
| A-12 | 14 |
| A-21 | 4 |
| A-5 | 8 |

### Gel-Based Anti-Palmitoylation Assay

The mixture of 2-fluoro-5-nitrobenzonitrile (1 g, 6.0 mmol), cyclohexanamine (0.61 μM His-tag TEAD-YBD recombinant protein was incubated with inhibitors at the indicated concentrations at 37° C. for 2 h followed by the addition of palmitoyl alkyne-coenzyme A (Cayman chemical, no. 15968) in a total volume of 50 μL. After 30 min reaction, 5 μL 10% SDS were added and 5 μL click reagents were added to start click reaction. After another 1 h, 4× loading buffer were added to the reaction mixture and the samples subjected for western blot analysis. IRDye 800CW Streptavidin (LI-COR, no. 92632230) and His-Tag Mouse mAb (Cell Signaling, no. 2366S) was used for biotin detection and His-tag detection. The blots were imaged on Odyssey CLx Imager (LI-COR).

### Antiproliferation Assay

The mixture of 2-fluoro-5-nitrobenzonitrile (1 g, 6.0 mmol), cyclohexanamine (0.6For 2D adherent cell viability experiment, the cells were seeded at 384-well plate (Corning, no. 3570) at the density of 200 cells/well. The next day, compounds were added using Janus workstation (PerkinElmer). After 5 days treatment, the cell viability was measured by CellTiter-Glo kit (Promega, no. G7570) as the manufacturer recommended.

### Example 2. Synthesis of Compounds

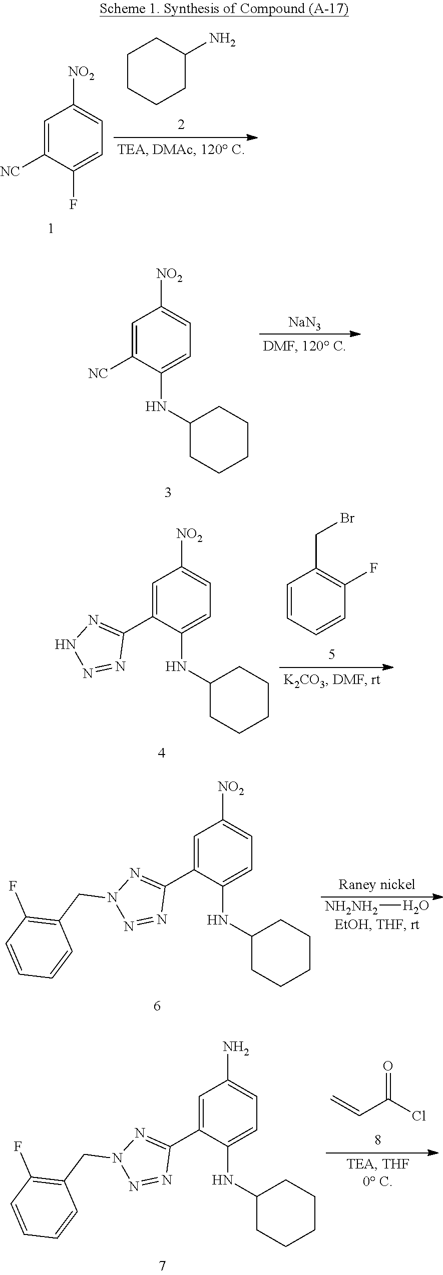

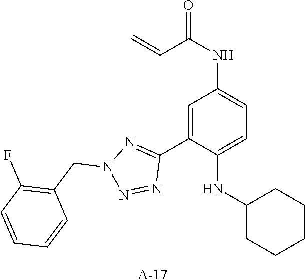 [SMILES: C=CC(=O)Nc1ccc(NC2CCCCC2)c(-c2nnn(Cc3ccccc3F)n2)c1 ; "A-17"]

### Step 1: Synthesis of 2-(cyclohexylamino)-5-nitrobenzonitrile (Compound 3)

The mixture of 2-fluoro-5-nitrobenzonitrile (1 g, 6.0 mmol), cyclohexanamine (0.6 g, 6.0 mmol) and TEA (1.2 g, 12.0 mmol) in DMAc (5 mL) was heated at 120° C. under N₂ for 16 hours, until all starting material was consumed as showed by LCMS. The mixture was then concentrated and purified by flash column chromatography on silica gel (ethyl acetate in petroleum ether=50% v/v) to afford the target compound 3 as solid (1.4 g, yield 94.9%). LC-MS (ESI) m/z: 246 [M+H]⁺.

### Step 2: Synthesis of N-cyclohexyl-4-nitro-2-(2H-tetrazol-5-yl) aniline (Compound 4)

The mixture of 2-(cyclohexylamino)-5-nitrobenzonitrile (1.3 g, 5.3 mmol) and NaN₃ (0.52 g, 7.9 mmol) in DMF (30 mL) was heated at 120° C. under N₂ for 16 hours, until all starting material was consumed as showed by LCMS. The mixture was concentrated and purified by flash column chromatography on silica gel (ethyl acetate in petroleum ether=20% v/v) to obtain the target compound 4 as solid (800 mg, yield 52.3%). LC-MS (ESI) m/z: 289 [M+H]⁺.

### Step 3: Synthesis of N-cyclohexyl-2-(2-(2-fluorobenzyl)-2H-tetrazol-5-yl)-4-nitroaniline (Compound 6)

The mixture of N-cyclohexyl-4-nitro-2-(2H-tetrazol-5-yl)aniline (700 mg, 2.43 mmol), 1-(bromomethyl)-2-fluorobenzene (456 mg, 2.43 mmol) and K₂CO₃ (670 mg, 4.86 mmol) in DMF (20 mL) was stirred at rt under N₂ for 3 hours. The mixture was concentrated and purified by flash column chromatography on silica gel (ethyl acetate in petroleum ether=40% v/v) to obtain the target compound 6 as solid (600 mg, yield 62.3%). LC-MS (ESI) m/z: 397 [M+H]⁺.

### Step 4: Synthesis of N1-cyclohexyl-2-(2-(2-fluorobenzyl)-2H-tetrazol-5-yl)benzene-1,4-diamine (Compound 7)

To the solution of N-cyclohexyl-2-(2-(2-fluorobenzyl)-2H-tetrazol-5-yl)-4-nitroaniline (500 mg, 1.26 mmol) in EtOH (15 mL) and THF (15 mL) was added Raney nickel (53 mg, 0.13 mmol), and then N₂H₄·H₂O (63 mg, 1.26 mmol) was added. The mixture was stirred at rt for 1 hour and filtered, the filtrate was concentrated under reduced pressure to leave the crude compound 7 as oil (400 mg, yield 86.5%). LC-MS (ESI) m/z: 367 [M+H]⁺.

### Step 5: Synthesis of N-(4-(cyclohexylamino)-3-(2-(2-fluorobenzyl)-2H-tetrazol-5-yl)phenyl)acrylamide (A-17)

To the mixture of N1-cyclohexyl-2-(2-(2-fluorobenzyl)-2H-tetrazol-5-yl)benzene-1,4-diamine (450 mg, 1.23 mmol) and Et₃N (249 mg, 2.46 mmol) in THF (15 mL) was added acryloyl chloride (110 mg, 1.23 mmol), the mixture was stirred at 0° C. for 1 hour, concentrated and purified by preparative HPLC (MeCN/H₂O/TFA) to afford the target A-17 as solid (490 mg, yield 95.0%). LC-MS (ESI) m/z: 421.3 [M+H]⁺. ¹H-NMR (400 MHz, DMSO-d₆) δ (ppm): 10.00 (s, 1H), 8.34 (d, J=2.5 Hz, 1H), 7.65 (dd, J=9.0, 2.5 Hz, 1H), 7.57 (t, J=7.6 Hz, 1H), 7.53-7.43 (m, 1H), 7.29 (dd, J=15.6, 7.9 Hz, 2H), 6.82 (dd, J=13.5, 8.5 Hz, 2H), 6.39 (dd, J=16.9, 10.1 Hz, 1H), 6.21 (dd, J=17.0, 2.0 Hz, 1H), 6.08 (s, 2H), 5.69 (dd, J=10.1, 2.0 Hz, 1H), 3.48 (s, 1H), 2.0-1.75 (br, 2H), 1.70-1.50 (m, 3H), 1.47-1.32 (m, 2H), 1.32-1.15 (m, 3H). ¹⁹F-NMR (376 MHz, DMSO-d₆) δ (ppm): 117.43.

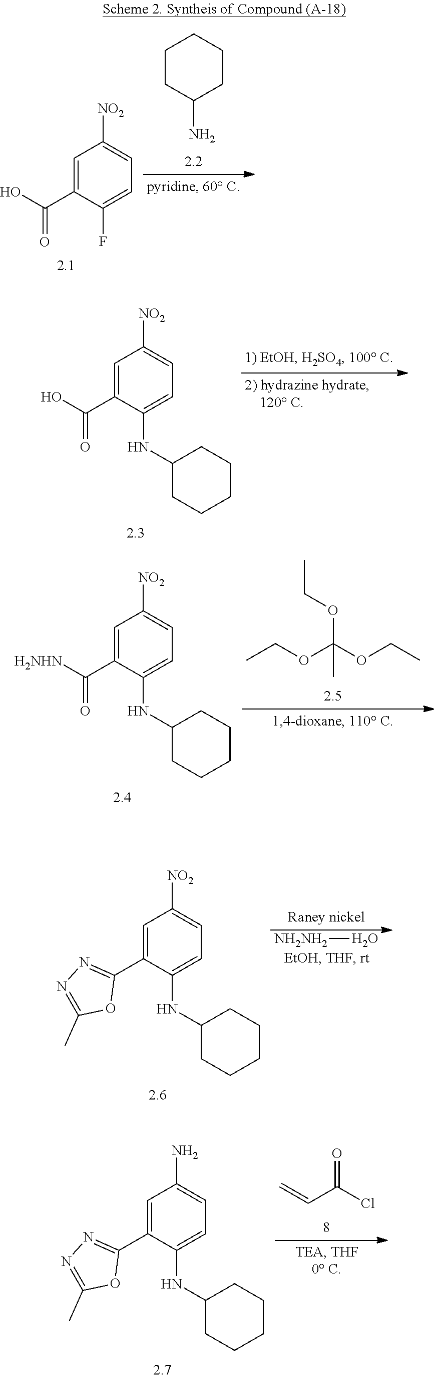

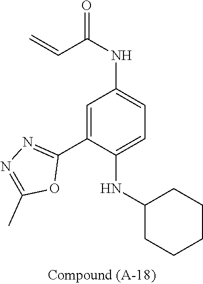 [SMILES: C=CC(=O)Nc1ccc(NC2CCCCC2)c(-c2nnc(C)o2)c1]

### Step 1: Synthesis of 2-(cyclohexylamino)-5-nitrobenzoic Acid (Compound 2.3)

The mixture of 2-fluoro-5-nitrobenzoic acid (1 g, 5.4 mmol) and cyclohexanamine (535 mg, 5.4 mmol) in pyridine (20 mL) was heated at 60° C. Under N₂ for 16 hours. The resulting mixture was concentrated and purified by flash column chromatography on silica gel (ethyl acetate in petroleum ether=30% v/v) to afford the target compound 2.3 as solid (1.5 g, yield 99.9%). LC-MS (ESI) m/z: 265 [M+H]⁺.

### Step 2: Synthesis of 2-(cyclohexylamino)-5-nitrobenzohydrazide (Compound 2.4)

The mixture of 2-(cyclohexylamino)-5-nitrobenzoic acid (1.4 g, 0.19 mmol) in EtOH (30 mL) and H₂SO₄ (30 mL) was heated at 100° C. for 16 hours. The resulting mixture was concentrated in vacuum, the residue was adjusted to pH 9 with concentrated ammonia solution and extracted with ether (100 mL×2), the combined extracts were dried over anhydrous Na₂SO₄, filtered and concentrated in vacuum, to the residue was added hydrazine hydrate (98%, 1 mL), the resulting mixture was heated at 120° C. for 3 hours, concentrated and purified by flash column chromatography on silica gel (ethyl acetate in petroleum ether=30% v/v) to obtain the target compound 2.4 as oil (700 mg, yield 36.6%). LC-MS (ESI) m/z: 279 [M+H]⁺.

### Step 3: Synthesis of N-cyclohexyl-2-(5-methyl-1,3,4-oxadiazol-2-yl)-4-nitroaniline (Compound 2.6)

The mixture of 2-(cyclohexylamino)-5-nitrobenzohydrazide (650 mg, 2.34 mmol) and 1,1,1-triethoxyethane (379 mg, 2.34 mmol) in 1,4-dioxane (30 mL) was heated at 110° C. for 16 hours. The resulting mixture was concentrated and purified by flash column chromatography on silica gel (ethyl acetate in petroleum ether=50% v/v) to obtain the target compound 2.6 as solid (600 mg, yield 84.9%). LC-MS (ESI) m/z: 303 [M+H]⁺.

### Step 4: Synthesis of N1-cyclohexyl-2-(5-methyl-1,3,4-oxadiazol-2-yl)benzene-1,4-diamine (Compound 2.7)

To the solution of N-cyclohexyl-2-(5-methyl-1,3,4-oxadiazol-2-yl)-4-nitroaniline (550 mg, 1.82 mmol) in EtOH (15 mL) and THF (15 mL) was added Raney nickel (88 mg, 0.18 mmol), and then N₂H₄·H₂O (110 mg, 1.82 mmol). The resulting mixture was stirred at rt for 1 hour and filtered, the filtrate was concentrated under reduced pressure to leave the crude target compound 2.7 as oil (500 mg, yield 99.9%). LC-MS (ESI) m/z: 273 [M+H]⁺.

### Step 5: Synthesis of N-(4-(cyclohexylamino)-3-(5-methyl-1,3,4-oxadiazol-2-yl)phenyl)acrylamide (Compound (A-18))

To the mixture of N1-cyclohexyl-2-(5-methyl-1,3,4-oxadiazol-2-yl)benzene-1,4-diamine (450 mg, 1.65 mmol) and Et₃N (100 mg, 3.31 mmol) in THF (20 mL) was added acryloyl chloride (64 mg, 1.65 mmol), the resulting mixture was stirred at 0° C. for 1 hour, concentrated and monitored by preparative HPLC (MeCN/H₂O/TFA) to obtain the target compound (A-18) as solid (314 mg, yield 58.2%). LC-MS (ESI) m/z: 327 [M+H]⁺. ¹H-NMR (400 MHz, DMSO-d₆) δ (ppm): 10.03 (s, 1H), 8.22 (d, J=2.5 Hz, 1H), 7.57 (dd, J=9.1, 2.5 Hz, 1H), 7.32 (d, J=7.7 Hz, 1H), 6.90 (d, J=9.2 Hz, 1H), 6.39 (dd, J=16.5, 9.6 Hz, 1H), 6.22 (dd, J=17.0, 2.1 Hz, 1H), 5.71 (dd, J=10.0, 2.1 Hz, 1H), 3.55-3.50 (br, 1H), 2.59 (s, 3H), 1.97 (d, J=9.7 Hz, 2H), 1.75-1.65 (m, 2H), 1.60-1.50 (m, 1H), 1.50-1.35 (m, 2H), 1.35-1.26 (m, 3H).

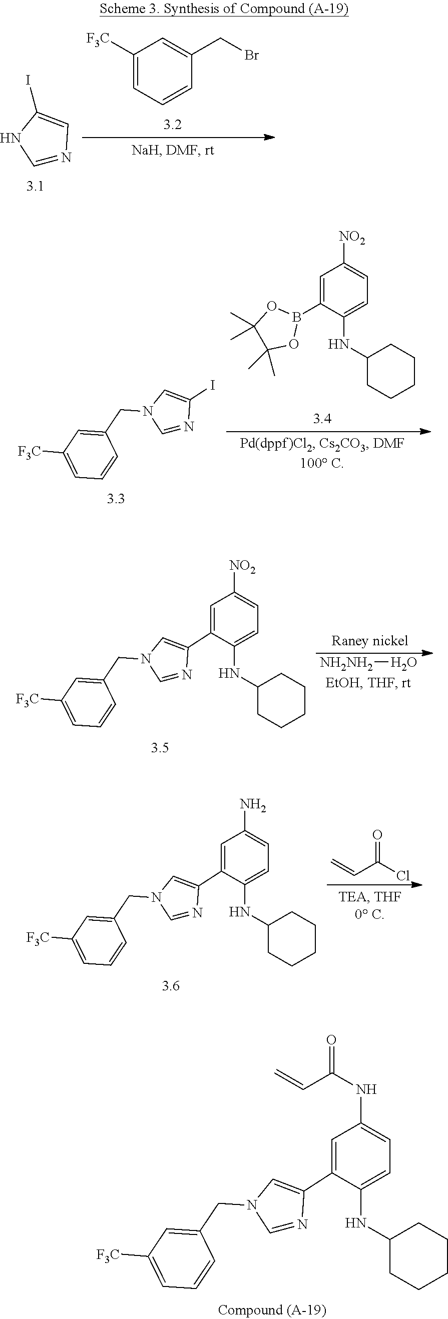

### Step 1: Synthesis of 4-iodo-1-(3-(trifluoromethyl)benzyl)-1H-imidazole (Compound 3.3)

To the solution of 5-iodo-1H-imidazole (1 g, 5.18 mmol) in DMF (20 mL) was added NaH (248 mg, 6.2 mmol) under N₂, the mixture was stirred at 0° C. for 0.5 hour, and then 1-(bromomethyl)-3-(trifluoromethyl)benzene (1.24 g, 5.18 mmol) was added, the resulting mixture was stirred at rt for 1 hours, concentrated and purified by flash column chromatography on silica gel (ethyl acetate in petroleum ether=30% v/v) to obtain the target compound 3.3 as solid (1.5 g, yield 82.8%). LC-MS (ESI) m/z: 353 [M+H]⁺.

### Step 2: Synthesis of N-cyclohexyl-4-nitro-2-(1-(3-(trifluoromethyl)benzyl)-1H-imidazol-4-yl)aniline (Compound 3.5)

The mixture of 4-iodo-1-(3-(trifluoromethyl)benzyl)-1H-imidazole (300 mg, 0.96 mmol), N-cyclohexyl-4-nitro-2-(4,4,5,5-tetramethyl-1,3,2-dioxaborolan-2-yl)aniline (300 mg, 0.96 mmol), Cs₂CO₃ (828 mg, 2.52 mmol) and Pd(dppf)Cl₂ (62.4 mg, 0.096 mmol) in DMF (2 mL) was heated at 100° C. under N₂ for 16 hours. The resulting mixture was concentrated and purified by flash column chromatography on silica gel (ethyl acetate in petroleum ether=30% v/v) to obtain the target compound 3.5 as solid (120 mg, yield 31.7%). LC-MS (ESI) m/z: 445 [M+H]⁺.

### Step 3: Synthesis of N1-cyclohexyl-2-(1-(3-(trifluoromethyl)benzyl)-1H-imidazol-4-yl)benzene-1,4-diamine (Compound 3.6)

To the solution of N-cyclohexyl-4-nitro-2-(1-(3-(trifluoromethyl)benzyl)-1H-imidazol-4-yl)aniline (90 mg, 0.18 mmol) in EtOH (10 mL) and THF (10 mL) was added Raney nickel (12 mg, 0.03 mmol), followed by N₂H₄·H₂O (9 mg, 0.18 mmol). The mixture was stirred at rt for 1 hour and filtered, the filtrate was concentrated under reduced pressure to leave crude target compound 3.6 as oil (60 mg, yield 71.5%). LC-MS (ESI) m/z: 415 [M+H]⁺.

### Step 4: Synthesis of N-(4-(cyclohexylamino)-3-(1-(3-(trifluoromethyl)benzyl)-1H-imidazol-4-yl)phenyl)acrylamide (Compound (A-19))

To the mixture of N1-cyclohexyl-2-(1-(3-(trifluoromethyl)benzyl)-1H-imidazol-4-yl)benzene-1,4-diamine (50 mg, 0.12 mmol) and Et₃N (24 mg, 0.24 mmol) in THF (10 mL) was added acryloyl chloride (18 mg, 0.12 mmol), the mixture was stirred at 0° C. for 1 hour, concentrated and purified by preparative HPLC (MeCN/H₂O/TFA) to afford the target Compound (A-19) as solid (5 mg, yield 8.8%). LC-MS (ESI) m/z: 469 [M+H]⁺. ¹H-NMR (400 MHz, CD₃OD) δ (ppm): 7.89 (s, 1H), 7.70-7.54 (m, 5H), 7.40 (s, 1H), 7.32 (dd, J=8.8, 2.4 Hz, 1H), 6.76 (d, J=8.4 Hz, 1H), 6.41 (dd, J=16.8, 10.0 Hz, 1H), 6.31 (dd, J=16.8, 2.0 Hz, 1H), 5.73 (dd, J=9.8, 2.1 Hz, 1H), 5.40 (s, 2H), 2.06-1.96 (m, 2H), 1.80-1.70 (m, 2H), 1.68-1.58 (m, 1H), 1.50-1.21 (m, 6H).

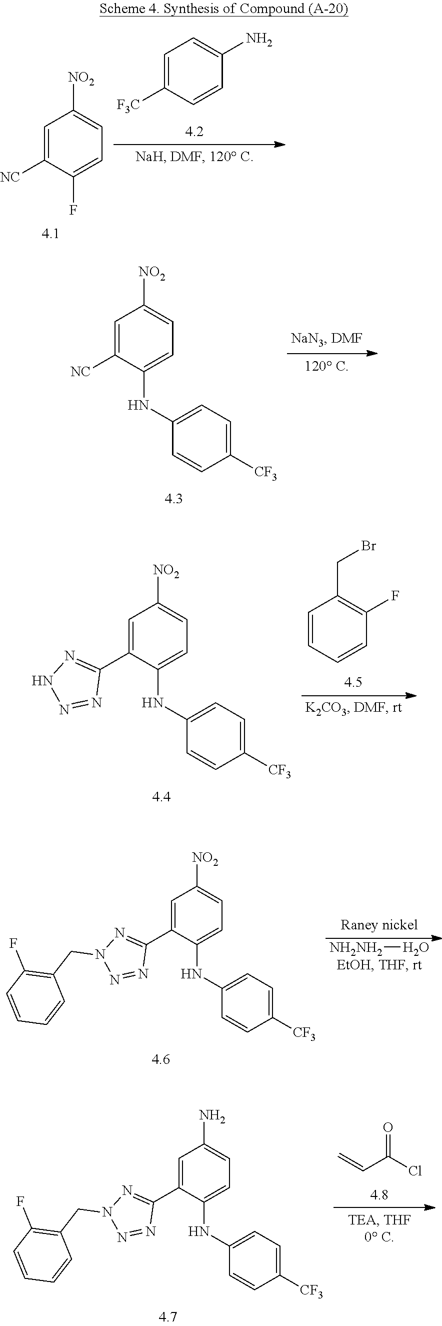

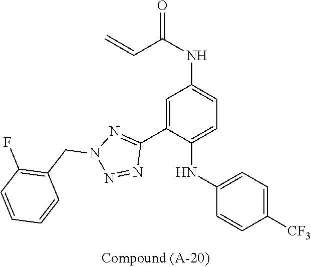 [SMILES: C=CC(=O)Nc1ccc(Nc2ccc(C(F)(F)F)cc2)c(-c2nnn(Cc3ccccc3F)n2)c1]

### Step 1: Synthesis of 5-nitro-2-(4-(trifluoromethyl)phenylamino)benzonitrile (Compound 4.3)

To the solution of 4-(trifluoromethyl) aniline (0.96 mg, 6.0 mmol) in DMF (20 mL) was added NaH (0.29 g, 7.2 mmol), the mixture was stirred at 0° C. under N₂ for 0.5 hour, and then 2-fluoro-5-nitrobenzonitrile (1 g, 6.0 mmol) was added. The resulting mixture was stirred at 120° C. for 16 hours, concentrated and purified by flash column chromatography on silica gel (ethyl acetate in petroleum ether=30% v/v) to afford the target compound 4.3 as solid (500 mg, yield 27.1%). LC-MS (ESI) m/z: 308 [M+H]⁺.

### Step 2: Synthesis of 4-nitro-2-(2H-tetrazol-5-yl)-N-(4-(trifluoromethyl)phenyl)aniline (Compound 4.4)

The mixture of 5-nitro-2-(4-(trifluoromethyl)phenylamino)benzonitrile (400 mg, 1.30 mmol) and NaN₃ (127 mg, 1.95 mmol) in DMF (20 mL) was heated at 120° C. for 16 hours. The resulting mixture was concentrated and purified by flash column chromatography on silica gel (ethyl acetate in petroleum ether=30% v/v) to obtain the target compound 4.4 as solid (300 mg, yield 65.7%). LC-MS (ESI) m/z: 351 [M+H]⁺.

### Step 3: Synthesis of 2-(2-(2-fluorobenzyl)-2H-tetrazol-5-yl)-4-nitro-N-(4-(trifluoromethyl)phenyl)aniline (Compound 4.6)

The mixture of 4-nitro-2-(2H-tetrazol-5-yl)-N-(4-(trifluoromethyl)phenyl)aniline (250 mg, 0.71 mmol), 1-(bromomethyl)-2-fluorobenzene (134 mg, 0.71 mmol) and K₂CO₃ (196 mg, 1.42 mmol) in DMF (15 mL) was stirred at rt for 3 hours. The resulting mixture was concentrated and purified by flash column chromatography on silica gel (ethyl acetate in petroleum ether=40% v/v) to obtain the target compound 4.6 as solid (300 mg, yield 91.7%). LC-MS (ESI) m/z: 459 [M+H]⁺.

### Step 4: Synthesis of 2-(2-(2-fluorobenzyl)-2H-tetrazol-5-yl)-N-1-(4-(trifluoromethyl)phenyl)benzene-1,4-diamine (Compound 4.7)

To the solution of 2-(2-(2-fluorobenzyl)-2H-tetrazol-5-yl)-4-nitro-N-(4-(trifluoromethyl)phenyl)aniline (250 mg, 0.55 mmol) in EtOH (10 mL) and THF (10 mL) was added Raney nickel (20 mg, 0.05 mmol), and then N₂H₄·H₂O (27 mg, 0.55 mmol) was added. The mixture was stirred at rt for 1 hour and filtered, the filtrate was concentrated under reduced pressure to leave the crude compound 4.7 as oil (200 mg, yield 71.4%). LC-MS (ESI) m/z: 429 [M+H]⁺.

### Step 5: Synthesis of N-(3-(2-(2-fluorobenzyl)-2H-tetrazol-5-yl)-4-(4-(trifluoromethyl)phenylamino)phenyl)acrylamide (Compound (A-20))

To the mixture of 2-(2-(2-fluorobenzyl)-2H-tetrazol-5-yl)-N1-(4-(trifluoromethyl) phenyl)benzene-1,4-diamine (200 mg, 0.47 mmol) and Et₃N (97 mg, 0.94 mmol) in THF (15 mL) was added acryloyl chloride (42 mg, 0.47 mmol), the mixture was stirred at 0° C. for 1 hour, concentrated and purified by preparative HPLC (MeCN/H₂O/TFA) to obtain the target Compound (I-13) as solid (171 mg, yield 76.0%). LC-MS (ESI) m/z: 483 [M+H]⁺. ¹H-NMR (400 MHz, DMSO-d₆) δ (ppm) 10.32 (s, 1H), 8.59 (s, 1H), 8.37 (d, J=2.4 Hz, 1H), 7.83 (dd, J=9.1, 2.3 Hz, 1H), 7.49 (dd, J=8.1, 3.8 Hz, 5H), 7.34-7.18 (m, 2H), 7.03 (d, J=8.7 Hz, 2H), 6.43 (dd, J=17.0, 10.1 Hz, 1H), 6.27 (dd, J=17.0, 2.0 Hz, 1H), 6.04 (s, 2H), 5.77 (dd, J=10.1, 2.0 Hz, 1H). ¹⁹F-NMR (376 MHz, DMSO-d₆) δ (ppm): 117.53.

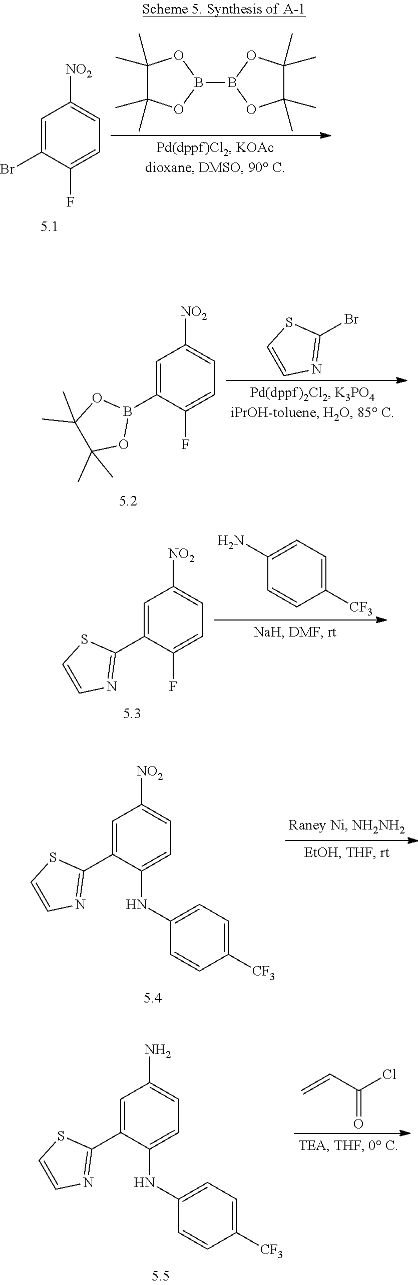

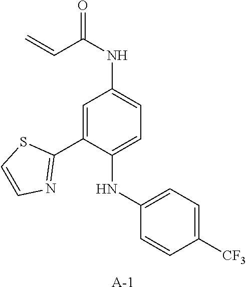 [SMILES: C=CC(=O)Nc1ccc(Nc2ccc(C(F)(F)F)cc2)c(-c2nccs2)c1 ; "A-1"]

### Step 1: Synthesis of 2-(2-fluoro-5-nitrophenyl)-4,4,5,5-tetramethyl-1,3,2-dioxaborolane (Compound 5.2)

The mixture of 2-bromo-1-fluoro-4-nitrobenzene (2 g, 9.2 mmol), 4,4,4′,4′,5,5,5′,5′-octamethyl-2,2′-bi(1,3,2-dioxaborolane) (1.2 g, 10 mmol), KOAc (1.8 g, 18.4 mmol) and Pd(dppf)Cl₂ (336 mg, 0.44 mmol) in 1,4-dioxane (50 mL) and DMSO (5 mL) was heated at 90° C. under N₂ for 14 hours. The mixture was diluted with water (100 mL) and extracted with EtOAc (100 mL×3), the combined organics were washed with brine (100 mL), dried over anhydrous Na₂SO₄, filtered and concentrated to leave the crude compound 5.2 as oil (1.6 g, yield 65.6%). LC-MS (ESI) m/z: No mass.

### Step 2: Synthesis of 2-(2-fluoro-5-nitrophenyl)thiazole (Compound 5.3)

The mixture of 2-(2-fluoro-5-nitrophenyl)-4,4,5,5-tetramethyl-1,3,2-dioxaborolane (800 mg, 3.0 mmol), 2-bromopyrimidine (486 mg, 3.0 mmol), K₃PO₄ (1.9 g, 9.0 mmol), Pd₂(dppf)Cl₂ (108 mg, 0.15 mmol) in Me₂CHOH (10 mL), PhMe (6 mL) and H₂O (6 mL) was heated at 85° C. for 4 hours. The resulted mixture was concentrated and purified by flash column chromatography on silica gel (ethyl acetate in petroleum ether=25% v/v) to obtain compound 5.3 as solid (150 mg, yield 22%). LC-MS (ESI) m/z: 225 [M+H]⁺.

### Step 3: Synthesis of 2-(thiazol-2-yl)-N1-(4-(trifluoromethyl)phenyl)benzene-1,4-diamine (Compound 5.4)

To the mixture of 2-(2-fluoro-5-nitrophenyl)thiazole (150 mg, 0.67 mmol) in DMF (5 mL) was added NaH (60%, 53 mg, 1.34 mmol), the mixture was stirred at 0° C. under N₂ for 0.5 hour, and then 4-(trifluoromethyl)aniline (107 mg, 0.67 mmol) was added in one portion. The mixture was stirred at rt for 16 hours, concentrated and purified by flash column chromatography on silica gel (ethyl acetate in petroleum ether-30% v/v) to obtain the target compound 5.4 as solid (30 mg, yield 12%). LC-MS (ESI) m/z: 366 [M+H]⁺.

### Step 4: Synthesis of 2-(thiazol-2-yl)-N1-(4-(trifluoromethyl)phenyl)benzene-1,4-diamine (Compound 5.5)

To the solution of 4-nitro-2-(thiazol-2-yl)-N-(4-(trifluoromethyl)phenyl)aniline (30 mg, 0.082 mmol) in EtOH (2 mL) and THF (2 mL) was added Raney nickel (16 mg, 0.04 mmol), and then N₂H₄·H₂O (20 mg, 0.42 mmol) was added, the mixture was stirred at rt for 1 hour, filtered and concentrated at reduced pressure to leave the crude compound 5.5 as oil (22 mg, yield 80%). LC-MS (ESI) m/z: 336 [M+H]⁺.

### Step 5: Synthesis of N-(3-(thiazol-2-yl)-4-(4-(trifluoromethyl)phenylamino)phenyl)acrylamide (A-1)

To the mixture of 2-(thiazol-2-yl)-N1-(4-(trifluoromethyl)phenyl)benzene-1,4-diamine (22 mg, 0.06 mmol) and Et₃N (12 mg, 0.12 mmol) in THF (10 mL) was added acryloyl chloride (6 mg, 0.06 mmol), the mixture was stirred at 0° C. for 1 hour, and then concentrated and purified by prep-HPLC (MeCN/TFA/H₂O) to obtain the target compound A-1 as solid (15 mg, yield 64%). LC-MS (ESI) m/z: 390 [M+H]⁺. ¹H NMR (400 MHz, CD₃OD) δ (ppm) 8.39 (d, J=2.4 Hz, 1H), 7.93 (d, J=3.4 Hz, 1H), 7.66-7.48 (m, 5H), 7.20 (d, J=8.5 Hz, 2H), 6.43 (qd, J=17.0, 5.9 Hz, 2H), 5.81 (dd, J=9.5, 2.4 Hz, 1H).

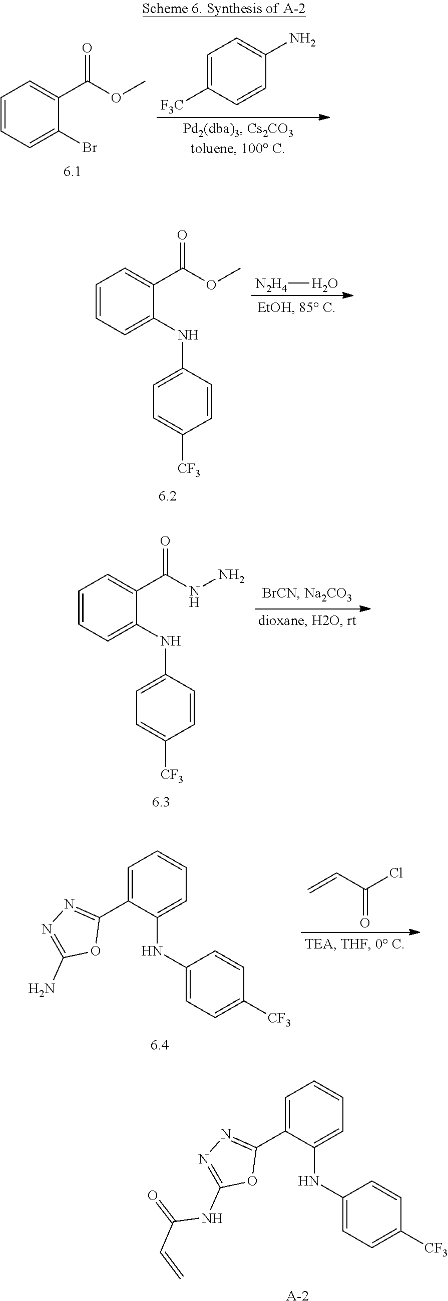

### Step 1: Synthesis of methyl 2-(4-(trifluoromethyl)phenylamino)benzoate (Compound 6.2)

The mixture of 4-(trifluoromethyl)aniline (2 g, 9.3 mmol), methyl 2-bromobenzoate (1.2 g, 7.5 mmol), Cs₂CO₃ (3.34 mg, 10.2 mmol) and Pd₂(dba)₃ (0.085 mg, 0.093 mmol) in toluene (20 mL) was stirred at 100° C. under N₂ for 16 hours. The resulting mixture was concentrated and purified by flash column chromatography on silica gel (ethyl acetate in petroleum ether-30% v/v) to obtain the target compound 6.2 as solid (2.4 g, yield 72.2%). LC-MS (ESI) m/z: 296 [M+H]⁺.

### Step 2: Synthesis of 2-(4-(trifluoromethyl)phenylamino)benzohydrazide (Compound 6.3)

To the solution of methyl 2-(4-(trifluoromethyl)phenylamino)benzoate (2.3 g, 7.8 mmol) in EtOH (30 mL) was added N₂H₄·H₂O (0.39 g, 7.8 mmol). The mixture was stirred at 85° C. under N₂ for 16 hours, concentrated and purified by flash column chromatography on silica gel (ethyl acetate in petroleum ether=30% v/v) to obtain the target compound 6.3 as oil (2 g, yield 83.3%). LC-MS (ESI) m/z: 296 [M+H]⁺.

### Step 3: Synthesis of 5-(2-(4-(trifluoromethyl)phenylamino)phenyl)-1,3,4-oxadiazol-2-amine (Compound 6.4)

To the solution of 2-(4-(trifluoromethyl)phenylamino)benzohydrazide (1.9 g, 6.4 mmol) in 1,4-dioxane (10 mL) and H₂O (10 mL) was added BrCN (0.68 g, 6.4 mmol) and NaHCO₃ (0.54 g, 6.4 mmol). The resulting mixture was stirred at rt under N₂ for 16 hours, concentrated and purified by flash column chromatography on silica gel (ethyl acetate in petroleum ether=30% v/v) to obtain the target compound 6.4 as solid (800 mg, yield 38.8%). LC-MS (ESI) m/z: 321 [M+H]⁺.

### Step 4: Synthesis of N-(5-(2-(4-(trifluoromethyl)phenylamino)phenyl)-1,3,4-oxadiazol-2-yl)acrylamide (Compound A-2)

To the solution of 5-(2-(4-(trifluoromethyl)phenylamino)phenyl)-1,3,4-oxadiazol-2-amine (700 mg, 2.19 mmol) and Et₃N (442 mg, 4.38 mmol) in THF (10 mL) was added acryloyl chloride (196 mg, 2.19 mmol). The mixture was stirred at 0° C. for 1 hour, concentrated and purified by prep-HPLC (MeCN/TFA/H₂O) to obtain the target compound A-2 as solid (35 mg, yield 3.7%). LC-MS (ESI) m/z: 375 [M+H]⁺. ¹H-NMR (400 MHz, DMSO-d₆) δ (ppm) 12.12 (s, 1H), 9.16 (s, 1H), 7.82 (dd, J=7.9, 1.4 Hz, 1H), 7.65 (d, J=8.5 Hz, 2H), 7.60-7.49 (m, 2H), 7.39 (d, J=8.5 Hz, 2H), 7.14 (t, J=7.5 Hz, 1H), 6.46 (qd, J=17.0, 5.8 Hz, 2H), 5.95 (dd, J=9.9, 1.9 Hz, 1H).

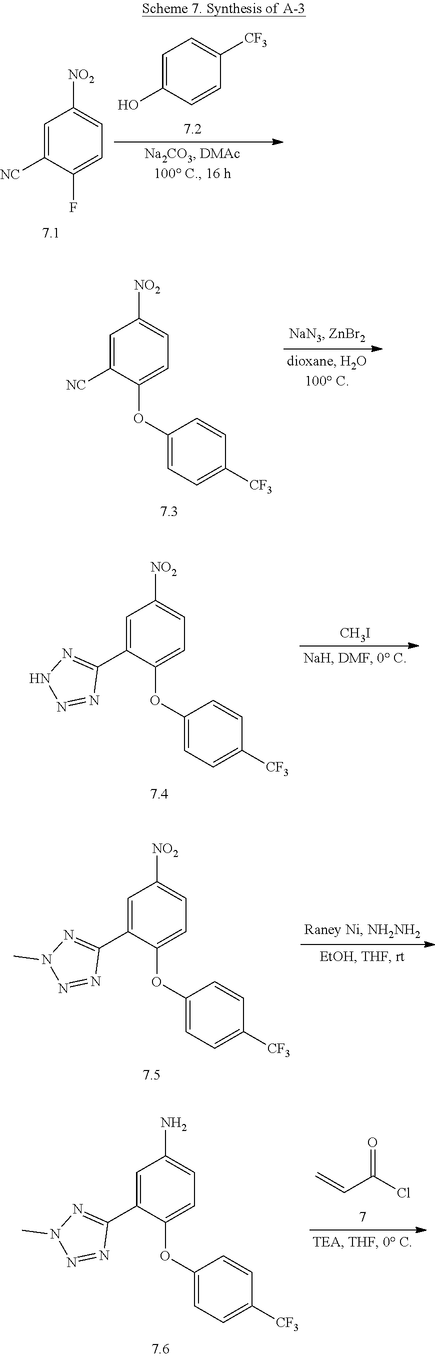

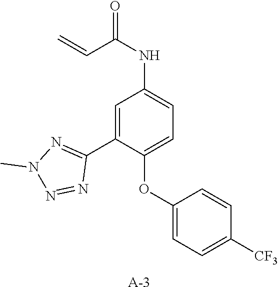 [SMILES: C=CC(=O)Nc1ccc(Oc2ccc(C(F)(F)F)cc2)c(-c2nnn(C)n2)c1 ; "A-3"]

### Step 1: Synthesis of 5-nitro-2-(4-(trifluoromethyl)phenoxy)benzonitrile (Compound 7.3)

The mixture of 2-fluoro-5-nitrobenzonitrile (1 g, 6.02 mmol), 4-(trifluoromethyl) phenol (0.976 g, 6.02 mmol) and Na₂CO₃ (0.83 mg, 12.04 mmol) in DMAc (20 mL) was stirred at 100° C. for 16 hours. The mixture was diluted with water (100 mL) and extracted with EtOAc (100 mL×3), the combined organics were washed with brine (100 mL), dried over anhydrous Na₂SO₄, filtered and concentrated to obtain the target compound 7.3 as solid (1.7 g, yield 91.6%). LC-MS (ESI) m/z: No mass. ¹H-NMR (400 MHz, DMSO-d₆) δ (ppm): 8.93 (dd, J=9.2, 2.9 Hz, 1H), 8.46 (dd, J=9.3, 2.8 Hz, 1H), 7.93 (d, J=8.6 Hz, 2H), 7.56 (t, J=7.1 Hz, 2H), 7.21 (t, J=9.8 Hz, 1H).

### Step 2: Synthesis of 5-(5-nitro-2-(4-(trifluoromethyl)phenoxy)phenyl)-2H-tetrazole (Compound 7.4)

To the solution of 5-nitro-2-(4-(trifluoromethyl)phenoxy)benzonitrile (1.6 g, 5.2 mmol) in 1,4-dioxane (20 mL) and H₂O (10 mL) was added NaN₃ (0.67 g, 10.4 mmol) and ZnBr₂ (0.58 g, 2.6 mmol). The mixture was stirred at 100° C. for 16 hours, concentrated and purified by flash column chromatography on silica gel (ethyl acetate in petroleum ether=30% v/v) to obtain the target compound 7.4 as solid (500 mg, yield 27.4%). LC-MS (ESI) m/z: 352 [M+H]⁺.

### Step 3 Synthesis of 2-methyl-5-(5-nitro-2-(4-(trifluoromethyl)phenoxy)phenyl)-2H-tetrazole (Compound 7.5)

To the solution of 5-(5-nitro-2-(4-(trifluoromethyl)phenoxy)phenyl)-2H-tetrazole (400 mg, 1.14 mmol) in DMF (10 mL) was added NaH (91.2 mg, 2.28 mmol), the mixture was stirred at 0° C. under N₂ for 0.5 hour, and then CH₃I (160 mg, 1.14 mmol) was added. The resulting mixture was stirred at rt for 1 hour, concentrated and purified by flash column chromatography on silica gel (ethyl acetate in petroleum ether=30% v/v) to obtain the target compound 7.5 as solid (100 mg, yield 24.0%). LC-MS (ESI) m/z: 366 [M+H]⁺.

### Step 4: Synthesis of 3-(2-methyl-2H-tetrazol-5-yl)-4-(4-(trifluoromethyl)phenoxy)aniline (Compound 7.6)

To a solution of 2-methyl-5-(5-nitro-2-(4-(trifluoromethyl)phenoxy)phenyl)-2H-tetrazole (80 mg, 0.22 mmol) in EtOH (10 mL) and THF (10 mL) was added Raney nickel (9 mg, 0.022 mmol), and then N₂H₄·H₂O (11 mg, 0.22 mmol) was added. The mixture was stirred at rt for 1 hour, filtered and concentrated in vacuum, the residue was purified by flash column chromatography on silica gel (ethyl acetate in petroleum ether-30% v/v) to obtain the target compound 7.6 as oil (50 mg, yield 54.5%). LC-MS (ESI) m/z: 336 [M+H]⁺.

### Step 5: Synthesis of N-(3-(2-methyl-2H-tetrazol-5-yl)-4-(4-(trifluoromethyl)phenoxy)phenyl)acrylamide (Compound A-3)

To a solution of 3-(2-methyl-2H-tetrazol-5-yl)-4-(4-(trifluoromethyl)phenoxy)aniline (50 mg, 0.15 mmol) in THF (10 mL) was added Et₃N (31 mg, 0.30 mmol) and acryloyl chloride (14 mg, 0.15 mmol), the mixture was stirred at 0° C. for 1 hour, concentrated and purified by prep-HPLC (MeCN/H₂O/TFA) to obtain the target compound A-3 as solid (4.5 mg, yield 7.7%). LC-MS (ESI) m/z: 390 [M+H]⁺. 1H-NMR (400 MHz, DMSO-d₆) δ (ppm): 8.45 (d, J=2.4 Hz, 1H), 7.91 (dd, J=8.8, 2.6 Hz, 1H), 7.60 (d, J=8.6 Hz, 2H), 7.24 (d, J=8.8 Hz, 1H), 7.04 (d, J=8.6 Hz, 2H), 6.51-6.40 (m, 2H), 5.84 (dd, J=9.3, 2.3 Hz, 1H), 4.61 (s, 1H), 4.34 (s, 3H).

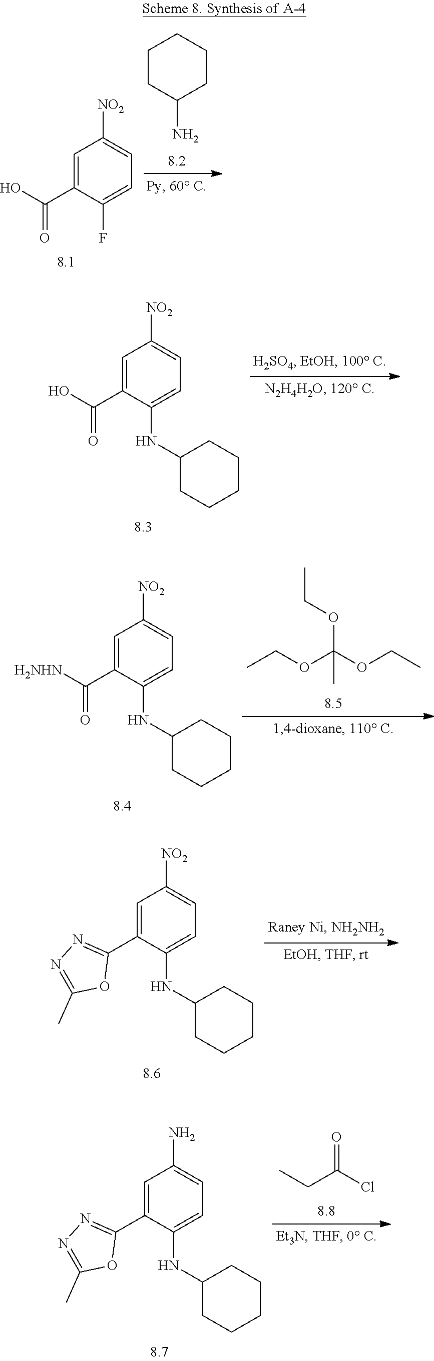

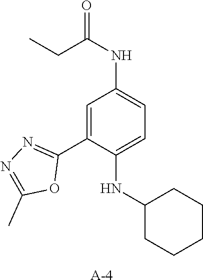 [SMILES: CCC(=O)Nc1ccc(NC2CCCCC2)c(-c2nnc(C)o2)c1 ; "A-4"]

### Step 1: Synthesis of 2-(cyclohexylamino)-5-nitrobenzoic Acid (Compound 8.3)

The mixture of 2-fluoro-5-nitrobenzoic acid (1000 mg, 5.4 mmol) and cyclohexanamine (535 mg, 5.4 mmol) in pyridine (20 mL) was stirred at 60° C. for 16 hours. The mixture was concentrated and purified by flash column chromatography on silica gel (ethyl acetate in petroleum ether-50% v/v) to obtain the target compound 8.3 as solid (1 g, yield 70.0%). LC-MS (ESI) m/z: 265 [M+H]⁺.

### Step 2: Synthesis of 2-(cyclohexylamino)-5-nitrobenzohydrazide (Compound 8.4)

To the solution of 2-(cyclohexylamino)-5-nitrobenzoic acid (1 g, 3.79 mmol) in EtOH (30 mL) was added H₂SO₄ (30 mL), the mixture was stirred at 100° C. for 16 hours. The solvent was removed, ammonia solution (30 mL) was added to the residue to render the resulting solution strongly alkaline. The mixture was extracted with ether (100 mL), the organic was dried over anhydrous Na₂SO₄, filtered and concentrated inn vacuum. N₂H₄H₂O (1 mL) was added to the residue and the resulting mixture was stirred at 120° C. for 3 hours. The mixture was diluted with water (100 mL) and extracted with EtOAc (3×50 mL), the combined organics were washed with brine (100 mL), dried over anhydrous Na₂SO₄, concentrated and purified by flash column chromatography on silica gel (ethyl acetate in petroleum ether=50% v/v) to obtain the target compound 8.4 as oil (300 mg, yield 28.5%). LC-MS (ESI) m/z: 279 [M+H]⁺.

### Step 3: Synthesis of N-cyclohexyl-2-(5-methyl-1,3,4-oxadiazol-2-yl)-4-nitroaniline (Compound 8.6)

The mixture of 2-(cyclohexylamino)-5-nitrobenzohydrazide (300 mg, 1.08 mmol) and 1,1,1-triethoxyethane (175 mg, 1.08 mmol) in 1,4-dioxane (10 mL) was stirred at 110° C. for 16 hours. The mixture was concentrated and purified by flash column chromatography on silica gel (ethyl acetate in petroleum ether=50% v/v) to obtain the target compound 8.6 as solid (250 mg, yield 76.7%). LC-MS (ESI) m/z: 303 [M+H]⁺.

### Step 4: Synthesis of N1-cyclohexyl-2-(5-methyl-1,3,4-oxadiazol-2-yl)benzene-1,4-diamine (Compound 8.7)

To a solution of N-cyclohexyl-2-(5-methyl-1,3,4-oxadiazol-2-yl)-4-nitroaniline (250 mg, 0.83 mmol) in EtOH (10 mL) and THF (10 mL) was added Raney nickel (33 mg, 0.08 mmol), and then N₂H₄·H₂O (41 mg, 0.83 mmol) was added. The mixture was stirred at rt for 1 hour and filtered through celite, the filtrate was concentrated and purified by flash column chromatography on silica gel (ethyl acetate in petroleum ether-50% v/v) to obtain the target compound 8.7 as oil (200 mg, yield 89.3%). LC-MS (ESI) m/z: 273 [M+H]⁺.

### Step 5: Synthesis of N-(4-(cyclohexylamino)-3-(5-methyl-1,3,4-oxadiazol-2-yl)phenyl)propionamide (Compound A-4)

To a solution of N1-cyclohexyl-2-(5-methyl-1,3,4-oxadiazol-2-yl)benzene-1,4-diamine (180 mg, 0.66 mmol) and Et₃N (133 mg, 1.32 mmol) in THF (10 mL) was added propionyl chloride (61 mg, 0.66 mmol). The mixture was stirred at 0° C. for 1 hour, concentrated and purified by prep-HPLC (MeCN/TFA/H₂O to obtain the target compound A-4 as solid (64 mg, yield 29.4%). LC-MS (ESI) m/z: 329 [M+H]⁺. ¹H NMR (400 MHz, DMSO-d₆) δ (ppm): 9.70 (s, 1H), 8.12 (d, J=2.5 Hz, 1H), 7.47 (dd, J=9.1, 2.5 Hz, 1H), 7.26 (d, J=7.7 Hz, 1H), 6.87 (d, J=9.2 Hz, 1H), 3.52 (d, J=7.9 Hz, 1H), 2.58 (s, 3H), 2.27 (q, J=7.6 Hz, 2H), 1.97 (d, J=9.3 Hz, 2H), 1.76-1.65 (m, 2H), 1.57 (s, 1H), 1.42 (dd, J=22.4, 11.7 Hz, 2H), 1.36-1.26 (m, 3H), 1.08 (t, J=7.6 Hz, 3H).

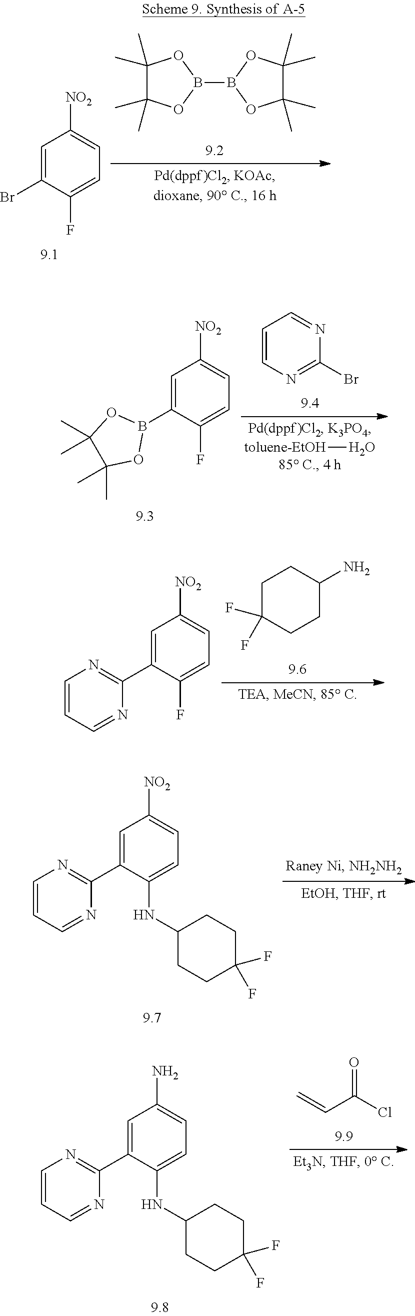

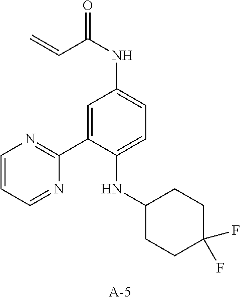 [SMILES: C=CC(=O)Nc1ccc(NC2CCC(F)(F)CC2)c(-c2ncccn2)c1 ; "A-5"]

### Step 1: Synthesis of 2-(2-fluoro-5-nitrophenyl)-4,4,5,5-tetramethyl-1,3,2-dioxaborolane (Compound 9.3)

To a solution of 2-bromo-1-fluoro-4-nitrobenzene (1000 mg, 4.6 mmol) in 1,4-dioxane (25 ml) and DMSO (0.5 mL) was added 4,4,4′,4′,5,5,5′,5′-octamethyl-2,2′-bi(1,3,2-dioxaborolane) (1280 mg, 5.0 mmol), KOAc (900 mg, 9.2 mmol) and Pd(dppf)Cl₂ (168 mg, 0.22 mmol). The mixture was stirred at 90° C. under N₂ for 16 hours, concentrated and purified by flash column chromatography on silica gel (ethyl acetate in petroleum ether=50% v/v) to obtain the target compound 9.3 as oil (800 mg, yield 65.3%). LC-MS (ESI) m/z: 268 [M+H]⁺.

### Step 2: Synthesis of 2-(2-fluoro-5-nitrophenyl)pyrimidine (Compound 9.5)

To a solution of 2-(2-fluoro-5-nitrophenyl)-4,4,5,5-tetramethyl-1,3,2-dioxaborolane (800 mg, 3.00 mmol) in Me₂CHOH (5 ml), PhMe (5 mL) and H₂O (5 mL) was added 2-bromopyrimidine (473 mg, 3.00 mmol), K₃PO₄ (1908 mg, 9.00 mmol) and Pd(dppf)Cl₂ (110 mg, 0.15 mmol). The mixture was stirred at 85° C. under N₂ for 4 hours. The mixture was diluted with water (100 mL) and extracted with EtOAc (3×50 mL), the combined organics were washed with brine (100 mL), dried over anhydrous Na₂SO₄, concentrated and purified by flash column chromatography on silica gel (ethyl acetate in petroleum ether=50% v/v) to obtain the target compound 9.5 as solid (600 mg, yield 91.4%). LC-MS (ESI) m/z: 220 [M+H]⁺.

### Step 3: Synthesis of N-(4,4-difluorocyclohexyl)-4-nitro-2-(pyrimidin-2-yl)aniline (Compound 9.7)

To a solution of 2-(2-fluoro-5-nitrophenyl)pyrimidine (513 mg, 2.17 mmol) in ACN (20 mL) was added N-(4,4-difluorocyclohexyl)-4-nitro-2-(pyrimidin-2-yl)aniline (450 mg, 1.98 mmol) and Et₃N (603 mg, 5.94 mmol), the mixture was stirred at 85° C. under N₂ for 16 hours. The mixture was concentrated and purified by flash column chromatography on silica gel (ethyl acetate in petroleum ether=50% v/v) to obtain the target compound 9.7 as solid (300 mg, yield 49.1%). LC-MS (ESI) m/z: 335 [M+H]⁺.

### Step 4: Synthesis of N1-(4,4-difluorocyclohexyl)-2-(pyrimidin-2-yl)benzene-1,4-diamine (Compound 9.8)

To the solution of N-(4,4-difluorocyclohexyl)-4-nitro-2-(pyrimidin-2-yl)aniline (250 mg, 0.75 mmol) in EtOH (10 mL) and THF (10 mL) was added Raney nickel (40 mg, 0.07 mmol), and then N₂H₄·H₂O (35 mg, 0.75 mmol) was added. The mixture was stirred at rt for 1 hour, concentrated and purified by flash column chromatography on silica gel (ethyl acetate in petroleum ether-50% v/v) to obtain the target compound 9.8 as oil (200 mg, yield 88.1%). LC-MS (ESI) m/z: 305 [M+H]⁺.

### Step 5: Synthesis of N-(4-(4,4-difluorocyclohexylamino)-3-(pyrimidin-2-yl)phenyl)acrylamide (Compound A-5)

To the solution of N1-(4,4-difluorocyclohexyl)-2-(pyrimidin-2-yl)benzene-1,4-diamine (180 mg, 0.59 mmol) and Et₃N (119 mg, 1.18 mmol) in THF (10 mL) was added acryloyl chloride (53 mg, 0.59 mmol). The mixture was stirred at 0° C. for 1 hour, concentrated and purified by prep-HPLC (MeCN/TFA/H₂O) to obtain the target compound A-5 as solid (49 mg, yield 23.2%). LC-MS (ESI) m/z: 359 [M+H]⁺. ¹H NMR (400 MHz, DMSO-d₆) δ (ppm): 9.99 (s, 1H), 9.02 (d, J=7.8 Hz, 1H), 8.90 (d, J=4.9 Hz, 2H), 8.69 (d, J=2.6 Hz, 1H), 7.75 (dd, J=9.1, 2.3 Hz, 1H), 7.38 (t, J=4.9 Hz, 1H), 6.88 (d, J=9.1 Hz, 1H), 6.41 (dd, J=17.0, 10.1 Hz, 1H), 6.21 (dd, J=17.0, 2.0 Hz, 1H), 5.69 (dd, J=10.1, 2.1 Hz, 1H), 3.75 (s, 1H), 2.05 (dd, J=15.6, 5.9 Hz, 6H), 1.60 (d, J=8.2 Hz, 2H).

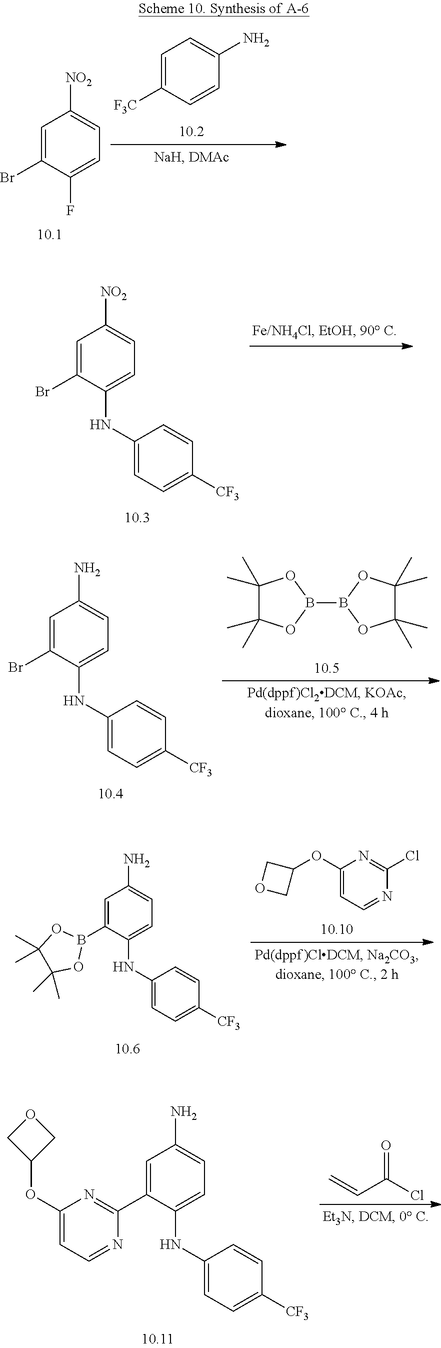

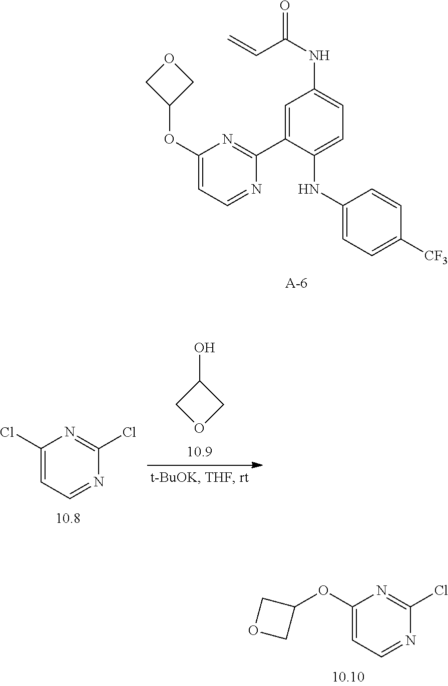 [SMILES: C=CC(=O)Nc1ccc(Nc2ccc(C(F)(F)F)cc2)c(-c2nccc(OC3COC3)n2)c1 ; "A-6" ; OC1COC1 ; Clc1ccnc(Cl)n1 ; Clc1nccc(OC2COC2)n1]

### Step 1: Synthesis of 2-bromo-4-nitro-N-(4-(trifluoromethyl)phenyl)aniline (Compound 10.3)

To the solution of 4-(trifluoromethyl)aniline (11.0 g, 50.0 mmol) in DMAc (150.0 mL) was added NaH (60%, 4 g, 100.0 mmol). The resulting mixture was stirred at 0° C. for 30 minutes, and then 2-bromo-1-fluoro-4-nitrobenzene (8.055 g, 50.0 mmol) was added. The mixture was stirred at 130° C. for 16 hours. The reaction mixture was cooled down to room temperature, diluted with H₂O (200 mL) and extracted with EtOAc (3×200 mL), the combined organic layer was dried over anhydrous Na₂SO₄, concentrated and purified by flash column chromatography on silica gel (eluting with 30% EA in PE) to afford the desired product 3 (2.431 g, 13.5% yield) as a yellow solid. LC-MS (ESI) m/z: 361 [M+H]⁺.

### Step 2: Synthesis of 2-bromo-N-(4-(trifluoromethyl)phenyl)benzene-1,4-diamine (Compound 10.4)

The mixture of compound 10.3 (2.431 g, 6.73 mmol), Fe power (3.769 g, 67.3 mmol) and NH₄Cl (1.740 g, 33.65 mmol) in EtOH (50.0 mL) and H₂O (5.0 mL) was stirred at 90° C. for 2 hours. After the completion of the reaction, the mixture was filtered, the filtrate was concentrated and purified by flash column chromatography on silica gel (eluting with 30% EA in PE) to afford the desired product 4 (2.197 g, 98.6% yield) as a brown solid. LC-MS (ESI) m/z: 331 [M+H]⁺.

### Step 3: Synthesis of 2-chloro-4-(oxetan-3-yloxy)pyrimidine (Compound 10.6)

To a three necked flask was added compound 10.4 (4.0 g, 12.12 mmol) and 1,4-dioxane (80 mL), followed by 4,4,4′,4′,5,5,5′,5′-octamethyl-2,2′-bi(1,3,2-dioxaborolane) (3.3858 g, 13.33 mmol), Pd(dppf)Cl₂·CH₂Cl₂ (494.9 mg, 0.606 mmol) and KOAc (2.3789 g, 24.24 mmol). The flask was evacuated, and refilled with N₂ for three times. The resulting mixture was stirred at 100° C. for 2 hours and monitored by LC-MS. After the completion of the reaction, the mixture was concentrated and purified by flash column chromatography on silica gel (eluting with 30% EA in PE) to afford the desired product (3.5 g, 76% yield) as a brown solid. LC-MS (ESI) m/z: 379 [M+H]⁺.

### Step 4: Synthesis of 2-bromo-N-(4-(trifluoromethyl)phenyl)benzene-1,4-diamine (Compound 10.10)

To the mixture of 2,4-dichloropyrimidine (2.235 g, 15.0 mmol) and oxetan-3-ol (1.112 g, 15.0 mmol) in THF (100.0 mL) was added t-BuOK (3.366 g, 30.0 mmol). The resulting mixture was stirred at room temperature for 16 hours and monitored by LC-MS. After the completion of the reaction, the mixture was concentrated and purified by flash column chromatography on silica gel (eluting with 30% EA in PE) to afford the desired product (2.309 g, 82.8% yield) as a white solid. LC-MS (ESI) m/z: 187 [M+H]⁺.

### Step 5: Synthesis of 2-(4-(oxetan-3-yloxy)pyrimidin-2-yl)-N1-(4-(trifluoromethyl)phenyl)benzene-1,4-diamine (Compound 10.11)

To a three necked flask was added compound 10.6 (1.512 g, 4.0 mmol), 1,4-dioxane (20 mL) and H₂O (2.0 mL), followed by the addition of compound 10.10 (781.2 mg, 4.2 mmol), Pd(dppf)Cl₂·CH₂Cl₂ (163.3 mg, 0.2 mmol) and Na₂CO₃ (864.0 mg, 8.0 mmol). The flask was evacuated, and refilled with N₂ for three times. The resulting mixture was stirred at 100° C. for 2 hours and monitored by LC-MS. the mixture was concentrated and purified by flash column chromatography on silica gel (eluting with 1% MeOH in DCM) to afford the desired product 11 (210 mg, 13.1% yield) as a yellow solid. LC-MS (ESI) m/z: 403 [M+H]⁺.

### Step 6: Synthesis of N-(3-(4-(oxetan-3-yloxy)pyrimidin-2-yl)-4-((4-(trifluoromethyl)phenyl)amino)phenyl)acrylamide (Compound A-6)

To the solution of compound 10.11 (200.0 mg, 0.5 mmol) and Et₃N (101.2 mg, 1.0 mmol) in DCM (5.0 mL) was added acryloyl chloride (45.3 mg, 0.5 mmol) dropwise at 0° C. The resulting solution was stirred at 0° C. for 20 min and monitored by LC-MS. The solvent was removed under reduce pressure to leave the crude product, which was purified by flash column chromatography on silica gel (eluting with 1% MeOH in DCM) to afford the desired product A-6 (135.0 mg, 59.2% yield) as a light yellow solid. LC-MS (ESI) m/z: 457 [M+H]⁺. ¹H NMR (400 MHz, DMSO-d₆) δ (ppm): 10.79 (s, 1H), 10.24 (s, 1H), 8.74-8.72 (m, 2H), 7.72 (dd, J=8.8, 2.4 Hz, 1H), 7.58 (d, J=8.4 Hz, 2H), 7.49 (d, J=8.8 Hz, 1H), 7.31 (d, J=8.4 Hz, 2H), 6.99 (d, J=5.6 Hz, 1H), 6.48 (dd, J=16.8, 10.0 Hz, 1H), 6.29 (dd, J=17.2, 2.0 Hz, 1H), 5.78 (dd, J=10.0, 2.0 Hz, 1H), 5.74-5.67 (m, 1H), 5.05 (t, J=7.2 Hz, 2H), 4.67 (dd, J=7.6, 5.6 Hz, 2H). ¹⁹F NMR (376 MHz, DMSO-d₆) δ (ppm): −59.75

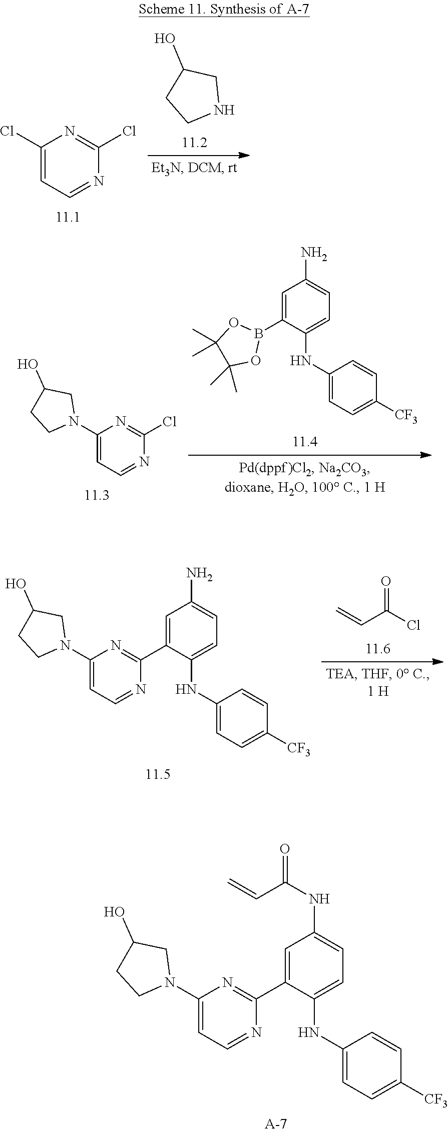

### Step 1: Synthesis of 1-(2-chloropyrimidin-4-yl)pyrrolidin-3-ol (Compound 11.3)

A mixture of 2,4-dichloropyrimidine (500 mg, 3.4 mmol), pyrrolidin-3-ol (290 mg, 3.4 mmol) and Et₃N (690 mg, 6.8 mmol) in DCM (10 mL) was stirred at RT under N₂ for 16 hours. The mixture was concentrated and purified by flash column chromatography on silica gel (ethyl acetate in petroleum ether=50% v/v) to obtain the target compound 11.3 as solid (400 mg, yield 59.1%). LC-MS (ESI) m/z: 199 [M+H]⁺.

### Step 2: Synthesis of 1-(2-(5-amino-2-(4-(trifluoromethyl)phenylamino)phenyl)pyrimidin-4-yl)pyrrolidin-3-ol (Compound 11.5)

A mixture of 1-(2-chloropyrimidin-4-yl)pyrrolidin-3-ol (70 mg, 0.35 mmol), 2-(4,4,5,5-tetramethyl-1,3,2-dioxaborolan-2-yl)-N1-(4-(trifluoromethyl)phenyl)benzene-1,4-diamine (130 mg, 0.35 mmol), Na₂CO₃ (111 mg, 1.05 mmol) and Pd(dppf)Cl₂ (30 mg, 0.04 mmol) in H₂O (1 mL) and 1,4-dioxane (10 mL) was stirred at 100° C. under N₂ for 1 hour. The mixture was concentrated and purified by flash column chromatography on silica gel (MeOH in DCM=15% v/v) to obtain the target compound 11.5 as solid (100 mg, yield 68.9%). LC-MS (ESI) m/z: 415 [M+H]⁺.

### Step 3: Synthesis of N-(3-(4-(3-hydroxypyrrolidin-1-yl)pyrimidin-2-yl)-4-(4-(trifluoromethyl)phenylamino)phenyl)acrylamide (A-7)

To a solution of 1-(2-(5-amino-2-(4-(trifluoromethyl)phenylamino)phenyl)pyrimidin-4-yl)pyrrolidin-3-ol (90 mg, 0.22 mmol) in THF (10 mL) was added acryloyl chloride (20 mg, 0.22 mmol) and Et₃N (45 mg, 0.44 mmol). The mixture was stirred at 0° C. under N₂ for 1 hour, concentrated and purified by prep-HPLC to obtain the target compound A-7 as solid (20 mg, yield 19.4%). LC-MS (ESI) m/z: 469 [M+H]⁺. ¹H NMR (400 MHz, DMSO-d₆) δ (ppm): 10.77 (s, 1H), 10.23 (s, 1H), 8.80-8.69 (m, 2H), 7.72 (dd, J=8.9, 2.5 Hz, 1H), 7.54 (dd, J=35.3, 8.7 Hz, 3H), 7.30 (d, J=8.5 Hz, 2H), 7.00 (d, J=5.8 Hz, 1H), 6.47 (dd, J=17.0, 10.1 Hz, 1H), 6.29 (dd, J=17.0, 1.8 Hz, 1H), 5.83-5.63 (m, 2H), 5.05 (t, J=7.1 Hz, 2H), 4.76-4.58 (m, 2H).

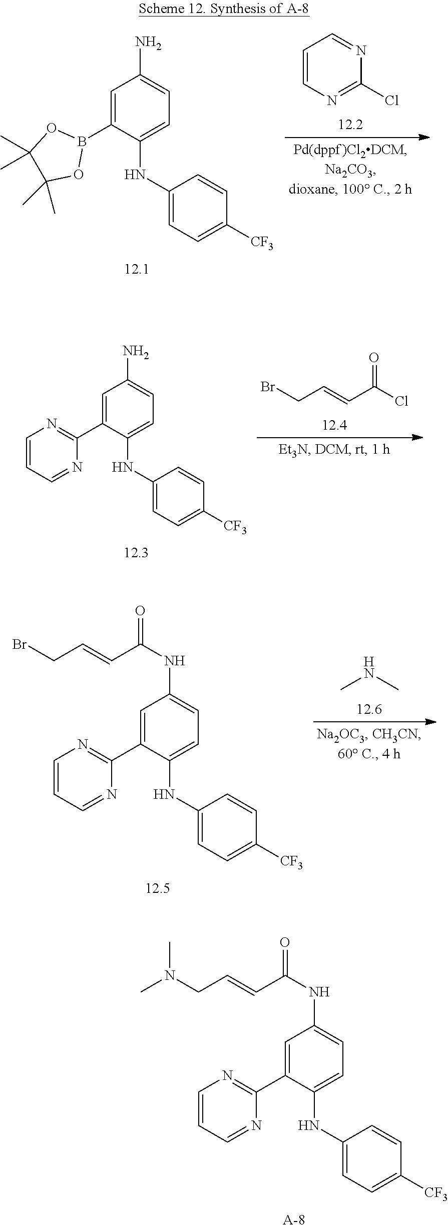

### Step 1: Synthesis of 2-(pyrimidin-2-yl)-N1-(4-(trifluoromethyl)phenyl)benzene-1,4-diamine (Compound 12.3)

To a three necked flask was added compound 12.1 (45.6 mg, 0.40 mmol), 1,4-dioxane (5.0 mL) and H₂O (1.0 mL), followed by 2-chloropyrimidine (151.2 mg, 0.40 mmol), Pd(dppf)Cl₂·CH₂Cl₂ (40.8 mg, 0.02 mmol) and Na₂CO₃ (84.8 mg, 0.8 mmol). The flask was evacuated, and refilled with N₂ for three times. The resulting mixture was stirred at 100° C. under N₂ for 16 hours. After the completion of the reaction, the mixture was concentrated and purified by column chromatography on silica gel (eluting with 5% MeOH in DCM) to afford the desired product (50 mg, 37.8% yield) as a brown solid. LC-MS (ESI) m/z: 331 [M+H]⁺.

### Step 2: Synthesis of (E)-4-bromo-N-(3-(pyrimidin-2-yl)-4-((4-(trifluoromethyl)phenyl)amino)phenyl)but-2-enamide (Compound 12.5)

To the solution of compound 12.3 (33.0 mg, 0.10 mmol) and Et₃N (12.9 mg, 0.11 mmol) in DCM (2.0 mL) was added (E)-4-bromobut-2-enoyl chloride (12.9 mg, 0.10 mmol, prepared by stirring of 1.0 equiv of (E)-4-bromobut-2-enoic acid and 2.0 equiv oxalyl chloride in DCM at 50° C. for 2 hours). The mixture was stirred at room temperature for 1 hour, concentrated and purified by column chromatography on silica gel (eluting with 2% MeOH in DCM) to give the desired product (34 mg, 71.4% yield) as a brown solid. LC-MS (ESI) m/z: 477 [M+H]⁺.

### Step 3: Synthesis of (E)-4-(dimethylamino)-N-(3-(pyrimidin-2-yl)-4-((4-(trifluoromethyl)phenyl)amino)phenyl)but-2-enamide (A-8)

A mixture of compound 12.5 (24.0 mg, 0.021 mmol), dimethylamine (2M in THF, 100 uL, 0.20 mmol) and Na₂CO₃ (10.6 mg, 0.10 mmol) in DCM (2.0 mL) was stirred at 0° C. for 2 hours. The mixture was concentrated and purified by prep-HPLC to afford the desired product A-8 (1.6 mg, 7.3% yield) as a viscous liquid. LC-MS (ESI) m/z: 442 [M+H]⁺, ¹H NMR (400 MHz, CD₃OD) δ (ppm): 8.82 (d, J=4.9 Hz, 2H), 8.62 (d, J=2.5 Hz, 1H), 7.68 (dd, J=8.9, 2.5 Hz, 1H), 7.44 (dd, J=8.7, 2.9 Hz, 3H), 7.28 (t, J=4.9 Hz, 1H), 7.22 (d, J=8.4 Hz, 2H), 6.80-6.71 (m, 1H), 6.48 (d, J=15.4 Hz, 1H), 3.91 (d, J=7.1 Hz, 2H), 2.85 (s, 6H). ¹⁹F NMR (376 MHz, CD₃OD) δ (ppm): −63.08, −76.99.

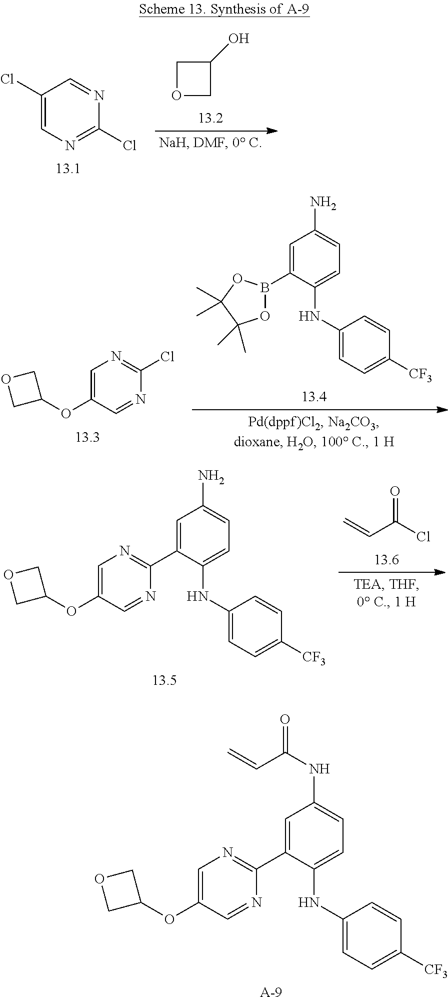

### Step 1: Synthesis of 2-chloro-5-(oxetan-3-yloxy)pyrimidine (Compound 13.3)

To a solution of oxetan-3-ol (0.55 g, 7.43 mmol) in DMF (10 mL) was added NaH (60%, 0.54 g, 13.52 mmol), the mixture was stirred at 0° C. under N₂ for 0.5 hour, and then 2,5-dichloropyrimidine (1 g, 6.76 mmol) was added. The resulting mixture was stirred at RT for 16 hours, diluted with water (100 mL) and extracted with EtOAc (50 mL×3), the combined organic was dried over anhydrous Na₂SO₄, concentrated and purified by flash column chromatography on silica gel (ethyl acetate in petroleum ether-50% v/v) to obtain the target compound 13.3 as oil (1.2 g, yield 95%). LC-MS (ESI) m/z: 186 [M+H]⁺.

### Step 2: Synthesis of 2-(5-(oxetan-3-yloxy)pyrimidin-2-yl)-N1-(4-(trifluoromethyl)phenyl)benzene-1,4-diamine (Compound 13.5)

A mixture of 2-chloro-5-(oxetan-3-yloxy)pyrimidine (150 mg, 0.80 mmol), 2-(4,4,5,5-tetramethyl-1,3,2-dioxaborolan-2-yl)-N1-(4-(trifluoromethyl)phenyl)benzene-1,4-diamine (300 mg, 0.80 mmol), Na₂CO₃ (255 mg, 2.4 mmol) and Pd(dppf)Cl₂ (59 mg, 0.08 mmol) in H₂O (1 mL) and 1,4-dioxane (10 mL) was stirred at 100° C. under N₂ for 1 hour. The mixture was concentrated and purified by flash column chromatography on silica gel (ethyl acetate in petroleum ether=15% v/v) to obtain the target compound 13.5 as solid (100 mg, yield 31.1%). LC-MS (ESI) m/z: 402 [M+H]⁺.

### Step 3: Synthesis of N-(3-(5-(oxetan-3-yloxy)pyrimidin-2-yl)-4-(4-(trifluoromethyl)phenylamino)phenyl)acrylamide (Compound A-9)

To a solution of 2-(5-(oxetan-3-yloxy)pyrimidin-2-yl)-N1-(4-(trifluoromethyl)phenyl)benzene-1,4-diamine (90 mg, 0.23 mmol) in THF (10 mL) was added acryloyl chloride (21 mg, 0.23 mmol) and Et₃N (47 mg, 0.46 mmol). The mixture was stirred at 0° C. under N₂ for 1 hour, concentrated and purified by prep-HPLC to obtain the target compound A-9 as solid (2 mg, yield 2%). LC-MS (ESI) m/z: 456 [M+H]⁺. ¹H NMR (400 MHz, DMSO-d₆) δ (ppm): 10.31 (s, 1H), 8.58 (s, 2H), 8.16 (s, 1H), 7.83-7.67 (m, 2H), 7.37 (dd, J=11.9, 8.7 Hz, 3H), 6.74 (d, J=8.5 Hz, 2H), 6.45 (dd, J=16.9, 10.2 Hz, 1H), 6.27 (d, J=16.9 Hz, 1H), 5.78 (d, J=11.8 Hz, 1H), 5.55 (t, J=5.6 Hz, 1H), 4.88 (t, J=6.9 Hz, 2H), 4.66-4.52 (m, 2H).

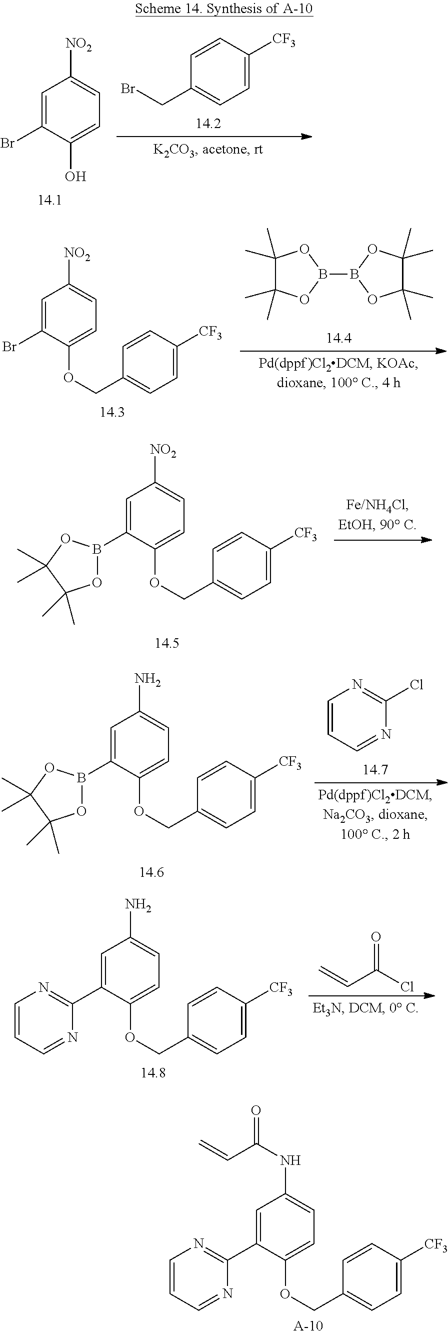

### Step 1: Synthesis of 2-bromo-4-nitro-1-((4-(trifluoromethyl)benzyl)oxy)benzene (Compound 14.3)

A mixture of 2-bromo-4-nitrophenol (4.36, 20.0 mmol), 1-(bromomethyl)-4-(trifluoromethyl)benzene (5.00 g, 21 mmol) and K₂CO₃ (5.52 mg, 40.0 mmol) in acetone (10.0 mL) was stirred at 50° C. for 3 hours. After the completion of the reaction, the mixture was filtered, the filter cake was washed with cooled H₂O and EtOH, and dried under vacuum to afford the desired product as solid (7.1 g, 94.7%), which was used directly for the next step without further purification. LC-MS (ESI) m/z: 376 [M+H]⁺.

### Step 2: Synthesis of 4,4,5,5-tetramethyl-2-(5-nitro-2-((4-(trifluoromethyl)benzyl)oxy)phenyl)-1,3,2-dioxaborolane (Compound 14.5)

To a three necked flask was added compound 14.3 (3.0 g, 8.0 mmol) and 1,4-dioxane (60.0 mL), followed by f 4,4,4′,4′,5,5,5′,5′-octamethyl-2,2′-bi(1,3,2-dioxaborolane) (2.134 g, 8.4 mmol), Pd(dppf)Cl₂·CH₂Cl₂ (327 mg, 0.40 mmol) and KOAc (1.568 g, 16.0. mmol). The flask was evacuated, and refilled with N₂ for three times. The resulting mixture was stirred at 100° C. under N₂ for 16 hours. After the completion of the reaction, the solvents were removed under reduce pressure to leave the crude product, which was purified by column chromatography on silica gel (eluting with 20% EtOAc in PE) to give the desired product (2.865 g, 84.7% yield) as a brown solid. LC-MS (ESI) m/z: 424 [M+H]⁺.

### Step 3: Synthesis of 3-(4,4,5,5-tetramethyl-1,3,2-dioxaborolan-2-yl)-4-((4-(trifluoromethyl)benzyl)oxy)aniline (Compound 14.6)

To the solution of compound 14.5 (846 mg, 2.0 mmol) and NH₄Cl (535 mg, 10.0 mmol) in EtOH (2.0 mL) and H₂O (1.0 mL) was added Fe powder (1.12 g, 20 mmol). The resulting mixture was stirred at 90° C. for 2 hours. The mixture was filtered through celite, the filtrate was concentrated under reduce pressure to leave the crude compound 14.6, which was used directly for the next step without further purification. LC-MS (ESI) m/z: 393 [M+H]⁺.

### Step 4: Synthesis of 3-(pyrimidin-2-yl)-4-((4-(trifluoromethyl)benzyl)oxy)aniline (Compound 14.8)

To a three necked flask was added compound 14.6 (500 mg, 1.27 mmol), 1,4-dioxane (10 mL) and H₂O (2.0 mL), followed by 2-chloropyrimidine (152.8 mg, 1.34 mmol), Pd(dppf)Cl₂·CH₂Cl₂ (52.3 mg, 0.64 mmol) and Na₂CO₃ (269.2 mg, 2.54 mmol). The flask was evacuated, and refilled with N₂ for three times. The resulting mixture was stirred at 100° C. under N₂ for 16 hours. After the completion of the reaction, the solvents were removed under reduce pressure to leave the crude product, which was purified by column chromatography on silica gel (eluting with 5% MeOH in DCM) to afford the desired product (0.21 g, 46% yield) as a yellow solid. LC-MS (ESI) m/z: 346 [M+H]⁺.

### Step 5: Synthesis of N-(3-(pyrimidin-2-yl)-4-((4-(trifluoromethyl)benzyl)oxy)phenyl)acrylamide (Compound A-10)

To the solution of compound 14.8 (150.0 mg, 0.382 mmol) and Et₃N (77.2 mg, 0.764 mmol) in DCM (5.0 mL) was added acryloyl chloride (84.5 mg, 0.764 mmol). The mixture was stirred at 0° C. for 2 hours and monitored by LC-MS. After the completion of the reaction the mixture was concentrated and purified by prep-HPLC to afford the desired pure product A-10 (45 mg, 30%) as a yellow solid. LC-MS (ESI) m/z: 346 [M+H]⁺. ¹H NMR (400 MHz, DMSO-d₆) δ (ppm) 10.21 (s, 1H), 8.96 (d, J=5.2 Hz, 2H), 8.04 (d, J=2.6 Hz, 1H), 7.77-7.68 (m, 5H), 7.49 (t, J=4.9 Hz, 1H), 7.21 (d, J=8.8 Hz, 1H), 6.43 (dd, J=16.8, 10.0 Hz, 1H), 6.25 (dd, J=16.8, 2.0 Hz, 1H), 5.75 (dd, J=10.0, 2.0 Hz, 1H), 5.28 (s, 2H). ¹⁹F NMR (376 MHz, DMSO-d₆) δ (ppm): −60.87.

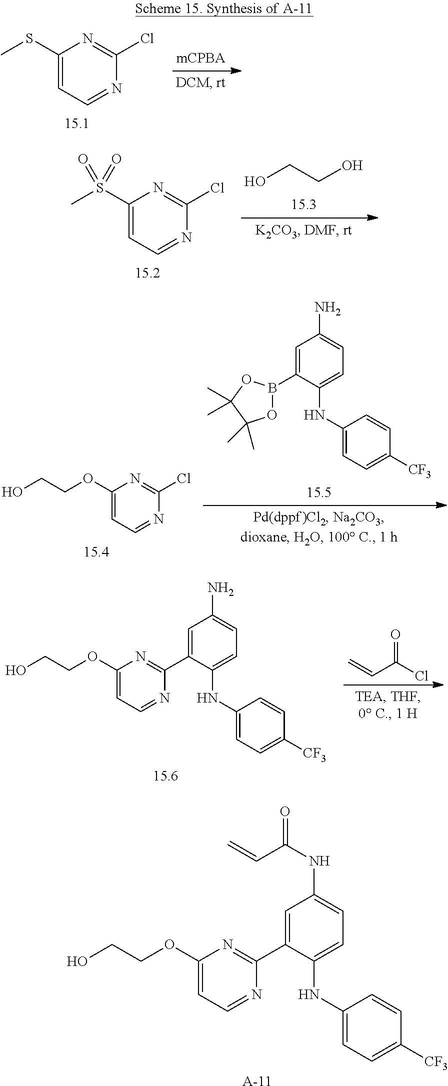

### Step 1: Synthesis of 2-chloro-4-(methylsulfonyl)pyrimidine (Compound 15.2)

To a solution of 2-chloro-4-(methylthio)pyrimidine (500 mg, 3.2 mmol) in DCM (100 mL) was added mCPBA (540 mg, 3.2 mmol). The mixture was stirred at RT under N₂ for 16 hours. The mixture was washed with saturated NaHCO₃ solution (100 mL), dried over anhydrous Na₂SO₄, filtered and concentrated under vacuum to obtain the target compound 15.2 as solid (500 mg, yield 81%). LC-MS (ESI) m/z: 192 [M+H]⁺.

### Step 2: Synthesis of 2-(2-chloropyrimidin-4-yloxy)ethanol (Compound 15.4)

To a solution of 2-chloro-4-(methylsulfonyl)pyrimidine (450 mg, 2.34 mmol) in DMF (10 mL) was added ethane-1,2-diol (291 mg, 4.68 mmol) and K₂CO₃ (496 mg, 4.68 mmol). The mixture was stirred at RT under N₂ for 2 hours. The mixture was concentrated and purified by flash column chromatography on silica gel (ethyl acetate in petroleum ether=20% v/v) to obtain the target compound 15.4 as oil (200 mg, yield 49.2%). LC-MS (ESI) m/z: 174 [M+H]⁺.

### Step 3: Synthesis of 2-(2-(5-amino-2-(4-(trifluoromethyl)phenylamino)phenyl) pyrimidin-4-yloxy)ethanol (Compound 15.6)

The mixture of 2-(2-chloropyrimidin-4-yloxy)ethanol (200 mg, 0.5 mmol), 2-(4,4,5,5-tetramethyl-1,3,2-dioxaborolan-2-yl)-N1-(4-(trifluoromethyl)phenyl)benzene-1,4-diamine (100 mg, 0.5 mmol), Na₂CO₃ (160 mg, 1.5 mmol) and Pd(dppf)Cl₂ (40 mg, 0.05 mmol) in H₂O (1 mL) and 1,4-dioxane (10 mL) was stirred at 100° C. under N₂ for 1 hour. The mixture was concentrated and purified by flash column chromatography on silica gel (ethyl acetate in petroleum ether=20% v/v) to obtain the target compound 15.6 as solid (100 mg, yield 51.3%). LC-MS (ESI) m/z: 390 [M+H]⁺.

### Step 4: Synthesis of N-(3-(4-(2-hydroxyethoxy)pyrimidin-2-yl)-4-(4-(trifluoromethyl) phenylamino)phenyl)acrylamide (Compound A-11)

To a solution of 2-(2-(5-amino-2-(4-(trifluoromethyl)phenylamino)phenyl) pyrimidin-4-yloxy)ethanol (90 mg, 0.23 mmol) in THF (10 mL) was added acryloyl chloride (21 mg, 0.23 mmol) and Et₃N (47 mg, 0.46 mmol). The mixture was stirred at 0° C. under N₂ for 1 hour, concentrated and purified by prep-HPLC (MeCN/TFA/H₂O) to obtain the target compound A-11 as solid (5 mg, yield 5%). LC-MS (ESI) m/z: 444 [M+H]⁺. ¹H NMR (400 MHz, DMSO-d₆) δ (ppm): 10.69 (s, 1H), 10.26 (s, 1H), 8.67 (dd, J=8.1, 4.2 Hz, 2H), 8.41 (s, 1H), 7.86 (s, 1H), 7.57 (d, J=8.6 Hz, 2H), 7.50 (d, J=9.0 Hz, 1H), 7.29 (d, J=8.5 Hz, 2H), 6.91 (d, J=5.8 Hz, 1H), 6.46 (dd, J=16.7, 9.7 Hz, 1H), 6.26 (d, J=16.8 Hz, 1H), 5.76 (d, J=12.1 Hz, 1H), 4.99 (s, 1H), 4.55-4.42 (m, 2H), 3.78 (s, 1H).

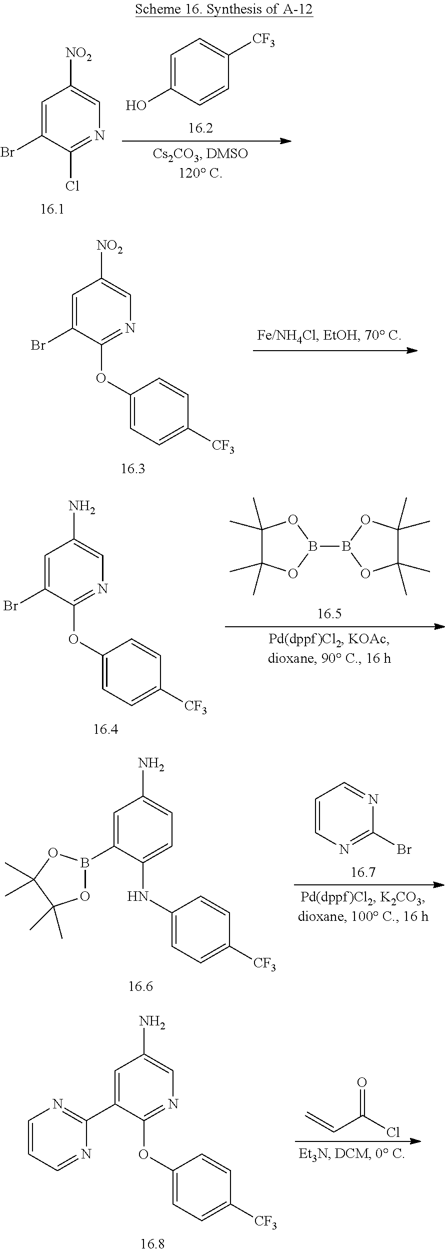

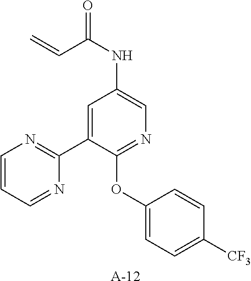 [SMILES: C=CC(=O)Nc1cnc(Oc2ccc(C(F)(F)F)cc2)c(-c2ncccn2)c1 ; "A-12"]

### Step 1: Synthesis of 3-bromo-5-nitro-2-(4-(trifluoromethyl)phenoxy)pyridine (Compound 16.3)

A mixture of 3-bromo-2-chloro-5-nitropyridine (1500 mg, 6.75 mmol), 4-(trifluoromethyl)phenol (1093 mg, 6.75 mmol) and Cs₂CO₃ (2413 mg, 7.4 mmol) in DMSO (20 mL) was stirred at 120° C. under N₂ overnight. The reaction mixture was cooled down to room temperature, diluted with H₂O (200 mL) and extracted with ethyl acetate (300 mL×2), the combined organic was washed with water (500 mL), dried over anhydrous Na₂SO₄, filtered and concentrated to leave the crude product 3 as yellow solid (2 g, yield 82%). LC-MS (ESI) m/z: No Mass.

### Step 2: Synthesis of 5-bromo-6-(4-(trifluoromethyl)phenoxy)pyridin-3-amine (Compound 16.4)

A mixture of compound 16.3 (1000 mg, 2.76 mmol), Fe (1546 mg, 27.6 mmol) and NH₄Cl (1462 mg, 27.6 mmol) in EtOH (20 mL) was stirred at 70° C. under N₂ for 5 hours. The reaction mixture was filtered, the filtrate was concentrated to leave the crude product 4 as yellow solid (800 mg, yield 87%). LC-MS (ESI) m/z: 333 [M+H]⁺.

### Step 3: Synthesis of 5-(4,4,5,5-tetramethyl-1,3,2-dioxaborolan-2-yl)-6-(4-(trifluoromethyl)phenoxy)pyridin-3-amine (Compound 16.6)

A mixture of compound 16.4 (200 mg, 0.6 mmol), 4,4,4′,4′,5,5,5′,5′-octamethyl-2,2′-bi(1,3,2-dioxaborolane) (228 mg, 0.9 mmol), KOAc (176 mg, 1.8 mmol) and Pd(dppf)Cl₂ (44 mg, 0.06 mmol) in 1,4-dixoane (10 mL) was stirred at 90° C. under N₂ overnight. The reaction mixture was filtered, the filtrate was concentrated to leave the crude compound 16.6 (200 mg, yield 87%) as oil, which was used directly for next step without further purification. LC-MS (ESI) m/z: 381 [M+H]⁺.

### Step 4: Synthesis of 5-(pyrimidin-2-yl)-6-(4-(trifluoromethyl)phenoxy)pyridin-3-amine (Compound 16.8)

A mixture of compound 16.6 (100 mg, 0.25 mmol), 2-bromopyrimidine (40 mg, 0.25 mmol), K₂CO₃ (105 mg, 0.75 mmol) and Pd(dppf)Cl₂ (20 mg, 0.025 mmol) in 1,4-dioxane (5 mL) and H₂O (1 mL) was stirred at 90° C. under N₂ overnight. The reaction mixture was concentrated and purified by prep-HPLC to obtain compound 16.8 as white solid (50 mg, yield 60%). LC-MS (ESI) m/z: 333 [M+H]⁺.

### Step 5: Synthesis of N-(5-(pyrimidin-2-yl)-6-(4-(trifluoromethyl)phenoxy)pyridin-3-yl)acrylamide (Compound A-12)

A mixture of compound 16.8 (40 mg, 0.12 mmol), acryloyl chloride (12 mg, 0.12 mmol) and TEA (24 mg, 0.24 mmol) in DCM (10 mL) was stirred at 0° C. for 2 hours. The mixture was concentrated and purified by prep-HPLC to obtain the desired A-12 as white solid (10 mg, yield 22%). LC-MS (ESI) m/z: 387 [M+H]⁺. ¹H NMR (400 MHz, CD₃OD) δ (ppm): 8.90 (d, J=5.0 Hz, 2H), 8.65 (dd, J=60.5, 2.7 Hz, 2H), 7.65 (d, J=8.5 Hz, 2H), 7.47 (t, J=5.0 Hz, 1H), 7.23 (d, J=8.5 Hz, 2H), 6.43 (t, J=6.1 Hz, 2H), 5.83 (dd, J=8.9, 2.9 Hz, 1H).

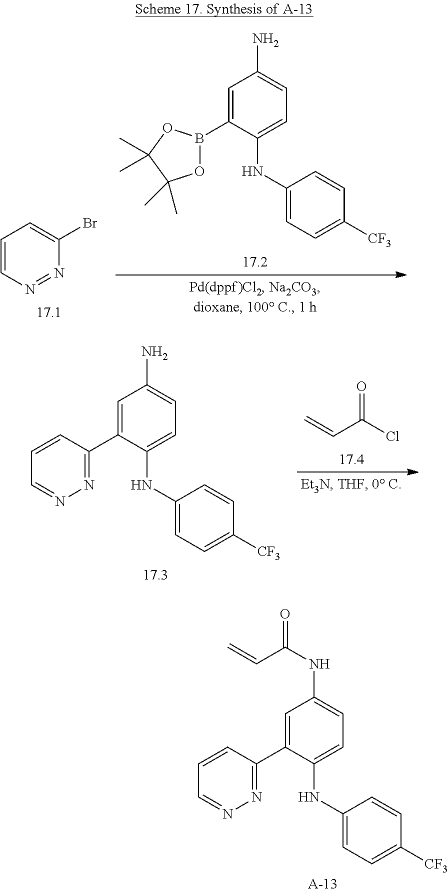

### Step 1: Synthesis of 2-(pyridazin-3-yl)-N1-(4-(trifluoromethyl)phenyl)benzene-1,4-diamine (Compound 17.3)

To the solution of 2-(4,4,5,5-tetramethyl-1,3,2-dioxaborolan-2-yl)-N1-(4-(trifluoromethyl)phenyl)benzene-1,4-diamine (200 mg, 0.5 mmol) in H₂O (1 mL) and 1,4-dioxane (10 mL) was added 3-bromopyridazine (80 mg, 0.5 mmol), Na₂CO₃ (160 mg, 1.5 mmol) and Pd(dppf)Cl₂ (40 mg, 0.05 mmol)). The mixture was stirred at 100° C. under N₂ for 1 hour. After cooled down to rt the mixture was diluted with water (100 mL) and extracted with EtOAc (3×100 mL), the combined organics were washed with brine (100 mL), dried over anhydrous Na₂SO₄, concentrated and purified by flash column chromatography on silica gel (ethyl acetate in petroleum ether=50% v/v) to obtain the target compound 17.3 as solid (100 mg, yield 57.5%). LC-MS (ESI) m/z: 330 [M+H]⁺.

### Step 2: Synthesis of N-(3-(pyridazin-3-yl)-4-(4-(trifluoromethyl)phenylamino)phenyl) acrylamide (Compound A-13)

To the solution of 2-(pyridazin-3-yl)-N1-(4-(trifluoromethyl)phenyl) benzene-1,4-diamine (90 mg, 0.37 mmol) in THF (10 mL) was added Et₃N (75 mg, 0.74 mmol) and acryloyl chloride (25 mg, 0.37 mmol), the mixture was stirred at 0° C. for 1 hour, concentrated and purified by prep-HPLC (MeCN/H₂O/TFA) to obtain the target compound A-13 as solid (20 mg, yield 19.1%). LC-MS (ESI) m/z: 384 [M+H]⁺. ¹H NMR (400 MHz, DMSO-d₆) δ (ppm): 10.34 (s, 1H), 9.17 (dd, J=4.9, 1.6 Hz, 1H), 8.75 (s, 1H), 8.11 (d, J=2.4 Hz, 1H), 7.82 (ddd, J=8.6, 4.4, 2.0 Hz, 2H), 7.69 (dd, J=8.6, 4.9 Hz, 1H), 7.50-7.37 (m, 3H), 6.86 (d, J=8.6 Hz, 2H), 6.45 (dd, J=17.0, 10.1 Hz, 1H), 6.28 (dd, J=17.0, 2.0 Hz, 1H), 5.78 (dd, J=10.1, 1.9 Hz, 1H).

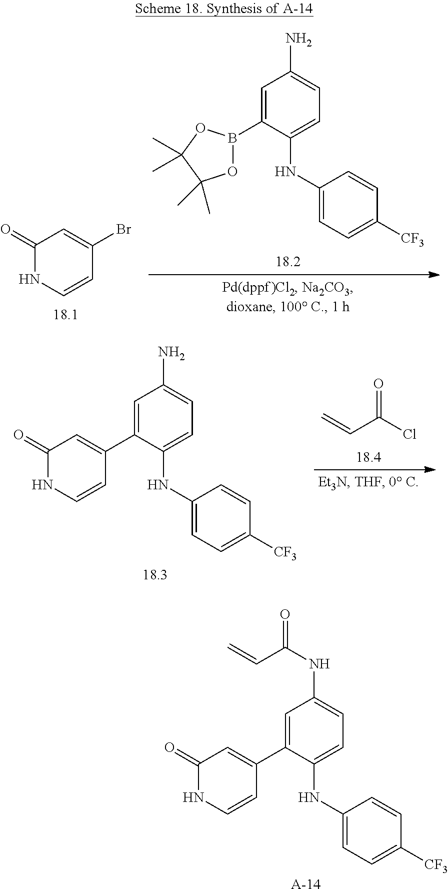

### Step 1: Synthesis of 4-(5-amino-2-(4-(trifluoromethyl)phenylamino)phenyl)pyridin-2(1H)-one (Compound 18.3)

To the solution of 2-(4,4,5,5-tetramethyl-1,3,2-dioxaborolan-2-yl)-N1-(4-(trifluoromethyl)phenyl)benzene-1,4-diamine (200 mg, 0.5 mmol) in H₂O (1 mL) and 1,4-dioxane (10 mL) was added 4-bromopyridin-2(1H)-one (90 mg, 0.5 mmol), Na₂CO₃ (160 mg, 1.5 mmol) and Pd(dppf)Cl₂ (40 mg, 0.05 mmol). The mixture was stirred at 100° C. under N₂ for 1 hour. After cooled down to rt the mixture was diluted with water (100 mL) and extracted with EtOAc (3×100 mL), the combined organics were washed with brine (100 mL), dried over anhydrous Na₂SO₄, concentrated and purified by flash column chromatography on silica gel (ethyl acetate in petroleum ether=50% v/v) to obtain the target compound 18.3 as solid (100 mg, yield 54.8%). LC-MS (ESI) m/z: 345 [M+H]⁺.

### Step 2: Synthesis of N-(3-(2-oxo-1,2-dihydropyridin-4-yl)-4-(4-(trifluoromethyl) phenylamino)phenyl)acrylamide (Compound A-14)

To the solution of 4-(5-amino-2-(4-(trifluoromethyl)phenylamino)phenyl)pyridin-2(1H)-one (90 mg, 0.26 mmol) in THF (10 mL) was added Et₃N (53 mg, 0.52 mmol) and acryloyl chloride (24 mg, 0.26 mmol), the mixture was stirred at 0° C. for 1 hour, concentrated and purified by prep-HPLC (MeCN/H₂O/TFA) to obtain the target compound A-14 as solid (33 mg, yield 31.7%). LC-MS (ESI) m/z: 399 [M+H]⁺. ¹H NMR (400 MHz, DMSO-d₆) δ (ppm) 10.29 (s, 1H), 8.15 (s, 1H), 7.79 (d, J=2.4 Hz, 1H), 7.70 (dd, J=8.6, 2.4 Hz, 1H), 7.40 (d, J=8.6 Hz, 2H), 7.33 (t, J=7.7 Hz, 2H), 6.79 (d, J=8.5 Hz, 2H), 6.44 (dd, J=17.0, 10.1 Hz, 1H), 6.37-6.21 (m, 2H), 6.14 (dd, J=6.8, 1.6 Hz, 1H), 5.78 (dd, J=10.1, 2.0 Hz, 1H).

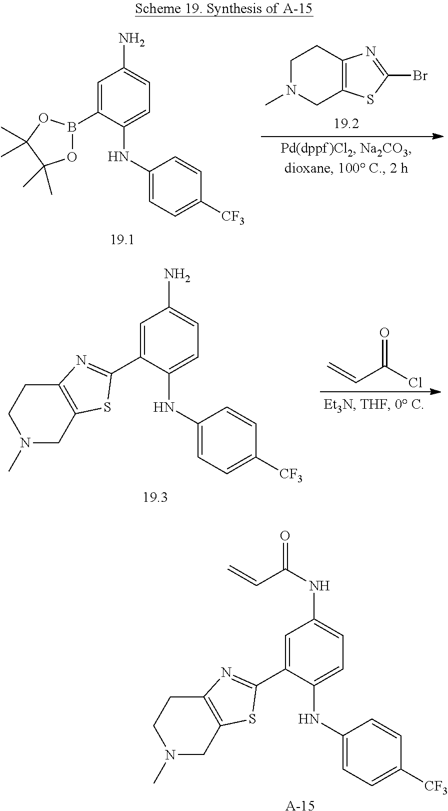

### Step 1: Synthesis of 2-(5-methyl-4,5,6,7-tetrahydrothiazolo[5,4-c]pyridin-2-yl)-N1-(4-(trifluoromethyl)phenyl)benzene-1,4-diamine (Compound 19.3)

To a three necked flask was added compound 19.1 (756 mg, 2.0 mmol), 1,4-dioxane (40 mL) and H₂O (10 mL), followed by the addition of 2-bromo-5-methyl-4,5,6,7-tetrahydrothiazolo[5,4-c]pyridine 2 (466 mg, 2.0 mmol), Pd(dppf)Cl₂ (72 mg, 0.1 mmol) and Na₂CO₃ (426 mg, 4.0 mmol). The flask was evacuated, and refilled with N₂ for three times. The resulting mixture was stirred at 100° C. for 2 hours and monitored by LC-MS. After the completion of the reaction, the solvents were removed under reduce pressure to leave the crude product, which was purified by column chromatography on silica gel (eluting with 1% MeOH in DCM) to afford the desired compound 19.3 (600 mg, 74.3% yield) as a yellow solid. LC-MS (ESI) m/z: 405 [M+H]⁺.

### Step 2: Synthesis of N-(3-(5-methyl-4,5,6,7-tetrahydrothiazolo[5,4-c]pyridin-2-yl)-4-((4-(trifluoromethyl)phenyl)amino)phenyl)acrylamide (Compound A-15)

To the solution of compound 19.3 (202.0 mg, 0.50 mmol) and Et₃N (101.2 mg, 1.0 mmol) in DCM (15.0 mL) was added acryloyl chloride (45.2 mg, 0.50 mmol). The resulting mixture was stirred at 0° C. for 20 minutes and monitored by LC-MS. After the completion of the reaction, the solvent was removed under reduce pressure to leave the crude product, which was purified by column chromatography on silica gel (eluting with 1% MeOH in DCM) to afford the desired product A-15 (52.0 mg, 22.7% yield) as a yellow solid. ¹H NMR (400 MHz, DMSO-d₆) δ (ppm) 10.38 (s, 1H), 9.08 (s, 1H), 8.48 (d, J=2.4 Hz, 1H), 7.83 (dd, J=8.7, 2.4 Hz, 1H), 7.50 (d, J=8.6 Hz, 2H), 7.39 (d, J=8.7 Hz, 1H), 6.92 (d, J=8.5 Hz, 2H), 6.46 (dd, J=17.0, 10.1 Hz, 1H), 6.29 (dd, J=17.0, 2.0 Hz, 1H), 5.79 (dd, J=10.0, 2.0 Hz, 1H), 3.58 (s, 2H), 2.84 (t, J=5.3 Hz, 2H), 2.73 (t, J=5.6 Hz, 2H), 2.37 (s, 3H). ¹⁹F NMR (376 MHz, DMSO-d₆) δ (ppm) −59.46.

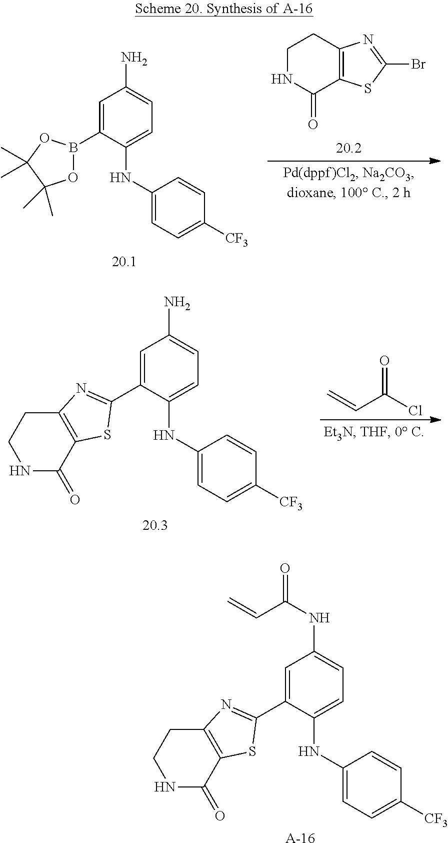

### Step 1: Synthesis of 2-(5-amino-2-((4-(trifluoromethyl)phenyl)amino)phenyl)-6,7-dihydrothiazolo[5,4-c]pyridin-4(5H)-one (Compound 20.3)

To a solution of compound 20.1 (375.0 mg, 0.917 mmol) in 1,4-dioxane (8 mL) and H₂O (2 mL) was added 2-bromo-6,7-dihydrothiazolo[5,4-c]pyridin-4(5H)-one (203.4 mg, 0.873 mmol), Pd(dppf)Cl₂ (32.2 mg, 0.044 mmol) and Na₂CO₃ (185.1 mg, 1.746 mmol). The flask was evacuated, and refilled with N₂ for three times. The resulting mixture was stirred at 100° C. for 2 hours and monitored by LC-MS. After the completion of the reaction, the solvents were removed under reduce pressure to leave the crude product, which was purified by flash column chromatography on silica gel (eluting with 1% MeOH in DCM) to give the desired compound 20.3 (310 mg, 76.7% yield) as a yellow solid. LC-MS (ESI) m/z: 405 [M+H]⁺.

### Step 2: Synthesis of N-(3-(4-oxo-4,5,6,7-tetrahydrothiazolo[5,4-c]pyridin-2-yl)-4-((4-(trifluoromethyl)phenyl)amino)phenyl)acrylamide (Compound A-16)

To the solution of compound 20.3 (202.0 mg, 0.50 mmol) and Et₃N (101.2 mg, 1.0 mmol) in DCM (15.0 mL) was added dropwise acryloyl chloride (45.2 mg, 0.50 mmol). The mixture was stirred at 0° C. for 20 minutes and monitored by LC-MS. After the completion of the reaction, the solvent was removed under reduce pressure to leave the crude product, which was purified by column chromatography on silica gel (eluting with 1% MeOH in DCM) to give the desired product A-16 (56 mg, 24.5% yield) as a yellow solid. ¹H NMR (400 MHz, DMSO-d₆) δ (ppm) 10.45 (s, 1H), 8.81 (s, 1H), 8.66 (d, J=2.4 Hz, 1H), 7.93 (dd, J=8.7, 2.5 Hz, 1H), 7.88 (s, 1H), 7.50 (d, J=8.6 Hz, 2H), 7.41 (d, J=8.7 Hz, 1H), 6.84 (d, J=8.5 Hz, 2H), 6.47 (dd, J=16.9, 10.1 Hz, 1H), 6.31 (dd, J=17.0, 1.9 Hz, 1H), 5.80 (dd, J=10.1, 1.9 Hz, 1H), 3.50 (td, J=7.0, 2.4 Hz, 2H), 3.03 (t, J=7.0 Hz, 2H). ¹⁹F NMR (376 MHz, DMSO-d₆) δ (ppm) −59.45.

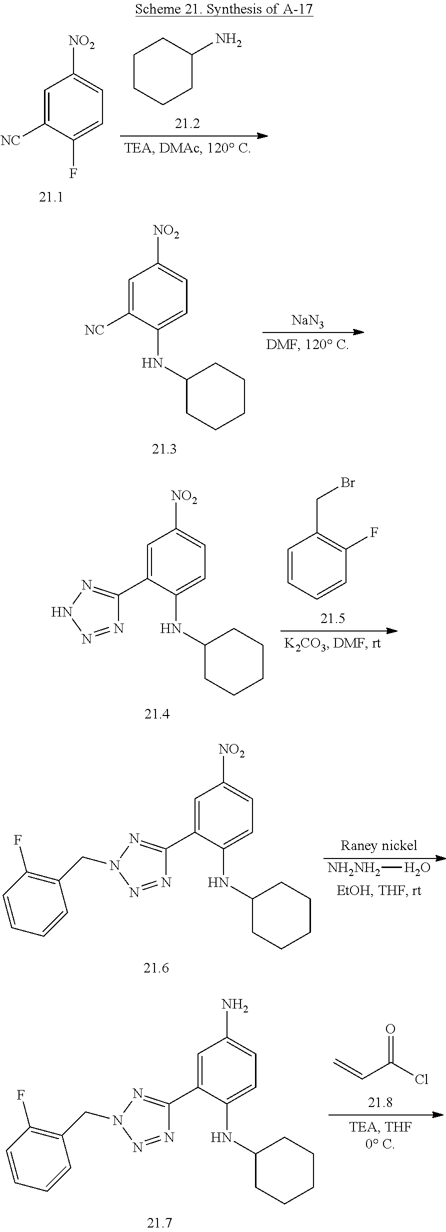

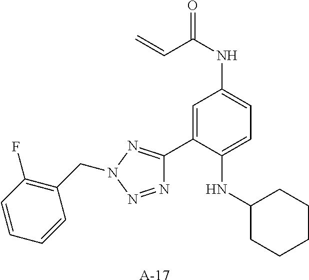 [SMILES: C=CC(=O)Nc1ccc(NC2CCCCC2)c(-c2nnn(Cc3ccccc3F)n2)c1 ; "A-17"]

### Step 1: Synthesis of 2-(cyclohexylamino)-5-nitrobenzonitrile (Compound 21.3)

The mixture of 2-fluoro-5-nitrobenzonitrile (1 g, 6.0 mmol), cyclohexanamine (0.6 g, 6.0 mmol) and TEA (1.2 g, 12.0 mmol) in DMAc (5 mL) was heated at 120° C. under N₂ for 16 hours, until all starting material was consumed as showed by LCMS. The mixture was concentrated and purified by flash column chromatography on silica gel (ethyl acetate in petroleum ether=50% v/v) to afford the target compound 21.3 as solid (1.4 g, yield 94.9%). LC-MS (ESI) m/z: 246 [M+H]⁺.

### Step 2: Synthesis of N-cyclohexyl-4-nitro-2-(2H-tetrazol-5-yl) aniline (Compound 21.4)

The mixture of 2-(cyclohexylamino)-5-nitrobenzonitrile (1.3 g, 5.3 mmol) and NaN₃ (0.52 g, 7.9 mmol) in DMF (30 mL) was heated at 120° C. under N₂ for 16 hours, until all starting material was consumed as showed by LCMS. The mixture was concentrated and purified by flash column chromatography on silica gel (ethyl acetate in petroleum ether=20% v/v) to obtain the target compound 21.4 as solid (800 mg, yield 52.3%). LC-MS (ESI) m/z: 289 [M+H]⁺.

### Step 3: Synthesis of N-cyclohexyl-2-(2-(2-fluorobenzyl)-2H-tetrazol-5-yl)-4-nitroaniline (Compound 21.6)

The mixture of N-cyclohexyl-4-nitro-2-(2H-tetrazol-5-yl)aniline (700 mg, 2.43 mmol), 1-(bromomethyl)-2-fluorobenzene (456 mg, 2.43 mmol) and K₂CO₃ (670 mg, 4.86 mmol) in DMF (20 mL) was stirred at rt under N₂ for 3 hours. The mixture was concentrated and purified by flash column chromatography on silica gel (ethyl acetate in petroleum ether=40% v/v) to obtain the target compound 21.6 as solid (600 mg, yield 62.3%). LC-MS (ESI) m/z: 397 [M+H]⁺.

### Step 4: Synthesis of N1-cyclohexyl-2-(2-(2-fluorobenzyl)-2H-tetrazol-5-yl)benzene-1,4-diamine (Compound 21.7)

To the solution of N-cyclohexyl-2-(2-(2-fluorobenzyl)-2H-tetrazol-5-yl)-4-nitroaniline (500 mg, 1.26 mmol) in EtOH (15 mL) and THF (15 mL) was added Raney nickel (53 mg, 0.13 mmol), and then N₂H₄·H₂O (63 mg, 1.26 mmol) was added. The mixture was stirred at rt for 1 hour and filtered, the filtrate was concentrated under reduced pressure to leave the crude compound 21.7 as oil (400 mg, yield 86.5%). LC-MS (ESI) m/z: 367 [M+H]⁺.

### Step 5: Synthesis of N-(4-(cyclohexylamino)-3-(2-(2-fluorobenzyl)-2H-tetrazol-5-yl)phenyl)acrylamide (Compound A-17)

To the mixture of N1-cyclohexyl-2-(2-(2-fluorobenzyl)-2H-tetrazol-5-yl)benzene-1,4-diamine (450 mg, 1.23 mmol) and Et₃N (249 mg, 2.46 mmol) in THF (15 mL) was added acryloyl chloride (110 mg, 1.23 mmol), the mixture was stirred at 0° C. for 1 hour, concentrated and purified by preparative HPLC (MeCN/H₂O/TFA) to afford the target compound A-17 as solid (490 mg, yield 95.0%). LC-MS (ESI) m/z: 421.3 [M+H]⁺. ¹H-NMR (400 MHz, DMSO-d₆) δ (ppm): 10.00 (s, 1H), 8.34 (d, J=2.5 Hz, 1H), 7.65 (dd, J=9.0, 2.5 Hz, 1H), 7.57 (t, J=7.6 Hz, 1H), 7.53-7.43 (m, 1H), 7.29 (dd, J=15.6, 7.9 Hz, 2H), 6.82 (dd, J=13.5, 8.5 Hz, 2H), 6.39 (dd, J=16.9, 10.1 Hz, 1H), 6.21 (dd, J=17.0, 2.0 Hz, 1H), 6.08 (s, 2H), 5.69 (dd, J=10.1, 2.0 Hz, 1H), 3.48 (s, 1H), 2.0-1.75 (br, 2H), 1.70-1.50 (m, 3H), 1.47-1.32 (m, 2H), 1.32-1.15 (m, 3H). ¹⁹F-NMR (376 MHz, DMSO-d₆) δ (ppm): 117.43.

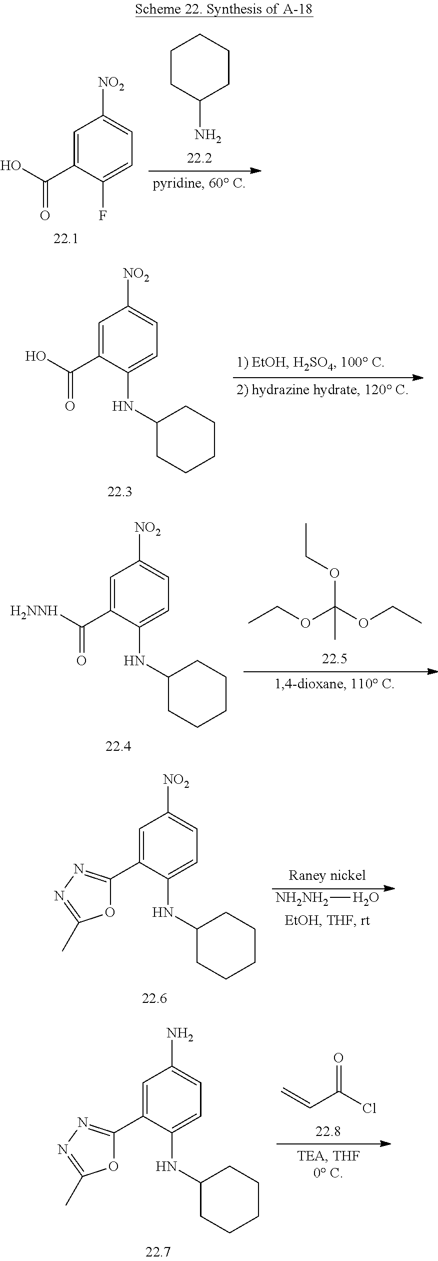

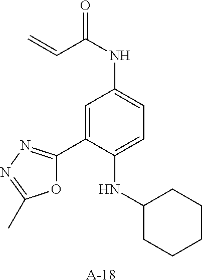 [SMILES: C=CC(=O)Nc1ccc(NC2CCCCC2)c(-c2nnc(C)o2)c1 ; "A-18"]

### Step 1: Synthesis of 2-(cyclohexylamino)-5-nitrobenzoic Acid (Compound 22.3)

The mixture of 2-fluoro-5-nitrobenzoic acid (1 g, 5.4 mmol) and cyclohexanamine (535 mg, 5.4 mmol) in pyridine (20 mL) was heated at 60° C. Under N₂ for 16 hours. The resulting mixture was concentrated and purified by flash column chromatography on silica gel (ethyl acetate in petroleum ether=30% v/v) to afford the target compound 22.3 as solid (1.5 g, yield 99.9%). LC-MS (ESI) m/z: 265 [M+H]⁺.

### Step 2: Synthesis of 2-(cyclohexylamino)-5-nitrobenzohydrazide (Compound 22.4)

The mixture of 2-(cyclohexylamino)-5-nitrobenzoic acid (1.4 g, 0.19 mmol) in EtOH (30 mL) and H₂SO₄ (30 mL) was heated at 100° C. for 16 hours. The resulting mixture was concentrated in vacuum, the residue was adjusted to pH 9 with concentrated ammonia solution and extracted with ether (100 mL×2), the combined extracts were dried over anhydrous Na₂SO₄, filtered and concentrated in vacuum, to the residue was added hydrazine hydrate (98%, 1 mL), the resulting mixture was heated at 120° C. for 3 hours, concentrated and purified by flash column chromatography on silica gel (ethyl acetate in petroleum ether=30% v/v) to obtain the target compound 22.4 as oil (700 mg, yield 36.6%). LC-MS (ESI) m/z: 279 [M+H]⁺.

### Step 3: Synthesis of N-cyclohexyl-2-(5-methyl-1,3,4-oxadiazol-2-yl)-4-nitroaniline (Compound 22.6)

The mixture of 2-(cyclohexylamino)-5-nitrobenzohydrazide (650 mg, 2.34 mmol) and 1,1,1-triethoxyethane (379 mg, 2.34 mmol) in 1,4-dioxane (30 mL) was heated at 110° C. for 16 hours. The resulting mixture was concentrated and purified by flash column chromatography on silica gel (ethyl acetate in petroleum ether=50% v/v) to obtain the target compound 22.6 as solid (600 mg, yield 84.9%). LC-MS (ESI) m/z: 303 [M+H]⁺.

### Step 4: Synthesis of N1-cyclohexyl-2-(5-methyl-1,3,4-oxadiazol-2-yl)benzene-1,4-diamine (Compound 22.7)

To the solution of N-cyclohexyl-2-(5-methyl-1,3,4-oxadiazol-2-yl)-4-nitroaniline (550 mg, 1.82 mmol) in EtOH (15 mL) and THF (15 mL) was added Raney nickel (88 mg, 0.18 mmol), and then N₂H₄·H₂O (110 mg, 1.82 mmol). The resulting mixture was stirred at rt for 1 hour and filtered, the filtrate was concentrated under reduced pressure to leave the crude target compound 22.7 as oil (500 mg, yield 99.9%). LC-MS (ESI) m/z: 273 [M+H]⁺.

### Step 5: Synthesis of N-(4-(cyclohexylamino)-3-(5-methyl-1,3,4-oxadiazol-2-yl)phenyl)acrylamide (Compound A-18)

To the mixture of N1-cyclohexyl-2-(5-methyl-1,3,4-oxadiazol-2-yl)benzene-1,4-diamine (450 mg, 1.65 mmol) and Et₃N (100 mg, 3.31 mmol) in THF (20 mL) was added acryloyl chloride (64 mg, 1.65 mmol), the resulting mixture was stirred at 0° C. for 1 hour, concentrated and monitored by preparative HPLC (MeCN/H₂O/TFA) to obtain the target compound A-18 as solid (314 mg, yield 58.2%). LC-MS (ESI) m/z: 327 [M+H]⁺. ¹H-NMR (400 MHz, DMSO-d₆) δ (ppm): 10.03 (s, 1H), 8.22 (d, J=2.5 Hz, 1H), 7.57 (dd, J=9.1, 2.5 Hz, 1H), 7.32 (d, J=7.7 Hz, 1H), 6.90 (d, J=9.2 Hz, 1H), 6.39 (dd, J=16.5, 9.6 Hz, 1H), 6.22 (dd, J=17.0, 2.1 Hz, 1H), 5.71 (dd, J=10.0, 2.1 Hz, 1H), 3.55-3.50 (br, 1H), 2.59 (s, 3H), 1.97 (d, J=9.7 Hz, 2H), 1.75-1.65 (m, 2H), 1.60-1.50 (m, 1H), 1.50-1.35 (m, 2H), 1.35-1.26 (m, 3H).

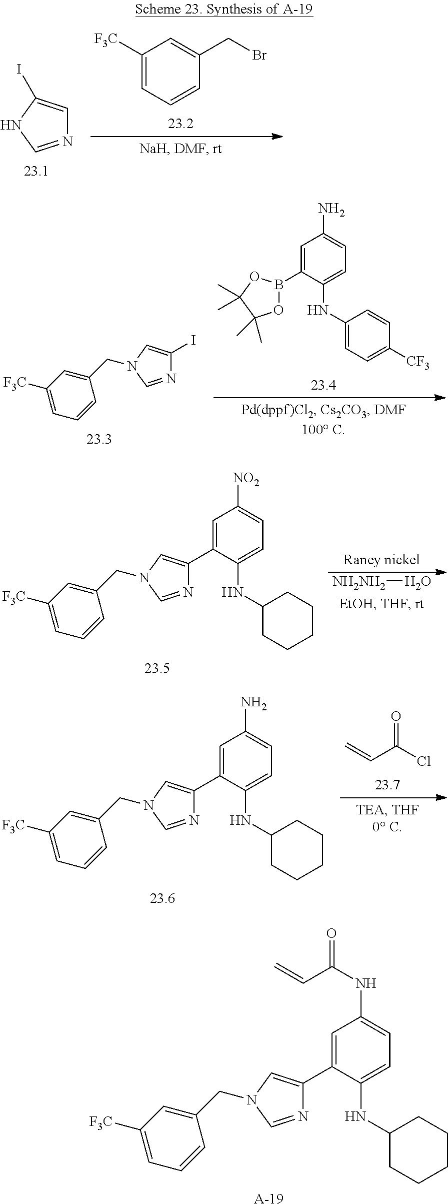

### Step 1: Synthesis of 4-iodo-1-(3-(trifluoromethyl)benzyl)-1H-imidazole (Compound 23.3)

To the solution of 5-iodo-1H-imidazole (1 g, 5.18 mmol) in DMF (20 mL) was added NaH (248 mg, 6.2 mmol) under N₂, the mixture was stirred at 0° C. for 0.5 hour, and then 1-(bromomethyl)-3-(trifluoromethyl)benzene (1.24 g, 5.18 mmol) was added, the resulting mixture was stirred at rt for 1 hours, concentrated and purified by flash column chromatography on silica gel (ethyl acetate in petroleum ether=30% v/v) to obtain the target compound 23.3 as solid (1.5 g, yield 82.8%). LC-MS (ESI) m/z: 353 [M+H]⁺.

### Step 2: Synthesis of N-cyclohexyl-4-nitro-2-(1-(3-(trifluoromethyl)benzyl)-1H-imidazol-4-yl)aniline (Compound 23.5)

The mixture of 4-iodo-1-(3-(trifluoromethyl)benzyl)-1H-imidazole (300 mg, 0.96 mmol), N-cyclohexyl-4-nitro-2-(4,4,5,5-tetramethyl-1,3,2-dioxaborolan-2-yl)aniline (300 mg, 0.96 mmol), Cs₂CO₃ (828 mg, 2.52 mmol) and Pd(dppf)Cl₂ (62.4 mg, 0.096 mmol) in DMF (2 mL) was heated at 100° C. under N₂ for 16 hours. The resulting mixture was concentrated and purified by flash column chromatography on silica gel (ethyl acetate in petroleum ether=30% v/v) to obtain the target compound 23.5 as solid (120 mg, yield 31.7%). LC-MS (ESI) m/z: 445 [M+H]⁺.

### Step 3: Synthesis of N1-cyclohexyl-2-(1-(3-(trifluoromethyl)benzyl)-1H-imidazol-4-yl)benzene-1,4-diamine (Compound 23.6)

To the solution of N-cyclohexyl-4-nitro-2-(1-(3-(trifluoromethyl)benzyl)-1H-imidazol-4-yl)aniline (90 mg, 0.18 mmol) in EtOH (10 mL) and THF (10 mL) was added Raney nickel (12 mg, 0.03 mmol), followed by N₂H₄·H₂O (9 mg, 0.18 mmol). The mixture was stirred at rt for 1 hour and filtered, the filtrate was concentrated under reduced pressure to leave crude target compound 23.6 as oil (60 mg, yield 71.5%). LC-MS (ESI) m/z: 415 [M+H]⁺.

### Step 4: Synthesis of N-(4-(cyclohexylamino)-3-(1-(3-(trifluoromethyl)benzyl)-1H-imidazol-4-yl)phenyl)acrylamide (Compound A-19)

To the mixture of N1-cyclohexyl-2-(1-(3-(trifluoromethyl)benzyl)-1H-imidazol-4-yl)benzene-1,4-diamine (50 mg, 0.12 mmol) and Et₃N (24 mg, 0.24 mmol) in THF (10 mL) was added acryloyl chloride (18 mg, 0.12 mmol), the mixture was stirred at 0° C. for 1 hour, concentrated and purified by preparative HPLC (MeCN/H₂O/TFA) to afford the target compound A-19 as solid (5 mg, yield 8.8%). LC-MS (ESI) m/z: 469 [M+H]⁺. ¹H-NMR (400 MHz, CD₃OD) δ (ppm): 7.89 (s, 1H), 7.70-7.54 (m, 5H), 7.40 (s, 1H), 7.32 (dd, J=8.8, 2.4 Hz, 1H), 6.76 (d, J=8.4 Hz, 1H), 6.41 (dd, J=16.8, 10.0 Hz, 1H), 6.31 (dd, J=16.8, 2.0 Hz, 1H), 5.73 (dd, J=9.8, 2.1 Hz, 1H), 5.40 (s, 2H), 2.06-1.96 (m, 2H), 1.80-1.70 (m, 2H), 1.68-1.58 (m, 1H), 1.50-1.21 (m, 6H).

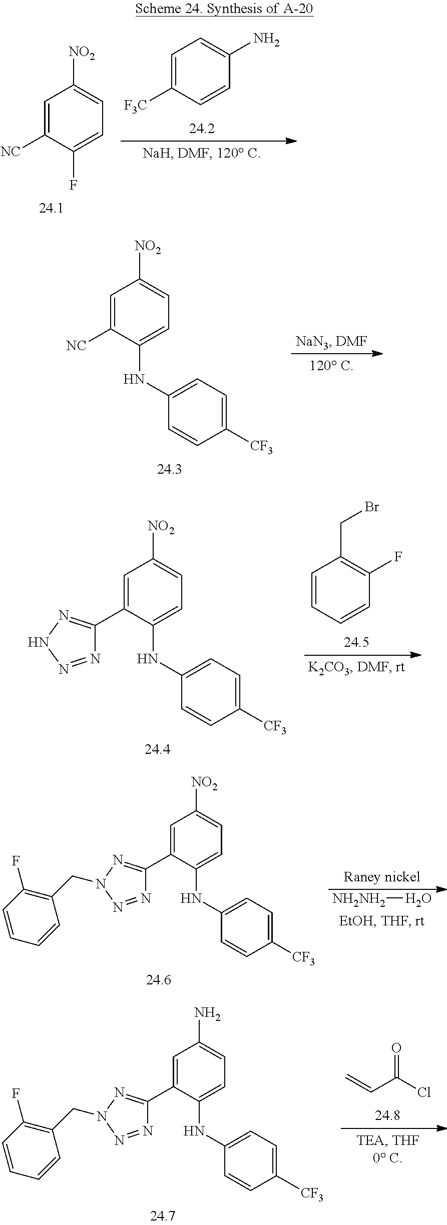

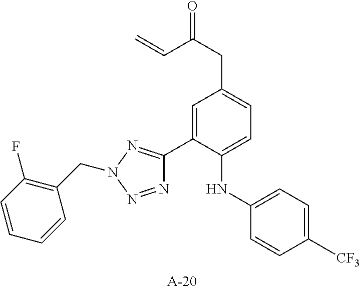 [SMILES: C=CC(=O)Cc1ccc(Nc2ccc(C(F)(F)F)cc2)c(-c2nnn(Cc3ccccc3F)n2)c1 ; "A-20"]

### Step 1: Synthesis of 5-nitro-2-(4-(trifluoromethyl)phenylamino)benzonitrile (Compound 24.3)

To the solution of 4-(trifluoromethyl) aniline (0.96 mg, 6.0 mmol) in DMF (20 mL) was added NaH (0.29 g, 7.2 mmol), the mixture was stirred at 0° C. under N₂ for 0.5 hour, and then 2-fluoro-5-nitrobenzonitrile (1 g, 6.0 mmol) was added. The resulting mixture was stirred at 120° C. for 16 hours, concentrated and purified by flash column chromatography on silica gel (ethyl acetate in petroleum ether=30% v/v) to afford the target compound 24.3 as solid (500 mg, yield 27.1%). LC-MS (ESI) m/z: 308 [M+H]⁺.

### Step 2: Synthesis of 4-nitro-2-(2H-tetrazol-5-yl)-N-(4-(trifluoromethyl)phenyl)aniline (Compound 24.4)

The mixture of 5-nitro-2-(4-(trifluoromethyl)phenylamino)benzonitrile (400 mg, 1.30 mmol) and NaN₃ (127 mg, 1.95 mmol) in DMF (20 mL) was heated at 120° C. for 16 hours. The resulting mixture was concentrated and purified by flash column chromatography on silica gel (ethyl acetate in petroleum ether=30% v/v) to obtain the target compound 24.4 as solid (300 mg, yield 65.7%). LC-MS (ESI) m/z: 351 [M+H]⁺.

### Step 3: Synthesis of 2-(2-(2-fluorobenzyl)-2H-tetrazol-5-yl)-4-nitro-N-(4-(trifluoromethyl)phenyl)aniline (Compound 24.6)

The mixture of 4-nitro-2-(2H-tetrazol-5-yl)-N-(4-(trifluoromethyl)phenyl)aniline (250 mg, 0.71 mmol), 1-(bromomethyl)-2-fluorobenzene (134 mg, 0.71 mmol) and K₂CO₃ (196 mg, 1.42 mmol) in DMF (15 mL) was stirred at rt for 3 hours. The resulting mixture was concentrated and purified by flash column chromatography on silica gel (ethyl acetate in petroleum ether=40% v/v) to obtain the target compound 24.6 as solid (300 mg, yield 91.7%). LC-MS (ESI) m/z: 459 [M+H]⁺.

### Step 4: Synthesis of 2-(2-(2-fluorobenzyl)-2H-tetrazol-5-yl)-N1-(4-(trifluoromethyl)phenyl)benzene-1,4-diamine (Compound 24.7)

To the solution of 2-(2-(2-fluorobenzyl)-2H-tetrazol-5-yl)-4-nitro-N-(4-(trifluoromethyl)phenyl)aniline (250 mg, 0.55 mmol) in EtOH (10 mL) and THF (10 mL) was added Raney nickel (20 mg, 0.05 mmol), and then N₂H₄·H₂O (27 mg, 0.55 mmol) was added. The mixture was stirred at rt for 1 hour and filtered, the filtrate was concentrated under reduced pressure to leave the crude compound 24.7 as oil (200 mg, yield 71.4%). LC-MS (ESI) m/z: 429 [M+H]⁺.

### Step 5: Synthesis of N-(3-(2-(2-fluorobenzyl)-2H-tetrazol-5-yl)-4-(4-(trifluoromethyl) phenylamino)phenyl)acrylamide (Compound A-20)

To the mixture of 2-(2-(2-fluorobenzyl)-2H-tetrazol-5-yl)-N1-(4-(trifluoromethyl) phenyl)benzene-1,4-diamine (200 mg, 0.47 mmol) and Et₃N (97 mg, 0.94 mmol) in THF (15 mL) was added acryloyl chloride (42 mg, 0.47 mmol), the mixture was stirred at 0° C. for 1 hour, concentrated and purified by preparative HPLC (MeCN/H₂O/TFA) to obtain the target compound A-20 as solid (171 mg, yield 76.0%). LC-MS (ESI) m/z: 483 [M+H]⁺. ¹H-NMR (400 MHz, DMSO-d₆) δ (ppm) 10.32 (s, 1H), 8.59 (s, 1H), 8.37 (d, J=2.4 Hz, 1H), 7.83 (dd, J=9.1, 2.3 Hz, 1H), 7.49 (dd, J=8.1, 3.8 Hz, 5H), 7.34-7.18 (m, 2H), 7.03 (d, J=8.7 Hz, 2H), 6.43 (dd, J=17.0, 10.1 Hz, 1H), 6.27 (dd, J=17.0, 2.0 Hz, 1H), 6.04 (s, 2H), 5.77 (dd, J=10.1, 2.0 Hz, 1H). ¹⁹F-NMR (376 MHz, DMSO-d₆) δ (ppm): −59.52, −117.53.

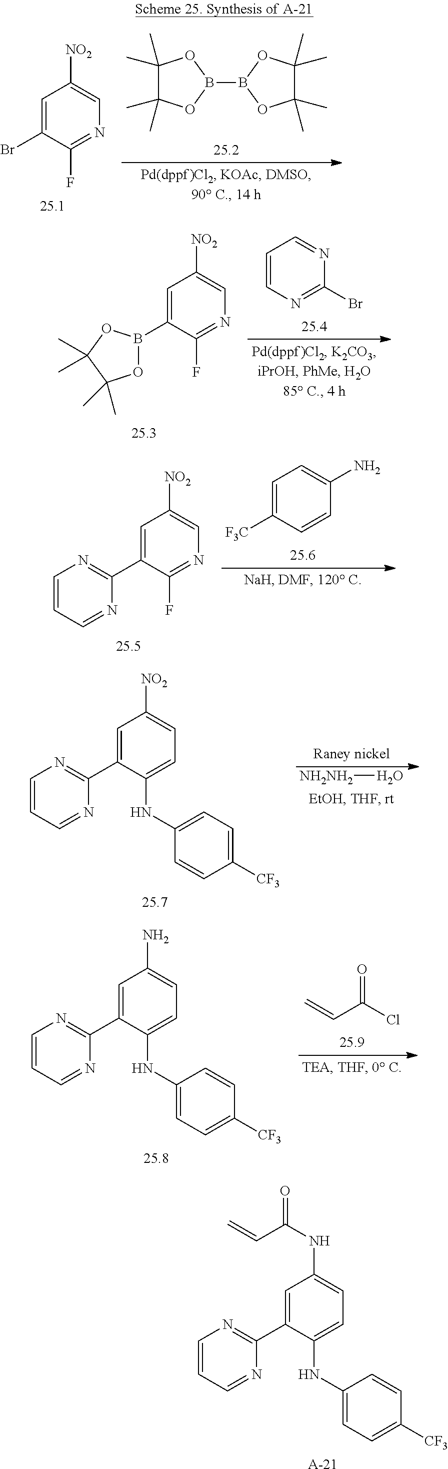

### Step 1: Synthesis of 2-(2-fluoro-5-nitrophenyl)-4,4,5,5-tetramethyl-1,3,2-dioxaborolane (Compound 25.3)

To the mixture of 2-bromo-1-fluoro-4-nitrobenzene (500 mg, 2.3 mmol), 4,4,4′,4′,5,5,5′,5′-octamethyl-2,2′-bi(1,3,2-dioxaborolane) (640 mg, 2.5 mmol) and KOAc (450 mg, 4.6 mmol) in 1,4-dioxane (25 mL) and DMSO (0.5 mL) was added Pd(dppf)Cl₂ (84 mg, 0.11 mmol), the mixture was heated at 90° C. under N₂ for 14 hours. The mixture was cooled down to rt, diluted with water (100 mL) and extracted with EtOAc (3×100 mL), the combined organics were washed with brine (200 mL), dried over anhydrous Na₂SO₄, filtered and concentrated to leave the crude compound 25.3 as oil (400 mg, yield 65.6%). LC-MS (ESI) m/z: 186.1 [M−82]⁺.

### Step 2: Synthesis of 2-(2-fluoro-5-nitrophenyl)pyrimidine (Compound 25.5)

To the mixture of 2-(2-fluoro-5-nitrophenyl)-4,4,5,5-tetramethyl-1,3,2-dioxaborolane (400 mg, 1.50 mmol), 2-bromopyrimidine (237 mg, 1.50 mmol) and K₃PO₄ (954 mg, 4.50 mmol) in iPrOH (5 mL), toluene (5 mL) and H₂O (5 mL) was added Pd(dppf)Cl₂ (54 mg, 0.075 mmol), the mixture was heated at 85° C. under N₂ for 4 hours and concentrated in vacuum, the residue was purified by flash column chromatography on silica gel (ethyl acetate in petroleum ether=30% in v/v) to obtain the target compound 25.5 as solid (300 mg, yield 91.4%). LC-MS (ESI) m/z: 220.1 [M+H]⁺.

### Step 3: Synthesis of 4-nitro-2-(pyrimidin-2-yl)-N-(4-(trifluoromethyl)phenyl)aniline (Compound 25.7)

To the mixture of 2-(2-fluoro-5-nitrophenyl)pyrimidine (250 mg, 1.14 mmol) in DMF (10 mL) was added NaH (54 mg, 2.25 mmol), the mixture was stirred at 0° C. under N₂ for 0.5 hour, and then 4-(trifluoromethyl)aniline (183 mg, 1.14 mmol) was added, the mixture was stirred at 120° C. for 16 hours and concentrated in vacuum, the residue was purified by flash column chromatography on silica gel (ethyl acetate in petroleum ether=30% in v/v) to obtain the target compound 25.7 as solid (200 mg, yield 48.7%). LC-MS (ESI) m/z: 361.0 [M+H]⁺.

### Step 4: Synthesis of 2-(pyrimidin-2-yl)-N1-(4-(trifluoromethyl)phenyl)benzene-1,4-diamine (Compound 25.8)

To the mixture of 4-nitro-2-(pyrimidin-2-yl)-N-(4-(trifluoromethyl)phenyl)aniline (150 mg, 0.42 mmol) and Raney nickel (16 mg, 0.04 mmol) in EtOH (10 mL) and THF (10 mL) was added N₂H₄·H₂O (80 mg, 1.682 mmol), the mixture was stirred at rt for 1 hour and filtered through celite, the filtrate was concentrated under reduced pressure to leave the crude compound 25.8 as oil (100 mg, yield 72.9%). LC-MS (ESI) m/z: 331.1 [M+H]⁺.

### Step 5: Synthesis of N-(3-(pyrimidin-2-yl)-4-(4-(trifluoromethyl)phenylamino)phenyl) Acrylamide (Compound A-21)

To the mixture of 2-(pyrimidin-2-yl)-N1-(4-(trifluoromethyl)phenyl) benzene-1,4-diamine (100 mg, 0.31 mmol) and Et₃N (62 mg, 0.62 mmol) in THF (10 mL) was added acryloyl chloride (28 mg, 0.31 mmol), the mixture was stirred at 0° C. for 1 hour. The resulting mixture was concentrated and purified by preparative HPLC (MeCN/H₂O/THF) to obtain the target compound A-21 as solid (45 mg, yield 38.7%). LC-MS (ESI) m/z: 385.2 [M+H]⁺. ¹H-NMR (400 MHz, DMSO-d₆) δ (ppm) 10.71 (s, 1H), 10.28 (s, 1H), 8.99 (d, J=4.7 Hz, 2H), 8.71 (s, 1H), 7.86 (d, J=8.3 Hz, 1H), 7.61-7.44 (m, 4H), 7.31 (d, J=8.1 Hz, 2H), 6.46 (dd, J=16.8, 9.9 Hz, 1H), 6.27 (d, J=16.6 Hz, 1H), 5.76 (d, J=9.5 Hz, 1H). ¹⁹F-NMR (376 MHz, DMSO-d₆) δ (ppm): −59.74.

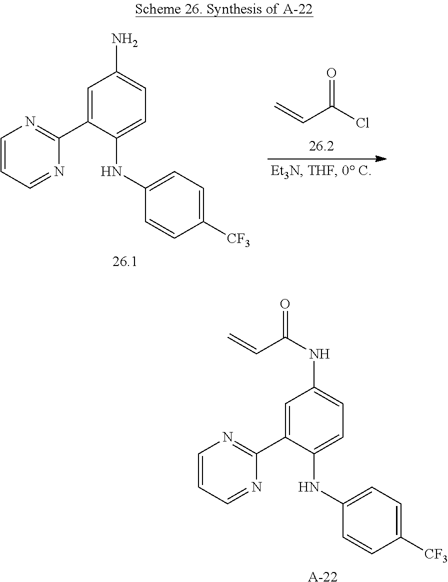

### Step 1: Synthesis of N-(3-(pyrimidin-2-yl)-4-(4-(trifluoromethyl)phenylamino) phenyl)propionamide (Compound A-22)

To the solution of 2-(pyrimidin-2-yl)-N1-(4-(trifluoromethyl)phenyl)benzene-1,4-diamine (180 mg, 0.55 mmol) in THF (20 mL) was added Et₃N (111 mg, 1.1 mmol) and propionyl chloride (50 mg, 0.55 mmol), the mixture was stirred at 0° C. for 1 hour, concentrated and purified by prep-HPLC (MeCN/H₂O/TFA) to obtain the target compound A-22 as solid (21 mg, yield 10.0%). LC-MS (ESI) m/z: 386 [M+H]⁺. ¹H NMR (400 MHz, DMSO-d₆) δ (ppm): 10.60 (s, 1H), 9.95 (s, 1H), 8.98 (d, J=4.9 Hz, 2H), 8.62 (d, J=2.5 Hz, 1H), 7.75 (dd, J=8.9, 2.5 Hz, 1H), 7.55 (d, J=8.6 Hz, 2H), 7.52-7.46 (m, 2H), 7.28 (d, J=8.5 Hz, 2H), 2.33 (q, J=7.5 Hz, 2H), 1.11 (t, J=7.6 Hz, 3H).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound described herein is of Formula (I″): 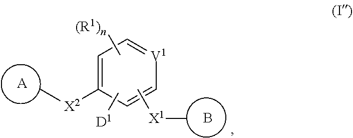
or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered optionally substituted heteroaryl, or optionally substituted heterocyclyl comprising at least one nitrogen atom, or a bicyclic heteroaryl;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —C(R^{c})₂O—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
X² is a bond, —C(R^{c})₂—; —CO—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
V¹ is ═N— or ═C(R^{d})—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl;
n is 0, 1, 2, or 3; and
D¹ is a warhead of any one of Formulae (i-1) to (i-43): 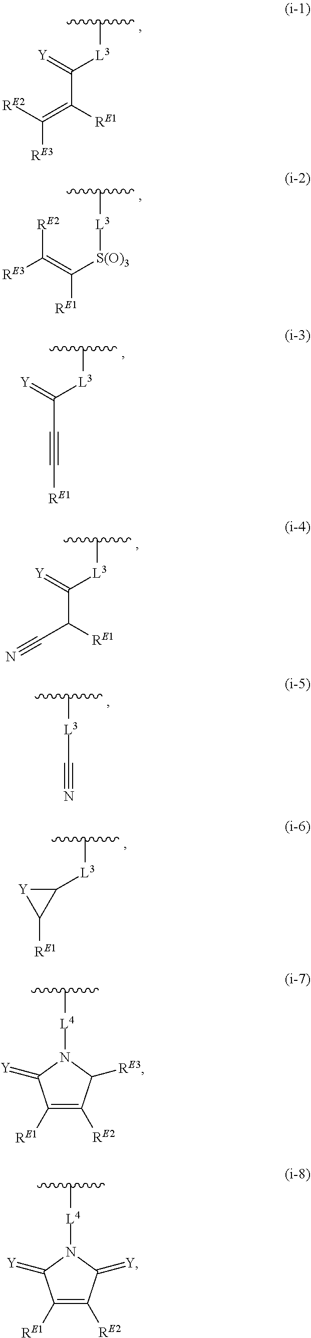 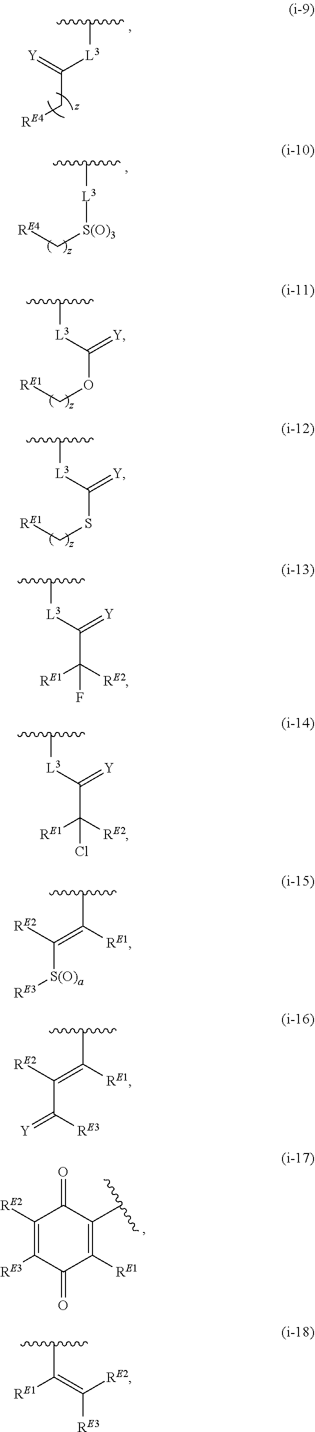 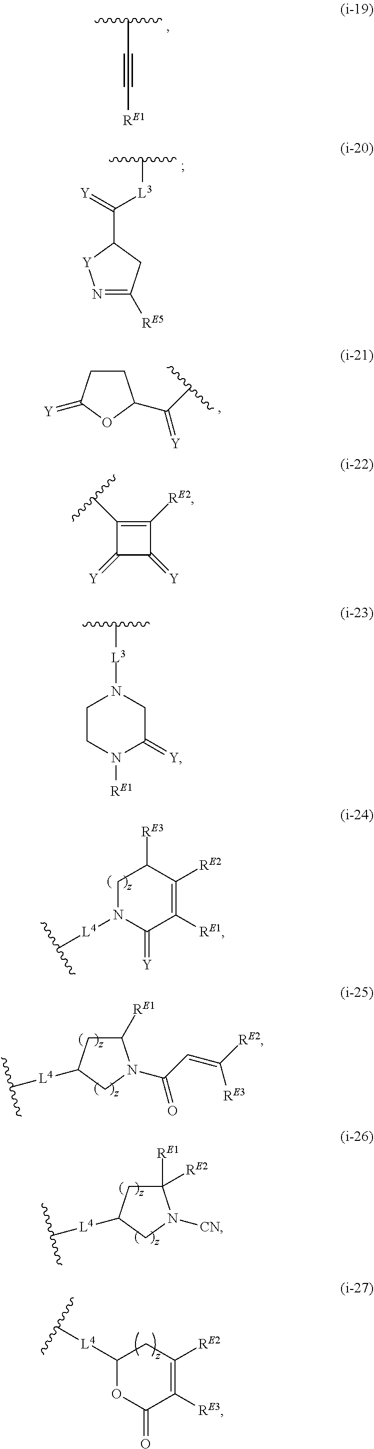 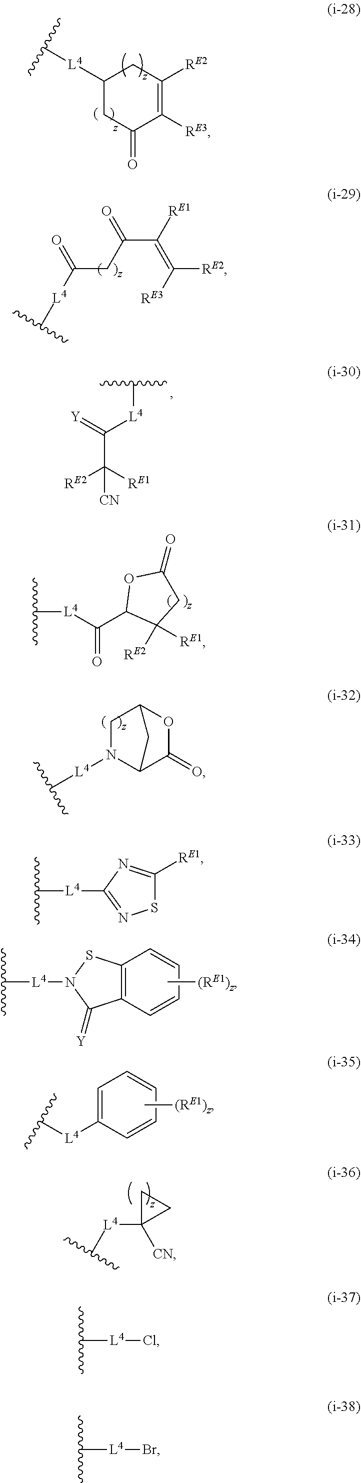 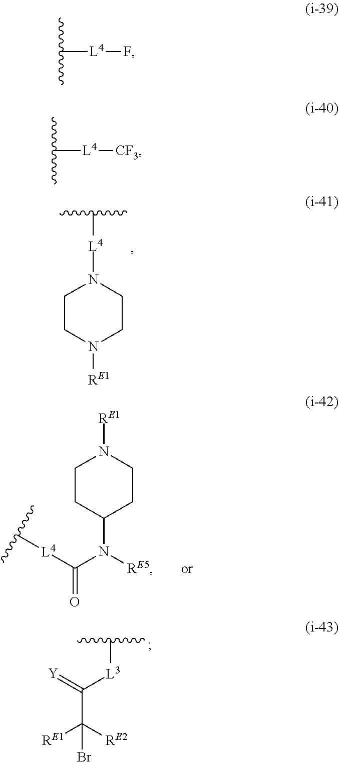
wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

2. The compound of claim 1, wherein X² is —C(R^{c})₂— or a bond.

3-4. (canceled)

5. The compound of claim 1, of Formula (I): 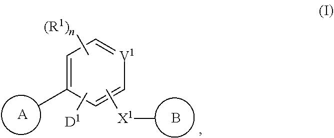
or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —C(R^{c})₂O—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
V¹ is ═N—, or ═C(R^{d})—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl;
n is 0, 1, 2, or 3; and
D¹ is a warhead of any one of Formulae (i-1) to (i-43): 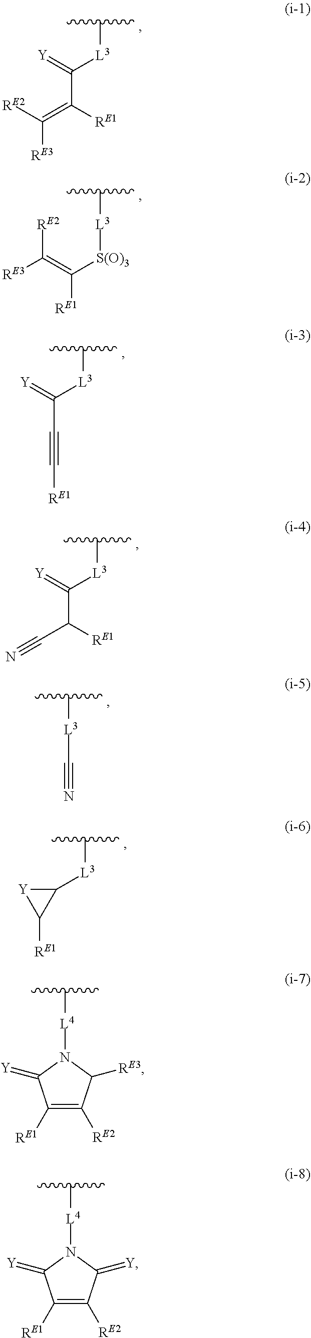 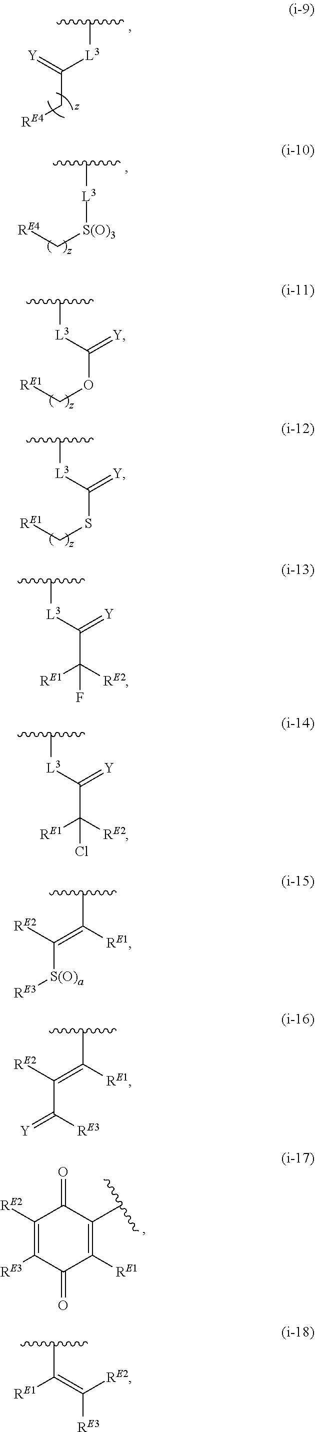 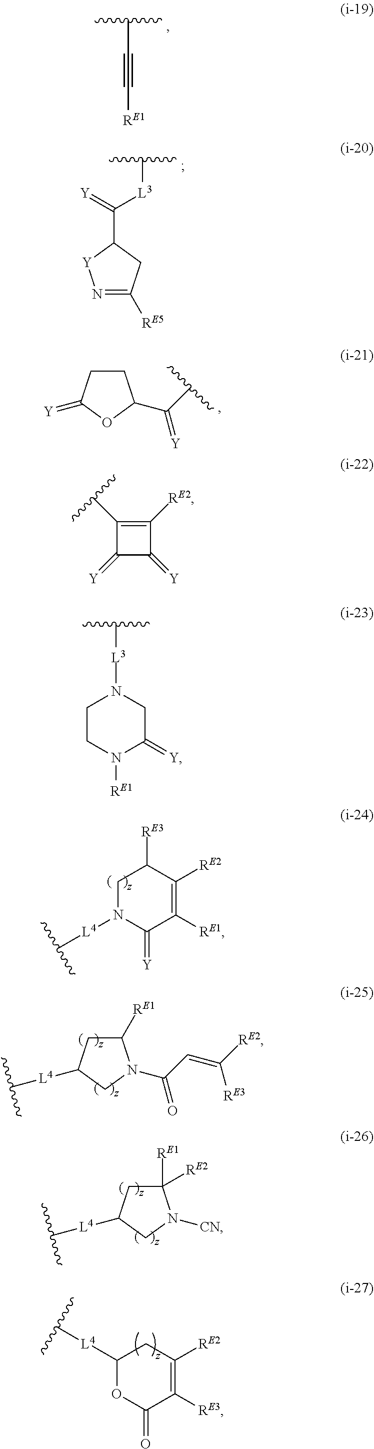 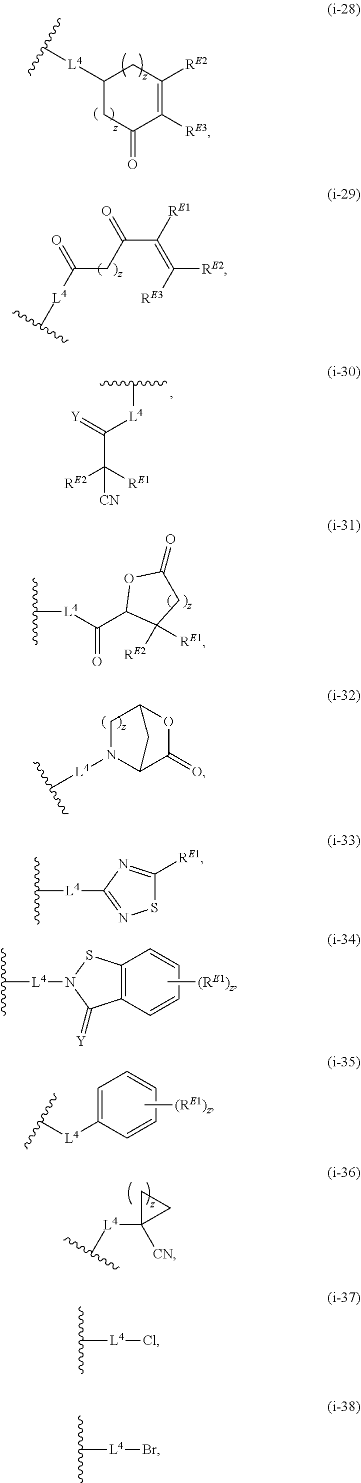 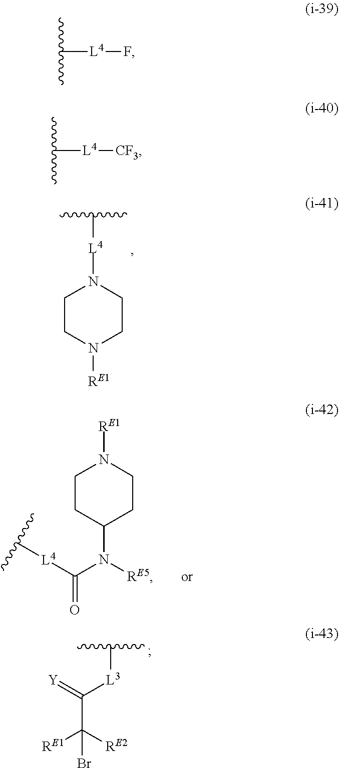
wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

6. The compound of claim 1, of the Formula (I-a), (I-b), (I-c), (I-d), (I-e), (I-f), (I-g), or (I-h): 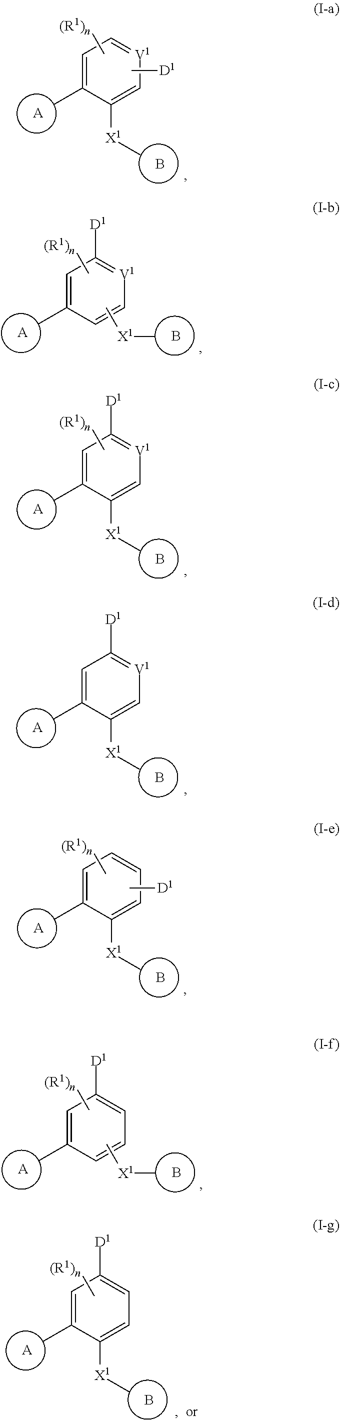 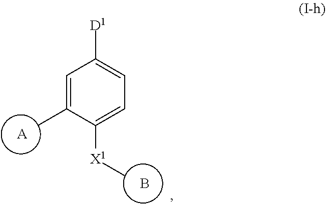
or a pharmaceutically acceptable salt, solvate, hydrate, polymorph, co-crystal, tautomer, stereoisomer, isotopically labeled compound, or prodrug thereof.

7-9. (canceled)

10. A compound of Formula (I′): 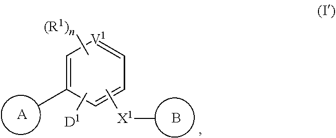
or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom, or a bicyclic heteroaryl;
Ring B is an optionally substituted bicyclic or monocyclic aryl, or an optionally substituted carbocyclic ring;
R¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X¹ is —C(R^{c})₂O—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
V¹ is ═N— or ═C(R^{d})—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl;
n is 0, 1, 2, or 3; and
D¹ is a warhead of any one of Formulae (i-1) to (i-43): 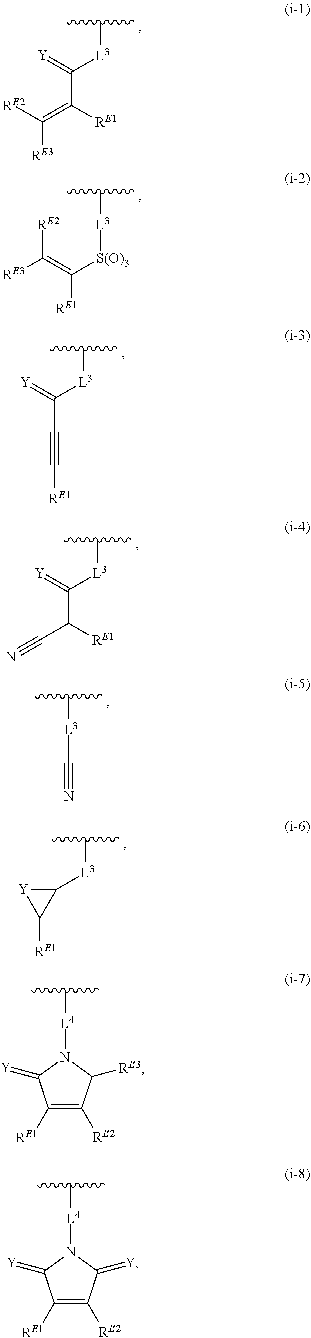 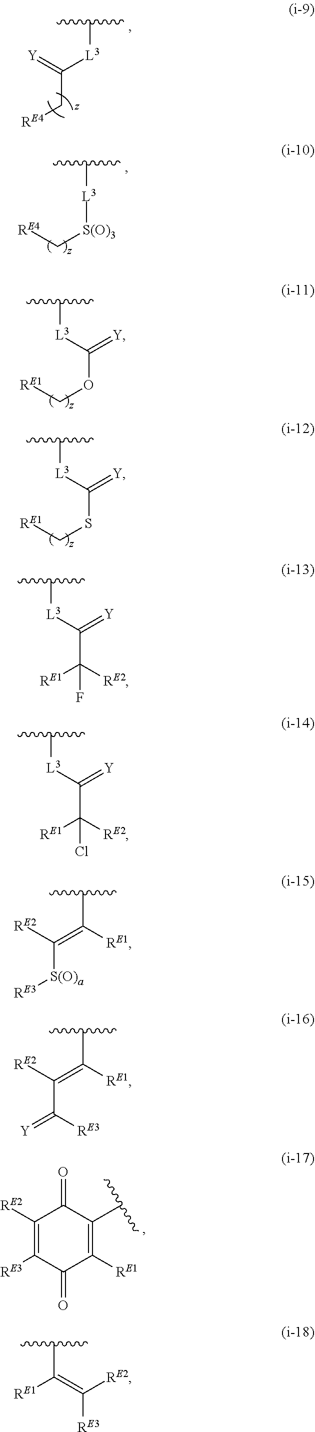 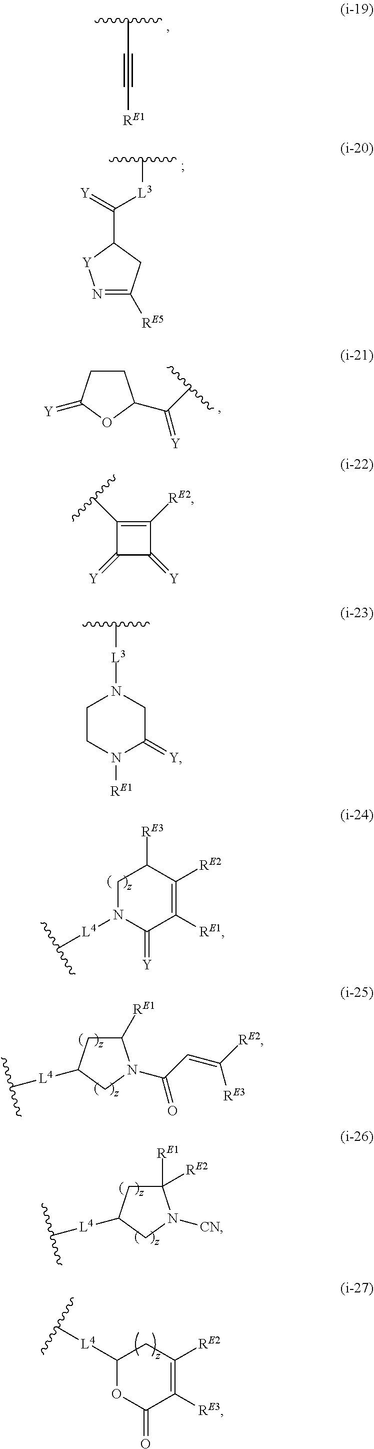 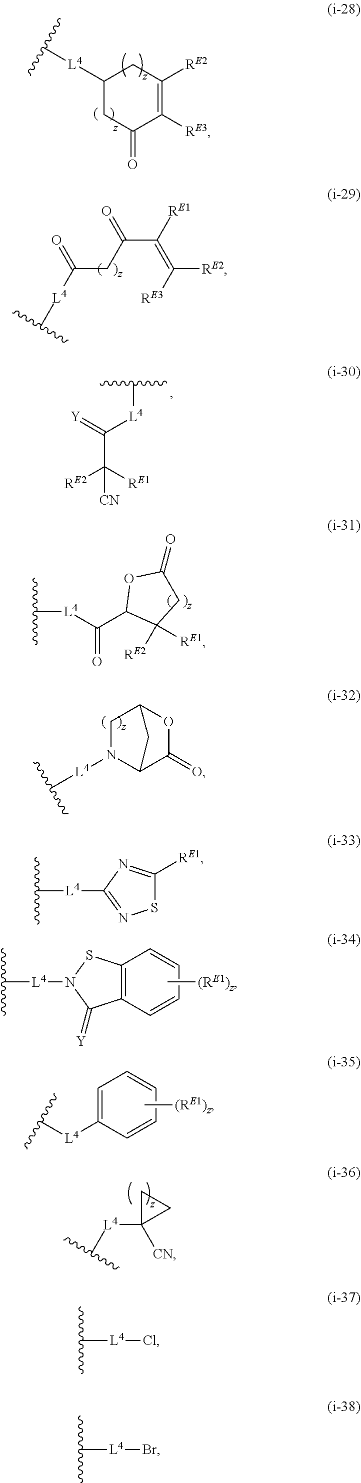 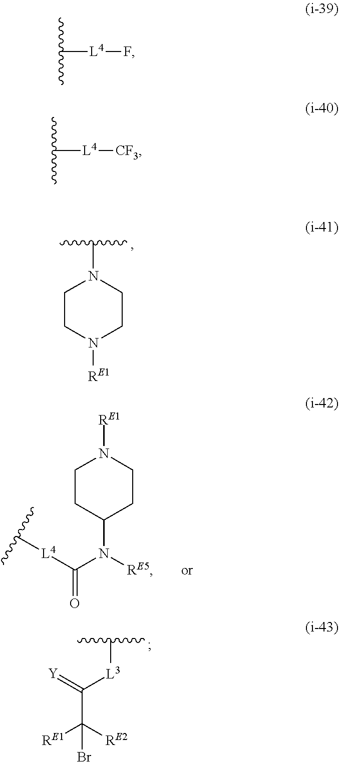
wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

11. The compound of claim 10, of Formula (I′-a), (I′-b), (I′-c), or (I′-d): 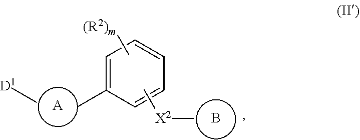
or a pharmaceutically acceptable salt, solvate, hydrate, polymorph, co-crystal, tautomer, stereoisomer, isotopically labeled compound, or prodrug thereof, wherein:
Ring A is an optionally substituted 5- or 6-membered heteroaryl comprising at least one nitrogen atom, or a bicyclic heteroaryl;
Ring B is an optionally substituted bicyclic or monocyclic aryl, or an optionally substituted carbocyclic ring;
R² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{b})₂, —SR^{a}, —C(═O)R^{a}, —C(═O)OR^{a}, —C(═O)SR^{a}, —C(═O)N(R^{b})₂, —OC(═O)R^{a}, —OC(═O)OR^{a}, —OC(═O)SR^{a}, —OC(═O)N(R^{b})₂, —N(R^{b})C(═O)R^{a}, —N(R^{b})C(═O)OR^{a}, —N(R^{b})C(═O)SR^{a}, —N(R^{b})C(═O)N(R^{b})₂, —SC(═O)R^{a}, —SC(═O)OR^{a}, —SC(═O)SR^{a}, —SC(═O)N(R^{b})₂, —C(═NR^{b})R^{b}, —C(═NR^{b})OR^{a}, —C(═NR^{b})SR^{a}, —C(═NR^{b})N(R^{b})₂, —OC(═NR^{b})R^{b}, —OC(═NR^{b})OR^{a}, —OC(═NR^{b})SR^{a}, —OC(═NR^{b})N(R^{b})₂, —N(R^{b})C(═NR^{b})R^{b}, —N(R^{b})C(═NR^{b})OR^{a}, —N(R^{b})C(═NR^{b})SR^{a}, —N(R^{b}) C(═NR^{b})N(R^{b})₂, —SC(═NR^{b})R^{b}, —SC(═NR^{b})OR^{a}, —SC(═NR^{b})SR^{a}, —SC(═NR^{b})N(R^{b})₂, —C(═S)R^{a}, —C(═S)OR^{a}, —C(═S)SR^{a}, —C(═S)N(R^{b})₂, —S(═O)R^{a}, —SO₂R^{a}, —NR^{b}SO₂R^{a}, —SO₂N(R^{b})₂, —CN, —SCN, or —NO₂;
X² is a bond, —C(R^{c})₂—; —CO—, —OC(R^{c})₂—, —O—, —N(R^{c})—, —S—, —C(═O)—, —C(═O)O—, —C(═O)N(R^{c})—, —OC(═O)—, or —N(R^{c})C(═O)—;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{b} or R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or two instances of R^{b}, when present, can be joined together with the heteroatom to which they are attached to form an optionally substituted heterocyclic ring; 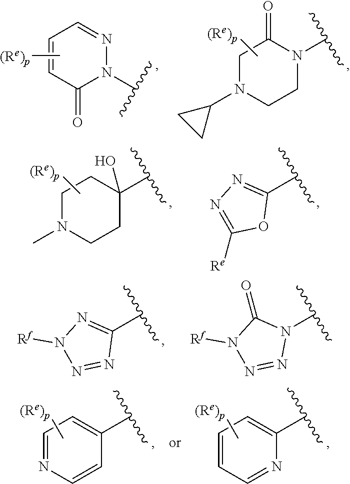
wherein each instance of R^{e} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}, —C(═O)R^{g}, —C(═O)OR^{g}, —C(═O)SR^{g}, —C(═O)N(R^{g})₂, —OC(═O)R^{g}, —OC(═O)OR^{g}, —OC(═O)SR^{g}, —OC(═O)N(R^{g})₂, —N(R^{g})C(═O)R^{g}, —N(R^{g})C(═O)OR^{g}, —N(R^{g})C(═O)SR^{g}, —N(R^{g})C(═O)N(R^{g})₂, —SC(═O)R^{g}, —SC(═O)OR^{g}, —SC(═O)SR^{g}, —SC(═O)N(R^{g})₂, —C(═NR^{g})R^{g}, —C(═NR^{g})OR^{g}, —C(═NR^{g})SR^{g}, —C(═NR^{g})N(R^{g})₂, —OC(═NR^{g})R^{g}, —OC(═NR^{g})OR^{g}, —OC(═NR^{g})SR^{g}, —OC(═NR^{g})N(R^{g})₂, —N(R^{g})C(═NR^{g})R^{g}, —N(R^{g})C(═NR^{g})OR^{g}, —N(R^{g})C(═NR^{g})SR^{g}, —N(R^{g}) C(═NR^{g})N(R^{g})₂, —SC(═NR^{g})R^{g}, —SC(═NR^{g})OR^{g}, —SC(═NR^{g})SR^{g}, —SC(═NR^{g})N(R^{g})₂, —C(═S)R^{g}, —C(═S)OR^{g}, —C(═S)SR^{g}, —C(═S)N(R^{g})₂, —S(═O)R^{g}, —SO₂R^{g}, —NR^{g}SO₂R^{g}, —SO₂N(R^{g})₂, —CN, —SCN, or —NO₂;
each occurrence of R^{f} or R^{g} is independently D¹, hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
p is 0, 1, 2, 3, or 4.

24-80. (canceled)

81. The compound of claim 1, wherein X¹ is —N(R^{c})— and R¹ is hydrogen.

82-83. (canceled)

84. A compound of Formula (II′):
m is 0, 1, 2, 3, or 4; and
D¹ is a warhead of any one of Formulae (i-1) to (i-43): 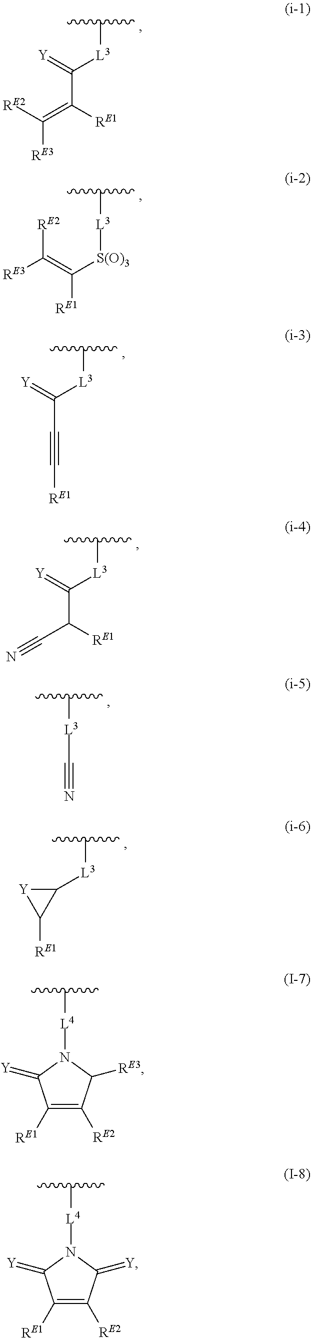 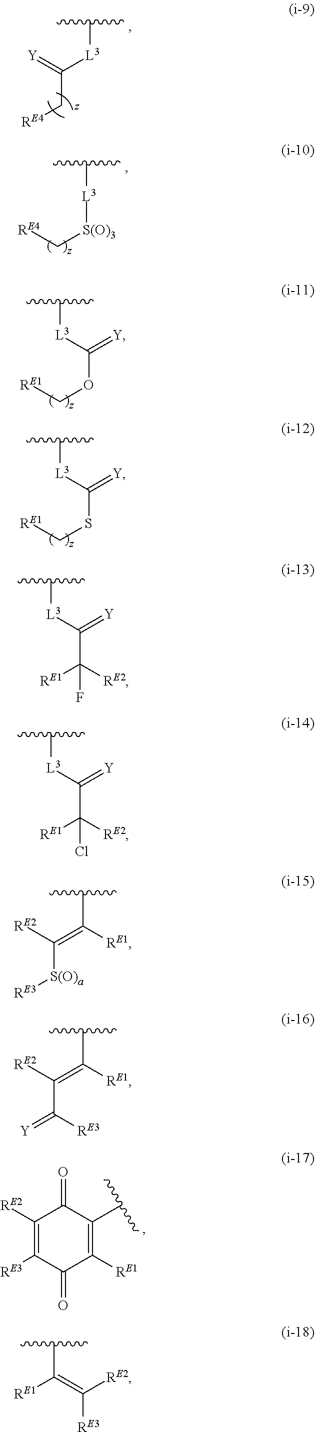 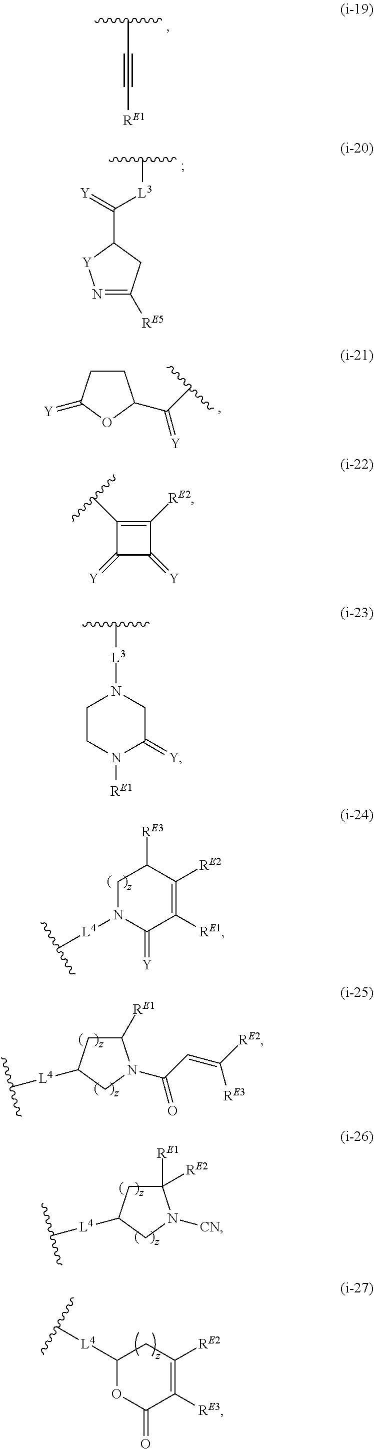 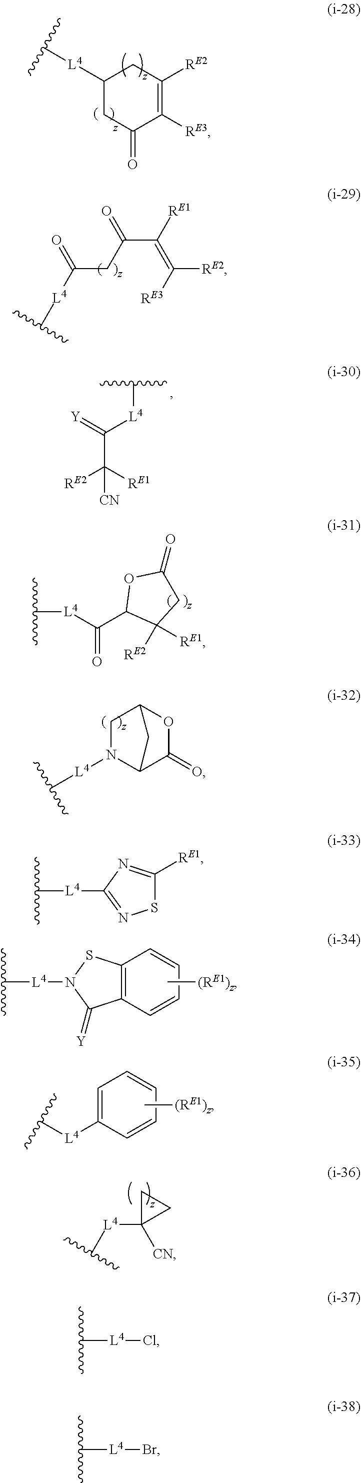 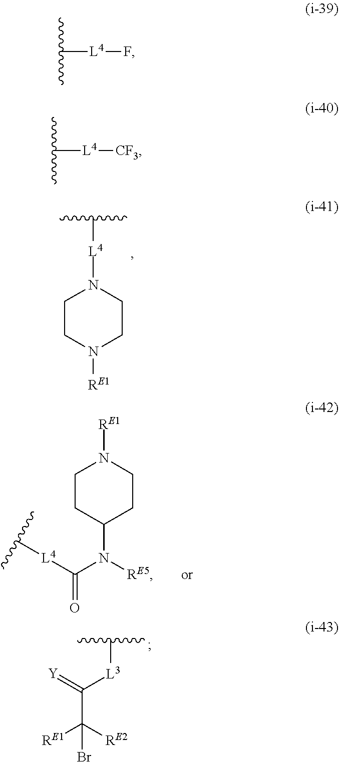
wherein:
L³ is a bond or an optionally substituted C₁₋₄ hydrocarbon chain, optionally wherein one or more carbon units of the hydrocarbon chain are independently replaced with —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans-CR^{L3b}═CR^{L3b}—, cis-CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{L3b} groups are joined to form an optionally substituted carbocyclic or optionally substituted heterocyclic ring;
L⁴ is a bond or an optionally substituted C₁₋₄ hydrocarbon chain;
R^{E1} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E1a}, —CH₂N(R^{E1a})₂, —CH₂SR^{E1a}, —OR^{E1a}, —N(R^{E1a})₂, —Si(R^{E1a})₃, and —SR^{E1a}, wherein each occurrence of R^{E1a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E1a} groups are joined to form an optionally substituted heterocyclic ring;
R^{E2} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E2a}, —CH₂N(R^{E2a})₂, —CH₂SR^{E2a}, —OR^{E2a}, —N(R^{E2a})₂, and —SR^{E2a}, wherein each occurrence of R^{E2a} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or two R^{E2a} groups are joined to form an optionally substituted heterocyclic ring;
R^{E3} is selected from the group consisting of hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{E3a}, —CH₂N(R^{E3a})₂, —CH₂SR^{E3a}, —OR^{E3a}, —N(R^{E3a})₂, and —SR^{E3a}, wherein each occurrence of R^{E3a} is independently selected from the group consisting of hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, and optionally substituted heteroaryl, or two R^{E3a} groups are joined to form an optionally substituted heterocyclic ring;
or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen; 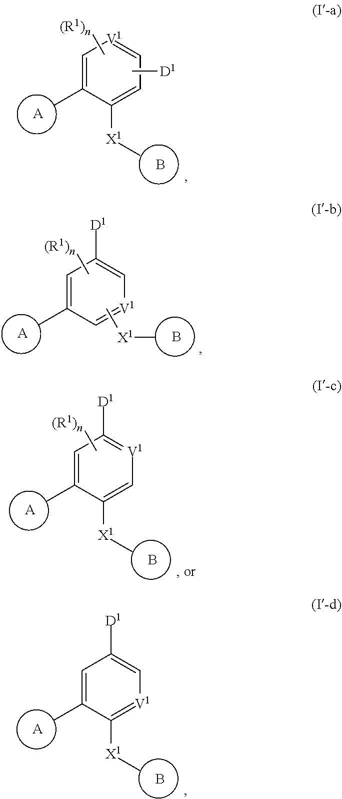
or a pharmaceutically acceptable salt, solvate, hydrate, polymorph, co-crystal, tautomer, stereoisomer, isotopically labeled compound, or prodrug thereof.

12-14. (canceled)

15. The compound of claim 1, wherein V¹ is ═N— or ═C(H)—.

16-22. (canceled)

23. The compound of claim 1, wherein Ring A is of the formula: 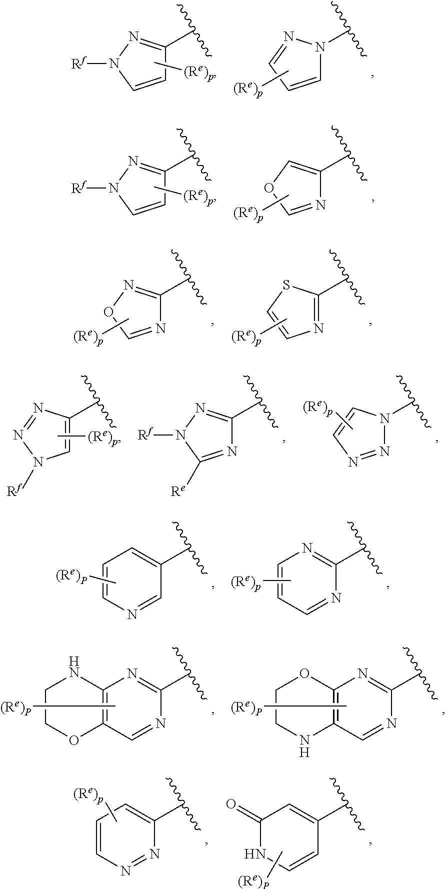
Y is —O—, —S—, or —NR^{E6}, wherein R^{E6} is hydrogen, substituted or unsubstituted C₁₋₆ alkyl, or a nitrogen protecting group;
a is 1 or 2; and
each instance of z is independently 0, 1, 2, 3, 4, 5, or 6.

85. The compound of claim 84, of the Formula (II′-a) or (II′-b): 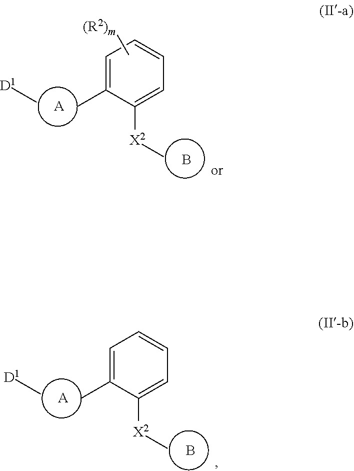
or a pharmaceutically acceptable salt, solvate, hydrate, polymorph, co-crystal, tautomer, stereoisomer, isotopically labeled compound, or prodrug thereof.

86-87. (canceled)

88. The compound of claim 84, wherein Ring A is of the Formula: 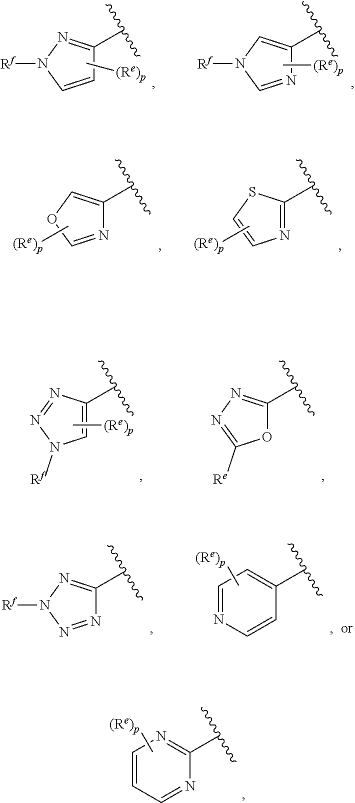
wherein each instance of R^{e} is independently D¹, hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{g}, —N(R^{g})₂, —SR^{g}, —C(═O)R^{g}, —C(═O)OR^{g}, —C(═O)SR^{g}, —C(═O)N(R^{g})₂, —OC(═O)R^{g}, —OC(═O)OR^{g}, —OC(═O)SR^{g}, —OC(═O)N(R^{g})₂, —N(R^{g})C(═O)R^{g}, —N(R^{g})C(═O)OR^{g}, —N(R^{g})C(═O)SR^{g}, —N(R^{g})C(═O)N(R^{g})₂, —SC(═O)R^{g}, —SC(═O)OR^{g}, —SC(═O)SR^{g}, —SC(═O)N(R^{g})₂, —C(═NR^{g})R^{g}, —C(═NR^{g})OR^{g}, —C(═NR^{g})SR^{g}, —C(═NR^{g})N(R^{g})₂, —OC(═NR^{g})R^{g}, —OC(═NR^{g})OR^{g}, —OC(═NR^{g})SR^{g}, —OC(═NR^{g})N(R^{g})₂, —N(R^{g})C(═NR^{g})R^{g}, —N(R^{g})C(═NR^{g})OR^{g}, —N(R^{g})C(═NR^{g})SR^{g}, —N(R^{g}) C(═NR^{g})N(R^{g})₂, —SC(═NR^{g})R^{g}, —SC(═NR^{g})OR^{g}, —SC(═NR^{g})SR^{g}, —SC(═NR^{g})N(R^{g})₂, —C(═S)R^{g}, —C(═S)OR^{g}, —C(═S)SR^{g}, —C(═S)N(R^{g})₂, —S(═O)R^{g}, —SO₂R^{g}, —NR^{g}SO₂R^{g}, —SO₂N(R^{g})₂, —CN, —SCN, or —NO₂;
each occurrence of R^{f} or R^{g} is independently D¹, hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
p is 0, 1, 2, 3, or 4.

89-103. (canceled)

104. The compound of claim 84, wherein X² is —N(R^{c})— and R² is hydrogen.

105-108. (canceled)

109. The compound of claim 1, wherein Ring B is of the formula: 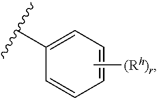
wherein each instance of R^{h} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —ORⁱ, —N(Rⁱ)₂, —SRⁱ, —C(═O)Rⁱ, —C(═O)ORⁱ, —C(═O)SRⁱ, —C(═O)N(Rⁱ)₂, —OC(═O)Rⁱ, —OC(═O)ORⁱ, —OC(═O)SRⁱ, —OC(═O)N(Rⁱ)₂, —N(Rⁱ)C(═O)Rⁱ, —N(Rⁱ)C(═O)ORⁱ, —N(Rⁱ)C(═O)SRⁱ, —N(Rⁱ)C(═O)N(Rⁱ)₂, —SC(═O)Rⁱ, —SC(═O)ORⁱ, —SC(═O)SRⁱ, —SC(═O)N(Rⁱ)₂, —C(═NRⁱ)Rⁱ, —C(═NRⁱ)ORⁱ, —C(═NRⁱ)SRⁱ, —C(═NRⁱ)N(Rⁱ)₂, —OC(═NRⁱ)Rⁱ, —OC(═NRⁱ)ORⁱ, —OC(═NRⁱ)SRⁱ, —OC(═NRⁱ)N(Rⁱ)₂, —N(Rⁱ)C(═NRⁱ)Rⁱ, —N(Rⁱ)C(═NRⁱ)ORⁱ, —N(Rⁱ)C(═NRⁱ)SRⁱ, —N(Rⁱ) C(═NRⁱ)N(Rⁱ)₂, —SC(═NRⁱ)Rⁱ, —SC(═NRⁱ)ORⁱ, —SC(═NRⁱ)SRⁱ, —SC(═NRⁱ)N(Rⁱ)₂, —C(═S)Rⁱ, —C(═S)ORⁱ, —C(═S)SRⁱ, —C(═S)N(Rⁱ)₂, —S(═O)Rⁱ, —SO₂Rⁱ, —NRⁱSO₂Rⁱ, —SO₂N(Rⁱ)₂, —CN, —SCN, or —NO₂;
each occurrence of Rⁱ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
r is 0, 1, 2, 3, 4, 5, 6, 7, or 8.

110-113. (canceled)

114. The compound of claim 1, wherein Ring B is of the formula: 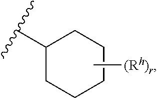
wherein each instance of R^{h} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —ORⁱ, —N(Rⁱ)₂, —SRⁱ, —C(═O)Rⁱ, —C(═O)ORⁱ, —C(═O)SRⁱ, —C(═O)N(Rⁱ)₂, —OC(═O)Rⁱ, —OC(═O)ORⁱ, —OC(═O)SRⁱ, —OC(═O)N(Rⁱ)₂, —N(Rⁱ)C(═O)Rⁱ, —N(Rⁱ)C(═O)ORⁱ, —N(Rⁱ)C(═O)SRⁱ, —N(Rⁱ)C(═O)N(Rⁱ)₂, —SC(═O)Rⁱ, —SC(═O)ORⁱ, —SC(═O)SRⁱ, —SC(═O)N(Rⁱ)₂, —C(═NRⁱ)Rⁱ, —C(═NRⁱ)ORⁱ, —C(═NRⁱ)SRⁱ, —C(═NRⁱ)N(Rⁱ)₂, —OC(═NRⁱ)Rⁱ, —OC(═NRⁱ)ORⁱ, —OC(═NRⁱ)SRⁱ, —OC(═NRⁱ)N(Rⁱ)₂, —N(Rⁱ)C(═NRⁱ) Rⁱ, —N(Rⁱ)C(═NRⁱ)ORⁱ, —N(Rⁱ)C(═NRⁱ)SRⁱ, —N(Rⁱ) C(═NRⁱ)N(Rⁱ)₂, —SC(═NRⁱ) Rⁱ, —SC(═NRⁱ)ORⁱ, —SC(═NRⁱ)SRⁱ, —SC(═NRⁱ)N(Rⁱ)₂, —C(═S)Rⁱ, —C(═S)ORⁱ, —C(═S)SRⁱ, —C(═S)N(Rⁱ)₂, —S(═O)Rⁱ, —SO₂Rⁱ, —NRⁱSO₂Rⁱ, —SO₂N(Rⁱ)₂, —CN, —SCN, or —NO₂;
each occurrence of Rⁱ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
r is 0, 1, 2, 3, 4, 5, 6, 7, or 8.

115-117. (canceled)

118. The compound of claim 1, wherein D¹ is

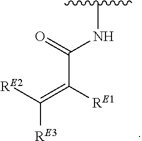

119-120. (canceled)

121. A compound of the formula: 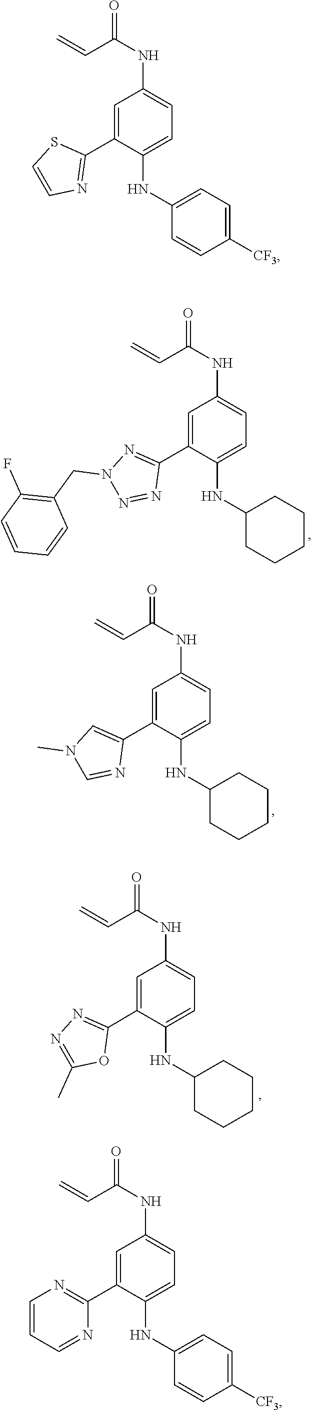 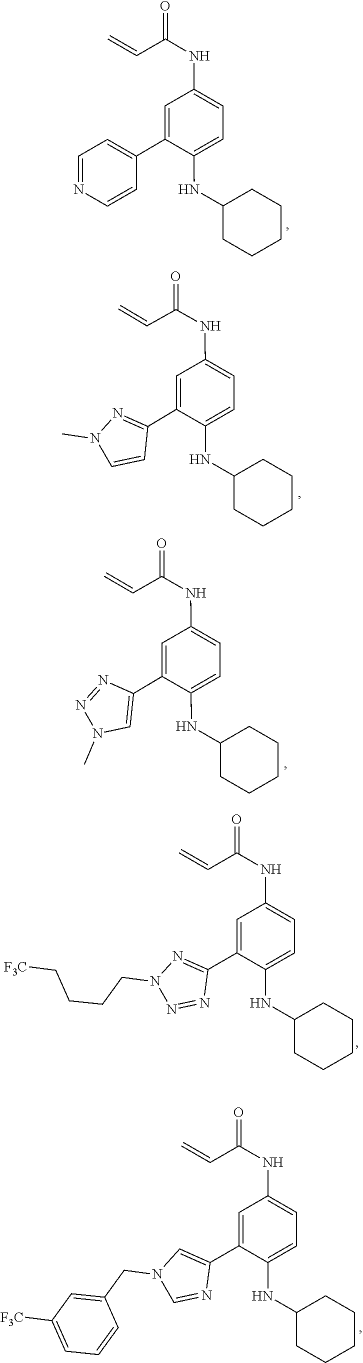 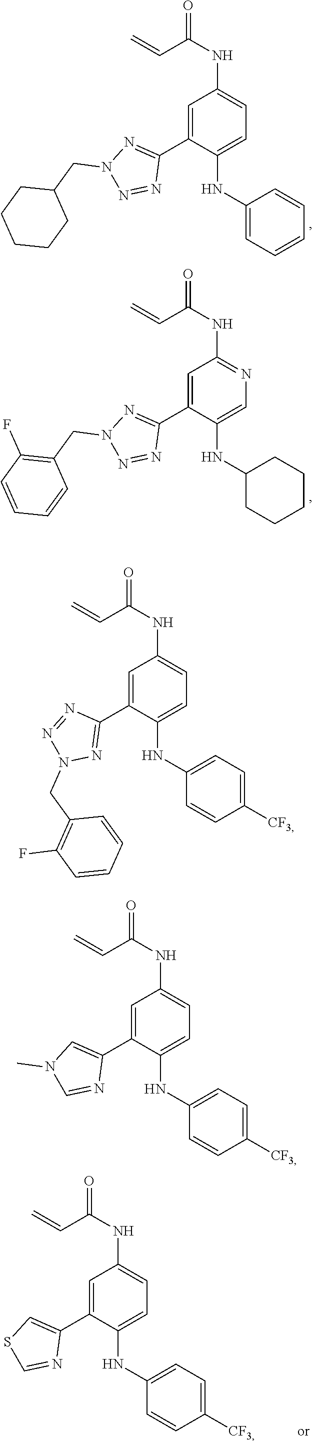 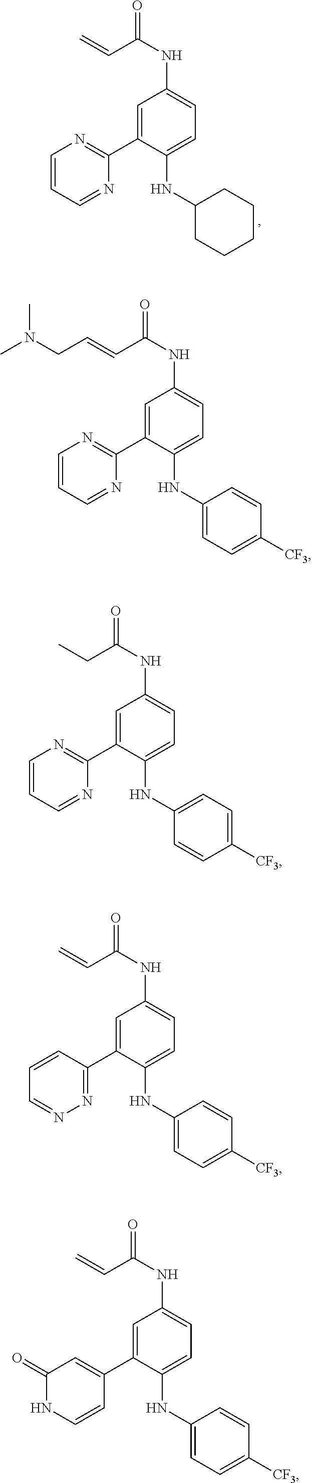 [SMILES: C=CC(=O)Nc1ccc(NC2CCCCC2)c(-c2ncccn2)c1 ; CN(C)C/C=C/C(=O)Nc1ccc(Nc2ccc(C(F)(F)F)cc2)c(-c2ncccn2)c1 ; CCC(=O)Nc1ccc(Nc2ccc(C(F)(F)F)cc2)c(-c2ncccn2)c1 ; C=CC(=O)Nc1ccc(Nc2ccc(C(F)(F)F)cc2)c(-c2cccnn2)c1 ; C=CC(=O)Nc1ccc(Nc2ccc(C(F)(F)F)cc2)c(-c2cc[nH]c(=O)c2)c1] 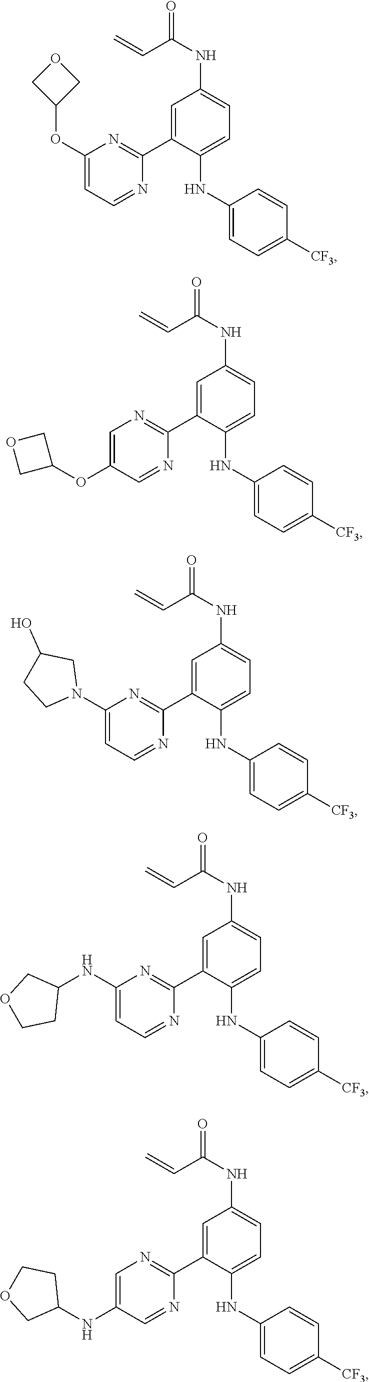 [SMILES: C=CC(=O)Nc1ccc(Nc2ccc(C(F)(F)F)cc2)c(-c2nccc(OC3COC3)n2)c1 ; C=CC(=O)Nc1ccc(Nc2ccc(C(F)(F)F)cc2)c(-c2ncc(OC3COC3)cn2)c1 ; C=CC(=O)Nc1ccc(Nc2ccc(C(F)(F)F)cc2)c(-c2nccc(N3CCC(O)C3)n2)c1 ; C=CC(=O)Nc1ccc(Nc2ccc(C(F)(F)F)cc2)c(-c2nccc(NC3CCOC3)n2)c1 ; C=CC(=O)Nc1ccc(Nc2ccc(C(F)(F)F)cc2)c(-c2ncc(NC3CCOC3)cn2)c1] 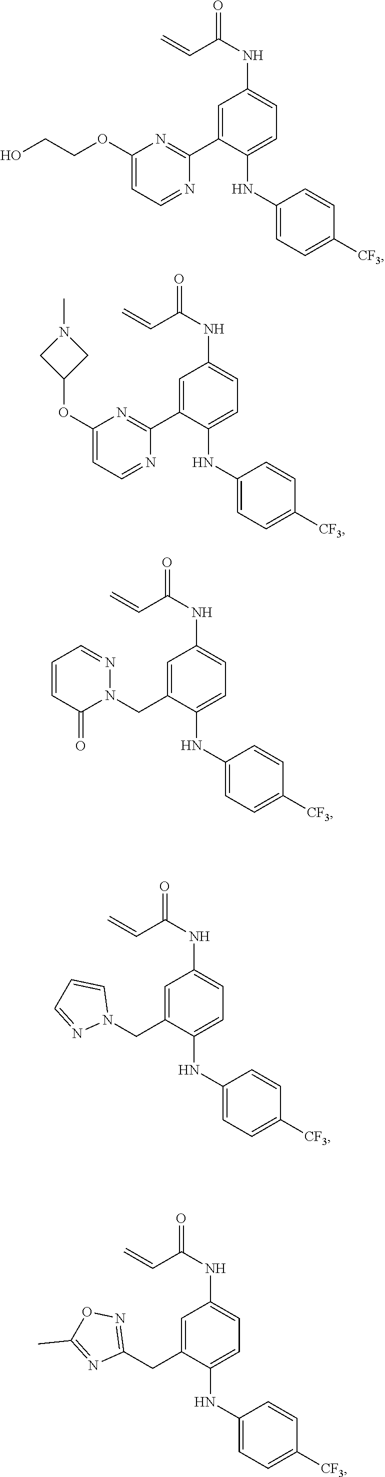 [SMILES: C=CC(=O)Nc1ccc(Nc2ccc(C(F)(F)F)cc2)c(-c2nccc(OCCO)n2)c1 ; C=CC(=O)Nc1ccc(Nc2ccc(C(F)(F)F)cc2)c(-c2nccc(OC3CN(C)C3)n2)c1 ; C=CC(=O)Nc1ccc(Nc2ccc(C(F)(F)F)cc2)c(Cn2ncccc2=O)c1 ; C=CC(=O)Nc1ccc(Nc2ccc(C(F)(F)F)cc2)c(Cn2cccn2)c1 ; C=CC(=O)Nc1ccc(Nc2ccc(C(F)(F)F)cc2)c(Cc2noc(C)n2)c1] 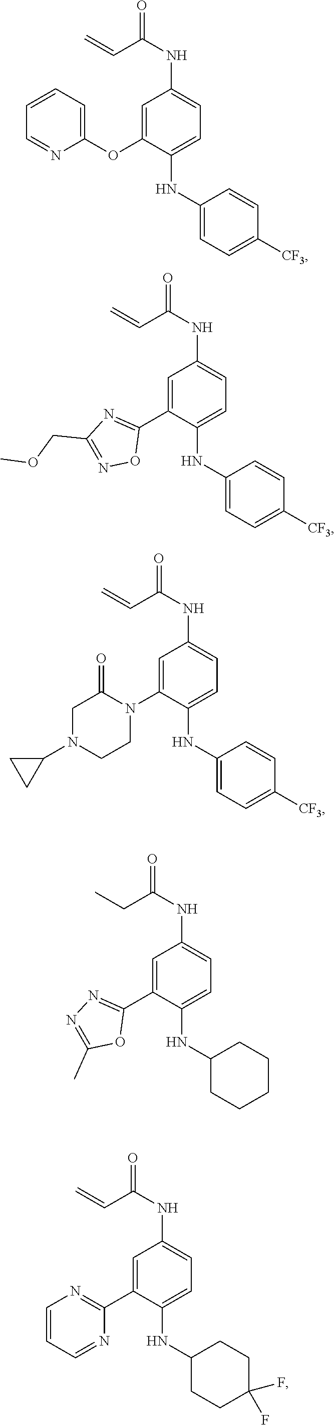 [SMILES: C=CC(=O)Nc1ccc(Nc2ccc(C(F)(F)F)cc2)c(Oc2ccccn2)c1 ; C=CC(=O)Nc1ccc(Nc2ccc(C(F)(F)F)cc2)c(-c2nc(COC)no2)c1 ; C=CC(=O)Nc1ccc(Nc2ccc(C(F)(F)F)cc2)c(N2CCN(C3CC3)CC2=O)c1 ; CCC(=O)Nc1ccc(NC2CCCCC2)c(-c2nnc(C)o2)c1 ; C=CC(=O)Nc1ccc(NC2CCC(F)(F)CC2)c(-c2ncccn2)c1] 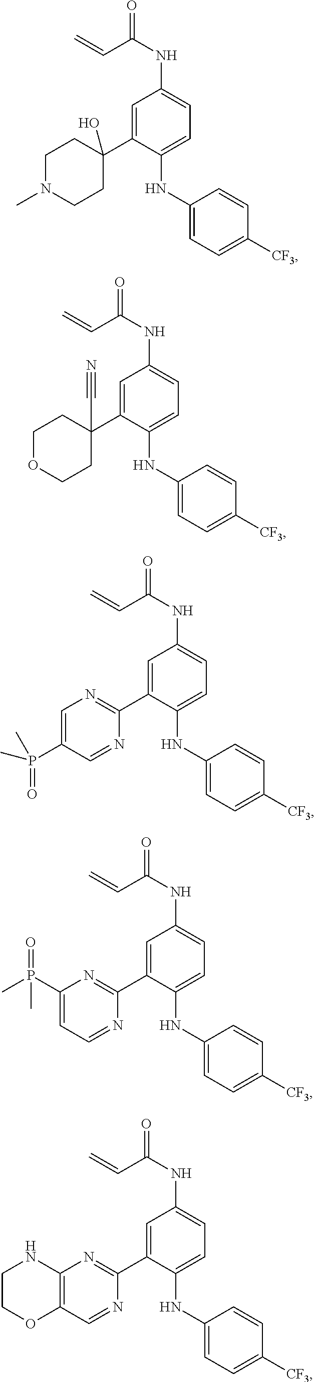 [SMILES: C=CC(=O)Nc1ccc(Nc2ccc(C(F)(F)F)cc2)c(C2(O)CCN(C)CC2)c1 ; C=CC(=O)Nc1ccc(Nc2ccc(C(F)(F)F)cc2)c(C2(C#N)CCOCC2)c1 ; C=CC(=O)Nc1ccc(Nc2ccc(C(F)(F)F)cc2)c(-c2ncc(P(C)(C)=O)cn2)c1 ; C=CC(=O)Nc1ccc(Nc2ccc(C(F)(F)F)cc2)c(-c2nccc(P(C)(C)=O)n2)c1 ; C=CC(=O)Nc1ccc(Nc2ccc(C(F)(F)F)cc2)c(-c2ncc3c(n2)NCCO3)c1] 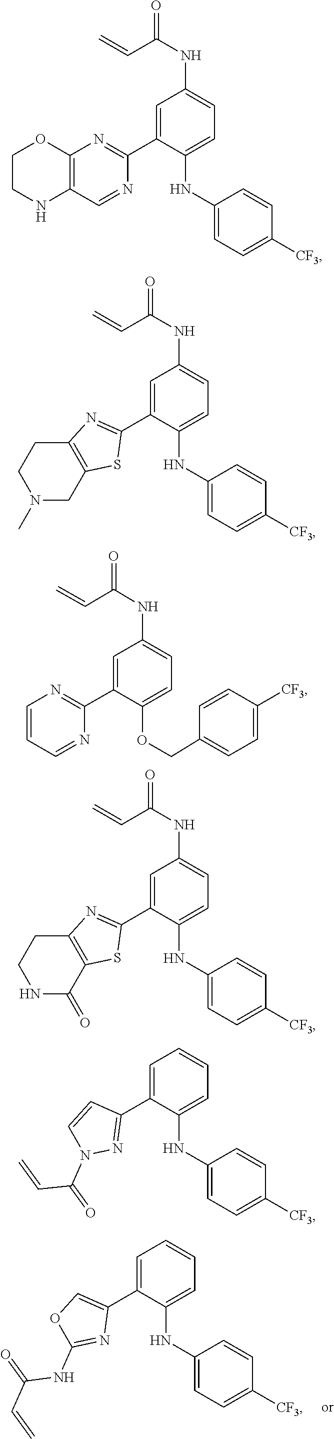 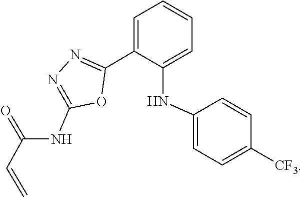 [SMILES: C=CC(=O)Nc1nnc(-c2ccccc2Nc2ccc(C(F)(F)F)cc2)o1]
or a pharmaceutically acceptable salt, solvate, hydrate, polymorph, co-crystal, tautomer, stereoisomer, isotopically labeled derivative, prodrug, or mixture thereof.

122-123. (canceled)

124. A pharmaceutical composition comprising a compound of claim 1, or a pharmaceutically acceptable salt, solvate, hydrate, polymorph, co-crystal, tautomer, stereoisomer, isotopically labeled derivative, prodrug, or mixture thereof, and optionally a pharmaceutically acceptable excipient.

125. (canceled)

126. A method of treating a proliferative disease in a subject in need thereof, the method comprising administering to the subject a therapeutically effective amount of a compound of claim 1, or a pharmaceutically acceptable salt, solvate, hydrate, polymorph, co-crystal, tautomer, stereoisomer, isotopically labeled derivative, prodrug, or mixture thereof, wherein the proliferative disease is cancer.

127. (canceled)

128. The method of claim 126, wherein the cancer is a sarcoma, lung cancer, breast cancer, liver cancer, prostate cancer, pancreatic cancer, colorectal cancer, ovarian cancer, skin cancer, esophageal cancer, or a carcinoma.

129-141. (canceled)

142. A method of inhibiting a transcription factor in a subject in need thereof, the method comprising:
administering to the subject a therapeutically effective amount of a compound of claim 1, or a pharmaceutically acceptable salt, solvate, hydrate, polymorph, co-crystal, tautomer, stereoisomer, isotopically labeled derivative, prodrug, or mixture thereof, wherein the transcription factor is TEAD1, TEAD2, TEAD3, or TEAD4.

143-152. (canceled)
